(19) United States
(12) Patent Application Publication
(10) Pub. No.: US 2006/0041962 A1
(43) Pub. Date: Feb. 23, 2006

(54) Genes and uses thereof to modulate secondary metabolite biosynthesis

(76) Inventors: Dirk G. Inze, Moorsel-Aalst (BE); Alain Goossens, Lokeren (BE); Kirsi-Marja Oksman-Caldentey, Helsinki (FI); Suvi T. Hakkinen, Espoo (FI); Into J. Laakso, Espoo (FI)

Correspondence Address: TRASK BRITT, P.O. BOX 2550, SALT LAKE CITY, UT, 84110, US

(21) Appl. No.: 10/991,285

(22) Filed: Nov. 16, 2004

Related U.S. Application Data
(63) Continuation of application No. PCT/EP03/50171, filed on May 16, 2003.

(30) Foreign Application Priority Data
May 17, 2002 (EP) 02076973.3
Jul. 4, 2002 (EP) 02077674.6

(51) Int. Cl. A01H 5/00; C07H 21/04; C12N 9/10; C12N 5/04; C12N 15/82

(52) U.S. Cl. 800/317.3; 435/419; 435/468; 536/23.2; 435/193

(57) ABSTRACT

The present invention relates to the use of a genome wide expression profiling technology in combination with the detection of the presence of secondary metabolites of interest to isolate genes that can be used to modulate the production of secondary metabolites in organisms and cell lines derived therefrom.

### CROSS-REFERENCE TO RELATED APPLICATION

This application is a continuation of PCT International Patent Application No. PCT/EP04/50171, filed on May 16, 2003, designating the United States of America, and published, in English, as PCT International Publication No. WO 03/097790 A2 on Nov. 27, 2003, the contents of the entirety of which is incorporated by this reference.

### SEQUENCE LISTING

Submitted with this application is a compact disc containing a SEQUENCE LISTING in a file entitled “V116.5T25 seq list” (524 KB, file created Nov. 17, 2004), the material contained in the compact disc being incorporated herein by this reference in its entirety. There are two identical compact discs submitted with this patent application (i.e., “Copy 1” and “Copy 2”), one being a copy of the other and each containing the single file “V116.5T25 seq list” (524 KB, file created Nov. 17, 2004).

### TECHNICAL FIELD

The present invention relates generally to biotechnology, and, more particularly, to the use of a genome wide expression profiling technology in combination with the detection of the presence of secondary metabolites of interest to isolate genes that can be used to modulate the production of secondary metabolites in organisms and cell lines derived thereof.

### BACKGROUND

Terrestrial micro-organisms, fungi, invertebrates and plants have historically been used as sources of natural products. However, apart from several well-studied groups or organisms, such as the actinomycetes, which have been developed for drug screening and commercial production, production problems still exist. For example, the antitumor agent taxol is a constituent of the bark of mature Pacific yew trees and its usage as a drug agent has caused concern about cutting too many of these trees and causing damage to the local ecological system. Taxol contains 11 chiral centers with 2048 possible diastereoisomeric forms so that its de novo synthesis on a commercial scale is unlikely. Furthermore, certain compounds appear in nature only when specific organisms interact with each other and the environment. Pathogens may alter plant gene expression and trigger synthesis of secondary metabolites such as phytoalexins that enable the plant to resist attack. Moreover, a lead compound discovered through random screening rarely becomes a drug because its bioavailability may not be adequate. Typically, a certain quantity of the lead compound is required so that it can be modified structurally to improve its initial activity. However, current methods for synthesis and development of lead compounds from natural sources, especially plants, are relatively inefficient. Other valuable phytochemicals are quite expensive because they are only produced at extremely low levels. These problems also delay clinical testing of new compounds and affect the economics of using these new sources of drug leads. The problems of obtaining useful metabolites from natural sources in high quantities may potentially be circumvented by cell cultures. For example the culture of plant cells has been explored since the 1960' as a viable alternative for the production of complex phytochemicals of industrial interest. However, despite promising features and developments, the production of plant-derived pharmaceuticals by plant cell cultures has not been fully commercially exploited. The main reasons for this reluctance are economical ones based on the slow growth and the low production levels of secondary metabolites by such plant cell cultures. However, little is known about how plants synthesize secondary metabolites and very little is known about how this synthesis is regulated. Certainly there is a need for a method to obtain higher levels of valuable secondary metabolite. The latter may include the identification of biosynthetic genes and regulatory genes involved in secondary metabolite biosynthetic pathways. Although genome sequencing of many organisms is now advancing at a frenetic pace, the metabolic pathways of most of the natural products are not understood. Traditional textbook representations of metabolic pathways neither capture the full number of potential network functions nor the network's resilience to disruption. Whereas algorithmic approaches to these latter problems have been proposed, many aspects of metabolic network function remain to be clearly delineated. Numerous studies have investigated the enzymes and regulatory factors controlling biosynthesis of specific secondary metabolites but little is known about the genetics controlling the quantitative and qualitative natural variation in secondary chemistry (QTL-approach, Kliebenstein et al. (2001) Genetics 159: 359, isolation of expressed sequence tags, Shelton et al. (2002) Plant Science 162, 9, Lange et al. (2000) Proc. Natl. Acad. Sci. 97, 2934, a proteomics approach, Decker et al. (2000) Electrophoresis 21, 3500).

### DISCLOSURE OF THE INVENTION

In the present invention, we provide a method that follows a genome wide approach and correlates gene expression with the production of secondary metabolites. Thus, through the combination of metabolic profiling and cDNA-AFLP based transcript profiling of elicited tobacco cells we have isolated genes that are involved in the production of alkaloids and phenylpropanoids. These genes can be used to modulate the production of secondary metabolites in plant cells.

### BRIEF DESCRIPTION OF THE FIGURES

FIG. 1: Semi-hypothetic scheme of the biosynthesis of nicotine alkaloids in Nicotiana tabacum leaves and BY-2 cells

FIG. 2: The growth curve of tobacco BY-2 cells, determined by packed cell volume (PVC)

FIG. 3: Molecular formulas of the tobacco alkaloids detected from BY-2 cells after elicitation with methyl jasmonate

FIG. 4: Nicotine and anabasine content [ug/g (d.w.)] after elicitation with 50 μM MeJA. Each sample was pooled together from three replicate shake flasks

FIG. 5: Anatabine and anatalline contents [ug/g (d.w.)] after elicitation with 50 μM MeJA. Each sample was pooled together from three replicate shake flasks

FIG. 6: Time-course of the accumulation of alkaloids in elicited BY-2 cells. Logarithmic scale

FIG. 7: The content of methyl putrecine in free pool of tobacco BY-2 cells.

FIG. 8: The content of polyamines (mean, SD, n=3) in free pool of tobacco BY-2 cells

FIG. 9: The content of soluble conjugated polyamines (mean, SD, n=3) in tobacco BY-2 cells

FIG. 10: The content of insoluble conjugated polyamines (mean, SD, n=3) in tobacco BY-2 cells

FIG. 11: Functional analysis. Nicotine content in elicitated (50 μM MeJA) BY-2 cells (N=3)

FIG. 12: Functional analysis. Anabasine content in elicitated (50 μM MeJA) BY-2 cells (N=3)

FIG. 13: Functional analysis. Anatabine content in elicitated (50 μM MeJA) BY-2 cells (N=3)

FIG. 14: Functional analysis. Anatalline (1 & 2) content in elicitated (50 μM MeJA) BY-2 cells (N=3)

### DETAILED DESCRIPTION OF THE INVENTION

There has always been interest in natural products for flavourings for food, perfumes, pigments for artwork and clothing, and tools to achieve spiritual enlightenment. Especially plant derived drugs are among the oldest drugs in medicine. For example alkaloids are originally described as structually diverse class of plant derived nitrogenous compounds, which often possess strong physiological activity. Plants synthesize alkaloids for various defence-related reactions, for example, actions against pathogens or herbivores. Over 15.000 alkaloids have been identified from plants. Alkaloids are classified into several biogenically related groups, but the enzymes and genes have been partly characterised only in groups of nicotine and tropane alkaloids, indole alkaloids and isoquinolidine alkaloids (Suzuki et al., 1999). Nicotine and tropane alkaloids share partly the same biosynthetic pathway. Many plants belonging to, for example, the Solanaceae family have been used for centuries because of their active substances: hyoscyamine and scopolamine. Also other Solanaceae plants belonging to the genera Atropa, Datura, Duboisia and Scopolia produce these valuable alkaloids. In medicine they find important applications in ophthalmology, anaesthesia, and in the treatment of cardiac and gastrointestinal diseases. Although a lot of information is available on the pharmacological effects of tropane alkaloids, surprisingly little is known about how plants synthesize these substances and almost nothing is known about how this synthesis is regulated. Nicotine is found in the genus Nicotiana and also other genera of Solanaceae and is also present in many other plants including lycopods and horsetails (Flores et al., 1991). Saitoh et al. (1985) performed an extensive study of the nicotine content in 52 of the 66 Nicotiana species and concluded that either nicotine or nornicotine is the predominant alkaloid in the leaves, depending on the species. However, in roots nicotine dominates in almost all species. In callus cultures, the nicotine content is mostly remarkably lower than in intact plants. The highest production has been found in the BY-2 cell line: 2.14% on dry weight basis which resembles the nicotine content in intact tobacco plants (Ohta et al., 1978). Although much is known of the alkaloid metabolite content in different organs of tobacco, surprisingly little is known about the biosynthesis, metabolism and regulation of various nicotine alkaloids in tobacco callus and cell cultures.

Many approaches have been developed to overcome the common problem of low product yield of alkaloid-producing plant cell cultures. One approach is the addition of elicitors. Elicitors are compounds capable of inducing defence responses in plants (Darvil and Albersheim, 1984). Other approaches to increase the product yield of secondary metabolites comprise the screening and selection of high-producing cell lines, the optimisation of the growth and product parameters and the use of metabolic engineering (Verpoorte et al., 2000). However, metabolic engineering implies detailed knowledge of the biosynthetic steps of the secondary metabolite(s) of interest. Progress in the elucidation of the biosynthetic pathways of plant secondary products has long been hampered by lack of good model systems. In the past two decades plant cell cultures have proven to be invaluable tools in the investigation of plant secondary metabolite biosynthetic pathways. The tobacco BY-2 (Nicotiana tabacum var. “Bright Yellow”) cell line is a very fast growing and highly synchronisable cell system and thus desirable for investigation of various aspects of plant cell biology and metabolism (Nagata and Kumagai, 1999). In the present invention the formation of various nicotine related alkaloids in tobacco BY-2 cells was taken as an example for the isolation of genes involved in the biosynthesis of alkaloids, phenylpropanoids and other secondary metabolites. We have used a genome wide approach and isolated genes which expression correlated with the occurrence of alkaloids and/or phenylpropanoids.

In one embodiment, the invention provides an isolated polypeptide modulating the production of at least one secondary metabolite in an organism or cell derived thereof selected from the group consisting of (a) polypeptide encoded by a polynucleotide comprising SEQ ID NO: 1, 2, 3, through 609, 610, 611 or SEQ ID NO: 612, 613, 614, through 869, 870, 871 of the accompanying and incorporated herein by reference SEQUENCE LISTING, (b) a polypeptide comprising a polypeptide sequence having a least 60% identity to at least one of the polypeptides encoded by a polynucleotide sequence having SEQ ID NO: 612, 613, 614 through 869, 870, 871, (c) a polypeptide comprising a polypeptide sequence having a least 90% identity to at least one of the polypeptides encoded by a polynucleotide sequence having SEQ ID NO: 1, 2, 3 through 609, 610, 611 and (d) fragments and variants of the polypeptides according to (a), (b) or (c) modulating the production of at least one secondary metabolite in an organism or cell derived thereof.

In another embodiment, the invention provides an isolated polypeptide according to wherein the polypeptide sequence is depicted in SEQ ID NO: 872, 873, 874 through 894 or 895 and polypeptide sequences having at least 90% identity to SEQ ID NO: 872, 873, 874 through 894 or 895.

In another embodiment, the invention provides an isolated polynucleotide selected from the groups consisting of (a) polynucleotide comprising a polynucleotide sequence having at least one of the sequences SEQ ID NO: 1, 2, 3 through 609, 610, 611 or SEQ ID NO: 612, 613, 614 through 869, 870, 871; (b) a polynucleotide comprising a polynucleotide sequence having at least 60% identity to at least one of the sequences having SEQ ID NO: 612, 613, 614, . . . , 869, 870, 871; (c) a polynucleotide comprising a polynucleotide sequence having at least 90% identity to at least one of the sequences having SEQ ID NO: 1, 2, 3 through 609, 610, 611; (d) fragments and variants of the polynucleotides according to (a), (b) or (c) modulating the production of at least one secondary metabolite in an organism or cell derived thereof.

Accordingly, the present invention provides 611 polynucleotide sequences (SEQ ID NO: 1, 2, 3 through 609, 610, 611) derived from tobacco BY2-cells for which a homologue exists in other species and 260 polynucleotide sequences (SEQ ID NO: 612, 613, 614 through 869, 870, 871) derived from tobacco BY2-cells for which no homologue exists in other species. As used herein, the word “polynucleotide” may be interpreted to mean the DNA and cDNA sequence as detailed by Yoshikai et al. (1990) Gene 87:257, with or without a promoter DNA sequence as described by Salbaum et al. (1988) EMBO J. 7(9):2807.

As used herein, “fragment” refers to a polypeptide or polynucleotide of at least about 9 amino acids or 27 base pairs, typically 50 to 75, or more amino acids or base pairs, wherein the polypeptide contains an amino acid core sequence. If desired, the fragment may be fused at either terminus to additional amino acids or base pairs, which may number from 1 to 20, typically 50 to 100, but up to 250 to 500 or more. A “functional fragment” means a polypeptide fragment possessing the biological property able to modulate the production of at least one secondary metabolite in an organism or cell derived thereof. In a particular embodiment the functional fragment is able to modulate the production of at least one secondary metabolite in a plant or plant cell derived thereof. The term ‘production’ includes intracellular production and secretion into the medium. The term ‘modulates or modulation’ refers to an increase or a decrease. Often an increase of at least one secondary metabolite is desired but sometimes a decrease of at least one secondary metabolite is wanted. The decrease can for example refer to the decrease of an undesired intermediate product of at least one secondary metabolite. With an increase in the production of one or more metabolites it is understood that the production may be enhanced by at least 20%, 30%, 40%, 50%, 60%, 70%, 80%, 90% or at least 100% relative to the untransformed plant or plant cell which was used to transform with an expression vector comprising an expression cassette further comprising at least one polynucleotide or homologue or variant or fragment thereof of the invention. Conversely, a decrease in the production of the level of one or more secondary metabolites may be decreased by at least 20%, 30%, 40%, 50%, 60%, 70%, 80%, 90% or at least 100% relative to the untransformed plant or plant cell which was used to transform with an expression vector comprising an expression cassette further comprising at least one polynucleotide or homologue or variant or fragment thereof of the invention. The terms ‘identical’ or percent ‘identity’ in the context of two or more nucleic acids or polypeptide sequences, refer to two or more sequences or subsequences that are the same or have a specified percentage of amino acid residues or nucleotides that are the same (i.e. 70% identity over a specified region), when compared and aligned for maximum correspondence over a comparison window, or designated region as measured using sequence comparison algorithms or by manual alignment and visual inspection. Preferably, the identity exists over a region that is at least about 25 amino acids or nucleotides in length, or more preferably over a region that is 50-100 amino acids or nucleotides or even more in length. Examples of useful algorithms are PILEUP (Higgins & Sharp, CABIOS 5:151 (1989), BLAST and BLAST 2.0 (Altschul et al., J. Mol. Biol. 215: 403 (1990). Software for performing BLAST analyses is publicly available through the National Center for Biotechnology Information www.ncbi.nlm.nih.gov. In the present invention the term ‘homologue’ also refers to ‘identity’. For example a homologue of SEQ ID NO: 1, 2, 3 through 609, 610 or 611 has at least 90% identity to one of these sequences. A homologue of SEQ ID NO: 612, 613, 614 through 869, 870 or 871 has at least 60% identity to one of these sequences.

According to still further features in the described preferred embodiments, the polynucleotide fragment encodes a polypeptide able to modulate the secondary metabolite biosynthesis, which may therefore be allelic, species and/or induced variant of the amino acid sequence set forth in SEQ ID NO: 1-871. It is understood that any such variant may also be considered a homologue.

The present invention accordingly provides, in one embodiment, a method for modulating the production of at least one secondary metabolite in biological cells or organisms, such as plants, by transformation of the biological cells with an expression vector comprising an expression cassette that further comprises at least one gene comprising a fragment, variant or homologue encoded by at least one sequence selected from SEQ ID NO: 1-871. With “at least one secondary metabolite” it is meant one particular secondary metabolite such as for example nicotine or several alkaloids related with nicotine or several unrelated secondary metabolites. Biological cells can be plant cells, fungal cells, bacteria cells, algae cells and/or animal cells. In a particular preferred embodiment the biological cells are plant cells. Generally, two basic types of metabolites are synthesised in cells, i.e. those referred to as primary metabolites and those referred to as secondary metabolites. A primary metabolite is any intermediate in, or product of the primary metabolism in cells. The primary metabolism in cells is the sum of metabolic activities that are common to most, if not all, living cells and are necessary for basal growth and maintenance of the cells. Primary metabolism thus includes pathways for generally modifying and synthesising certain carbohydrates, amino acids, fats and nucleic acids, with the compounds involved in the pathways being designated primary metabolites. In contrast hereto, secondary metabolites usually do not appear to participate directly in growth and development. They are a group of chemically very diverse products that often have a restricted taxonomic distribution. Secondary metabolites normally exist as members of closely related chemical families, usually of a molecular weight of less than 1500 Dalton, although some bacterial toxins are considerably longer. Secondary plant metabolites include e.g., alkaloid compounds (e.g., terpenoid indole alkaloids, tropane alkaloids, steroid alkaloids), phenolic compounds (e.g., quinines, lignans and flavonoids), terpenoid compounds (e.g., monoterpenoids, iridoids, sesquiterpenoids, diterpenoids and triterpenoids). In addition, secondary metabolites include small molecules, such as substituted heterocyclic compounds which may be monocyclic or polycyclic, fused or bridged. Many plant secondary metabolites have value as pharmaceuticals. Examples of plant pharmaceuticals include, for example, taxol, digoxin, scopolamine, diosgenin, codeine, morphine, quinine, shikonin, ajmalicine and vinblastine.

In another embodiment, the invention provides a recombinant DNA vector comprising at least one polynucleotide sequence, homologue, fragment or variant selected from at least one of the sequences comprising SEQ ID NO: 1-871. The vector may be of any suitable type including, but not limited to, a phage, virus, plasmid, phagemid, cosmid, bacmid or even an artificial chromosome. The at least one polynucleotide sequence preferably codes for at least one polypeptide that is involved in the biosynthesis and/or regulation of synthesis of at least one secondary metabolite (e.g., a transcription factor, a repressor, an enzyme that regulates a feed-back loop, a transporter, a chaperone). The term “recombinant DNA vector” as used herein refers to DNA sequences containing a desired coding sequence and appropriate DNA sequences necessary for the expression of the operably linked coding polynucleotide sequence in a particular host organism (e.g., plant cell). Plant cells are known to utilize promoters, polyadenlyation signals and enhancers.

In yet another embodiment, the invention provides a transgenic plant or derived cell thereof transformed with the recombinant DNA vector.

A recombinant DNA vector comprises at least one “Expression cassette”. Expression cassettes are generally DNA constructs preferably including (5′ to 3′ in the direction of transcription): a promoter region, a polynucleotide sequence, homologue, variant or fragment thereof of the present invention operatively linked with the transcription initiation region, and a termination sequence including a stop signal for RNA polymerase and a polyadenylation signal. It is understood that all of these regions should be capable of operating in biological cells, such as plant cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The polynucleotide sequence, homologue, variant or fragment thereof of the invention may be expressed in for example a plant cell under the control of a promoter that directs constitutive expression or regulated expression. Regulated expression comprises temporally or spatially regulated expression and any other form of inducible or repressible expression. Temporally means that the expression is induced at a certain time point, for instance, when a certain growth rate of the plant cell culture is obtained (e.g., the promoter is induced only in the stationary phase or at a certain stage of development). “Spatially” means that the promoter is only active in specific organs, tissues, or cells (e.g., only in roots, leaves, epidermis, guard cells or the like). Other examples of regulated expression comprise promoters whose activity is induced or repressed by adding chemical or physical stimuli to the plant cell. In a preferred embodiment the expression is under control of environmental, hormonal, chemical, and/or developmental signals. Such promoters for plant cells include promoters that are regulated by (1) heat, (2) light, (3) hormones, such as abscisic acid and methyl jasmonate (4) wounding or (5) chemicals such as salicylic acid, chitosans or metals. Indeed, it is well known that the expression of secondary metabolites can be boosted by the addition of for example specific chemicals, jasmonate and elicitors. In a particular embodiment the co-expression of several (more than one) polynucleotide sequence or homologue or variant or fragment thereof, in combination with the induction of secondary metabolite synthesis is beneficial for an optimal and enhanced production of secondary metabolites. Alternatively, the at least one polynucleotide sequence, homologue, variant or fragment thereof is placed under the control of a constitutive promoter. A constitutive promoter directs expression in a wide range of cells under a wide range of conditions. Examples of constitutive plant promoters useful for expressing heterologous polypeptides in plant cells include, but are not limited to, the cauliflower mosaic virus (CaMV) 35S promoter, which confers constitutive, high-level expression in most plant tissues including monocots; the nopaline synthase promoter and the octopine synthase promoter. The expression cassette is usually provided in a DNA or RNA construct which is typically called an “expression vector” which is any genetic element, for example, a plasmid, a chromosome, a virus, behaving either as an autonomous unit of polynucleotide replication within a cell (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages, cosmids, plant viruses and artificial chromosomes. The expression cassette may be provided in a DNA construct which also has at least one replication system. In addition to the replication system, there will frequently be at least one marker present, which may be useful in one or more hosts, or different markers for individual hosts. The markers may a) code for protection against a biocide, such as antibiotics, toxins, heavy metals, certain sugars or the like; b) provide complementation, by imparting prototrophy to an auxotrophic host: or c) provide a visible phenotype through the production of a novel compound in the plant. Exemplary genes which may be employed include neomycin phosphotransferase (NPTII), hygromycin phosphotransferase (HPT), chloramphenicol acetyltransferase (CAT), nitrilase, and the gentamicin resistance gene. For plant host selection, non-limiting examples of suitable markers are β-glucuronidase, providing indigo production, luciferase, providing visible light production, Green Fluorescent Protein and variants thereof, NPTII, providing kanamycin resistance or G418 resistance, HPT, providing hygromycin resistance, and the mutated aroA gene, providing glyphosate resistance.

The term “promoter activity” refers to the extent of transcription of a polynucleotide sequence, homologue, variant or fragment thereof that is operably linked to the promoter whose promoter activity is being measured. The promoter activity may be measured directly by measuring the amount of RNA transcript produced, for example by Northern blot or indirectly by measuring the product coded for by the RNA transcript, such as when a reporter gene is linked to the promoter. The term “operably linked” refers to linkage of a DNA segment to another DNA segment in such a way as to allow the segments to function in their intended manners. A DNA sequence encoding a gene product is operably linked to a regulatory sequence when it is ligated to the regulatory sequence, such as, for example a promoter, in a manner which allows modulation of transcription of the DNA sequence, directly or indirectly. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

In a particular embodiment the polynucleotides or homologues or variants or fragments thereof of the present invention can be introduced in plants or plant cells that are different from tobacco and the polynucleotides can be used for the modulation of secondary metabolite synthesis in plants or plant cells different from tobacco.

The term “heterologous DNA” and or “heterologous RNA” refers to DNA or RNA that does not occur naturally as part of the genome or DNA or RNA sequence in which it is present, or that is found in a cell or location in the genome or DNA or RNA sequence that differs from that which is found in nature. Heterologous DNA and RNA (in contrast to homologous DNA and RNA) are not endogenous to the cell into which it is introduced, but has been obtained from another cell or synthetically or recombinantly produced. An example is a gene isolated from one plant species operably linked to a promoter isolated from another plant species. Generally, though not necessarily, such DNA encodes RNA and proteins that are not normally produced by the cell in which the DNA is transcribed or expressed. Similarly exogenous RNA encodes for proteins not normally expressed in the cell in which the exogenous RNA is present. Heterologous DNA or RNA may also refer to as foreign DNA or RNA. Any DNA or RNA that one of skill in the art would recognize as heterologous or foreign to the cell in which it is expressed is herein encompassed by the term heterologous DNA or heterologous RNA. Examples of heterologous DNA include, but are not limited to, DNA that encodes proteins, polypeptides, receptors, reporter genes, transcriptional and translational regulatory sequences, selectable or traceable marker proteins, such as a protein that confers drug resistance, RNA including mRNA and antisense RNA and ribozymes.

In yet another embodiment, the invention provides for a method to identify genes which expression modulates the production of at least one secondary metabolite in an organism or cells derived thereof comprising the steps of (a) performing a genome wide expression profiling of the organism or cells on different times of growth, (b) isolating genes which expression is co-regulated either with the at least one secondary metabolite, or with a gene known to be involved in the biosynthesis of the secondary metabolite, (c) analysing the effect of over- or under-expression of the genes in the organism or cell on the production of the at least one secondary metabolite and (d) identifying genes that can modulate the production of the at least one secondary metabolite.

The wording “performing a genome wide expression profiling” means that the expression of genes and/or proteins is measured. Preferably, the expression is measured on different times of growth, on different treatments and the like. Usually a comparison of the expression is made between two or more samples (e.g., samples that are treated and non-treated, induced or non-induced). Gene expression can be measured by various methods known in the art comprising macro-array technology, micro-array technology, serial analysis of gene expression (SAGE), cDNA AFLP and the like. With array technology complete genes or parts thereof, EST sequences, cDNA sequences, oligonucleotides are attached to a carrier. Protein expression can be measured through various protein isolation, protein profiling and protein identification methods known in the art. The analysis of the effect of over- or under-expression of genes in for example plants or plant cells can be carried out by various well-known methods in the art.

In a further embodiment, the invention provides a method where the performance of the genome wide expression profiling is preceded by the step of inducing the production of the at least one secondary metabolite in the organism or cell derived thereof. The wording ‘inducing the production’ means that for example the cell culture, such as a plant cell culture, is stimulated by the addition of an external factor. External factors include the application of heat, the application of cold, the addition of acids, bases, metal ions, fungal membrane proteins, sugars and the like. One approach that has been given interesting results for better production of plant secondary metabolites is elicitation. Elicitors are compounds capable of inducing defence responses in plants (Darvil and Albersheim, 1984). These are usually not found in intact plants but their biosynthesis is induced after wounding or stress conditions. Commonly used elicitors are jasmonates, mainly jasmonic acid and its methyl ester, methyl jasmonate. Jasmonates are linoleic acid derivatives of the plasma membrane and display a wide distribution in the plant kingdom (for overview see Reinbothe et al., 1994). They were originally classified as growth inhibitors or promoters of senescence but now it has become apparent that they have pleiotropic effects on plant growth and development. Jasmonates appear to regulate cell division, cell elongation and cell expansion and thereby stimulate organ or tissue formation (Swiatek et al., 2002). They are also involved in the signal transduction cascades that are activated by stress situations such as wounding, osmotic stress, desiccation and pathogen attack (Creelman et al., 1992; Gundlach et al., 1992; Ishikawa et al., 1994). Methyl jasmonate (MeJA) is known to induce the accumulation of numerous defence-related secondary metabolites (e.g., phenolics, alkaloids and sesquiterpenes) through the induction of genes coding for the enzymes involved in the biosynthesis of these compounds in plants (Gundlach, et al., 1992; Imanishi et al., 1998; Mandujano-Chávez et al., 2000). Jasmonates can modulate gene expression from the (post)transcriptional to the (post)translational level, both in a positive as in a negative way. Genes that are upregulated are e.g., defence and stress related genes (PR proteins and enzymes involved with the synthesis of phytoalexins and other secondary metabolites) whereas the activity of housekeeping proteins and genes involved with photosynthetic carbon assimilation are down-regulated (Reinbothe et al., 1994). For example: the biosynthesis of phytoalexins and other secondary products in plants can also be boosted up by signal molecules derived from micro-organisms or plants (such as peptides, oligosaccharides, glycopeptides, salicylic acid and lipophilic substances) as well as by various abiotic elicitors like UV-light, heavy metals (Cu, VOSO4, Cd) and ethylene. The effect of any elicitor is dependent on a number of factors, such as the specificity of an elicitor, elicitor concentration, the duration of the treatment and growth stage of the culture.

Generally, secondary metabolites can be measured, intracellularly or in the extracellular space, by methods known in the art. Such methods comprise analysis by thin-layer chromatography, high pressure liquid chromatography, capillaryelectrophoresis, gas chromatography combined with mass spectrometric detection, radioimmuno-assay (RIA) and enzyme immuno-assay (ELISA).

In yet another embodiment, the method to identify genes which expression modulates the production of at least one secondary metabolite in an organism or cells derived thereof is used to identify genes that are involved in the alkaloid biosynthesis.

The definition of “Alkaloids”, of which more than 12,000 structures have been described already, includes all nitrogen-containing natural products which are not otherwise classified as peptides, non-protein amino acids, amines, cyanogenic glycosides, glucosinolates, cofactors, phytohormones or primary metabolites (such as purine and pyrimidine bases). The “calystegins” constitute a unique subgroup of the tropane alkaloid class (Goldmann et al. (1990) Phytochemistry, 29, 2125). They are characterized by the absence of an N-methyl substituent and a high degree of hydroxylation. Trihydroxylated calystegins are summarized as the calystegin A-group, tetrahydroxylated calystegins as the B-group, and pentahydroxylated derivates form the C-group. Calystegins represent a novel structural class of tropane alkaloids possessing potent glycosidase inhibitory properties next to longer known classes of the monocyclic pyrrolidones (e.g., dihydroxymethyldihydroxy pyrrolidine) pyrrolines and piperidines (e.g., deoxynojirimycin), and the bicyclic pyrrolizidines (e.g., australine) and indolizidines (e.g., swainsonine and castanospermine). Glycosidase inhibitors are potentially useful as antidiabetic, antiviral, antimetastatic, and immunomodulatory agents.

In another embodiment, the method to identify genes which expression modulates the production of at least one secondary metabolite in an organism or cells derived thereof is used to identify genes that are involved in the phenylpropanoid biosynthesis. “Phenylpropanoids” or “phenylpropanes” are aromatic compounds with a propyl side-chain attached to the aromatic ring, which can be derived directly from phenylalanine. The ring often carries oxygenated substituents (hydroxyl, methoxyl and methylenedioxy groups) in the para-position. Natural products in which the side-chain has been shortened or removed can also be derived from typical phenylpropanes. Most plant phenolics are derived from the phenylpropanoid and phenylpropanoid-acetate pathways and fulfil a very broad range of physiological roles in plants. For example polymeric lignins reinforce specialized cell wall. Closely related are the lignans which vary from dimers to higher oligomers. Lignans can either help defend against various pathogens or act as antioxidants in flowers, leaves and roots. The flavonoids comprise an astonishingly diverse group of more than 4500 known compounds. Among their subclasses are the anthocyanins (pigments), proanthocyanidins or condensed tannins (feeding deterrents and wood protectants), and isoflavonoids (defensive products and signalling molecules). The coumarins, furanocoumarins, and stilbenes protect against bacterial and fungal pathogens, discourage herbivory, and inhibit seed germination.

In yet another embodiment, the isolated polynucleotides of the invention, or homologues, or variants, or fragments thereof are used to modulate the biosynthesis of secondary metabolites in an organism or cell derived thereof. In a particular embodiment the isolated polynucleotides, homologues, variants or fragments thereof are used to modulate the biosynthesis of secondary metabolites in plants or plant cells derived thereof.

In yet another embodiment, the polynucleotides comprising SEQ ID NO: 10, 11, 19, 20, 35, 40, 41, 47, 65, 67, 70, 88, 89, 97, 98, 101, 102, 103, 106, 107, 108, 117, 118, 120, 121, 123, 124, 126, 128, 130, 131, 132, 136, 137, 142, 143, 144, 145, 146, 147, 148, 152, 154, 155, 159, 160, 161, 162, 163, 175, 176, 177, 181, 182, 183, 189, 197, 202, 207, 208, 209, 210, 217, 219, 220, 221, 233, 235, 236, 237, 239, 240, 241, 242, 243, 244, 261, 262, 264, 265, 268, 70, 272, 273, 274, 278, 279, 299, 300, 302, 303, 304, 305, 306, 316, 317, 318, 320, 321, 326, 329, 331, 332, 333, 334, 341, 344, 348, 349, 350, 351, 354, 355, 356, 358, 372, 373, 374, 375, 377, 382, 390, 391, 392, 395, 403, 405, 406, 414, 417, 418, 419, 420, 424, 430, 434, 439, 440, 441, 445, 446, 456, 463, 478, 485, 491, 497, 507, 508, 510, 518, 519, 527, 529, 531, 532, 534, 567, 569, 570, 575, 577, 579, 587, 593, 594, 598, 599, 601, 603, 608, 612, 613, 618, 619, 620, 628, 636, 642, 643, 647, 648, 649, 652, 653, 654, 655, 656, 657, 659, 660, 662, 664, 670, 671, 674, 675, 676, 677, 679, 680, 682, 683, 695, 696, 700, 701, 703, 707, 709, 710, 711, 712, 714, 719, 724, 727, 729, 732, 734, 735, 740, 741, 744, 746, 748, 749, 750, 751, 753, 754, 755, 757, 758, 759, 760, 761, 762, 763, 764, 766, 767, 772, 777, 784, 794, 809, 810, 811, 816, 817, 822, 823, 826, 827, 828, 829, 830, 832, 833, 834, 836, 837, 839, 840, 841, 850, 854, 855, 856, 858, 859, 861, 864, 865, 488, 489 and/or 490 or fragments or homologues thereof can be used to modulate the biosynthesis of alkaloids in an organism or cell derived thereof. In a particular embodiment the polynucleotides or fragments or homologues thereof can be used to modulate the biosynthesis of alkaloids in plants or plant cells derived thereof. The expression of the latter collection of SEQ ID Numbers correlates with the production of alkaloids in plants.

In yet another embodiment, the polynucleotides comprising SEQ ID NO: 3, 4, 5, 7, 15, 17, 21, 23, 29, 30, 32, 33, 39, 42, 44, 45, 46, 48, 49, 50, 51, 8, 61, 62, 72, 74, 79, 84, 92, 94, 95, 104, 105, 125, 134, 150, 170, 171, 179, 180, 184, 194, 195, 200, 201, 203, 204, 205, 213, 214, 215, 218, 245, 249, 250, 251, 252, 254, 255, 266, 275, 276, 281, 282, 285, 286, 287, 289, 291, 298, 301, 308, 309, 310, 311, 312, 313, 315, 319, 323, 324, 335, 343, 361, 363, 364, 370, 379, 380, 383, 384, 385, 386, 398, 401, 402, 407, 415, 416, 423, 432, 433, 437, 443, 444, 447, 448, 450, 451, 452, 455, 457, 460, 461, 462, 471, 474, 486, 487, 493, 494, 499, 500, 501, 502, 503, 504, 505, 506, 517, 522, 523, 524, 526, 528, 538, 541, 543, 544, 545, 546, 547, 553, 554, 555, 562, 568, 571, 572, 578, 580, 581, 582, 588, 605, 607, 616, 617, 621, 626, 627, 637, 638, 641, 644, 650, 651, 665, 666, 667, 681, 684, 685, 691, 697, 698, 704, 708, 713, 720, 721, 728, 730, 736, 745, 752, 756, 771, 776, 778, 782, 783, 792, 793, 795, 797, 798, 799, 800, 801, 808, 815, 818, 819, 820, 821, 835, 842, 843, 844, 845, 848, 851, 852, 853, 862, 868, 488, 489 and/or 490 or fragments or homologues thereof can be used to modulate the biosynthesis of phenylpropanoids in an organism or cell derived thereof. In a particular embodiment the polynucleotides or homologues or fragments derived thereof can be used to modulate the biosynthesis of phenylpropanoids in plants or plant cells derived thereof. The expression of the latter collection of SEQ ID Numbers correlates with the production of phenylpropanoids in plants.

The present invention can be practiced with any plant variety for which cells of the plant can be transformed with an expression cassette of the current invention and for which transformed cells can be cultured in vitro. Suspension culture, callus culture, hairy root culture, shoot culture or other conventional plant cell culture methods may be used (as described in: Drugs of Natural Origin, G. Samuelsson, 1999, ISBN 9186274813).

By “plant cells” it is understood any cell which is derived from a plant and can be subsequently propagated as callus, plant cells in suspension, organized tissue and organs (e.g., hairy roots). In the present invention the word “plant cell” also comprises cells derived from lower plants such as from the Pteridophytae and the Bryophytae.

Tissue cultures derived from the plant tissue of interest can be established. Methods for establishing and maintaining plant tissue cultures are well known in the art (see, for example, Trigiano R. N. and Gray D. J. (1999), “Plant Tissue Culture Concepts and Laboratory Exercises”, ISBN: 0-8493-2029-1; Herman E. B. (2000), “Regeneration and Micropropagation: Techniques, Systems and Media 1997-1999”, Agricell Report). Typically, the plant material is surface-sterilized prior to introducing it to the culture medium. Any conventional sterilization technique, such as chlorinated bleach treatment can be used. In addition, antimicrobial agents may be included in the growth medium. Under appropriate conditions plant tissue cells form callus tissue, which may be grown either as solid tissue on solidified medium or as a cell suspension in a liquid medium.

A number of suitable culture media for callus induction and subsequent growth on aqueous or solidified media are known. Exemplary media include standard growth media, many of which are commercially available (e.g., Sigma Chemical Co., St. Louis, Mo.). Examples include Schenk-Hildebrandt (SH) medium, Linsmaier-Skoog (LS) medium, Murashige and Skoog (MS) medium, Gamborg's B5 medium, Nitsch & Nitsch medium, White's medium, and other variations and supplements well known to those of skill in the art (see, for example, Plant Cell Culture, Dixon, ed. IRL Press, Ltd. Oxford (1985) and George et al., Plant Culture Media, Vol 1, Formulations and Uses Exegetics Ltd. Wilts, UK, (1987)). For the growth of conifer cells, particularly suitable media include 1/2 MS, 1/2 L. P., DCR, Woody Plant Medium (WPM), Gamborg's B5 and its modifications, DV (Durzan and Ventimiglia, In Vitro Cell Dev. Biol. 30:219-227 (1994)), SH, and White's medium.

In a particular embodiment, the current invention can be combined with other known methods to enhance the production and/or the secretion of secondary metabolites in plant cell cultures such as (1) by improvement of the plant cell culture conditions, (2) by the transformation of the plant cells with a transcription factor capable of upregulating genes involved in the pathway of secondary metabolite formation, (3) by the addition of specific elicitors to the plant cell culture, and 4) by the induction of organogenesis.

The term “plant” as used herein refers to vascular plants (e.g., gymnosperms and angiosperms). The method comprises transforming a plant cell with an expression cassette of the present invention and regenerating such plant cell into a transgenic plant. Such plants can be propagated vegetatively or reproductively. The transforming step may be carried out by any suitable means, including by Agrobacterium-mediated transformation and non-Agrobacterium-mediated transformation, as discussed in detail below. Plants can be regenerated from the transformed cell (or cells) by techniques known to those skilled in the art. Where chimeric plants are produced by the process, plants in which all cells are transformed may be regenerated from chimeric plants having transformed germ cells, as is known in the art. Methods that can be used to transform plant cells or tissue with expression vectors of the present invention include both Agrobacterium and non-Agrobacterium vectors. Agrobacterium-mediated gene transfer exploits the natural ability of Agrobacterium tumefaciens to transfer DNA into plant chromosomes and is described in detail in Gheysen, G., Angenon, G. and Van Montagu, M. 1998. Agrobacterium-mediated plant transformation: a scientifically intriguing story with significant applications. In K. Lindsey (Ed.), Transgenic Plant Research. Harwood Academic Publishers, Amsterdam, pp. 1-33 and in Stafford, H. A. (2000) Botanical Review 66: 99-118. A second group of transformation methods is the non-Agrobacterium mediated transformation and these methods are known as direct gene transfer methods. An overview is brought by Barcelo, P. and Lazzeri, P. A. (1998) Direct gene transfer: chemical, electrical and physical methods. In K. Lindsey (Ed.), Transgenic Plant Research, Harwood Academic Publishers, Amsterdam, pp. 35-55. Hairy root cultures can be obtained by transformation with virulent strains of Agrobacterium rhizogenes, and they can produce high contents of secondary metabolites characteristic to the mother plant. Protocols used for establishing of hairy root cultures vary, as well as the susceptibility of plant species to infection by Agrobacterium (Toivounen L. (1993) Biotechnol. Prog. 9, 12; Vanhala L. et al. (1995) Plant Cell Rep. 14, 236). It is known that the Agrobacterium strain used for transformation has a great influence on root morphology and the degree of secondary metabolite accumulation in hairy root cultures. It is possible that by systematic clone selection e.g., via protoplasts, to find high yielding, stable, and from single cell derived-hairy root clones. This is possible because the hairy root cultures possess a great somaclonal variation. Another possibility of transformation is the use of viral vectors (Turpen T H (1999) Philos Trans R Soc Lond B Biol Sci 354(1383): 665-73).

Any plant tissue or plant cells capable of subsequent clonal propagation, whether by organogenesis or embryogenesis, may be transformed with an expression vector of the present invention. The term ‘organogenesis’ means a process by which shoots and roots are developed sequentially from meristematic centers; the term ‘embryogenesis’ means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include protoplasts, leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyls meristem).

These plants may include, but are not limited to, plants or plant cells of agronomically important crops, such as tomato, tobacco, diverse herbs such as oregano, basilicum and mint. It may also be applied to plants that produce valuable compounds, for example, useful as for instance pharmaceuticals, as ajmalicine, vinblastine, vincristine, ajmaline, reserpine, rescinnamine, camptothecine, ellipticine, quinine, and quinidine, taxol, morphine, scopolamine, atropine, cocaine, sanguinarine, codeine, genistein, daidzein, digoxin, calystegins or as food additives such as anthocyanins, vanillin; including but not limited to the classes of compounds mentioned above. Examples of such plants include, but not limited to, Papaver spp., Rauwolfia spp., Taxus spp., Cinchona spp., Eschscholtzia californica, Camptotheca acuminata, Hyoscyamus spp., Berberis spp., Coptis spp., Datura spp., Atropa spp., Thalictrum spp., Peganum spp.

In yet another embodiment, suitable expression cassettes comprising the nucleotide sequences of the present invention can be used for transformation into other species (different from Tobacco). This transformation into other species or genera (different from the genus Nicotiana) can be carried out randomly or can be carried out with strategically chosen nucleotide sequences. The random combination of genetic material from one or more species of organisms can lead to the generation of novel metabolic pathways (for example through the interaction with metabolic pathways resident in the host organism or alternatively silent metabolic pathways can be unmasked) and eventually lead to the production of novel classes of compounds. This novel or reconstituted metabolic pathways can have utility in the commercial production of novel, valuable compounds.

The recombinant DNA and molecular cloning techniques applied in the below examples are all standard methods well known in the art and are, for example, described by Sambrook et al. (1989) Molecular cloning: A laboratory manual, second edition, Cold Spring Harbor Laboratory Press. Methods for tobacco cell culture and manipulation applied in the below examples are methods described in or derived from methods described in Nagata et al. (1992) Int. Rev. Cytol. 132, 1.

The invention is further explained with the aid of the following illustrative examples.

### EXAMPLES

1) Nicotine Alkaloids

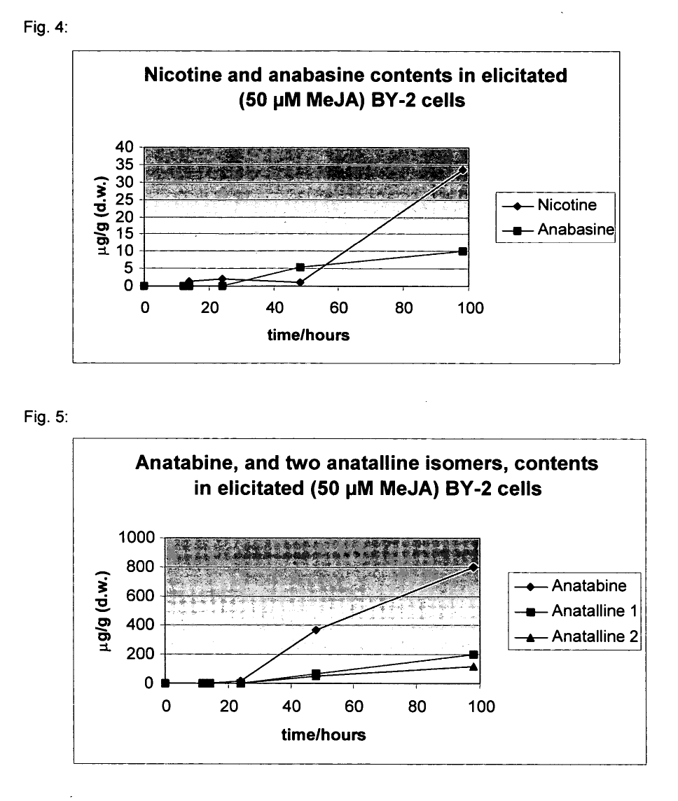

First, the identification of various tobacco alkaloids: nicotine, nornicotine, anatabine, myosmine, anabasine and N′-formylnornicotine was determined from leaves, where the occurrence of alkaloids is abundant. Identification was based on the GC-MS spectra and literature (see, FIG. 3). There were no alkaloids detected in the control samples of BY-2. Elicitation of BY-2 cells by methyl jasmonate leads to a marked increase in nicotine, anabasine, anatalline, and especially in anatabine content, the latter clearly being the main component (FIGS. 4 & 5). To our knowledge, this is the first time that besides nicotine, these other alkaloids has been detected in tobacco BY-2 cell cultures.

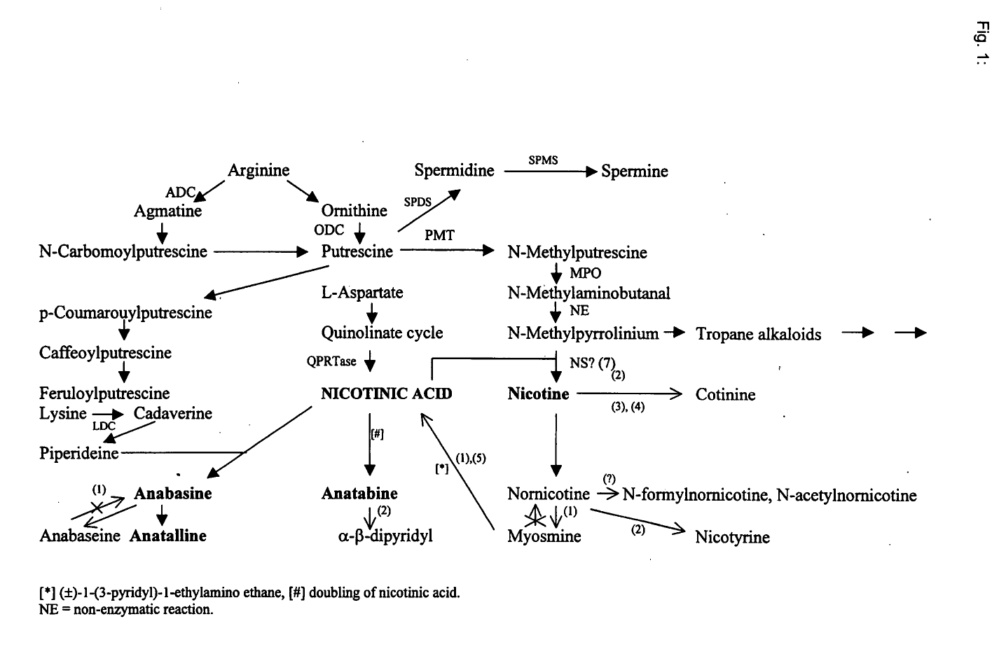

Elicitation with methyl jasmonate seems to induce the pathway through nicotinic acid (FIG. 1). Especially the concentration of anatabine was raised, which according to literature based on biosynthetic studies, is simply derived from nicotinic acid, but neither through the arginine pathway, which leads to nicotine, nor via the lysine pathway which, in turn, leads to anabasine. The elicited BY-2 samples also contained increased amounts of two isomeric alkaloids with m/z 239 as the molecular ion. It is called anatalline and it has been discovered earlier only in the roots of N. tabacum, and never in cell cultures. Yet it was not detected in tobacco leaves. Anatalline is composed of three pyridine ring units of which one has no double bonds (2,4-bis-3′-pyridyl-piperidine). Based on the mass spectra, anatalline may not be derived from anatabine, but rather from anabasine. This is also in accordance with the information found in the literature. In the growth medium of BY-2 cells no alkaloids could be detected.

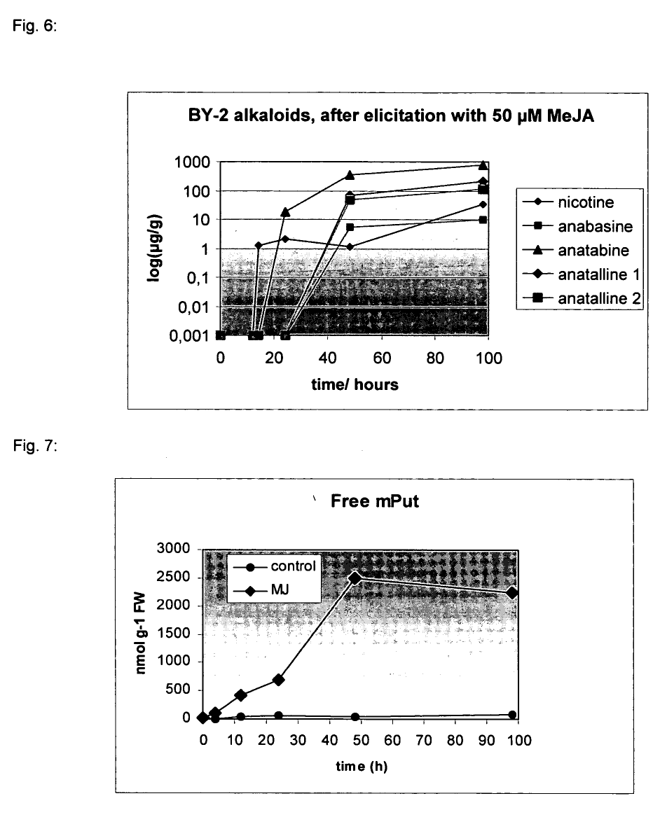

The elicitation with methyl jasmonate induces the accumulation of various nicotine alkaloids. The accumulation of alkaloid metabolites in the cells started after 14 hours and reached their maximum levels towards the end of the experimental period (FIG. 6). The accumulation of nicotine and anatabine started to take place after 14 and 24 hours, respectively. The contents of anabasine, and two isomers of anatalline in the cells increased only after 48 hours. The maximum concentration of nicotine was only 4% (on dry weight basis) of that of the main alkaloid anatabine, which reached the highest concentration of 800 μg/g (d.w.). The time-course of the onset of nicotine accumulation is in accordance with the data reported by Imanishi et al. (1998), who studied only nicotine alkaloid pattern after elicitation. Anatabine and nicotine are synthesized first, while anabasine and anatalline, which follow exactly the similar time-course patterns, accumulate later (FIG. 6).

Instead of nicotine, the level of alkaloids on the other branch of the biosynthetic pathway, for example, anatabine and anatalline was remarkably raised, both branches competing for the supply of nicotinic acid. This was the first time that anatalline was found to be synthesised in the cell suspension cultures of tobacco. The result indicates that nicotine, having two precursors, nicotinic acid and N-methylpyrrolinium, might not be synthesised if the latter is a limiting factor. Thus the pathway from nicotinic acid is directed towards the other biosynthetic routes (see FIG. 1).

2) Polyanines

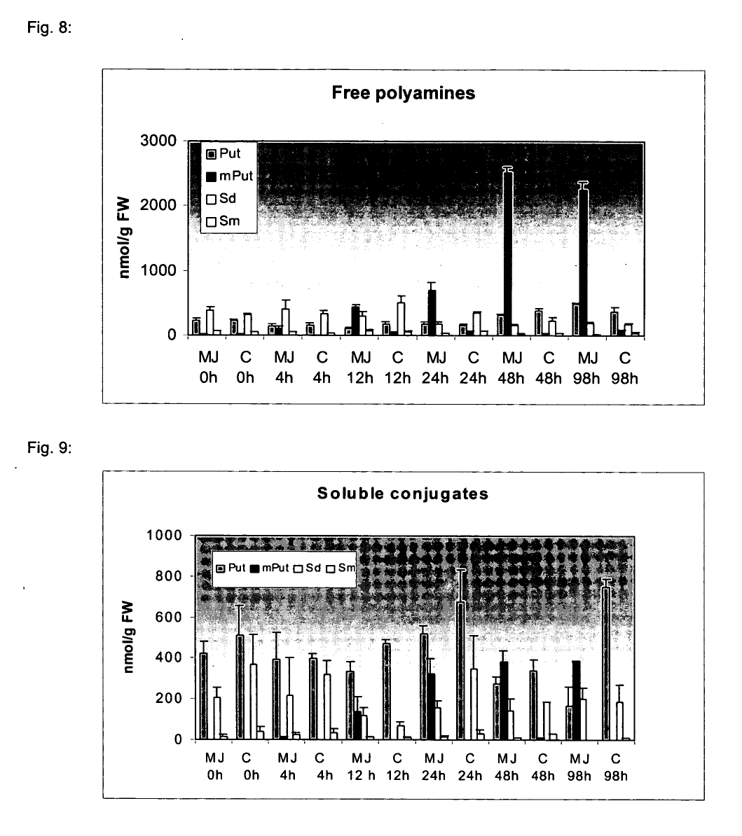

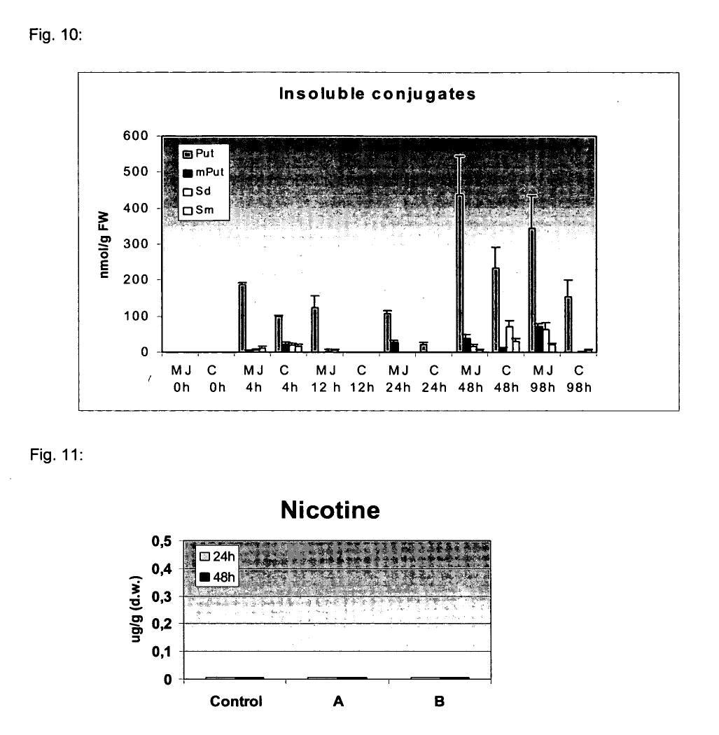

The detection of various polyamines in BY-2 cells including spermidine, spermine, putrescine and methylputrescine were detected by HPLC (Scaramagli et al., 1999). In free pool there were no significant changes between elicited and control samples, except for methyl putrescine which accumulates dramatically in elicited cells (FIG. 7, FIG. 8). Soluble conjugates, which are amines conjugated with phenolic acid, mainly cinnamic acid derivatives did not change much except for methyl putrescine, which accumulates in elicited cells from 12 hours onwards (FIG. 9). Insoluble conjugates which are mainly polyamines associated in cell walls showed that especially putrescine and also methyl putrescine accumulate in elicited cells (FIG. 10). In short, it seems that elicitor treatment induces the accumulation of intermediates putrescine and methyl putrescine in nicotine pathway.

3) Sesquiterpenes

The preliminary experiment indicated the presence of various oxygenated sesquiterpenoid alkaloids, detected in the elicitated cells of tobacco BY-2. Presumably they are structurally aristolochene-like sesquiterpenes, with the molecular weight of 224. Aristolochenes are compounds found in the early steps of the biosynthetic pathway of sesquiterpenes, for example, capsidiol, lubimine, solavetivone, phytuberin and phytuberol.

4) Phenylpropanoids

TLC analysis of BY-2 cells and culture filtrates clearly shows that apart form nicotine, jasmonates also are able to induce the production of (several) phenylpropanoid-like substances.

5) Quantitative Analysis of Jasmonate-Modulated Gene Expression

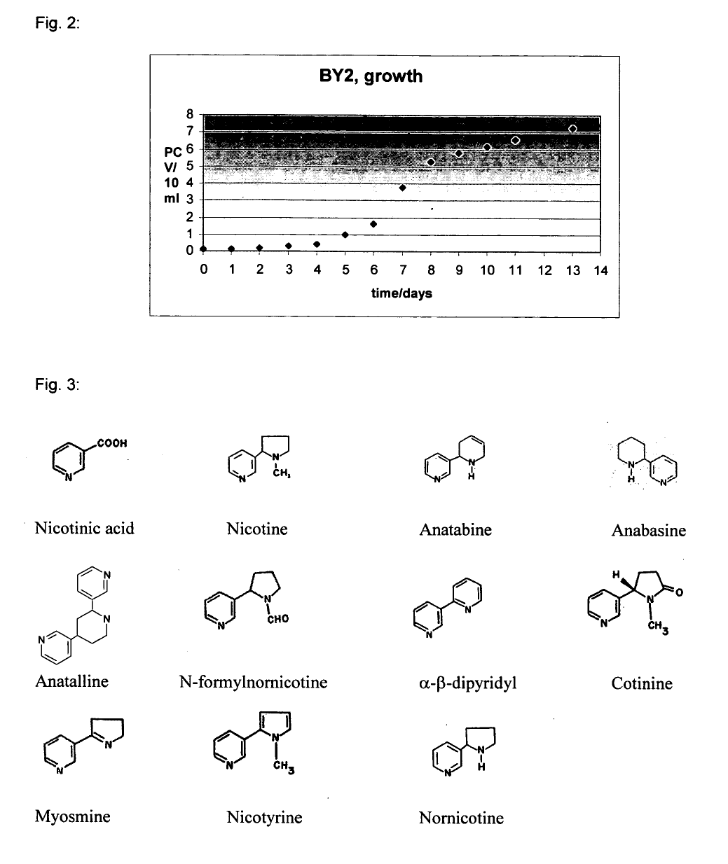

By using the combination of metabolic profiling and cDNA-AFLP based transcript profiling of jasmonate-elicited tobacco BY-2 cells we were able to build an ample inventory of genes involved in plant secondary metabolism and other jasmonate-regulated cellular events. The growth curve of tobacco BY-2 cells is shown in FIG. 2. The culture was inoculated as every 7th day subculturing, 1:100. The growth reached the exponential phase in 6 days. Stationary phase was obtained after 10 days. The gene platform that was generated correlates also with earlier reports and reviews on jasmonate-modulated cellular and metabolic events, pointing to the accuracy and the reliability of the profiling analysis. Examples are the observed up-regulation of genes involved in the biosynthesis of jasmonates (an auto-regulatory event) and genes involved in defense responses such as proteinase inhibitors and transposases. At the same time numerous novel genes, either without existing homologues or with homologues of known or unknown function, were identified as jasmonate responsive and correlates with the production of alkaloids and phenylpropanoids. Some of them point to cellular or metabolic events that have been not related with jasmonates before.

Tobacco BY-2 cells were elicited with 50 μM methyl jasmonate and transcript profiles were compared with the transcript profiles of DMSO-treated cells. Quantitative temporal accumulation patterns of approximately 20,000 transcript tags were determined and analyzed. In total, 591 differential transcript tags were obtained. Sequencing of the PCR products gave good-quality sequences for approximately 80% of the fragments. To the remaining 20%, a unique sequence could not unambiguously be attributed because the fragments were contaminated with co-migrating bands. These bands have been cloned and PCR products from four individual colonies were sequenced. For most of these fragments, two to three different sequences were obtained from the individual colonies. Homology searches with the sequences from the unique gene tags revealed that 64% of these tags displayed similarity with genes of known functions, and 18% of the tags matched a cDNA or genomic sequence without allocated function. In contrast, no homology with a known sequence was found for 18% of the tags.

By average linkage hierarchical clustering of the expression profiles, the genes could be grouped in two main clusters: induced and repressed by jasmonate elicitation. The group of jasmonate repressed genes comprises ca. 18% of the isolated gene tags. The vast majority of jasmonate modulated genes is upregulated by jasmonate elicitation and can be subdivided in three categories: early induced (within 1 hour after the elicitation), intermediate (after two to 4 hours) and late induced (after 6 hours or more). These subcategories respectively comprise ca. 31%, 27% and 24% of the isolated gene tags.

Among the early induced subgroup figure, all the genes that are known to be involved with nicotine biosynthesis in Nicotiana species, i.e., arginine decarboxylase (ADC), ornithine decarboxylase (ODC) and quinolate phosphoribosyltransferase (QPRT). The fourth gene known to be involved in nicotine biosynthesis, putrescine methyl transferase (PMT), could not be picked up with the cDNA-AFLP method used here as its nucleotide sequence does not harbor a BstYI restriction site. Nonetheless, RT-PCR analysis clearly shows that PMT expression is also upregulated as early as one hour after jasmonate treatment and thus demonstrates the co-regulation of the PMT gene(s) with the other nicotine metabolic genes mentioned above. Interestingly, two other gene tags coregulated with the above mentioned genes show homology with putative (amine) oxidases and potentially encode the still undiscovered methyl putrescine oxidase (MPO). Other gene tags that are found in this subgroup are the genes involved with jasmonate biosynthesis such as allene oxide synthase, allene oxide cyclase, 12-oxophytodienoate reductase and lipoxygenases.

In the subsequent induction wave (within two to four hours) another group of genes is found that putatively encode enzymes involved in flavonoid metabolism. Amongst these figure phenylalanine ammonia-lyase, chalcone synthase-like proteins, isoflavone synthase-like proteins, leucoanthocyanidin dioxygenase-like proteins and various cytochrome P450 enzymes.

6) Functional Analysis of Candidate Genes.

Selected genes were introduced in appropriate vectors for over-expression and/or down-regulation using the Gateway™ technology (InVitrogen Life Technologies). To this end a set of Gateway compatible binary vectors for plant transformation was developed (Karimi et al., 2002). For over-expression the pK7WGD2 vector is used in which the gene is put under the control of the p35S promoter. Down-regulation is based on the post-transcriptional gene silencing effect (PTGS, Smith et al., 2000) and to this end the pK7GWIWG2 is used. For plant cell transformations the ternary vector system (van der Fits et al., 2000) was applied. The plasmid pBBR1MCS-5.virGN54D was used as a ternary vector. The binary plasmid was introduced into Agrobacterium tumefaciens strain LBA4404 already bearing the ternary plasmid by electro-transformation. For hairy root transformation the binary plasmid was introduced in the Agrobacterium rhizogenes strain LBA9402.

Fresh BY-2 culture was established before the transformation with the particular construct. Five-day-old BY-2 was inoculated 1:10 and grown for three days (28° C., 130 rpm, dark). The liquid culture of Agrobacterium tumefaciens transformed with pK7WGD2-GUS, pK7WGD2-NtCYP1 (insert from SEQ ID No 465) or pK7WGD2-NtORC1 (insert from SEQ ID No 285) was established two days before the transformation of BY-2. A loopfull of bacteria from the solid medium was inoculated in 5 ml of liquid LB medium with the antibiotics (rifampicin, gentamycin, streptomycin and spectinomycin). The culture was grown for two days (28° C., 130 rpm).

The transformation of BY-2 was performed in empty petri dish (Ø4.6 cm) with the cocultivation method. Three-day-old BY-2 (3 ml) was pipetted into plate and either 50 or 200 μl of bacterial suspension was added. The plates were gently mixed and left to stand in the laminar bench in the dark for three days. After cocultivation the cells were plated on the solid BY-2-medium with the selections (50 μg/ml kanamycin, and 500 μg/ml vancomycin and 500 μg/ml carbenicillin to kill the excess of bacteria). The plates were sealed with millipore tape and incubated at 28° C. in the dark for approximately two weeks after which the calli became visible. The transformation was visualised by checking the expression of GFP (green fluorescent protein) under the microscope.

The suspension culture of the transformed BY-2 was started by taking a clumb of calli (appr. Ø 1 cm) into 20 ml liquid BY-2 medium with the selection. After several subcultures the suspension volume was increased. When the growth of the culture reached the normal growth pattern of BY-2 (subculturing every 7th day), the elicitation experiment was performed as described earlier. Before washing the culture in the beginning of the experiment, the selection (kanamycin) was still present. The density of the culture as well as the GFP expression and viability of the cells were checked before starting the experiment.

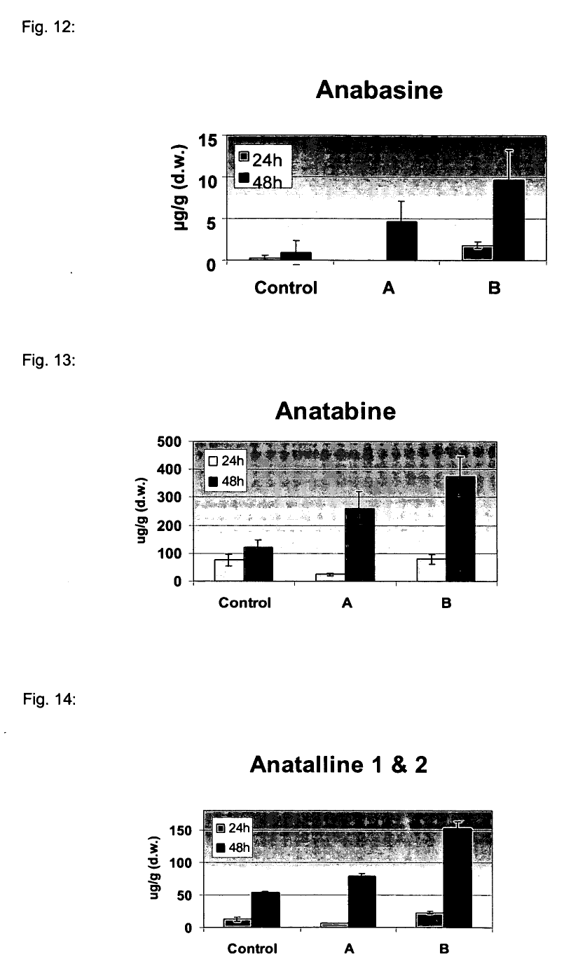

The nicotine alkaloids were detected 24 h and 48 h after elicitation with MeJA (50 μM). Trace amounts of nicotine was detected in all samples and no effect of transformed constructs (pK7WGD2-NtCYP1 and pK7WGD2-NtORC1) compared to the control (pK7WGD2-GUS) was observed (FIG. 11). Anabasine concentration increased in a function of time and a marked increase compared to the control was observed with pK7WGD2-NtORC1-transformed line, bearing the ORCA homologue gene (FIG. 12). Considering the major alkaloid anatabine, no difference in alkaloid accumulation was observed 24 h after elicitation, but at 48 h both transformed constructs, bearing either cyclophilin or AP2 transcription factor, showed clear increase in anatabine levels compared to the control (FIG. 13). The two anatalline isomers followed the similar pattern as anatabine, the transformed lines bearing the putatively functional constructs accumulated notably higher levels of both isomers than the control line (FIG. 14). The overall levels of accumulated alkaloids were in each transformed line lower than in untransformed BY-2, suggesting that the transformation protocol itself might have an inhibitory effect on alkaloid production. The effect of excess of antibiotics possibly still present during the elicitation is also to be tested for their contribution to lower accumulation of alkaloids. However, these results indicate that the above mentioned constructs had a considerable positive effect on the alkaloid accumulation compared to the control line, bearing no functional construct.

7) Isolation of Full-Length Genes and Homologues

MAP3 (SEQ ID NO: 285 and SEQ ID NO: 872): sequence information for an AP2-domain transcription factor, induced after 1 hour by methyl jasmonate in tobacco BY-2 cells.
   Best Homologues found: (lowest blastx 3e-22):
emb|CAB96899.1| AP2-domain DNA-binding protein [Catharanthus roseus]
emb|CAB93940.1| AP2-domain DNA-binding protein [Catharanthus roseus]
gb|AAM45475.1| ethylene-responsive element binding protein 1 [Glycine max]
ref|NP_{—}182011.1| putative ethylene response element binding protein (EREBP) At2g44840 [Arabidopsis thaliana]
pir∥T02432 ethylene-responsive transcription factor ERF1 [Nicotiana tabacum]
pir∥T07686 transcription factor Pti4 [Lycopersicon esculentum]
C330 (SEQ ID NO: 148 and SEQ ID NO: 873): sequence information for an AP2-domain transcription factor induced after 1 hour by methyl jasmonate in tobacco BY-2 cells.
   Best Homologues found:(lowest blastx 2e-27):
ref|NP_{—}199533.1| ethylene responsive element binding factor 2 (EREBP-2) [A. thaliana]
dbj|BAA87068.2| ethylene-responsive element binding protein1 homolog [Matricaria chamomilla]
gb|AAF63205.1| AF245119_{—}1 AP2-related transcription factor [Mesembryanthemum crystallinum]
pir∥T07686 transcription factor Pti4 [Lycopersicon esculentum]
pir∥T02590 ethylene-responsive element binding protein [Nicotiana tabacum]
   Both MAP3 and C330 encode transcription factors belonging to the AP2-domain transcription factor family, to which also for instance the ORCA genes belong, known to regulate the jasmonate responsive biosynthesis of terpenoid indole alkaloids in Catharanthus roseus (Memelink et al., Trends Plant Sci. 2001, 6(5):212-219). Since both MAP3 and C330 are induced before or concomitantly with the nicotine biosynthetic genes PMT, ADC, ODC, QPRT, AP and SAMS, this clearly mirrors a potential role as activators of nicotine biosynthesis for these genes. This was confirmed by assessment of nicotine alkaloid accumulation levels (for MAP3 and reporter gene expression analysis (for C330).
C484a (SEQ ID N° 275 and SEQ ID NO: 874): a C3HC4-type RING zinc finger protein induced after 1 hour by methyl jasmonate in tobacco BY-2 cells.
   Best Homologues found: (lowest blastx 8e-30)>
ref|NP_{—}181135.2| putative RING zinc finger protein At2g35910 [A. thaliana]
ref|NP_{—}196267.1| C3HC4-type RING zinc finger protein At5g06490 [A. thaliana]
   Zinc finger proteins can be transcriptional regulators reported to interact for instance with the promoter regions of some genes involved in the biosynthesis of terpenoid indole alkaloids in Catharanthus roseus (Ouwerkerk et al., Mol. Gen. Genet. 1999, 261(4-5):610-622). They can also interact with components of the SCF (Skp1/Cullin/F-box protein)-type E3 ubiquitin ligase complex involved in protein degradation (e.g., Liu et al, Plant Cell 2002, 14(7):1483-1496). Such a complex has shown to be of extreme importance in jasmonate-mediated signaling cascades (Turner et al., Plant Cell. 2002, 14 Suppl:S153-S164) and thus participates as well in the regulation of plant secondary metabolism.
   C360 (SEQ ID NO: 180 and SEQ ID NO: 875): sequence information for a protein with similarity to the putative protein At4g14710 [A. thaliana] induced after 4 hour by methyl jasmonate in tobacco BY-2 cells.
   Best Homologues found: (lowest blastx 2e-87)>
ref|NP_{—}567441.1| Expressed protein At4g14710 [A. thaliana]
ref|NP-567443.1| Expressed protein At4g14716 [A. thaliana]
ref|NP_{—}180208.1| unknown protein At2g26400 [A. thaliana]
pir∥T02918 probable submergence induced, nickel-binding protein 2A [Oryza sativa]
dbj|BAB61039.1| iron-deficiency induced gene [Hordeum vulgare]
>pir∥T02787 probable submergence induced protein 2 [Oryza sativa]

This protein contains an ARD/ARD′ family motif, found in two acireductone dioxygenase enzymes (ARD and ARD′, previously known as E-2 and E-2′) from Klebsiella pneumoniae. The two enzymes share the same substrate, 1,2-dihydroxy-3-keto-5-(methylthio)pentene, but yield different products. ARD′ yields the alpha-keto precursor of methionine (and formate), thus forming part of the ubiquitous methionine salvage pathway that converts 5′-methylthioadenosine (MTA) to methionine. This pathway is responsible for the tight control of the concentration of MTA, which is a powerful inhibitor of polyamine biosynthesis and transmethylation reactions [1,2]. ARD yields methylthiopropanoate, carbon monoxide and formate, and thus prevents the conversion of MTA to methionine. The role of the ARD catalysed reaction is unclear: methylthiopropanoate is cytotoxic, and carbon monoxide can activate guanylyl cyclase, leading to increased intracellular cGMP levels (Duai et al., J. Biol. Chem. 1999, 274(3):1193-1195; Dai et al., Biochemistry 2001, 40(21):6379-6387). This family also contains other members, whose functions are not well characterized. The gene isolated here might probably regulate/interact with polyamine biosynthesis and thus nicotine biosynthesis, for which polyamines are precursors.
C165 (SEQ ID NO: 64 and SEQ ID NO: 876): sequence information for a putative ligand-gated ion channel protein induced after 6 hour by methyl jasmonate in tobacco BY-2 cells.
   Best Homologues found: (lowest blastx 2e-80)>
ref|NP_{—}172012.1| putative ligand-gated ion channel protein At1g05200 [A. thaliana]
ref|NP_{—}565743.1| putative ligand-gated ion channel protein At2g32390 [A. thaliana]
dbj|BAC57657.1| putative ionotropic glutamate receptor homolog GLR4 [Oryza sativa (japonica cultivar-group)]
dbj|BAC10393.1| putative ligand-gated channel-like protein [Oryza sativa (japonica cultivar-group)]
   Ligand-gated ion channels are important players in plant hormone induced signaling cascades. They have been found to be involved for instance in abscisic acid signalling (Pei et al., Nature 2000, 406(6797):731-734; Walden, Curr. Opin. Plant Biol. 1998, 1(5):419-423). Abscisic acid, as well as ethylene and jasmonates have also been proposed to play a role in wound signalling, which in many plants leads to the induction of plant secondary metabolic pathways (Leon et al., J. Exp. Bot. 2001 52(354):1-9).
C353a (SEQ ID NO: 172 and SEQ ID NO: 877): sequence information for a GTP-binding protein induced after 6 hour by methyl jasmonate in tobacco BY-2 cells.
   Best Homologues found: (lowest blastx e-102)>
emb|CAA69701.1| small GTP-binding protein [Nicotiana plumbaginifolia]
emb|CAC39050.1| putative GTP-binding protein [Oryza sativa]
dbj|BAA76422.1| rab-type small GTP-binding protein [Cicer arietinum]
emb|CAA98160.1| RAB1C [Lotus japonicus]
pir∥B38202 GTP-binding protein YPTM2 [Zea Mays]
dbj|BAA02116.1| GTP-binding protein [Pisum sativum]
emb|CAA98161.1| RAB1D [Lotus japonicus]
gb|AAF65510.1| small GTP-binding protein [Capsicum annuum]
emb|CAA98162.1| RAB1E [Lotus japonicus]
ref|NP_{—}193486.1| ras-related small GTP-binding protein RAB1c At4g17530.1 [A. thaliana]
MT101 (SEQ ID NO: 355 and SEQ ID NO: 878): Sequence information for a GTP-binding-like protein induced after 1 hour by methyl jasmonate in tobacco BY-2 cells.
   Best Homologues found: (lowest blastx e-177)>
ref|NP_{—}195662.1| GTP-binding-like protein; protein id: At4g39520.1 [A. thaliana]
dbj|BAC22346.1| putative GTP-binding protein [Oryza sativa (japonica cultivar-group)]
   GTP-binding proteins have been reported to be involved in the induction of phytoalexin biosynthesis in cultured carrot cells (Kurosaki et al., Plant Sci. 2001 161(2):273-278) and in the fungal elicitor-induced beta-thujaplicin biosynthesis in Cupressus lusitanica cell cultures (Zhao & Sakai, J. Exp. Bot. 2003, 54(383):647-656). They are supposed to interact with receptors, kinases and phosphatases amongst others and as such participate in many stimulus induced signaling pathways in plants (Clark et al., Curr. Sci. 2001, 80(2):170-177), and possibly as well in the onset of secondary metabolite biosynthetic pathways.
T21 (SEQ ID NO: 465 and SEQ ID NO: 879): Sequence information for a cyclophilin induced after 8 hour by methyl jasmonate in tobacco BY-2 cells.
   Best Homologues found: (lowest blastx 4e-78)>
gb|AAA63543.1| cyclophilin [Lycopersicon esculentum]
>pir∥CSTO peptidylprolyl isomerase (EC 5.2.1.8) [Lycopersicon esculentum]
>pir∥T50771 peptidylprolyl isomerase (EC 5.2.1.8) [Solanum tuberosum subsp. tuberosum]
emb|CAC80550.1| cyclophilin [Ricinus communis]
gb|AAB51386.1| stress responsive cyclophilin [Solanum commersonii]
pir∥T50768 cyclophylin [Digitalis lanata]
   Cyclophylins or FK506-binding proteins belong to the large family of peptidyl-prolyl cis-trans isomerases, which are known to be involved in many cellular processes, such as cell signalling, protein trafficking and transcription (Harrar et al., Trends Plant Sci 2001, 6(9):426-431), and as such might be involved in regulating plant secondary metabolism.
C476a (SEQ ID NO: 264 and SEQ ID NO: 880): sequence information for a MAP kinase induced after 1 hour by methyl jasmonate in tobacco BY-2 cells.
   Best Homologues found: (lowest blastx 2e-75)>
ref|NP_{—}177492.1| MAP kinase At1g73500 [A. thaliana]
ref|NP_{—}173271.1| MAP kinase kinase 5 At1g18350 [A. thaliana]
ref|NP_{—}188759.1| MAP kinasekinase 5 At3g21220 [A. thaliana]
ref|NP_{—}175577.1| MAP kinase kinase 4 (ATMKK4) At1g51660 [A. thaliana]
gb|AAG53979.1|AF325168_{—}1 mitogen-activated protein kinase 2 [Nicotiana tabacum]
   MAP kinases have been reported to be both differentially induced by defense signals such as nitric oxide, salicylic acid, ethylene, and jasmonic acid as to represent key components of the signaling cascades induced by these defense signals (e.g., Petersen et al., Cell 2000, 103(7):1111-1120; Kumar & Klessig, Mol. Plant Microbe Interact. 2000, 13(3):347-351; Seo et al., Science. 1995, 270(5244):1988-1992), and as such might be involved in the activation of plant secondary metabolism.
MC204 (SEQ ID NO: 315 and SEQ ID NO: 881): sequence information for a sequence with similarity to the putative protein At5g47790 [A. thaliana] induced after 6 hour by methyl jasmonate in tobacco BY-2 cells.
   Best Homologues found: (lowest blastx e-111)
dbj|BAC22308.1| OJ1136_A10.4 [Oryza sativa (japonica cultivar-group)]
ref|NP_{—}199590.1| unknown protein At5g47790 [A. thaliana]
   This protein contains a Forkhead-associated (FHA) domain. The forkhead-associated domain is a phosphopeptide recognition domain found in many regulatory proteins. It displays specificity for phosphothreonine-containing epitopes but will also recognize phosphotyrosine with relatively high affinity. It spans approximately 80-100 amino acid residues folded into an 11-stranded sandwich, which sometimes contain small helical insertions between the loops connecting the strands. The domain is present in a diverse range of proteins, such as kinases, phosphatases, kinesins, transcription factors, RNA-binding proteins and metabolic enzymes which take part in many different cellular processes, such as signal transduction, vesicular transport and protein degradation (Durocher et al., Mol. Cell 1999, 4(3):387-394; Hofmann & Bucher, Trends Biochem. Sci. 1995, 20(9):347-349), and as such might regulate plant secondary metabolism.
T323 (SEQ ID NO: 509 and SEQ ID NO: 882): Sequence information for a putative endo-1,4-beta-glucanase induced after 10 hour by methyl jasmonate in tobacco BY-2 cells.
   Best Homologues found: (lowest blastx 2e-84)>
emb|CAD41248.1| OSJNBa0067K08.12 [Oryza sativa (japonica cultivar-group)]
ref|NP_{—}176738.1| glycosyl hydrolase family 9 (endo-1,4-beta-glucanase) At1g65610 [A. thaliana]
ref|NP_{—}199783.1| cellulase [A. thaliana]
emb|CAB51903.1| cellulase; endo-1,4-beta-D-glucanase [Brassica napus]
pir∥T07612 cellulase [Lycopersicon esculentum]
   The Arabidopsis mutant cev1 links cell wall signaling to jasmonate and ethylene responses (Ellis et al., Plant Cell 2002, 14(7):1557-1566). CEV1 encodes a cellulose synthase. The cev1 mutant has constitutive expression of stress response genes and has increased production of jasmonate and ethylene. Conversely, as such glucanase and cellulase-like proteins might participate in the onset of plant secondary metabolism by providing cell wall derived molecules, necessary to elicit secondary metabolic pathways.
T464 (SEQ ID NO: 595 and SEQ ID NO: 883): Sequence information for an epimerase/dehydratase-like protein induced after 10 hour by methyl jasmonate in tobacco BY-2 cells.
   Best Homologues found: (lowest blastx 0.0)>
gb|AAM08784.1|AC016780_{—}14 Putative epimerase/dehydratase [Oryza sativa]
ref|NP_{—}198236.1| epimerase/dehydratase-like protein At5g28840.1 [A. thaliana]
   It has been shown that phytoalexin production elicited by exogenously applied jasmonic acid in rice leaves (Oryza sativa L.) is under the control of cytokinins and ascorbic acid (Tamogami et al., FEBS Lett. 1997, 412(1):61-64). MJM tag T464 encodes the homologue of the GDP-mannose 3″,5″-epimerase of A. thaliana, a key enzyme of the plant vitamin C pathway (Wolucka et al., Proc. Natl. Acad. Sci. USA 2001, 98(26):14843-14848). Consequently, increased ascorbate production might stimulate alkaloid and phenylpropanoid biosynthesis as well, and plant secondary metabolism in general.
C127 (SEQ ID NO: 38 and SEQ ID NO: 884): Sequence information for an auxin-responsive GH3-like protein induced after 2 hour by methyl jasmonate in tobacco BY-2 cells.
   Best Homologues found: (lowest blastx e-180)>
ref|NP-200262.1| auxin-responsive-like protein At5g54510 [A. thaliana]
ref|NP_{—}194456.1| GH3 like protein At4g27260 [A. thaliana]
dbj|BAB92590.1| putative auxin-responsive GH3 [Oryza sativa (japonica cultivar-group)]
gb|AAD32141.1|AF 123503_{—}1 Nt-gh3 deduced protein [Nicotiana tabacum]
dbj|BAB63594.1| putative auxin-responsive GH3 protein [Oryza sativa (japonica cultivar-group)]
ref|NP_{—}179101.1| putative auxin-regulated protein At2g14960.1 [A. thaliana]
pir∥S17433 auxin-regulated protein GH3 [Glycine max]
C175 (SEQ ID NO: 71 and SEQ ID NO: 885): Sequence information for an auxin-responsive GH3-like protein induced after 2 hour by methyl jasmonate in tobacco BY-2 cells.
   Best Homologues found: (lowest blastx)
ref|NP_{—}200262.1| auxin-responsive-like protein At5g54510 [A. thaliana]
ref|NP_{—}1194456.1| GH3 like protein At4g27260 [A. thaliana]
dbj|BAB92590.1| putative auxin-responsive GH3 [Oryza sativa (japonica cultivar-group)]
gb|AAD32141.1|AF123503_{—}1 Nt-gh3 deduced protein [Nicotiana tabacum]
dbj|BAB63594.1| putative auxin-responsive GH3 protein [Oryza sativa (japonica cultivar-group)]
ref|NP_{—}179101.1| putative auxin-regulated protein At2g14960.1 [A. thaliana]
pir∥S17433 auxin-regulated protein GH3 [Glycine max]
   The Arabidopsis jasmonate (JA) response mutant jar1-1 is defective in the gene JAR1, one of 19 closely related Arabidopsis genes that are similar to the auxin-induced soybean GH3 gene. Analysis of fold predictions for this protein family suggested that JAR1 might belong to the acyl adenylate-forming firefly luciferase superfamily. These enzymes activate the carboxyl groups of a variety of substrates for their subsequent biochemical modification. An ATP-PPi isotope exchange assay was used to demonstrate adenylation activity in a glutathione S-transferase-JAR1 fusion protein. Activity was specific for JA, suggesting that covalent modification of JA is important for its function. Six other Arabidopsis genes were specifically active on indole-3-acetic acid (IAA), and one was active on both IAA and salicylic acid. These findings suggest that the JAR1 gene family is involved in multiple important plant signaling pathways (Staswick et al., Plant Cell 2002, 14(6):1405-1415). The MJM genes C127 and C175 cluster together with the Arabidopsis genes At5g54510 and At4g27260, of which the protein products display activity on IAA. They might participate in the conversion of free, active IAA in inactive storage forms or conjugates, and as such relieve the inhibitory effect of active auxins on secondary metabolism, shown for instance for nicotine production in tobacco cells (Imanishi et al., Plant Mol. Biol. 1998, 38(6):1101-1111) and terpenoid indole alkaloid production in Catharanthus roseus cells (Gantet et al., Plant Cell Physiol., 1998, 39(2):220-225).
T424b (SEQ ID NO: 570 and SEQ ID NO: 886): sequence information for an auxin-induced reductase-like protein induced after 1 hour by methyl jasmonate in tobacco BY-2 cells.
   Best Homologues found: (lowest blastx e-144)>
pir∥S16390 auxin-induced protein PCNT115 [Nicotiana tabacum]
ref∥NP_{—}564761.1| auxin-induced protein At1g60710 [A. thaliana]
ref∥NP_{—}176268.1| auxin-induced protein At1g60690 (aldo/keto reductase family) [A. thaliana]
pir∥T12582 auxin-induced protein [Helianthus annuus]
ref∥NP_{—}176267.1| auxin-induced protein At1 g60680.1 [A. thaliana]
ref|NP_{—}172551.1| putative auxin-induced protein [A. thaliana]
   This gene might encode a reductase protein capable of reducing free, active IAA into the inactive form indole-ethanol (Brown & Purves, J. Biol. Chem. 1976, 251(4):907-913). As such, it might also be involved in the relieve of the inhibitory effect of active auxins on secondary metabolism, shown for instance for nicotine production in tobacco cells (Imanishi et al., Plant Mol. Biol. 1998, 38(6):1101-1111) and terpenoid indole alkaloid production in Catharanthus roseus cells (Gantet et al., Plant Cell Physiol., 1998, 39(2):220-225).
T164 (SEQ ID NO: 446 or SEQ ID NO: 887): sequence information for a probable glutathione S-transferase induced after 1 hour by methyl jasmonate in tobacco BY-2 cells.
   Best Homologues found: (lowest blastx e-115)>
emb|CAA56790.1| auxin-regulated par glutathione S-transferase protein STR246C [Nicotiana tabacum]
pir∥JQ1606 multiple stimulus glutathione S-transferase response protein [Nicotiana plumbaginifolia]
   This GST protein is induced also by auxins and might be involved in the transport of IAA-conjugates, detoxification of secondary metabolites or even in functions distinct from conventional GSTs (as suggested by some characteristics of parA, Takahashi et al., Planta 1995, 196(1):111-117) such as an involvement in transcriptional regulation.
MAP2 (SEQ ID NO: 284 and SEQ ID NO: 888): sequence information for a protein with similarity to the putative protein At5g28830 [A. thaliana] induced after 6 hour by methyl jasmonate in tobacco BY-2 cells.
   Best Homologues found: (lowest blastx 3e-82)>
ref∥NP_{—}198235.1| putative protein At5g28830 [A. thaliana]
   This protein contains a Ca-binding EF-hand motif. The EF-hands can be divided into two classes: signaling proteins and buffering/transport proteins. The first group is the largest and includes the most well-known members of the family such as calmodulin, troponin C and S100B. These proteins typically undergo a calcium-dependent conformational change which opens a target binding site. The latter group is represented by calcium binding D9k and do not undergo calcium dependent conformational changes. As calmodulins and Ca-molecules have been postulated to be involved in jasmonate signaling cascades (Leon et al., J. Exp. Bot. 2001, 52(354):1-9; Yang & Poovaiah, J. Biol. Chem. 2002, 277(47):45049-45058), possibly connected to the onset of secondary metabolic pathways (Memelink et al., Trends Plant Sci. 2001, 6(5):212-219), they might be involved in nicotine alkaloid or phenylpropanoid biosynthesis as well.

C1 (SEQ ID NO: 8 and SEQ ID NO: 889): Sequence information for a 1,4-benzoquinone reductase-like induced after 12 hour by methyl jasmonate in tobacco BY-2 cells.

Best Homologues found: (lowest blastx 5e-79)>

ref|NP_{—}200261.1| quinone reductase At5g54500.1 [A. thaliana]
emb|CAD31838.1| putative quinone oxidoreductase [Cicer arietinum]
gb|AAD38143.1|AF 139496_{—}1 unknown [Prunus armeniaca]
ref|NP_{—}194457.1| quinone reductase family protein At4g27270.1 [A. thaliana]
gb|AAG53945.1|AF304462_{—}1 quinone-oxidoreductase QR2 [Triphysaria versicolor]
dbj|BAB92583.1| putative 1,4-benzoquinone reductase [Oryza sativa (japonica cultivar-group)]
   This reductase-like protein might be directly and actively involved in the biosynthetic pathway of one of the nicotine alkaloids.
T210 (SEQ ID NO: 466 and SEQ ID NO: 890): Sequence information for a protein with similarity to the putative protein P0638D12 [Oryza sativa] induced after 6 hour by methyl jasmonate in tobacco BY-2 cells.
   Best Homologues found: (lowest blastx 5e-60)>
dbj|BAB55502.1| P0638D12.10 [Oryza sativa (japonica cultivar-group)]
ref|NP_{—}565816.1| expressed protein At2g35680 [A. thaliana]
gb|AAK31276.1|AC079890_{—}12 unknown protein [Oryza sativa]
ref|NP-200472.1| putative protein At5g56610 [A. thaliana]
   This protein contains a dual specificity protein phosphatase motif. Ser/Thr and Tyr dual specificity phosphatases are a group of enzymes (EC: 3.1.3.16) removing the serine/threonine or tyrosine-bound phosphate group from a wide range of phosphoproteins, including a number of enzymes which have been phosphorylated under the action of a kinase (Fauman & Saper, Trends Biochem. Sci. 1996, 21(11):413-417). As such, they might be involved in the regulation of plant secondary metabolic pathways.
C112 (SEQ ID NO: 22 and SEQ ID NO: 891): Sequence information for a protein with similarity to the putative protein At3g11810 [A. thaliana] induced after 12 hour by methyl jasmonate in tobacco BY-2 cells.
   Best Homologues found: (lowest blastx 1e-10)
ref|NP_{—}187787.1| unknown protein At3g11810 [A. thaliana]
ref|NP_{—}178432.1| unknown protein; protein id: At2g03330.1 [A. thaliana]
   This protein contains a TonB motif. In Escherichia coli the TonB protein interacts with outer membrane receptor proteins that carry out high-affinity binding and energy-dependent uptake of specific substrates into the periplasmic space. These substrates are either poorly permeable through the porin channels or are encountered at very low concentrations. In the absence of tonB these receptors bind their substrates but do not carry out active transport (Buchanan et al., Nat. Struct. Biol. 1999, 6(1):56-63.). As such, this protein might be involved in the jasmonate-induced signaling cascades and thus in the regulation of plant secondary metabolic pathways.
C454 (SEQ ID NO: 244 and SEQ ID NO: 892): Sequence information for sequence a putative phosphatase 2C induced after 1 hour by methyl jasmonate in tobacco BY-2 cells.
   Best Homologues found: (lowest blastx 4e-85)>
ref|NP_{—}180455.1| unknown protein At2g28890 [A. thaliana]
ref|NP_{—}563791.1| expressed protein At1g07630 [A. thaliana]
ref|NP_{—}195860.1| putative protein At5g02400 [A. thaliana]
gb|AAO65883.1| putative protein phosphatase 2C [Oryza sativa (japonica cultivar-group)]
ref|NP_{—}187551.1| unknown protein At3g09400 [A. thaliana]
ref|NP_{—}182215.2| unknown protein; protein At2g46920 [A. thaliana]
T172 (SEQ ID NO: 450 and SEQ ID NO: 893): Sequence information for a protein phosphatase 2C induced after 4 hour by methyl jasmonate in tobacco BY-2 cells.
   Best Homologues found: (lowest blastx e-104)>
ref|NP_{—}177421.1| protein phosphatase 2C (AtP2C-HA) At1g72770 [A. thaliana]
ref|NP_{—}173199.1| protein phosphatase 2C At1g17550 [A. thaliana]
dbj|BAC05575.1| protein phosphatase 2C-like protein [Oryza sativa (japonica cultivar-group)]
ref|NP-200515.1| protein phosphatase 2C, ABI2 At5g57050.1 [A. thaliana]
ref|NP_{—}194338.1| protein phosphatase ABI1 At4g26080 [A. thaliana]
   Phosphatases have been postulated as important participants in the jasmonate modulated signaling cascades (Leon et al., J. Exp. Bot. 2001, 52(354):1-9) and as such represent potential powerful master regulators of plant secondary metabolism. T172 shows most homology to a group of 4 Arabidopsis PP2C phosphatases to which also ABI1 and ABI2 belong, acting in a negative feedback regulatory loop of the abscisic acid signalling pathway (Merlot et al., Plant J. 2001, 25(3):295-303). C454 shows most homology to a group of 5 Arabidopsis PP2C phosphatases to which also POLTERGEIST belongs, encoding a PP2C that regulates CLAVATA pathways controlling stem cell identity at Arabidopsis shoot and flower meristems (Yu et al., Curr Biol. 2003, 13(3):179-188). Both the T172 and C454 sequences are truncated clones and still lack the N-terminal sequence. However, the clones available cover the region corresponding to truncated mutant versions of both ABI (Sheen, Proc. Natl. Acad. Sci. USA 1998, 95(3):975-980) and Poltergeist phosphatases (Yu et al., Curr Biol. 2003, 13(3):179-188) that were shown to confer constitutive activity and thus are very well suitable for metabolic engineering purposes.
C477 (SEQ ID NO: 266 and SEQ ID NO: 894): Sequence information for a putative zinc transporter induced after 4 hour by methyl jasmonate in tobacco BY-2 cells.
   Best Homologues found: (lowest blastx e-121)>
gb|AAL25646.1|AF197329_{—}1 zinc transporter [Eucalyptus grandis]
ref|NP_{—}182203.1| putative zinc transporter At2g46800 [A. thaliana]
gb|AAK91869.2| putative vacuolar metal-ion transport protein MTP1 [Thlaspi goesingense]
gb|AAK91871.2| putative vacuolar metal-ion transport protein MTP1t2 [Thlaspi goesingense]
ref|NP 191440.1 | zinc transporter-like protein At3g58810 [A. thaliana]
gb|AAK69428.1|AF275750_{—}1 zinc transporter [Thlaspi caerulescens]
   Divalent cations are important both as cofactors for biosynthetic enzymes and as active participants in elicitor induced biosynthesis of plant secondary metabolites. For instance calcium molecules and transporters/channels have been shown to mediate fungal elicitor-induced beta-thujaplicin biosynthesis in Cupressus lusitanica cell cultures (Zhao & Sakai, J. Exp. Bot. 2003, 54(383):647-656). Zinc cations as well might be involved, either as a cofactor in enzymes or zinc finger proteins or as a secondary signal molecule, in elicitor-mediated induction of tobacco secondary metabolism.
C331 (SEQ ID NO: 149 and SEQ ID NO: 895): Sequence information for a protein with similarity to the putative protein At3g62270 [A. thaliana] induced after 12 hour by methyl jasmonate in tobacco BY-2 cells.
   Best Homologues found: (lowest blastx 7e-13)>
ref|NP_{—}191786.1| putative protein; protein At3g62270 [A. thaliana]
ref|NP_{—}182238.2| putative anion exchange protein At2g47160 [A. thaliana]
ref|NP_{—}187296.2| unknown protein At3g06450 [A. thaliana]
   This protein harbours a HCO3-transporter motif and might thus function as an anion exchanger. Bicarbonate (HCO3-) transport mechanisms are the principal regulators of the internal pH of animal cells. As intracellular pH shifts have been shown to be part of the signal mechanism leading to the elicitation of benzophenanthridine alkaloids biosynthesis in cultured cells of Eschscholtzia californica (Viehweger et al., Plant Cell 2002, 14(7):1509-1525; Roos et al., Plant Physiol. 1998, 118(2):349-364), this anion exchanger encoded by C331 might be involved in regulating tobacco secondary metabolism.
   8) Use of a Reporter Plant Cell Line as a Tool for Functional Analysis to Accelerate the Identification of Genes with a Role in Secondary Metabolism

The PMT gene encodes the enzyme putrescine N-methyltransferase, catalysing the first committed step in the production of nicotinic alkaloids. Transcripts of Nicotiana sp. PMT genes are reported to be up regulated by methyl jasmonate. When the flanking regions of Nicotiana sylvestris PMT genes were fused to the β-glucuronidase reporter gene and introduced into N. sylvestris, the reporter transgenes were found to be inducible by methyl jasmonate treatment (Shoji et al., Plant Cell Physiol. 2000, 41(7):831-839). We have applied this knowledge and constructed a new reporter construct, called pHGWFS7-ppmt2, harbouring a EGFP-GUS fusion reporter gene (in Gateway® vector pHGWFS7; Karimi et al., Trends Plant Sci. 2002, 7(5):193-195), driven by the NsPMT2 promoter. To this end, primers were designed for the Adapter attB PCR protocol (InVitroGen) to amplify the NsPMT2 5′flanking region covering nucleotides −1713 to +3 (Table 3).

The pHGWFS7-ppmt2 construct was subsequently introduced in the ternary Agrobacterium tumefaciens transformation system, LBA4404.pBBR1-MCS-5.virGN54D (van der Fits et al., Plant Mol. Biol. 2000, 43(4):495-502), allowing efficient transformation of tobacco BY-2 cell cultures. Different independent transgenic lines were established and the jasmonate inducibility of the promoter in these transgenic BY-2 cells was confirmed (Table 4).

These transgenic reporter cell lines are used as a tool to identify potential master regulatory genes of plant secondary metabolism (and speed up this process). Overexpression of a single gene most often does not affect significantly the final production levels of the target metabolite(s). Therefore, when accumulation levels are employed as the only criteria to evaluate the potential involvement of regulatory genes in plant secondary metabolism, one might easily miss eventually promising candidates.

To illustrate the potential of this approach, BY-2-pmt2 cell line 7 was double transformed with the pK7WGD2-C330 construct, harbouring the MJM tag with SEQ ID No 148, an AP2-domain transcription factor encoding gene (also designated as C330 in this application), driven by the constitutive p35S promoter. Expression analysis of the reporter proteins demonstrated clearly that overexpression of the C330 gene induces the NsPMT2 promoter, without the necessity to use elicitors like methyl jasmonate (Table 5).

In a next step, we evaluated if there was a correlation between the GUS-activity in the BY-2 reporter cell line (line 7) and nicotine alkaloid accumulation. Table 6A shows a perfect correlation between GUS expression and nicotine alkaloids (as measured for nicotine, anatabine and anabasine). Table 6B shows the nicotine alkaloid content of the BY-2 reporter cell line (line 7) super-transformed with an expression vector comprising the C330 gene (SEQ ID NO: 148). Measurements in tables 6A and 6B were carried out in the presence or absence of synthetic auxins. “−2,4 D” means in the absence of dichlorophenoxy-acetic acid. “NAA” means in the presence of alfa-naphtalene-acetic acid. “DW” means dry weight, “MeJA” is with the addition of the elicitor methyl jasmonate, “DMSO” means with the addition of dimethylsulfoxide instead of MeJA.

9) Functional Analysis in Hairy Roots of Hyoscyamus muticus

Sterilized leaves of H. muticus were infected with a recombinant Agrobacterium rhizogenes strain (LBA9402) transformed with an expression vector comprising the C330 gene (SEQ ID NO: 148). As a negative control we compared the infection with the LBA9402 wild type strain. The hairy roots appeared in the infected sites approximately 3 weeks after infection. The different root clones were separated and they were grown on plates in B50 medium added with cefotaxim to kill the excess of Agrobacteria. The hairy roots transformed with C330 (4 clones: A, B, C and D) and the control LBA9402 (one clone) were accurately weighed and the same amount was added into each of the flasks (50+3 mg) then 20 ml B50 medium was added. For each of the clones three flasks were prepared. After growing for 21 days (16 h light, 8 h dark, 21° C.), the roots were filtered and lyophilized. The tropane alkaloid extraction and analysis was performed by a modified method of Fliniaux et al. (1993) J. Chromatography 644: 193. For analysis the three flasks of each clone were pooled together and 50 mg dry weight (DW) was withdrawn for an extraction. For the GC-MS analysis, the samples were evaporated to dryness and 50 μl of CH₂Cl₂ was added. The injected volume was 3 μl. The whole sample set was analysed in exactly the same way, which makes it possible to compare between the samples. In our analysis the hyoscyamine content was measured as the sum of hyoscyamine and its isomer littorine, because of the difficult separation of these isomers in analytical systems. We observed no significant changes in the growth pattern between the transformed and untransformed roots. The contents of hyoscyamine in the hairy roots after 21 d was calculated and it was found that the hyoscyamine content was on average 25-fold higher in transformed roots compared to control roots, varying from 12-fold (clone C) to 62-fold (clone B). In addition to possessing extremely high hyoscyamine content, in the chromatogram of clone B also several (5-10) new peaks were found which are currently being identified.

### Materials and Methods

Alkaloid Analysis

Nicotiana tabacum BY-2 cells were cultured in modified Linsmaier-Skoog (LS) medium (Linsmaier & Skoog, 1965), as described by Nagata & Kumagai (1999). First, the growth curve of BY-2 cell culture was determined (FIG. 2) and the late exponential phase was used in elicitation experiments. Since the ability of high auxin concentration to inhibit the biosynthesis of nicotine is well known (Hibi et al., 1994; Ishikawa, et al., 1994), the six-day-old culture was prior elicitation washed and diluted 10-fold with fresh hormone free medium. After 12 hours, the cells were treated with methyl jasmonate (MeJA). MeJA (cis-form, Duchefa M0918) dissolved in dimethyl sulfoxide (DMSO) and was added to the culture medium at a final concentration of 50 μM. Same amount of DMSO alone served as a control. Samples for cDNA-AFLP analysis were taken at 0, 1, 2, 3, 4, 5, 6, 7, 8, 9, 10, 11, 12, 13, 14, 15, 16, 18, 20, 24, 36, 48, and 98 hours after jasmonate addition or at 0, 2, 4, 6, 8, 10, 12, 14, 16, 24, 36, 48, and 98 hours after DMSO addition, respectively. For alkaloid analysis, the samples were taken at 0, 12, 14, 24, 48 and 98 hours. Three replicate shake flasks pooled together yielded the total culture volume of 75 ml. After filtering (Miracloth) under vacuum the cells were lyophilized. Lyophilized cell samples were extracted for GC-MS analysis by a modified method described by Furuya et al. (1971). Cells were weighed and 25 μg of internal standard (5α-cholestan) was added. The samples were made alkaline with ammonia (10% (v/v), 1 ml) and water (2 ml) was added. Alkaloids were extracted by vortexing with 2 ml of dicloromethane. After 30 min the samples were centrifuged (2000 rpm, 10 min) and the lower organic layer was separated and transferred into glass vials. The samples were concentrated to 50 μl and 3 μl aliquots were injected to GC-MS. In some cases (for derivatization of free fatty acids and more polar compounds) the samples were silylated prior to GC-MS analysis. After evaporation to dryness, 25 μl of dichloromethane was added and silylation was performed by N-methyl-N-(trimethylsilyl)-trifluoro-acetamide (Pierce, Rockford, USA) at 120° C. for 20 min.

Analysis of Polyamines

Approx. 200 mg FW cells were homogenised using a mortar and pestle with 10 vol 4% (v/v) perchloric acid (PCA), and the homogenate left on ice for 60 min then centrifuged at 20 000 g for 30 min. The pellets were washed twice by resuspending in PCA and centrifugation at 15 000 g for 5 min. The washed pellets were resuspended in the original volume of PCA. Aliquots (0.3 ml) of the supernatants and resuspended pellets were hydrolysed by adding an equal volume of 12 N HCl at 110° C. overnight in order to release PCA-soluble and -insoluble conjugates, respectively. Hydrolysed samples were taken to dryness and resuspended in 0.3 ml 4% PCA. Aliquots (0.2 ml) of the supernatants and of the hydrolysed supernatants and pellets were derivatised with dansyl chloride (Sigma) after alkalinisation with 1.5 M Na₂CO₃ (1 h at 60° C.), and dansylated amines extracted in toluene. Standard putrescine, methylputrescine, spermidine and spermine solutions (1 mM in 4% PCA) were subjected to the same procedure. Samples were injected into a fixed 20-PI loop of an HPLC (Jasco) for loading onto a reverse-phase C18 column (Spherisorb S5 ODS2, 5-μm particle size 4.6×250 mm. Phase Sepand eluted with a programmed acetonitrile-water 5-step gradient as follows: 60 to 70% acetonitrile in 5.5. min, 70 to 80% in 1.5 min, 80 to 100% in 2 min, 100% for 2 min, 100 to 70% in 2 min and 70 to 60% in 2 min, at a flow rate of 1.0 ml min⁻¹. Eluted peaks were detected by a spectrofluorometer (excitation 365 nm, emission 510 nm), and their retention times and areas recorded and integrated by an attached computer using the Borwin 1.21.60 software package.

Analysis of Sesquiterpenes

The sesquiterpenoid alkaloids were detected by GC-MS. The extraction was performed as described in the section of alkaloid analysis. The preliminary identification is based on the MS fragmentation pattern.

Detection of Phenylpropanoids by TLC

Phenylpropanoids (coumarins and flavonoids) were extracted from elicited BY-2 cells or form the culture filtrate as described by Sharan et al. (1998). The methanol solutions obtained were concentrated and evaluated qualitatively by TLC using silica gel plates with fluorescent indicator UV₂₅₄ (Polygram® SIL G/UV₂₅₄, Macherey-Nagel, Düren, Germany) developed with ethylacetate:methanol:water (75:15:10). Spots were visualized under UV₂₆₀ after staining with AlCl₂ (by spraying with a 1% ethanolic solution).

RNA Extraction and cDNA Synthesis

Total RNA was prepared by LiCl precipitation (Sambrook, 1989). Starting from 5 μg total RNA, first-strand cDNA was synthesized by reverse transcription with a biotinylated oligo-dT₂₅ primer (Genset, Paris, France) and Superscript II (Life Technologies, Gaithersburg, Md.). Second-strand synthesis was performed by strand displacement with Escherichia coli ligase (Life Technologies), DNA polymerase I (USB, Cleveland, Ohio) and RNAse-H (USB).

cDNA-AFLP Analysis

500 nanograms of double-stranded cDNA was used for AFLP analysis as described (Vos et al., 1995; Bachem et al., 1996) with modifications. The restriction enzymes used were BstYI and MseI (Biolabs) and the digestion was performed in two separate steps. After the first restriction digest with one of the enzymes, the 3′ end fragments were collected on Dyna beads (Dynal, Oslo, Norway) by their biotinylated tail, while the other fragments were washed away. After digestion with the second enzyme, the released restriction fragments were collected and used as templates in the subsequent AFLP steps. The adapters used were as follows: for BstYI, 5′-CTCGTAGACTGCGTAGT-3′ (SEQ ID NO:_) and 5′-GATCACTACGCAGTCTAC-3′ (SEQ ID NO:_), and for MseI, 5′-GACGATGAGTCCTGAG-3′ (SEQ ID NO:_) and 5′-TACTCAGGACTCAT-3′ (SEQ ID NO:_); the primers for BstYI and MseI were 5′-GACTGCGTAGTGATC(T/C)N₁₋₂-3′ (SEQ ID NO:_) and 5′-GATGAGTCCTGAGTAAN₁₋₂-3′ (SEQ ID NO:_), respectively. For preamplifications, an MseI primer without selective nucleotides was combined with a BstYI primer containing either a T or a C as nucleotide at the 3′ extremity. PCR conditions were as described (Vos et al., 1995). The obtained amplification mixtures were diluted 600-fold and 5 μl was used for selective amplifications using a ³²P-labeled BstYI primer and the Amplitaq-Gold polymerase (Roche Diagnostics, Brussels, BE). Amplification products were separated on 5% polyacrylamide gels using the Sequigel system (Biorad). Dried gels were exposed to Kodak Biomax films as well as scanned in a phospholmager (Amersham Pharmacia Biotech, Little Chalfont, UK).

Quantitative Measurements of the Expression Profiles and Data Analysis

Scanned gel images were quantitatively analyzed using the AFLP QuantarPro image analysis software (Keygene N. V., Wageningen, N L). This software was designed for accurate lane definition, fragment detection, and quantification of band intensities. All visible AFLP fragments were scored and individual band intensities in each lane were measured. The raw data obtained were first corrected for differences in total lane intensities which may occur due to loading errors or differences in the efficiency of PCR amplification with a given primer combination for one or more time points. The correction factors were calculated based on constant bands throughout the time course. For each primer combination, a minimum of 10 invariable bands were selected and the intensity values were summed per lane. Each summed value was divided by the maximal summed value to give the correction factors. Finally, all raw values generated by QuantarPro were divided by these correction factors. A coefficient of variation (CV) was calculated by dividing the maximum value across the time course by the minimum value. This CV was used to establish a cut-off value and expression profiles with a CV less than 4.0 were considered to be constitutive throughout the time course. Although differential and constant bands can be discriminated by visual scoring, QuantarPro-mediated analysis is more sensitive and reliable. As such, transcript tags that had been identified as jasmonate-modulated after visual scoring were excluded from the final data set because they had a CV lower than our threshold level. Vice versa additional jasmonate-modulated transcripts were identified that had been missed by the visual scoring. Subsequently, each individual gene expression profile was variance-normalized by standard statistical approaches as used for microarray-derived data (Tavazoie et al., 1999). For each transcript, the mean expression value across the time course of the DMSO-treated samples was subtracted from each individual data point after which the obtained value was divided by the standard deviation. The Cluster and TreeView software (Eisen et al., 1998) was used for average linkage hierarchical clustering.

Characterization of AFLP Fragments.

Bands corresponding to differentially expressed transcripts were cut out from the gel and the DNA was eluted and reamplified under the same conditions as for selective amplification. Sequence information was obtained by direct sequencing of the reamplified PCR product with the selective BstYI primer or after cloning the fragments in pGEM-T easy (Promega, Madison, Wis.) and sequencing individual clones. The sequences obtained were compared against nucleotide and protein sequences in the publicly available databases by BLAST sequence alignments (Altschul et al., 1997).

Isolation of Full-Length cDNA Clones.

Two strategies were followed to obtain full-length cDNA clones corresponding to the short sequence tags isolated in the cDNA-AFLP analysis. In the first method the use of gene-specific primers, RT-PCR, 5′- and 3′-RACE (InVitroGen Life Technologies) techniques were combined to yield a full-length cDNA clone. For the second strategy a cDNA library from elicitor treated BY-2 cells was generated in the pCMV-SPORT6 vector (Gateway™, InVitrogen Life Technologies) using a mixture of samples taken at different time points after jasmonate elicitation. This library was screened by PCR or colony hybridization using gene-specific primers or probes respectively.

TABLE 1

Sequences with homology to known gene

| | | | |
|---|---|---|---|
| Seq code | SEQUENCE | Annotation | SEQ ID N° |

| | | | |
|---|---|---|---|
| BAP1a | TTATCTCGGCGGCGAATCTACCCCACTCTTCGAAGA | envelope | SEQ ID N° 1 |
| | TAACGCTCATTTTGTTACCATACTCACCTCTCTGAA | polyprotein like | |
| | CAAACACACAAATACACACGAACTCACAGTCCAAA | protein | |
| | TAGCTAAAACAAAGGTTTTTGAATTGAAATTGAAG | | |
| | CTCAGATC | | |
| BAP1b | GATCCTCTGAGGCTATTATGCTTGCTGGATTAGCTT | glutamate | SEQ ID N° 2 |
| | TCAAGAGAAAATGGCAAAATAAAACGAAAGCCCA | decarboxylase | |
| | AGGCAAGCCCTGTGACAAGCCCAATATTGTCACTG | | |
| | GTGCCAATGTCCAGGTGTGGTTGGGGCAAATTCGCC | | |
| | GCCGAGATA | | |
| BAP2 | GATCCAGACCATGCACACAAACACAAGATAGAAG | abscisic stress | SEQ ID N° 3 |
| | AAGAGATAGCAGCAGCTGCTGCAGTTGGGGCAAAT | ripening protein | |
| | TCGCCGCCGAGATA | | |
| BAP4a | CAGAGCATGCACACAAACACAAGATAGAGGAAGA | abscisic stress | SEQ ID N° 4 |
| | GATAGCAGCTGCTGCTGCAGTTGCGTAGACGGCGT | ripening protein | |
| | AGTGATCCAGAGCATGCACACAAACACAAGATAGA | | |
| | GGAAGAGATAGCAGCTGCTGCTGCAGTTTGGGGCAA | | |
| | ATTCGCCGCCGAGATCAG | | |
| BAP4b | GAGAAGACCA AGAAGAAGCA AAGGAAGAAT | AP2-domain DNA- | SEQ ID N° 5 |
| | CTTTATAGGG GAATCCGACA GCGTCCATGG | binding protein | |
| | GGAAAATTCG CCGCCGAGAT GAG | | |
| BMAP1 | AGGAGCTGAACACACACCAACACCAACACTAACA | putative protein | SEQ ID N° 6 |
| | GGAGCTCCGTGGAGCACTGGCTTATTCGATTGTCAT | At1g52200 [A. | |
| | TTTGGACCAAACTAATGCTACTACGACAGCATTTTTA | thaliana] | |
| | CCTTGTGTGACATGTGGACCGTCGGCTGCATA | | |
| BMAP2a | CTAGTTTGGAATATGAGTTCTCTGCTCTTCGAGAAG | putative potassium | SEQ ID N° 7 |
| | CCACAGAATCTGGATTTACATATTTGCTTGGACATG | transporter | |
| | TGGACCGTCGGCTGCAT | | |
| C1 | GGGGGAGAAG CGAAGGTCTA AATCTAACCA | 1,4-benzoquinone | SEQ ID N° 8 |
| | AATCCCCAAA ATGGCTACCA AAGTTTACAT | reductase-like | |
| | CGTATACTAT TCAATGTATG GTCATGTGGA | | |
| | GAAACTAGCA GAAGAGATAA AGAAAGGGGC | | |
| | AGCTTCTGTT GAAGGAGTTG AAGCTAAATT | | |
| | GTGGCAAGTA CCTGAAACGC TGTCGGAAGA | | |
| | TGTGCTAGCA AAAATGAGTG CACCTCCAAA | | |
| | GAGTGATGTG GCTGTTATAA CACCTCAAGA | | |
| | GCTTGCTGAA GCAGATGGTA TCATTTTTGG | | |
| | ATTCCCTACG AGATTCGGAA TGATGGCTGC | | |
| | TCAGTTTAAA GCATTCCTTG ATGCAACTGG | | |
| | AGGTCTATGG AGAACACAAC AACTAGCTGG | | |
| | CAAGGCTGCC GGCATATTCT ATAGCACTGG | | |
| | ATCCCAAGGC GGTGGCCAAG AAACTACACC | | |
| | GTTGACTGCG ATAACTCAGC TTGTTCACCA | | |
| | CGGGATGATC TTTGTACCTA TCGGATACAC | | |
| | ATTCGGTGCT GGTATGTTTG AAATGGAGAA | | |
| | AGTGAAAGGA GGAAGTCCAT ATGGGGCGGG | | |
| | AACATTTGCT GGGGATGGCT CGAGACAGCC | | |
| | ATCCGATCTT GAATTGCAGC AGGCGTTTCA | | |
| | CCAAGGTAAA TACATTGCCG GTATTGCCAA | | |
| | GAAACTCAAG GGTGCAGCCT AATTTCTCTC | | |
| | CTGCAAAGAT AATCTTTGCA TTCACACATT | | |
| | TCTTATAAAA TTTGAAAAAA GTACAAAATT | | |
| | TATCTTTGTG ATTGTTGAAG TCTTTTTTTT | | |
| | TTCCTTTATT GGGTATGAAA TCTCATCTAT | | |
| | ATGTGTCTGA TTCACAGTAA TTGTGTGTGT | | |
| | CAAAAGTACC AAATTGTGTT TTAAAATGGT | | |
| | TGCAAATACA A | | |
| C10 | GATCCCAGAA TAGCGTTGAG ATAGATGATC | cystatin | SEQ ID N° 9 |
| | TTGCACGTTT TGCTATCCAA GATTATAACA | | |
| | AAAAACAGAA TGCTCTTTTG GAGTTTGGAA | | |
| | AGGTTGTGAA TGTGAAACAA CAGGTAGTTG | | |
| | CTGGAACCAT ATACTATATA ACACTCGAGG | | |
| | CAATTGAGGG CGGAAAGAAG AAAGTATATG | | |
| | AAGCCAAGAT ATGGGTTA | | |
| C101 | GATCCAATCG TTGGAATTTT GACAAGGCAT | chloride channel | SEQ ID N° 10 |
| | GACTTTATGC CAGAGGATAT AAAGGGACTG | protein C1C-1 | |
| | TACCCACATT TGGTCCATCA CAAGTAGCAG | | |
| | AGAGAAGCTA GCTCTTCCAA CAGGCAATCG | | |
| | GGCAACCATT ATTTGGGGAG TGTTATACAC | | |
| | ACATTCCACA TTGAGCTCTG TACACAATCT | | |
| | TCCCAAATTT TCTCATTGAC AAAATTGAAT | | |
| | TTAGTAGTCT CAATTAGAGC AAAAATTCTC | | |
| | CCTTACTTTG AATTGTTGAA CTTTCTTGTT | | |
| | TTTGGTGGTT TA | | |
| C102 | TGACGATGAG TCCCGAGTAA ACAAAATTGC | putative protein | SEQ ID N° 11 |
| | CATCTCCATC ACATCCTAGT GACACTAGTT | At5g47690 [A. | |
| | CACAGAGTTT GGCATCATGG ATGTCCAGAC | thaliana] | |
| | AATTGTGTAG ACCGACTGAA ATATGTCTGT | | |
| | TTATGAACTA AACACAAACT AATGACTTTC | | |
| | CTACATGTGG CGCTAATTGA AGAGAAGAGA | | |
| | TCCAAATACC CGTTATGAAG GCATATCAAC | | |
| | ACTACTACCA ATGAGTGTAT GGAACTTATA | | |
| | GAGCATTTAT CATCCTCTTC ATCTCAGTGG | | |
| | ACCTCCTTGG ATCACTACGC AGTCA | | |
| C104 | GATCCAAGTG ACACCACTAA GCAACAATGA | methylcrotonoyl- | SEQ ID N° 12 |
| | CTATAGAGTT GAAGTCAATG GTCTAAGCCT | CoA carboxylase | |
| | GAATGTCTGC TTGGCTGCTT ATTCCAAGGA | | |
| | TCAAATTGAG CATATTCACA TCTGGCAGGG | | |
| | CAACTGCCAG CATCACTTCA AACAAAGGAT | | |
| | GGGCCTTGAA ATCTTTGATG ATGATGAAAC | | |
| | GATAGACAAG CCTGCTCGCA TGGCAACATC | | |
| | TTATCCTTCT GGCACAGTGG TTGCACCCAT | | |
| | GGCTGGTCTA CTGGTTTA | | |
| C105 | GATCCAAGAA GAGAAAATGT CTGGTGAAGA | 40S ribosomal | SEQ ID N° 13 |
| | GGCTGTTGTT GCTGAGACCC CAGCTCCCGC | protein S12 | |
| | TGCCGCTCTT GGTGAGCCCA TGGATATCAT | | |
| | GACGGCATTG CAACTTGTCC TCAGGAAATC | | |
| | ACGGGCTCAT GGTGGGTTAG CTAAAGGCCT | | |
| | TCACGAGGGT GCAAAGGTCA TCGAGAAGCA | | |
| | TGCTGCCCAA CTTTGTGTAT TGGCAGAGGA | | |
| | CTGCGACCAA CCAGACTATG TGAAATTGGT | | |
| | CAAAGCGCTA TGTGCTGATC ACAATGTTA | | |
| C106 | GATCCAACCCAATAACACCTTCAAATGCCACATGG | putative protein | SEQ ID N° 14 |
| | TCCAGCTGAATGTTTTTTGGACACTTTAGAGGGTTG | At1g07080 [A. | |
| | TGCAATTTGATGCCTGGCCAGATTTGAATGAACATTT | thaliana] | |
| | TCCTTTCATTTACTGTGTGGAAAGTTTGGTCTACCA | | |
| | TAAGAATTATACCCAGTGGGAAACATGTTTTTGAAA | | |
| | AACTGAATTTGAAGGCAAAGCTTGTTA | | |
| C107 | TTTGAACCCTGATAACAAAGCTGGGAGGATTACAA | 3-deoxy-D-arabino- | SEQ ID N° 15 |
| | AATTTACCAGAATGGGAGCAGAGAACATGAGGGTT | heptulosonate 7- | |
| | A | phoshate synthase | |
| C108 | AATTACAATA CTTATAGTTT CGATGGAAAG | putative protein | SEQ ID N° 16 |
| | AAGAAGCTTG TGCTTTCTAC AACTAGCTGG | At1g54320 [A. | |
| | ATTGGCGGAA AGAATGATTT TCTTGGTATT | thaliana] | |
| | GCTTATCTTA CTGTAGGTGG ATTGTGTTTC | | |
| | TTTCTGGCCA TGGCTTTCAC GATCGTGTAT | | |
| | CTAGTTAAGC CAAGGCAGCT TGGAGATCCA | | |
| | ACATACTTTG CGTGGAACCG GAACCCGGGA | | |
| | GGTAACTAGT ATGCAAATGA AGTCTTTTGG | | |
| | CTTGAGCGCT TTACCATCTA AGGTTGATGT | | |
| | TGACAAAGCT TGTGTCTTGT AGCAGCTATC | | |
| | TGTCTACAAG TTCTTTTTTT TTGAAATGTT | | |
| | CTGCATATAC TTTTAAACTC AATTTGCTAG | | |
| | GAAAACAATG ATATGTAATG AAGTATTTTC | | |
| | CCTTTGTTAA GTGTTTATCC AAAATTATGT | | |
| | ATGTACAATG GAAGTAATTG CTTAAAGGAC | | |
| | TTGAATGATG CC | | |
| C109 | GATCCAAGTGCGGACGGTGTTCACCATGTAAACCG | putative protein | SEQ ID N° 17 |
| | GTTCGAGTCTCCGTTCAACCTGGTTTTACCTCTACT | At3g22820 [A. | |
| | TTAGAGTACTACCCTGAAGCTTTGGAGGTGCAAGTG | thaliana] | |
| | TCGCAACAAACTCTTCATGCCTTA | | |
| C11 | GATCCCACAA TATTCATATG TAACTCCGAC | putative protein | SEQ ID N° 18 |
| | GAAATGGAAT TTGGTGACGT GGTTTCAGCC | At2g23690 [A. | |
| | ATAAGTGCCG ACGAGGAGCT TCAACCGGGT | thaliana] | |
| | CAACTTTACT TTGCGTTGCC TTTGAGCAAT | | |
| | CTGAAACGTA GGCTTCAGGC TGAGGAAATG | | |
| | GCAGCATTAG CCGTTA | | |
| C110b | TAAGGCTCTCTTCAGAAGCTACGTGTGCCGATGATC | CTR1-like protein | SEQ ID N° 19 |
| | CCAATTTCTTGGATC | kinase | |
| C110c | TAAGGTGGTTGAGTTTGAACTTCCACGGCAACAAT | putative protein | SEQ ID N° 20 |
| | GTATAGTCTACTTGGATC | At2g46260 [A. | |
| | | thaliana] | |
| C111 | GATCCAAGAA TAAAGGGTCT ATTTTTTCAC | putative protein | SEQ ID N° 21 |
| | CAAACAACAT TCAGTATTGG CTTGTCCAAA | At2g46750 [A. | |
| | GTAAAAAACT TTATACAAGA TGTGCAAAAA | thaliana] | |
| | CTTGTGGTTT TACAGCCTAA GGCATTATGT | | |
| | GGTTTAGACC TATACAGTGG AATCCTAATG | | |
| | AGGTATGTCA CAGCTTCAAA TGCTTACTTG | | |
| | GGACATCAAG AAGATGCAGT GGATTTTGAT | | |
| | ATAACATATT ATAGAAGCAA AAATCCATTG | | |
| | ACTCCTAGGT TATATGAAGA TATTCTTGAA | | |
| | GAAATAGAGC AAATGGCGAT GTTCAAATAT | | |
| | GGAGCAGAAC CTCACTGGGG GAAGAATCGT | | |
| | AATGTGGCAT TCATTGATGT GATTA | | |
| C112 | TAGCGGATAACAATTTCACACAGGAAACAGCTATG | myosin-heavy-chain | SEQ ID N° 22 |
| | ACCATTAGGCCTATTTAGGTGACACTATAGAACAA | kinase-like protein | |
| | GTTTGTACAAAAAAGCAGGCTGGTACCGGTCCGGA | | |
| | ATTCCCGGGATTTCTTCTTCATCATCGATTTTTAGCT | | |
| | CAAATGTCGTCTGCTTCTACAGAAAATCGTAGCCTT | | |
| | TGGACAGAGATCCGAGAATCAATAAGGAGCATATT | | |
| | GAAAGCTAATTGTGGCCATTTTCATACTCTTTTTAT | | |
| | CCTCTTCCTCTTGCCTATCTTTTTCTCTCTCGTCGTT | | |
| | TATCCTTCTTTCCACCTTGCCCTCTTTCATCCGGACT | | |
| | ATGATTTCACTCAACCAGTTCAATTTTCACACTTTT | | |
| | TAAGTTCACACTTCGAAATTATTGTACCCATAGTAT | | |
| | TTACTCTGTTTCTGGTCCTCCTTTTCCTCTGTGCTGT | | |
| | AGCCACGATACATACAGCGCGCTTCATGTATCCTA | | |
| | TGGTAGACCTATCAACCTCGTTTCCTCTATTAAATC | | |
| | TATCAGAAATTCCTTCTTCCCCCTTCTCTCCACCTTT | | |
| | ATCGTTTCGCATACCATTTTCATTTCAATCGCTCTC | | |
| | GTTTTCTCCCTTGTCTTGGTTTTTTTAGTCCAGGTTC | | |
| | TTCAAACTCTTGGATTAATTGAACTAAAATACGACT | | |
| | CGAATCATTTCTTGTTTTTGGTTATTCCCGCGTTGAT | | |
| | TGTGCTCGTGCCAGTTTTGATATGGTTGCAGGTTAA | | |
| | TTGGTCATTAGCTTATGTGATAGCAGTAGTCGAATC | | |
| | GAAATGGGGTTTCGAAACACTAAGGAGAAGTGCCT | | |
| | ATTTGGTAAAGGGGAAGAGATCGGTAGCTTTGTCG | | |
| | ATGATGCTGTTATACGGGCTTTTGATGGGAATAATG | | |
| | GTGGTTTTAGGTGCCATGTATTTAGTCATTATGGAT | | |
| | GCAGCGAAGGGTCGTCAATGGAGAAGTTCAGGGGT | | |
| | AATATTACAGACTGCTATGAGTTTCAATAACTAGCT | | |
| | ATCTCATGATGAGTCAATTTCTTGTGGGGAATGTTG | | |
| | TTTTATATCTGCGTTGCAACGACTTGAATGGTGAAA | | |
| | AATTGCCCTTGGAAATCGAGCATCTTCTTCTTCATC | | |
| | AATCTTTAGCTAATGATCACCCACCTCCAATGTTGT | | |
| | CAGCTTCAACGAAAAATCTTAGCCTATGGACAGAG | | |
| | GTCGTAGAATCAGCAATGAGCATATTCAAAGCCAA | | |
| | TTCTGGCCATTTCCATGCTCTTTCAATCCTCTTCCTC | | |
| | TTGCCTATCTCTTTCTTTCTCGTCGTGTATCCTTCTT | | |
| | TCCACCTTGCTCTCTTTCATCCGAACTATGATTTCAT | | |
| | CAGTTTCGCTCAACGCCATCTTTTCCTTTCAAATTTC | | |
| | GAAATTATTGTACCAACATCGTACTCTTTGTTTTTG | | |
| | GTCCTCCTTTTCCTATGCGCCGTAGCCACAACTACA | | |
| | TATAGCGCGGTTCATGCATCCTATAGTAGACCTATA | | |
| | AACCTCGTTTTGTCGATAAAATCGATCAGAAAGTCT | | |
| | TTGTTCCCCCTTCTCTCCACCTTACTCGTTTCGCATA | | |
| | CCATTTTCATTTCAATCACTCTTGTTTTCACCCTAGT | | |
| | CTTGACTATTTTAGTTTCAAATTCTTCAACCTCTCGG | | |
| | ACTAATTGAAATCAAATACGATTCGGATCACTTCTT | | |
| | GCTTTTGGCTATTCCTGCTTTGGTAGTGCTCGTGCC | | |
| | AGTTCTGCTATGGCTACATGTTAACTGGTCATTAGC | | |
| | TTATGTGATAGCAGTAATTGAATCGAAATGGGGTT | | |
| | ACGAAACATTGAGGAGAAGTTCCTATTTTGGTGAAG | | |
| | GGGCAAAGATGGGTAGCTTTTGGGATATATTTTATA | | |
| | TTACGGGCTTTCAATGGGAATAATGATGGTTTGTGG | | |
| | TTCAATGTTTTTTGTCATTATGGGTGTAGCGAAGGG | | |
| | TAATAAGTGGAGGAGCTTGGACGTGATACTGCAGA | | |
| | CTGCGCTAGTTTCAGTGATGGGATATCTGACGATG | | |
| | AATCAATATCTTGTGGCGAACGTGGTTTTGTATATG | | |
| | AAATGCAAGGATTTGAGCGTTGAAAAATTGCAGTC | | |
| | GGAAACTGGAGGCGAGTACGTTCCCCTGCCCTTGG | | |
| | ATGAGAAGAATCAAGCTATTTGAATAATLTGTAAAC | | |
| | AGTGAATCTGGTAGGCTATTTGTGTAACACTTCCTT | | |
| | TGATTAATGCTTTGTACGAGTATAATGTTTGGTTGTC | | |
| | TTTGTAGAAAGTTAAACGTGTGTGCTAAATGTTCTG | | |
| | CTCGTCTTTCCTGTTTGTTGAATATTTGAATAAAAAC | | |
| C114 | GATCCAAAAGTATGCACGATCTTTCAAGCCATGAT | diacylglycerol | SEQ ID N° 23 |
| | AATCATGATGGTGATGATGGGGATAGTGGTGAAGA | kinase | |
| | GGATTCGGTTGTGGAAGAGCAGAGGAAGTTTGGGG | | |
| | CAGCAGACACATTTCAAAATTTCCTGATGAAGTTTGAC | | |
| | ATTTCTCATCTCAGTTGATTCTGTTATCTCTCGTCGT | | |
| | TCAAAATTTTGCTTTCTACTACAACCTCCATATTA | | |
| C116a | GATCCAAGATGGGAAGAGGATTTTACTTTTTGTGTTTG | putative calcium | SEQ ID N° 24 |
| | GAGGAGCCTCCTGTGAATGATAGGCTGCATTTGGA | lipid binding protein | |
| | AGTTCTCAGCACCTCAATGAGGATTGGCCTATTGCA | | |
| | TCCTAAGGAGGTATTGGGTTATATTGATATAAGCCT | | |
| | TTCCGATGTTGTTA | | |
| C116b | GATCCAAGCCAAAGTTGGAACAAGGCTCTCAAACT | subtilisin-like serine | SEQ ID N° 25 |
| | ATCACAAATAGCATCGACCAAGAAGGAGTTTGAAG | protease | |
| | CGCTTGGTGTTTTATTTTCTAGTCATTATTATATGAG | | |
| | TACAATGACAATATGAACAATAAAGTATTGTATAG | | |
| | TATGGTTTTATATTA | | |
| C117a | TAATGCCTAAAGTGTCATCTTATAATGCTTTGGATC | homeodomain | SEQ ID N° 26 |
| | ACTTGTCATTATTTTCTTCAACTTACACTCAGTTATT | protein | |
| | GGATC | | |
| C117c | GATCCAAGTTGTGGCGGCAAGTTGGCGAGTCGTTT | putative DNA- | SEQ ID N° 27 |
| | A | binding protein | |
| C117d | GATCCAAGTTCTTTGAGCAGGGTCTAAATAATCTAT | putative protein | SEQ ID N° 28 |
| | CATTGGAGGAAAAGGCCAACCGGAAGGATTCGGC | P0410E01 [Oryza | |
| | GATATTA | sativa] | |
| C118c | GATCCAAGCAGATATTGAGATGAAATGTTTTCAGT | putative eukaryotic | SEQ ID N° 29 |
| | TTGATCGGTGTTATTCACATTA | translation initiation | |
| | | factor 2 alpha | |
| C118d | GATCCAACGAAAAACAAGAAGCGCCCTGATTTTGT | putative cellulose | SEQ ID N° 30 |
| | GAAGGATCGACGTTGGATTA | synthase | |
| C119 | GATCCACAATCTCTTGGAATGGATTGCAGTGACAC | putative ABC | SEQ ID N° 31 |
| | TATTCTCGGAAATCCAACAGAATGTGAACTATACA | transporter | |
| | AAGCCCTTGGAAGTACAATTCACAACAACTTGTCT | | |
| | GGCTTTAGCGAGAATACTGTTAGAAAATCCATCTA | | |
| | TACTGATATCGTAGTGTTTA | | |
| C12 | GATCCCAACT ATTGACACCA TACCCCGGAA | aldehyde oxidase | SEQ ID N° 32 |
| | TTTCAACGTT CATTTGGTAA ACAGCGGACA | | |
| | TCATGAAAAA CGGGTTCTCT CTTCCAAAGC | | |
| | ATCTGGTGAA CCGCCACTGC TATTGGCAGC | | |
| | TTCAGTCCAT TGTGCAACAA GAGCAGCCGT TA | | |
| C120 | TAACGAAGTTGCCAAGGGTTTTGGTGGATC | 40S ribosomal | SEQ ID N° 33 |
| | | protein S2 | |
| C121 | GATCCACACCCACATGTGCTACTCCAACTTCAACG | methionine synthase | SEQ ID N° 34 |
| | ACATTATCCACTCCATCATAGACATGGATGCTGATG | | |
| | TGATCACCATTGAGAACTCACGTTCTGATGAGAAA | | |
| | CTCCTCTCAGTTTTCAGGGAGGGAGTGAAGTACGG | | |
| | AGCTGGCATTGGTCCCGGTGTCTATGACATCCACTC | | |
| | TCCAAGAATACCATCCACAGAGGAGCATAGCTGAT | | |
| | GAGGTTA | | |
| C124b | GATCCACTAATTATTGGAACACAAGTAAAGCCACG | membrane protein | SEQ ID N° 35 |
| | CGATGAATTGTTTTGGTTTGGGAAACCGAAGATAC | Mlo4 | |
| | TATTACGGTTA | | |
| C125 | TTACGTTTCTGTTTCTGAGTCTGGTTCTCAGGACT | methionine synthase | SEQ ID N° 36 |
| | CATCGTCAAGAACTCACGTTCTGATCAGAAACTCCT | | |
| | CTCAGTTTTCAGGGAGGGAGTGAAGTACCGGAGCT | | |
| | GGCATTGGTCCCGGTGTCTATGACATCCACTCTCCA | | |
| | AAGAATACCATNCACAGAGGAGATAG | | |
| C126 | TAAGCCCGCACGAGAAGGTGATTTGGAGGGAATTC | cathepsin B-like | SEQ ID N° 37 |
| | CACTTCTAACTCATCCTAAACTTTCGGAGCTACCAA | cysteine proteinase | |
| | AAGAGTTTGATGCACGAAAAGCTTGGCCTCAATGT | | |
| | AGCACTATCGGAAGAATTCTGGATCAGGGACATTG | | |
| | CGGTTCTTGTTGGGCTTTTGGTGCTGTTGAATCGTT | | |
| | GTCTGATCGTTTCTGTATCCATCACAACTTGAATAT | | |
| | CTCTCTGTCTGTAAATGATCTGCTAGCATGCTGTGG | | |
| | CTTTTTATGTGGATC | | |
| C127 | AGCAGGCTGGTACCGGTCCGGAATTCCCGGGATTG | auxin-responsive | SEQ ID N° 38 |
| | TGTGTACAAATTACTAATATAGTTTCTTCACAATTA | GH3-like protein | |
| | TGGAAAGAAACGTAGCTAATGAGGCACCAAAGGC | | |
| | CACAATAATGGCGGAGGATTACAAGAAGGATCTTG | | |
| | AGTTCATTGAAGAGGTGACTAGCAATGTTGATGAG | | |
| | GTCCAAATGAGAGTTCTTGCTGAAATCCTCTCCCAG | | |
| | AATGCACATGTTGAGTACTTGCAACGCCATAATCTC | | |
| | AATGGCAGCACTGATAGAGAGACATTCAAGAAAGT | | |
| | CGTACCTGTCATCACTTATGAAGATATTCAGCCTGA | | |
| | TATCAAACGCATAGCCTATGGTGATAAATCTCCTAT | | |
| | CCTCTGCTCCCAACCCATCTCTGAATTATTGTCAAG | | |
| | TTCTGGCACCTCTGGAGGGGAGAGCAAATTGATAC | | |
| | CAACAACAGAGCCAGAGATTGGGAAGAGACTACA | | |
| | GCTTCACAAACTTGTGATGTCTGTGTTGAGCCAAGT | | |
| | GGCTCCAGATTCTGGAAAGGGCAAAGGAATGTATT | | |
| | TCATGTTCATAAGCCCTGAACAAAAGACCCCAGGA | | |
| | GGATTATAGCTCGCTTTCTTACAACTAGTTATTTAC | | |
| | AATAGTCCTTACTLTCAACTACAGTCGTCTTCATAAC | | |
| | CCCCATTGTAACTACACTAGTCCAACTGCAGCCATT | | |
| | CTCTGCCCAGACTCTTACCAAAGCATGTATTTCCCAA | | |
| | ATGCTTTGTGGCCTCTGCCAAAACAACCAAGTCCTC | | |
| | CGTGTTGGCTCCTTTTTTGCGACCAGCTTCGTTCGT | | |
| | GCCATCCGATTCCTGGAGAAGCACTGGTCTCTACTT | | |
| | TGTAACGATATCCGAAGCGGAACCATTAACACTCA | | |
| | AATAACTGATCCTTTAGTGAGAGAGGCAGTGATGG | | |
| | AAGTCCTCAAACCTGACCCAACATTAGCTGATTTC | | |
| | TTGAGGTTGAATGCACCAAAGATTCATGGCAAGGG | | |
| | ATCATCACTAGGTTATGGCGTAATACCAAGTATGT | | |
| | GGATGTTTATTGTGACTGGATCCATGTCACAATATAT | | |
| | ACCGATACTTGATTATTACAGCAACAATCTCCCTCT | | |
| | TATCAGTACTCTGTATGCTTCCTCGGAAAGCCACTT | | |
| | TGGAATCAACTTGAACCCTTTTTGTAAGCCCAGTGA | | |
| | TGTCTCTTACACCCTTATTCCCACCATGTGCTATTTT | | |
| | GAGTTCTTACCGTATCGCGGAAACAGTGGAGTCAT | | |
| | TGATTCTATATCCATGCCCAAGTCGCTTAATGAGAA | | |
| | AGAACAACAACAATTGGTTGATTTGGCTGATGTCA | | |
| | AGATTGGCCAGGAGTACGAGCTTGTTGTTACCACA | | |
| | TATTCTGGACTCTACAGATATAGAGTCGGTGATGTG | | |
| | CTTCAGGTTGCTGGATACAAGAACAACGCGCCTCG | | |
| | ATTCAACTTCCTATGCCGGGAAAATGTAGTCTTGAG | | |
| | TATTGGTGCTGACTTCACTAATGAAGTTTGAGCTACA | | |
| | AAACGCAGTGAAAAATGCAGTGGGCAATCTGGTGC | | |
| | CATTTGATTCTCAGGTAACCGAGTACACCAGCTATG | | |
| | TCGATATTACCACCATTCCAAGCCACTATGTCATAT | | |
| | TCTGGGAACTGAATGCGAATGACTCTACCCTGGTTC | | |
| | CTCCTTTCAGTCTTTGAAGATTGTTGCCTCACAATTG | | |
| | AAGAATCTCTTAACTACTTCTACCGCGAGGGCCGT | | |
| | GCGTCTAATGAATCCATCGGGCCTCTAGAAATTAG | | |
| | GGTGTTGGAAATTGGAACTTTTGACAAGCTCATGG | | |
| | ACTACTGCATGAGCTTAGGTGCTTCCATGAACCAAT | | |
| | ACAAGACGCCCCGCTGTTTGAAATATGCACCCCTT | | |
| | ATTGAGCTATTGAACTCTAGGGTCGTGTCCAGCTAC | | |
| | TTCAGTCCCATGTGTCCAAAATGGGTTCCTGGCTAC | | |
| | AAGAAATGGGACGGCAACAATTAAATGTCAAACTT | | |
| | CCGATTTCCCTGCTTGTACCTTCATTCACTATCCAG | | |
| | AAAAAAGACAACCATTTGTGGATTATTTAGTCAAT | | |
| | CGTCATCCTAGCTAAGTTAGTCTTTCGTGAACATGG | | |
| | TATGGATTTGTATTTGTCACAAATAAAATATGGCAC | | |
| | TTTTTATTTTCAAAAAAAAAAAAAAA | | |
| C129b | GATCCACCAAGAAGAAAGCATATGGTGTATCTTGG | actin related protein | SEQ ID N° 39 |
| | AGGTGCGGTTCTGGCAGGAATTATGAAGGATGCCC | | |
| | CTGAGTTTTTGGATCAATAGACAAGATTATTTAGAA | | |
| | GAGGGAGTTGCTTGCTTA | | |
| C130 | GATCCACACAAAGCAGCTAGAGTTTGGTTAGGCAC | putative AP2 | SEQ ID N° 40 |
| | ATTTGATACAGCTGAAGCTGCCGCTAGAGCTTATG | domain containing | |
| | ATGAAGCTGCTCTTCGATTCAGAGGAAACAGAGCT | protein | |
| | AAGCTCAATTTCCCCGAAAATGTCCGCTTATTACCA | | |
| | CAACAACAACAACAATATCAACCCACAACAAGATC | | |
| | AGCC ATTCCAGCT | | |
| | CCTCAGCAGCTTCACAATTCCCATTA | | |
| C131 | TAATCCTTTG AGCGAACGTA TAGTGGAGCT | H+-transporting | SEQ ID N° 41 |
| | TCAATATGAT ATACGACTGA AATTAGGAGC | ATP synthase | |
| | CTTGATGCCT AAGGAGAGTG CCCAAAAAGT | protein 6 | |
| | TTTGGAAGCT TCCGAAGCTT TACATGGGGA | | |
| | AAGCAACAAT ATCGCCTTTC TTGAATACCT | | |
| | TTTGGAAGAT TTGCAGCAAA ACGGAGTAGG | | |
| | GGGAGAAGCC TATAAAGATG CGGTGGATC | | |
| C133 | GATCCACAAGTGATCCATCATTCTAAAGGCCATAC | putative protein | SEQ ID N° 42 |
| | CATACCAAAATTAGATGATAGCAGCCTTGAAATAA | At4g24380 [A. | |
| | TGCTTGGGTTTATTGAAAAAATTCAAAACCTGTGA | thaliana] | |
| | GACTGCACGAGGAATTA | | |
| C134 | GATCCACACCCCATATTGTTCACGCTCACCTCACTG | putative protein | SEQ ID N° 43 |
| | ACGAGCCACCATTA | PH1760 [P. | |
| | | horikoshii] | |
| C135a | GATCCAGTATTTGATAGTAGAAATGTCGCGTAAGG | high affinity sulfate | SEQ ID N° 44 |
| | AATTTCCAAAAACTATATTCTTCGAATTTTCTGTCC | transporter | |
| | CTGAGGTTTTCATTGAGTAACTTGATTCTTGCTCTC | | |
| | TTGCAGCTGTTACTGATATAGATACAAGTGGAATTC | | |
| | ATTCCTTAGAGGATTTGTTTA | | |
| C135b | GATCCAGGAAGTTGGAAGATATTGGTAATCAGTAC | NBS-LRR type | SEQ ID N° 45 |
| | TTTGATGAGTTACTATCAAGGTTTTGCTTCCTAGAT | resistance protein | |
| | GTGGTACAAGCTTTTGATGGAGAAATATTGGCTTGT | | |
| | AAGTTACACAATCTTGTGCATGATCTTGCACAGTCA | | |
| | GTGGCAGGTTCTGAATGTTTA | | |
| C136 | GATCCAGGTA GTTTCAAGAC ATTTGATCTT | Peroxidase | SEQ ID N° 46 |
| | AGCTATTACA AGCTTCTGCTCAAAAGGAGA | | |
| | GGCCTATTCC AATCTGATGC AGCTTTA | | |
| C137 | GATCCAGGAA AGGAGCATTG AGAAGGTGTA | Tobacco | SEQ ID N° 47 |
| | AAATGGATAT TGTGATATCTCAAAGGCCCC | retrotransposon | |
| | TCAGGTATGG CACTTTGTTT A | Ttol | |
| C140 | TGACTGCGTAGTGATCCAGGCAGCACTGGCTGAGT | glutaredoxin | SEQ ID N° 48 |
| | GGACTGGTCAGCGCACTGTGCCAAACGTCTTGATT | | |
| | GGCGGGAAGCACATTGGTGGCTGCGACGCCACAAC | | |
| | TGCGTTGCACAGGGAAGGGAAGCTTGTTCCTCTGC | | |
| | TAACTGAGGCTGGAGCACTTGCTAAATCTTCTTCTG | | |
| | CTTAGAGGATCAAATAGTCAGTTGTTTTTTTTAGTA | | |
| | AATCAGTCTCGTGAACTTA | | |
| C143 | CCTGACTCGGTTTCGTGATGCTAGCTCGTGAACCAAT | putative chorismate | SEQ ID N° 49 |
| | CATTTCCTCGAACCGACCGGCCATTCAAAACAAGCA | mutase/prephenate | |
| | TCGTATTCGCACATCACGAAGGAACAAGCGTCCTT | dehydratase | |
| | TTCAAAGTTCTATCGGCGTTTGCATTCAGAAACATA | | |
| | AGCTTA | | |
| C144 | TAAGCAAAAGAAACTCCAAGTATAGCACCCACAGA | caudal protein | SEQ ID N° 50 |
| | TGAGAAATGGGGCTCACCAAAACAATCCTCTCAAA | | |
| | CCAACAATACCTCAACCGTCGAGTGGCGTCTCAAC | | |
| | ACCTGGATC | | |
| C145 | GATCCAGGTG GCTTGACCAT TCTCCTGCCG | leucoanthocyanidin | SEQ ID N° 51 |
| | GACCAGGACG TCGCCGGCCTTCAGGTCCGC | dioxygenase-like | |
| | CGCAACCGCG ATTTGGATCAC TGTAAAGCCA | protein | |
| | GCTTCTCATGCTTTCATTTGT CAATATAGGT | | |
| | GATCAGATTC AGGTATTA | | |
| C147b | TAATCAGGGGCAATGTTGCTGTGCTGGATC | aldehyde | SEQ ID N° 52 |
| | | dehydrogenase | |
| C147c | GATCCCCTCATCAAGGCCAATGACACCATTA | ribosomal protein | SEQ ID N° 53 |
| | | S4 | |
| C149 | TGTGATCACAATTGAGAACTCACGTTCCAATGAGA | methionine synthase | SEQ ID N° 54 |
| | AGCTCCTCTCAGTTTTCAGGGAGGGAGTGAAGTAT | | |
| | GGTGCTGGAATTGGCCCTGGTGTCTACGACATCCA | | |
| | CTCTCCAAGAATACCATCAACCGAAGAAATTGCTG | | |
| | ATAGAGTGAACAAGATGCTTGCTGTTCTTGACACC | | |
| | AACATCTTGTGGGTCAACCCTGACTGTGGTCTCAAG | | |
| | ACCCGCGAGT | | |
| C150 | CCAGGTGGTTTACATACAAAACATATTCCAGCTGTC | putative | SEQ ID N° 55 |
| | AGCAGTTTACAGGAGCATATAGTTCGGAATCCAAC | aminotransferase- | |
| | ACAGGCAAGATATAATAGTACAGAGGCATCTTTGC | like protein | |
| | AAAATGATATTCCAGCAACTGATAATAGAGGGTTT | | |
| | AGGGGTCATGATATGTTGGCACCCTTCACTGCTGG | | |
| | GTGGCAAAGTACTGATGTGGATC | | |
| C157 | GATCCACAGA AATAGGAGGA AAAAATGAGA | putative protein | SEQ ID N° 56 |
| | AAATATCTTC TGCTTAGAGTGTTGTCAAAG | At1g31040 [A. | |
| | CTTTTGCCCT CACTGCCTTC CTTCTCATCA | thaliana] | |
| | TTTTTGTCCTCTTCTCTTGG TCTCTCTCTG | | |
| | TATAATTATG TAGTAGATAA AAGC | | |
| C159 | GATCCACAAC AATGCATCAC AACTATGGAT | putative protein 103 | SEQ ID N° 57 |
| | TCCAATTATT CGATTTTTTC TTTCCCTCGC | [Nicotiana tabacum | |
| | AATATGATCT A | chloroplast] | |
| C15b | GATCCCACAAATGGAGGGTATATTTGACAACTATTT | Chaperonin | SEQ ID N° 58 |
| | CCGTGAAGCGTCAGATTGTTA | | |
| C16 | TTGATTCGGATTGAGGGAGTGAATACTAAAGAAGA | putative ribosomal | SEQ ID N° 59 |
| | AGTGGATTGGTACTTAGGAAAGCGTCTGGCTTATA | protein | |
| | TTTACAAGGCCAAAACAAAGAAGAATAATTCAGCA | | |
| | TTATCGTTGTATTTGGGGTAAAGTTTGTAGGCCACA | | |
| | TGGTAACAGTGGTGTTGTTA | | |
| C160 | GATCCAGGTCTGGTTTTATGATATTGAAATGAAGGA | subtilisin-like serine | SEQ ID N° 60 |
| | TTACGTGAATTTTCTTTGCGCCATTGGTTATGACCC | proteinase | |
| | CAAAAGGATTTCACCGTTCGTGAAAGATACTTCTTC | | |
| | GTGAATTTGCAGTGAAAAGAGTTTTAGTTAGTCCAG | | |
| | GGGATTTGAATTATCCGTCGTTCTCAGTTGTTTTTA | | |
| | GCAGTGAGAGTGTGGTAAATAC | | |
| C162a | GATCCAGCACCATGAATACATGGGCTTCGAGAACC | putative protein | SEQ ID N° 61 |
| | GCAAATATGATCCTTA | At2g25740 [A. | |
| | | thaliana] | |
| C162b | GATCCACAGAGTATTTGCAGCCAAGAGTCGTAGAGA | putative protein | SEQ ID N° 62 |
| | ACGGATCAGTGAACGCCTTA | At5g37800 [A. | |
| | | thaliana] | |
| C163 | GATCCAGACC CAACAAAGAT GAATGTGCCT | glycosylated gag | SEQ ID N° 63 |
| | TTTGTCGAGA AAAAGGGCACTGGAAGAAAA | protein | |
| | GACTGTCCGA AGTTGAAGAA TAAGGCCAAA | | |
| | TATAATAATGGAAAGGCCAT TATGGATTGA | | |
| | AATGTAGCTG ATTGTGATGA | | |
| | TTCAGACTTTCTCATTA | | |
| C165 | TGCATCCAAC GCGTTGGGAG CTCTCCCATA | putative ligand- | SEQ ID N° 64 |
| | TGGTCGACCT GCAGGCGGCC GCGAATTCAC | gated ion channel | |
| | TAGTGATTAG CGGATAACAA TTTCACACAG | protein | |
| | GAAACAGCTA TGACCATTAG GCCTATTTAG | | |
| | GTGACACTAT AGAACAAGTT TGTACAAAAA | | |
| | AGCAGGCTGG TACCGGTCCG GAATTCCCGG | | |
| | GATTTTTTAT TCTTTCAGGT TTAGTTTCTC | | |
| | AACAATGTTT TTGGCACACA GAGAAAACAC | | |
| | AATGAGCACC TTGGGACGCT TAGTGCTCAT | | |
| | CTTCTGGCTC TTTGTCGTTC TAATTATCAA | | |
| | TTCGAGCTAT ACAGCTAGCT TGACATCTAT | | |
| | CCTGACGGTG CAGCAGCTGT CTTCAGGAAT | | |
| | TCAAGGAATT GACAGTTTAA TTTCAAGTAG | | |
| | TGATCAAATA GGAGTCCAGG ATGGGTCATT | | |
| | TGCATATAAT TACCTCATTG AAGAGCTAGG | | |
| | TGTTTCAGAA TCACGGCTTC GTATATTGAA | | |
| | AACTGAAGAT GAATATGTCA GTGCCCTCGA | | |
| | GAAAGGTCCA CATGGTGGTG GTGTTGCTGG | | |
| | CATTGTCGAC GAGCTCCCTT ATGTTGAGCT | | |
| | CTTCTTATCC AACAACAAAT GCATATTCAG | | |
| | GACAGTAGGG CAGGAGTTCA TAAAGGGCGG | | |
| | ATGGGGCTTT GCATTTCAAA GGGACTCTCC | | |
| | GCTGGCTGTT GATCTGTCAA CTGCAATTCT | | |
| | TCAACGGTCA GAGAACGGTG AACTCCAAAG | | |
| | GATTCATGAC AAATGGCTAA CGAACAACGG | | |
| | ATGCTCTTCA CAAAACAACC AAGCTGATGA | | |
| | TACTCAGCTT TCTCTCAAGA GCTTCTGGGG | | |
| | CCTATTTCTC ATATGCGCCA TTGCTTGCGT | | |
| | CCTTGCTCTT ATAGTGTTTT TCTGCAGGGT | | |
| | ATACTGTCAA TTCCGGAGGT ATCACCCCGA | | |
| | GCCAGAGGAG CCGGAGATCA GTGAACCTGA | | |
| | ATCTGCACGA CCTAGTAGGC GTACCCTCCG | | |
| | CTCTGTTAGT TTTAAGGACT TGATAGACTT | | |
| | TGTCGATAGA AGAGAAAGTG AAATTAAGGA | | |
| | AATACTCAAG CGTAAGAGTA GTGATAACAA | | |
| | GAGACATCAA ACTCAGAACT CAGATGGGCA | | |
| | GCCGAGCTCG CCTGTTTGAA ACAAAAATTT | | |
| | GTGGTCGGGT TTGTTAGCTC TTGCTCAATA | | |
| | CACTTATGGT TGATATGTAA ATGATGCATG | | |
| | TACAATTTTA TTGTTGAATT ACCTCATTTC ACAC | | |
| C166 | GATCCATCGTCTTGCTCGCTATTACAAGAAAACAA | 40s ribosomal | SEQ ID N° 65 |
| | AGAAGCTCCCACCTGTCTGGAAATACGAATCAACC | protein S13 | |
| | ACTGCTAGCACGCTTGTGGCTTAGGGTGAGCCTTG | | |
| | GGCTGGAGTAGTTTTGGCTGATGGCAATATGTTGTT | | |
| | TTCTCGTGTCATGAATTACTTTGTTACTCAGGACTCA | | |
| | TCGAAGCTCCACTCGTTCTGCTCGGTGACCTCGTCG | | |
| | TCGTTGTCGTGTTTA | | |
| C169 | GATCCATGCA GCAATCAAGC GCTTTGAAGT | Glutathione S- | SEQ ID N° 66 |
| | TGACATGAAT CAATTCCCCA CTCTGTTGAG | transferase | |
| | GGTATTTGAG GCTTACCAAG AGCTGCCTGC | | |
| | TTTCCAGGAT GCTATGCCAG AAAAGCAGCC | | |
| | TGATGCCACT GCCTGAGGCA AGAATCTCAG | | |
| | GCTATCCATC TCCTTGAAAG TTCCCTTCTC | | |
| | AAACCGTTGA CATACCTGCT GGACTTGCAT | | |
| | TTCGGAGAAT TGTTAGCTTT TTCTATTTCT | | |
| | AAAGGCATTA TGACAAGGAT GAGGATGGCG | | |
| | CCTGGTTTCT TCAGGCTAGA | | |
| C17 | GATCCCAACC AGTGCTGCTC CGCCGTGGTG | putative protein | SEQ ID N° 67 |
| | CTATCCATCT CCGCCCCGAT TGACGCCGTG | At2g38310 [A. | |
| | TGGTCCCTAG TCCGCCGTTT CGACAACCCG | thaliana] | |
| | CAAGCGTACA AGCATTTCCT TA | | |
| C170a | GATCCATGGC GGCTGTTCAC TCAGTCCTCC | putative glycosyl | SEQ ID N° 68 |
| | GCCACGCGTC CTGTCCAGAACACGTCTTCT | transferase ? | |
| | TCCACTTCAT CGCCGCTGAG TTCGACGCGA | | |
| | CGAGCCCGCG AGTTTTGACA AAGCTGGTCC | | |
| | GATCCATTTT CCCTTCGCTC AACTTCAAAG | | |
| | TCTACATTTT CAGAGAAGAC ACAGTCCTAA | | |
| | ATCTCATCTC TTCATCGATC CGACAAGCTC | | |
| | TCGAAAACCC GTTA | | |
| C170b | GATCCATTTT GCCGACTTCC CTTGCCTACA | probable | SEQ ID N° 69 |
| | TTGTTCCATC GACCAGAGGCTGTTCACCTT | cytochrome P450 | |
| | GGAGACCTGA TGCGGTTATG AGTACGACCG | monooxygenase | |
| | GGCGTGGACG GCACTCGGTC CTCCGGATTT | | |
| | TCAAGGGCCG CCGGGGGCGC ACCGGACACC | | |
| | ACGCGACGTG CGGTGCTCTT CCAGCCGCTG | | |
| | GACCCTAGCC TCCGACTGAG TCGTTTCCAG | | |
| | GGTGGGCAGG CTGTTA | | |
| C174 | GATCCATGAA CCCTGCAAGG GCATTTGGGC | beta-tonoplast | SEQ ID N° 70 |
| | CTGCTCTCGT CGGCTGGAGGTGGAGGAACC | intrinsic protein | |
| | ACTGGATTTA CTGGTTGGGC CCTTTTGTGG | | |
| | GTGCAGCCTT GGCTGGACTT ATCTACGAGT | | |
| | ATGGAATCAT ACAGCATGAG GCCGTTCCGC | | |
| | GCCCGACCAC CCATCAGCCA TTGGCACCAG | | |
| | AAGATTACTA AATGCACTTC GATAGCAGTC | | |
| | TTCCATTTGT GAATAAGAGA GGATTGTGCT TA | | |
| C175 | ACAGCTATGACCATTAGGCCTATTTAGGTGACACT | auxin-responsive- | SEQ ID N° 71 |
| | ATAGAACAAGTTTGTACAAAAAAGCAGGCTGGTAC | like protein | |
| | CGGTCCGGAATTCCCGGGATGTACAAATTACTAAT | | |
| | ATAGTTTCTTCACAATTATGGAAAGAAGCGTAGCT | | |
| | AATGAGGCACCAAAGGCCACAATAATGGTGGAGG | | |
| | ACTACAAGAAGAATCTTGAGTTCATTGAAGAGGTG | | |
| | ACTAGCAATGTTGATGAGGTCCAAATGAGAGTTCT | | |
| | TGCTGAAATCCTCTCCCAGAATGCACATGTTGAGT | | |
| | CTTGCAACGCTATAATCTCAATGGCCGCACTGATA | | |
| | GAGAGACATTCAAGAAAGTCGTACCTGTCATCACT | | |
| | TATGAAGATATTCAGCCTGATATCAAACGTATAGC | | |
| | CTATGGTGATAAATCTCCTATTCTCTGCTCCCAACC | | |
| | CATCTCTGAATTATTGTCAAGTTCTGGCACGTCTGG | | |
| | AGGGGAGAGCAAATTGATACCATCAACAGAGGCA | | |
| | GCGCTTTGGGAGGAGATTACAGCTTCTAAAACTTCT | | |
| | GATGTCTGTGATGAGCCAAGTGGCTCCAGATTTTG | | |
| | GAAAGGGTAAAGGAATGTATTTCATGTTTCATAAGT | | |
| | TCTGAACAGAAGACCCCAGGAGGATTACTAGCACG | | |
| | CTTTTTTACAACTAGTTTTTACAAGAGTCCTTATAT | | |
| | CAACTGCGGATACCCCTGCAGGAAATTCACTAGTC | | |
| | CAACGGCAACCATTCTTTGCCAAGACTCTTACCAA | | |
| | AGTATGTACTCGCAAATGCTCTGTGGCCTCTGCCAA | | |
| | AACCAAGAAGTCCTCCGTGTTGGCTCGCTTTTTGCA | | |
| | ACCGGCTTCATTCGTGGCATCCGTTTCTTGGAGAAG | | |
| | CATTGGTCTCTACTTTGTAACGATATGCGAAACGGA | | |
| | ACCATTAACACCCAAATTACAGATCCTTCAGTGAG | | |
| | AGAAGCAGTGATGGAAATCCTCAAACCTGACCCAA | | |
| | ATTAGCTGATTTTCATTGAGGCTGAATGCAGCAAA | | |
| | GACTCATGGCAAGGAATCATCACTAGGTTGTGGCC | | |
| | TAATACCAAGTATGTGGATGCTATTTTGACTGGATC | | |
| | CATGTCACAATATATACCGATACTTGATTATTACAG | | |
| | CAATAGCCTCCCTCTTATCAGTACTTTGTATGGTTC | | |
| | CTCAGAATGCCACTTTGGAATCAACTTGAACCCTTT | | |
| | TTGTAAGCCCAGTGAAGTCTCTTACACCCTTATTCC | | |
| | CACCATGTGCTATTTTGAGTTCTTACCATATCACGG | | |
| | AAATAGTGGAGTCATTGATTCTATCTCCATGCCTAA | | |
| | GTCGCTTAATGAGAAAGAACAACAACAATTGGTTG | | |
| | ATTTGGCTGATGTCGAGATTGGCCAGGAGTACGAG | | |
| | CTTGTTGTTACCACATATTCTGGACTCTACAGATAT | | |
| | AGAGTCGGTGATGTGCTTCGGGTTGCTGGATACAA | | |
| | GAACAACGCGCCTCGATTCAACTTCCTATGCCGGG | | |
| | AAAATGTAATCTTGAGCATTGGTGCTGACTTCACTA | | |
| | ATGAAGTTGAGCTACAAAACGCAGTGAAAAATGCA | | |
| | GTGGGCAATCTGATGCCATTTGATTCTCAGGTAACC | | |
| | GAGTACACCGGCTATGTCGATATTACCACCATTTCC | | |
| | AGCCACTATGTCATATTCTGGGAGCTGAATGCGAA | | |
| | TGACTCTACCCCAGTTCCTCCTTCAGTCTTTGAAGA | | |
| | TTGCTGCCTCACAATTGAAGAATCTCTTAACTACTT | | |
| | CTACCGCGAGGGCCGTGCGTCTAATGCATCCATCG | | |
| | GGCCTCTAGAAATTAGGGTGGTGGAAATTGGAACT | | |
| | TTTGACAAGCTCATGGACTACTGCAGTAGCTTAGGT | | |
| | GCTTCCATGAACCAATACAAGACACCCCGTTGTGT | | |
| | CAAATATGCACCCCTTATTGAGCTATTGAACTCTAG | | |
| | GGTCGTCTCCAGATACTTCAGTCCCATGTGTCCAAA | | |
| | ATGGGTTCCTGGCTACAAGAAATGGAACAACACCA | | |
| | GTTAAATGTCAAGCTTCCAATTTCTCTACTTGAAGC | | |
| | TTCATTCTCTATCCCGAAAAAAGACAACCATTTGTG | | |
| | GATTATTTAGTCAATCGTCATCCTAGCTAAGTTGGT | | |
| | CTTTCGTGAACATGGTATGGATTTGTATTTGTCACA | | |
| | AATAAAATGTGGCACTTTTTATTTCTGTAATGGTTT | | |
| | TATTGTGTCAAGTAGTTTAGTGCAAAGACGAGGAG | | |
| | AAGAAGTCAAAAGAGAGGTTTGGTAGACACTTTTA | | |
| | GTGCCCATATTATGTTGGTGGTTTCACTTGTCTTTTC | | |
| | TATTGCATTTGTGAAGTCTGCTATATAATAAACATC | | |
| | CCGGCATCT | | |
| C177 | GATCCATGGC TCGGTTTTGG GCTAAATATG | glutathione S- | SEQ ID N° 72 |
| | TTGACGATAA GTCATATAATACCTGGAATG | transferase | |
| | TGTTTATGCA ACACTGGAGT C | | |
| C178 | TGGAACGGCGCTCCTTATTTGAGGAAAGTGGACCT | auxin-induced | SEQ ID N° 73 |
| | CAGAAACTATTCTGCATACCAGGAGCTCTCTTCTGC | protein IAA4 | |
| | TCTACGAAGAAAGATGTTTACCTGTTTTACTATTGG | | |
| | TCAATATGGATC | | |
| C18 | GATCCCAACG CATCAGGGTG AGTCCTTCAA | RNA-binding-like | SEQ ID N° 74 |
| | AAACACCAGT GAGGCCACGA CTTCCCCGTG | protein | |
| | CCATGATGCA GTAACCGATG CTTGTTCTCA | | |
| | TGACATGGAA AGAGTTCAGG AAAGCCTTCT | | |
| | TGGAAGACTT GAGGTCACCA TGGGAAGGCG | | |
| | AAACGAAATT CTGTTTCAGT AATTTCCACC | | |
| | TTTCTTTTCT TTTTTCTTTC TGTATTGCCA | | |
| | ACACAGTAAC TTTATTGGTA CTGAACATGG | | |
| | CATTA | | |
| C180b | TAAGGCTACAAGCGTAACTTTTAGTGATAGATCAT | ferredoxin-NADP | SEQ ID N° 75 |
| | CATGGATC | oxidoreductase | |
| C181a | TAAGGCTACAAGCGTAACTTTTAGTGATAGATCAT | ferredoxin-NADP | SEQ ID N° 76 |
| | CATGGATC | oxidoreductase | |
| C182 | GATCCATCAG TTGCTTCTAT AAAGCCATTG | patatin | SEQ ID N° 77 |
| | GACGTCAAAC AAGTTTTGCTGCTCTCATTA | | |
| | GGGACTGGCA CTACTGCAGA TTTTGCTGGG | | |
| | ACATACACAG CAAAGGAGGC AGATAATTGG | | |
| | GGTCTTGTTT CCTGGCTATT TCATAATAAT | | |
| | TCGAACCCTC TTATTGAAAT GTCATCTGAA | | |
| | GCAAGTGTTA TTATGAATGAT TATTACATC | | |
| | GCCACCATCT ATCGCGCTCT TGGTGCTGAA | | |
| | ACGAATTA | | |
| C183a | GATCCATCAA ACAAATCTGT GTCTGCAGGC | auxin induced like- | SEQ ID N° 78 |
| | AGCTCTTCTA ATAAGATCAGACAAATAGTT | protein | |
| | AGGCTTCAAC AGCTCCTCAA GAAATGGAAG | | |
| | AAGATAGCAGCTGCCTCCCC CTCCTCCACC | | |
| | CACCTCCATA ACAACCTCCT | | |
| | CAGTATAAACAACAGCACAA GCAGCAGCAC | | |
| | CAAAAGCATC AATAAGTTCC | | |
| | TCAAGAAAACCCTTTCATTC TCGGAAAAGG | | |
| | ACAGATCATC ACCTGCAGAG GTATGCAGCATTA | | |
| C185c | GATCCACCAA AACCCTCGGC AACTTCGTTA | rRNA intron- | SEQ ID N° 79 |
| | CTCAGGACTC ATCAGACTGA GAGCTCTTTC | encoded homing | |
| | TTGATTCTAT GGGTGGTGGT GCATGGCCGT | endonuclease | |
| | TCTTAGTTGG TGGAGCGATT TGTCTGGTTA | | |
| C2 | GATCCCAGAAGTTAGGACATACGTCCCTAACGTTG | lipase-like protein | SEQ ID N° 80 |
| | TCGCGGGGATTATGAGAGGCATCAAAGATGTGATTT | | |
| | CAGCTCGGAGCCACGCGCTTTTTGGTTCCAGGAATT | | |
| | ACCCACTCGGGTGCTTGCCGCTGTATCTCACATCA | | |
| | TTTCCTGATAATAATACAGGCGCGTACGACCAAAT | | |
| | GGGTTGCTTGAGGAACTACAATGACTTCGCTTCGT | | |
| | TCATAATAGATACGTGAGCAGGGCTATCGCGAATC | | |
| | TGCAGCGCGAATTCCCGAATGTTAGCATTGTGTAC | | |
| | GGGGATTTCTATGGTTCCCTTTTGACAGTTATGCGC | | |
| | AGTGCTTCTTCCTTTGGATTTGACCAGAACACGTTG | | |
| | CTTAGTGCATGTTGTGGAACTGGAGGGAGGTATAA | | |
| | CTTTA | | |
| C201a | GATCCCGAAT GACGACAAGC TTCAATCCAT | putative protein | SEQ ID N° 81 |
| | TACTGTAAAT GGTAGCAAAA TCCTACCCGA | At5g44670 [A. | |
| | TTGGGGATAC GGTAGAGTTT ATACTGTTTT | thaliana] | |
| | AGTTATCAAT TGCACTTTCC CTATTCCAGT | | |
| | TGGTACTGAA AATGGAGGAA AACTCGTAAT | | |
| | TCATGCCGCT ACTAACGGCG GCGGGGACAC | | |
| | TAAATTCAAC ACCGCCGACA CTTTCGTAGG GTTA | | |
| C201b | GATCCACCTG CCCTTTCAGA TGAGTCAATC | N-carbamyl-L- | SEQ ID N° 82 |
| | ACTAAGGCGA CAGAATTAGC ATGTCAACAG | amino acid | |
| | CTGAATTTGA CTCGCAAGAG AATGATTAGT | amidohydrolase | |
| | CGAGCCTATC ATGACTCCCT GTTTATGGCA | | |
| | AGAATATCCC CGATGGGCAT GATATTCATT | | |
| | CCTTGTTACA AGGGATATAG CCATAAGCGT | | |
| | GAAGAGTTTT CATCTGTTGA CGATATCGCG | | |
| | AACGGGGTAA AAGTTCTAGC GTTGACTCTT | | |
| | GCCAAGTTAT CTCTCTCATA ATCCCTTA | | |
| C202 | TTATAGATCAGAAATTTGAAGCCGGAGAAAATGGC | dihydrolipoamide | SEQ ID N° 83 |
| | GATAGGGAGCTTAGCAAGAAGAAAGACCACAACA | dehydrogenase | |
| | ATTTTATCTTCCAGATATCTCTATAGCACATCCAAA | | |
| | TATTCATTTTCTCTCAGCAGAAATTACTCTTCGGGA | | |
| | TC | | |
| C203 | GATCCCGAGT TGTACGCATG AGCTCGCAAA | carbonic anhydrase | SEQ ID N° 84 |
| | AGATCAAAGC CCAAAGTTTC TCGTATTCGC | | |
| | CTGCTCCGGC TCCACCAGCT GTGCCC | | |
| C207b | GATCCCTATC CAATAGATAT GGAATTTCGA | chlorophyll a | SEQ ID N° 85 |
| | CCACCTTGTA TAGTTCTATC AACCATTGGA | oxygenase | |
| | ATCTCAAAGC CAGGCAAGTT GGAAGGGCAG | | |
| | AGTACCAAAG AGTGCTCTAC ACACCTACAC | | |
| | CAACTTCATG TATGTTTACC TGCATCTAAA | | |
| | CAGAAGACAA GGTTGTTATA TAGGATGTCA | | |
| | CTGGATTTTG CTCCCGTGCT AAAACACATC | | |
| | CCTTTCATGC AATACGTGTG GAGGCATTTT | | |
| | GCTGAACAGG TTA | | |
| C207c | GATCCCTGAT GCATATGAGC GGCTGCTTCT | lysyl-tRNA | SEQ ID N° 86 |
| | TGATGCTATA GAAGGTGAAA GGCGGCTTTT | synthetase | |
| | CATCCGCAGT GATGAGCTGA ATGCTGCTTG | | |
| | GTCTCTTTTC ACACCAGTGT TACTCAGGAC | | |
| | TCATCAACAA GCATGAACTT TGCAATGCAT | | |
| | ACACTGAATT GAATGACCCT GTTGTCCAAC | | |
| | GCCAGCGTTT TGCTGATCAA CTCAAGGATC | | |
| | GACAATCAGG TGACGATGAA GCTATGGCAC | | |
| | TGGATGAGAA CTTTTGTACA GCTCTTGAAT | | |
| | ATGGATTACC TCCTACTGGT GGTTGGGGAT | | |
| | TGGGTATTGA CCGACTTGCG ATGTTTGTTA | | |
| C208 | GATCCCCACC ATCAGGTATT CCGAGCCGCA | translation | SEQ ID N° 87 |
| | ATAGGTGAAC CGGACCCTCT TGAAGATCAT | elongation factor | |
| | CGTATTCGAG ACCACCCCAA ACGACCCCTC | like protein | |
| | GTTCGATGTT GTTCTTCATT CCAACAACTG | | |
| | CTTCCAGCAG CCTAACGCCG GACACACGCA | | |
| | CACACGCACC AACAGGTCGT CGTTCTTCCA | | |
| | CACAAACCCG CCGGAACGGA CTCCTTCTCC | | |
| | ACCCAGAACA GACCCAGACC G | | |
| C212 | GATCCCTATG AACGAGCTTT AGCTCGTTCC | auxin-induced | SEQ ID N° 88 |
| | TGGGCTAATT T | glutathione S- | |
| | | transferase | |
| C213b | TAACAACGCAACCACACAGAATCGATCGTTACATA | A3 [Nicotiana | SEQ ID N° 89 |
| | AAGGGATC | tabacum] | |
| C214a | GATCCCTTGG ATGGTACTTG TTGGTGAACG | histidyl-tRNA | SEQ ID N° 90 |
| | CGAACTTAGC GAAGGAGTTG TAAAATTGAA | synthetase | |
| | GGATGTGTTT GCAGCTATTG ATTATGAAGT | | |
| | CCCCAGAGGT AACCTTGTGA ACGATTTATG | | |
| | CAGAGGATTA GGCATGTAAT ATCTCAAGTT | | |
| | ATTAGTATTG TTAGATTGAT ACAAGAATGC | | |
| | TTTTTTGGGG GGTGGGGGTT A | | |
| C214b | TAAGCGCAGA TGATAATGGT GAAGGGGGTA | potassium | SEQ ID N° 91 |
| | CATTCGCTCT TTACTCTTTG CTGTGTAGAC | transporter | |
| | ATGCAAAGTT TAGTCTACTT CCCAACCAAC | | |
| | AGGCAGCAGA TGAGGAGCTA TCTGCTTACA | | |
| | AATATGGATT CTCCGGGCAG TCGGCATCTT | | |
| | GTTTACCATT GAAGAGATTT CTTGAGAAGC | | |
| | ATAAGAAGTC ACGCACAATA CTGCTTATTG | | |
| | TTGTATTGTT AGGTGCTTGT ATGGTCATAG | | |
| | GAGATGGTGT TCTGACTCCT GCAATGTCAG | | |
| | TTATATCATC AATATCAGGG ATC | | |
| C215 | GATCCCTCTC TATTTGCATA AATGTTGATG | putative protein | SEQ ID N° 92 |
| | GATTTGAAGC AATGTTTTTC ATAGGAGTAA | At4g25640 [A. | |
| | ATGCTGCTAT AAGTGTTCGT GTCTCAAATG | thaliana] | |
| | AGCTTGGGCT AGGACGTGCC AGGGCAACCA | | |
| | AGTATAGCGT CTGTGTCACA GTGTTTCAGT | | |
| | CGCTTCTCAT TGGGATAGTA TGCATGATTG | | |
| | TAGTATTGGC AGTAAGAAAT CATCTGGCCA | | |
| | TTCTTTTCAC AAACAGCAAG GTTCTGCAAC | | |
| | GTCCCGTACC TGACCTGGCT TGGCTTGTAG | | |
| | GAATAA | | |
| C216b | GATCCCTAGG CATAAAACAA TGAGCAACGC | putative protein | SEQ ID N° 93 |
| | CGCAAGAGAT ATACGGAATC GCTGACCCCC | At2g20240 [A. | |
| | GAAAATTTTG ATCATTTTCA TTCTCTGATT | thaliana] | |
| | TTGAAAGAAT AGCAGCGCCG TTTTTGGAGC | | |
| | TTGGCAAACC GGACCCCATC CCCCTTTTTG | | |
| | TCGTCGTCTT TCTCAAACCA GACTCCCCTC | | |
| | CCTGATCATT TTTTCTTCTG GGAAAACAAA | | |
| | GCAGCATTTC CATGGTTTTG GCTTTA | | |
| C217 | GATCCCTCAA GTTGCACTTT GAATATGCTT | 60S ribosomal | SEQ ID N° 94 |
| | GTAATAAATA GAAGTAATAT AACAGTGCTT | protein L13a | |
| | TGTTCTCCAA GGCTTCAAGG TGTGACCATG | | |
| | TTGGATACAA TCTGAAAGTT GTGTTCCAAT | | |
| | CCACGTGATC TTTCTGGCTG TACACGCTAA | | |
| | TCCACCAGAC AACTTGCTTA CTCAGGACTC | | |
| | ATCAACTCGC CATTATTGCT CCAATCAAGT | | |
| | ACTGAAGTCT AAATATAGTT GTTTGAAGTA | | |
| | CAATTTTGCT GGAGATTGAT GTTTTGGCTT A | | |
| C22 | GATCCAACTTACGACATAGGCCTATTGGAATTGGA | ribonucleotide | SEQ ID N° 95 |
| | GTTCAGGGTCTTGCAGACACATTCATGTTGCTTGGC | reductase | |
| | ATGGCATTTGATTCTCGGGAGGCTCAGCAGCTAAA | | |
| | CAAGGACATATTTGAGACAATATACTACCATGCAT | | |
| | TA | | |
| C220 | TAGTGCTATGGCTGTGGACTCAGGTGCATTTGTACA | putative F1-ATP | SEQ ID N° 96 |
| | CAAAGAGGTATGAATGTACTAAAAATGTCACAGTC | synthase subunit | |
| | TCCCGCACTTCATATTCATCATTTTTGAAAGCGAGG | alpha | |
| | GGAAGGGATC | | |
| C224a | GATCCCTTTA CATCATCCAC ATATAATTCA | seven in absentia- | SEQ ID N° 97 |
| | TTCTCAATTC CCATCTTCAA AATCACCCCT TA | like protein | |
| C224b | GATCCCTGGC GACAAGCAAT GGAACAACAT | auxin-responsive | SEQ ID N° 98 |
| | GAATTGAATA GCCAATTTCT GTTAGTACC | GH3-like protein | |
| C227a | GATCCCTTCT TTCATATCTG AGATTCAAGC | lipase | SEQ ID N° 99 |
| | TGCAATCTGG GGCATATACA ATAACGGTGG | | |
| | GAAGAATTTC TGGGTTCATA ACACAGGACG | | |
| | CTTGGGTTGT TTGCCACAGA GGCTTGCCAC | | |
| | AAGAAATGGG AGCAATTTGA ACGATTATGG | | |
| | ATGCATTA | | |
| C227c | GATCCCTGTGGCTAGACTAACTGGCCGAGAGGGTT | mitochondrial | SEQ ID N° 100 |
| | AGCGAGGTTCCTGCTATGGTGAAGTGAAAGATCTT | ATPase subunit 9 | |
| | TCACTATAGTGGGAAGAAGACAGGTGGGAGCGAG | | |
| | CGGAGCGAGAGCAAAGCAAGCTCTAGTGGTGGGTT | | |
| | GTCTTCGCGGTCCCATTA | | |
| C228a | GATCCCTTCA ACGGCGTTGC TTGCTGATGG | arginine | SEQ ID N° 101 |
| | TGTCCGTGAG GCTGCTCAGA TTTATTGTGA ATTTA | decarboxylase | |
| C228b | GATCCCTACG AACTCGGGAA ATGGGCCAGT | putative protein | SEQ ID N° 102 |
| | CTTTCAGCTA TTTGATTAGA ATAATCACAC | At3g59770 [A. | |
| | CGATTA | thaliana] | |
| C230 | TAATCCATGT CAAACTCGAC TTTTTGCAGC | B12D protein | SEQ ID N° 103 |
| | CGTAGGCGTT GCTGTAGGGA TC | | |
| C232 | TAAACGTGAA TATCGGATTA CACCTCCGCC | proline-rich cell | SEQ ID N° 104 |
| | TCCGCTGTCA ACACAAGTGG GAGACATTCC | wall like-protein | |
| | TCGAAGCACA TTCAACTTTG ATTTTGACTT | | |
| | TGAGGGAAAG ATTCTGGCCG AAGCAGAAAA | | |
| | GGAAAGCCAG AATTGGAGCA GGCTAGGGCT | | |
| | GGAA | | |
| C237b | GATCCCGTCT ACCTTATTCT TTTCAGCAGC | putative protein | SEQ ID N° 105 |
| | CGCAACAGGC AAGTTTTTGC ACCATCTGTT TA | At1g22750 [A. | |
| | | thaliana] | |
| C238a | GATCCGTCAA GTTTGCATGG TGGTTGCCCT | prolyl 4- | SEQ ID N° 106 |
| | GTGATTA | hydroxylase | |
| C238b | GATCCCGTAG AAAATGCTTC TTTTATGCCT | cytochrome c-type | SEQ ID N° 107 |
| | TGGGTATTTA TATTATAATT TTCATTTTTT | biogenesis protein | |
| | GGTGTTTAGG ATTA | | |
| C238c | GATCCCGATGTGATTCATAACTTTCATCACACCCCT | vestigial protein ? | SEQ ID N° 108 |
| | CTCAATATCTTCAGCTGAAATTTGTTACTCCATTTA | | |
| C23b | GATCCCACCTCAGGAAAAAAAATCTGCTACGTGCA | cellulose synthase | SEQ ID N° 109 |
| | GTTTTCCACAAAGGTTTGATGGGATTGATCGTCACG | | |
| | ACAGATACTCAAACAGAAATGTCGTATTCTTTGAT | | |
| | ATTA | | |
| C24 | TAAAGCAACA AAATCAATTC ACAGCACCTC | amino acid transport | SEQ ID N° 110 |
| | ACTTTAGTGT AAGCAAGAAT CAAAAAGCAA | related protein | |
| | GTTGCAGGTA CAAATTCCAT AGTGCCAGCT | | |
| | GACCTACCAA AGTTGGGCAT AGCCCATAAC | | |
| | AATGTCAACA TTCTCAAAAG AAGATAAAAT | | |
| | CACATCTGTG TTCAACCACA TCATTGAATA | | |
| | TCAAAAGATA TAAGAACCTA TAAGCTGGGC | | |
| | GTTCTTGTTC CTTTTTTCCC TTTTGATGAA | | |
| | GGTATCTCTC CTATAAGGGT GGGGGGATC | | |
| C25 | TTCAACAGAA GAACTCCATC ATCAGCCACT | proline rich protein | SEQ ID N° 111 |
| | GAGGAGAGAA CGCCCAACCC CTGGACAAAA | | |
| | TAGAAAACAC ACAATATTGG CCGCGGACCC | | |
| | CAACTTCAAA AACAGAAATC GACCTTACCC | | |
| | AATTCCCAAT TTCCAAGAGC CTCTCACGCA | | |
| | CACACACCCC TGAAACCTAG TAAAAATAGA | | |
| | AGGTCTTTGC ACAAAACAAC ATCTCCAAAT | | |
| | GGCTCA | | |
| C28a | GATCCCCTGA ATATTGGGTA GCTGTTGTTA | T48 protein [Tupaia | SEQ ID N° 112 |
| | CTCAGGACTC ATCACATGCA GAGGTATCGC | herpesvirus] | |
| | GTGTTTGGAT TGTGTTA | | |
| C28b | GATCCCCTGA ATATTGGGTA GCTGTTGTTA | 50S ribosomal | SEQ ID N° 113 |
| | CTCAGGGCTC ATCGAAAGAA CCCCTCATCG | protein | |
| | GTTGTTTATC TGGTTTA | | |
| C29a | GATCCCCCTGAGTTCGCCAAGGACTTACTGCCCAA | heat shock | SEQ ID N° 114 |
| | GTATTTCAAGCACAATAACTTCTCCAGCTTCGTTCG | transcription factor | |
| | TCAGTTA | | |
| C3 | CATAAGGAGC AGCTGATCGG AGTCCAAAGA | NADPH | SEQ ID N° 115 |
| | GAATTCGAGA TGCTATAGCA CATATGAAAT | oxidoreductase | |
| | TCTGGGTAGC TCTGTTGTGT AAGGTGTTCT | homolog | |
| | GTACAATGAC AAACAGGATT TGTGATATTC | | |
| | GTTGTGTAAA AGGCAGCA | | |
| C30 | AGGTATTACA AAACGCATGG GGAGTAGTAG | putative protein | SEQ ID N° 116 |
| | TACAAGGGAA AGGGGTAGAA TGTTCACCAG | AT5g05250 [A. | |
| | CTTGTTATTT GTTGAAGACG AGTAGAGTTG | thaliana] | |
| | GTGCTGGTTT AGGAATGGGA TTGTTTTGCA | | |
| | CTCATTTCTG TTTAGCAAGA GTACAGAATT | | |
| | TTAGGG | | |
| C301 | TACCCGAAATCCGAACTCTTGCTCCGAATCAAGCC | ornithine | SEQ ID N° 117 |
| | AATGTTCGACGGCAACGCGAGGTGCCCAATGGGTC | decarboxylase | |
| | CAAAATACGGCGCGCTTCCAGAAGAAGTCGAGCCG | | |
| | CTGCTCCGGGCAGCTCAGGCCGCCCGGCTCACCGT | | |
| | CTCCGGTGTCTCCTTCCACATCGGCAGCGGAGATG | | |
| | CCGATTCAAACGCTTATCTCGGCGCCATAGCCGCG | | |
| | GCTAAGGAAGTGTTTGAAACAGCTGCTAAACTCGG | | |
| | TATGTCGAAAATGACTGTTCTAGACGTCGGCGGCG | | |
| | GGTTTACATCCGGCCACCAGTTCACAACCGCCGCC | | |
| | GTCGCTGTTAGATCAGCTTTA | | |
| C303 | GTGGATGAAATAATGGTCATGAGTTTTTCAAATCTG | putative protein | SEQ ID N° 118 |
| | TAGACTGGGATCTGATTATGCAACTTCCCAGGCCA | kinase | |
| | CCGCTTATACCTGTGCCGCACTGACGAGAATGTGA | | |
| | ATATTATGGAGGGAAATGAAGAAATTGCTGTGGAA | | |
| | TTATTTCGAACAGGGAGTGTTTA | | |
| C304 | TAAACCAAAA GCAACTGAAC TCAAGGGCCA | F1-ATPase alpha | SEQ ID N° 119 |
| | CCTCTGAGAG TGAGACATTG TATTGTGTCT | subunit | |
| | ATGTAGCGAT TGGACAGAAA CGCTCAACTG | | |
| | TGGCACAATT AGTTCAAATT CTTTCAGAAG | | |
| | CGAATGCTTT GGAATATTCT ATTCTTGTAG CAGCC | | |
| C305a | GATCCGAGGAAGACGAGACAGAAACACCAGCGGA | heat shock protein | SEQ ID N° 120 |
| | TACTTCAACAGAATCAGATGCAGGCTCTGCTGAAG | | |
| | TCTCAGAGGCACAAGTCGTCGAGCCATCAGAAGTA | | |
| | AGGACCGAGAGCAACGATTATTGGGAGTGATTTA | | |
| C305b | TATACAGGAC AACGACGACG ATGAGTCCTG | latex-abundant | SEQ ID N° 121 |
| | AGTAATCAAC CGTTTCGGAT TTTCTGAGGA | protein | |
| | AGATATTACT GTACTAATTG ATACTGATGA | | |
| | TTCTTACACA CAACCAACTG GTCGGAATAT | | |
| | ACGTAAAGCT TCGTCGGATC | | |
| C306 | GTACTCGCGGAGAGGACTATGAATCTGACGATGGG | putative protein | SEQ ID N° 122 |
| | GTGGAATCATGGGCCAAATAGTTCGACATCCGAAT | At1g26460 [A. | |
| | GGGCACAGAGTAACCGTGTGGAACATGCTGTTTA | thaliana] | |
| C308 | GATCCGAAAGCATCACCCGAAATCCGAACTGTTGC | ornithine | SEQ ID N° 123 |
| | TCCGCATCAAGCCCATGCTCGACGGCAACGCGAGA | decarboxylase | |
| | TGCCCAATGGGCCCGAAATACGGCGCGCTTCCAGA | | |
| | AGAAGTCGACCCGCTGCTCCGGGCAGCTCAAGCCG | | |
| | CCCGTCTCACCGTATCCGGCGTCTCATTCCACATCG | | |
| | GTAGCGGAGATGCCGATTCAAACGCTTATCTCGGC | | |
| | GCCATAGCCGCGGCTAAGGAAGTGTTTTGAAACAGC | | |
| | TGCTAAACTCGGGATGTCGAAAATGACTGTTCTAG | | |
| | ACGTCGGTGGCGGGTTTACATCCGGCCACCAGTTC | | |
| | ACAACCGCCGCCGTCGCCGTTA | | |
| C309 | ACATGGAGGTGCTTATATTGTGAGACACGCCGCGA | S-adenosyl-L- | SEQ ID N° 124 |
| | ATAGCGTGGTCGCAGCAGGACTTGCTCGCCGCTGC | methionine | |
| | ATTGTGCAGGTTTCTTATGCTATCGGTGCGGCTGTA | synthetase | |
| | CCACTGTGCGTGTTTGTTGACACTTACAAAACTGGA | | |
| | ACAATTCCAGACAAGGATATTTTGGCTCTGATCAA | | |
| | GGAGAACTTTGACTTCAGGCCTGGAATGATGTCAA | | |
| | TCAATCTTGACTTGTTA | | |
| C31 | GATCCCCTAT TGACTGCCTC TTGCTCTTGC | putative protein | SEQ ID N° 125 |
| | ACTTGCATAT ACGCTTATAT TCAGGAATAT | At1g71240 [A. | |
| | GCTGTCTTAT GTTTTCCCAG CAATCTTGAT | thaliana] | |
| | TGTCTTGGCT GCTGGCATGT TATTACTTTA | | |
| C310 | GATCCGACTT GCTTTGTCTC TTCGGACGAG | 40S ribosomal | SEQ ID N° 126 |
| | TTACTCAGGA GCATATGAAA AGGAATGTTG | protein S5 | |
| | CCATACTTTT GAGTAGCAGG AAATTTAGGA | | |
| | TCAGTAAAAG AGGCTTACTC AGGACTCATC | | |
| | GTCAGGCTGT TGATATTTCT CCACTTCGCC | | |
| | GTGTTA | | |
| C311 | AAACATGAGGACAAACTTAACATGAGGGGGATGC | putative heat shock | SEQ ID N° 127 |
| | AGGTTCGGACGAAGTCTAATGAGGTACAAGAAGTC | protein | |
| | GAGGCATCAGAAGTAAGGACCGAGAGCAACGATT | | |
| | ATTGGGAGTGATGGTTA | | |
| C312 | TAAGCCCCCA AACTAGAGTC TCCTCAGCTC | receptor-like protein | SEQ ID N° 128 |
| | CTAATCTTTG GCCTAAGAGT ATTTTGGTTG | kinase | |
| | TCAGAAATAC TTCAGCGCTG CTTTTTTTAC | | |
| | AAGAAAGTGG AAATTTGGTT TATGGTAACT | | |
| | GGGGTAGTTT CTTGAATCCA ACTGACACAT | | |
| | ATCTGCCAAA CCAGAACATC AATGGCTCAA | | |
| | ATGCAACTTC CAGTAATGGA AAATCCAG | | |
| C313a | GATCCGAGAC ATCCAGCCGA GTCCACAAAT | putative pyruvate | SEQ ID N° 129 |
| | GCAACCGATG AGTCAGTATT GAAGGTTGCA | kinase | |
| | CTGGACCATG GGAAAACAGC AGGTGTTATA | | |
| | AAGCCACATG ACCGAGTTGT TGTTTTCCAG | | |
| | AAAGTTGGTG ACTCATCTGT GGTGAAGATT | | |
| | ATTGAGCTTG AGAATTAGGT TTGTACATCT | | |
| | TTGTATGTTT CAATTGGCTG ACATTCTTAG CTTA | | |
| C314b | GATCCGAAAA AGAACAAGAC CAAAAGGTCT | putative protein | SEQ ID N° 130 |
| | TGAAAAAGAG AGTGACGAGC AGAAGAGAGG | KIAA0565 [Homo | |
| | AAACAGAAAA TACACAAAAA TTGGGAAGGC | sapiens] | |
| | AAAATAGTGA AATCTCCCAC AAATTTCAGC | | |
| | CTAAAACTAG CTTA | | |
| C314c | GATCCGATGG GAAGACCCGG TATGAGGATT | calmodulin | SEQ ID N° 131 |
| | TCATTGCCGG GATGGTTGCC AAGTGATTTT | | |
| | TGCATGTGAT TTGCATCTCA GGCTATATTA | | |
| | TTCATAGCAG TGAAAGAAGA GCTGACTTTT | | |
| | TCCCTTTGTA GCTTTA | | |
| C316 | AGGTCTATTTTTTCACCAAACAACATTCAGTATTGG | putative oxidase | SEQ ID N° 132 |
| | CTTTGTCCAAAGTAAAAAACTTTATACAAGATGTGC | | |
| | AAAAACTTGTGGTTTTACAGCCCAAGGCATTATGT | | |
| | GGTTTAGACCTATATAGTGGAATCCTAATGAGGTA | | |
| | TGTCACGGTTTCAAATGCTTACTTGGGACATCAAGA | | |
| | AGATGCAGTGGATTTTTGATATTACATATTATAGAA | | |
| | GCAAAAATCCATTGACTCCTAGGTTATATGAAGAT | | |
| | ATTCTTTGAAGAAATAGAGCAAATGGCAATGTTCAA | | |
| | ATATGGAGGAGAGCCTCACTGGGGGAAGAATCGTA | | |
| | ATGTGGCTTTCATTGATGTGATTA | | |
| C320 | TAATGGGGGAGGCTATAGCTACAATGAATCAAATG | ubiquitin-specific | SEQ ID N° 133 |
| | GAGGAAAATTTGGGTCCACAGTTATCTGGTCTTGTC | protease-like protein | |
| | GGGTCGGATC | | |
| C322 | TGCCCTGTTTATCGCTGCACTTTTCCCGAGATACAT | RING-H2 zinc | SEQ ID N° 134 |
| | CCGCTACCGCATCTTCACTAACGGTAACAGCATCCT | finger protein-like | |
| | CCAAACACTTTCCACGCGCCGCCGCCCTTCTGCTGC | | |
| | AACACGTGGACTCGACAATTCGGTCATCGACACTT | | |
| | TCCCCACCTTTTGCATACGCCGAAGTGAAGGATCAT | | |
| | CATATTGGCAAGGGTGGTTTGGAGTGCGCAGTATG | | |
| | CTTGAACGAGTTTGAAGACGACGAAAAGCTGCGGT | | |
| | TGATCCCAAAGTGTGATCACGTGTTCCACCCTGAAT | | |
| | GCATCGGTGCTTGGCTCAAGTCTCACGTCACTTGCC | | |
| | CCGTTTGTCGAGCTGACCTTACTACTCCTCAACCTG | | |
| | ATGTTA | | |
| C323 | ATCCCCATTGGCCTAGTTGGTTCTATGGTGATTACT | amino acid transport | SEQ ID N° 135 |
| | ACCACTATATACTGTATATTTGGCTATAACGCTCTGT | protein AAT1 | |
| | CTTTATGCAGCCTTATCAGAACATTGATCCTAATGCT | | |
| | CCGTTTTCTGTGGCGTTCAAAGCTGTTGGATGGAGT | | |
| | TGGGCGCAATACATTGTGGCTGCAGGTGCATTGAA | | |
| | AGGAATGACATCTGTATTGCTTTGTAGGCGCGGTTG | | |
| | GTCAGGCGCGTTATCTCACTCACATTGCACGGACTC | | |
| | ACATGATGCCTCCTTGGTTTTCCTATGTTGATGCAA | | |
| | AAACAGGAACGCCCGTTA | | |
| C324a | GATCCGGAGA GCCAAACATT TACGTAGTTT | 1- | SEQ ID N° 136 |
| | TCATCATCAT GAAATGGTTA CCTGAAACGA | aminocyclopropane- | |
| | TTGTCAGATT CTGTAATTTT GCTGAGTATA | 1-carboxylate | |
| | CAGAAGACAA TTTTGCATAT AGTGCTTCAT | oxidase | |
| | GCTCTTACAG TTTGTATGGA TCATTGTTCC | | |
| | TTATCGTTTT ATAATGTATT GTATCATTTT | | |
| | ATGAATTCAA TGTTTGGATA GATTTGTATT | | |
| | GTTTGTTATT GTTA | | |
| C324b | GATCCGGGGGTGTAGTTTGGATTGAATTGAACGGGG | putative protein | SEQ ID N° 137 |
| | AAGTGCATGAGTTTATTGCGTTTGATGGTTCACATG | At2g29760 [A. | |
| | CTAAGTCTGAATACATTTACACCGTTTTAGATAACC | thaliana] | |
| | TAGTCGGTCAAATACAACACATTTACTATTTTTCCAG | | |
| | ATGCTGATTCTTTAGTTCTTGAGAATAGCTGAAAGT | | |
| | AATCAGAGTTTTAGATATGCTGAACTTCCAATACAG | | |
| | CCTTAGTTA | | |
| C325 | TTCACGATATCGAAACTAGCGATTACGTGAATTTCC | putative subtilisin- | SEQ ID N° 138 |
| | TATGCTCCATTGGCTATGACGGCGACGATGTCGCC | like serine | |
| | GTGTTTCGTGAGAGATTCTTCTCGAGTGAATTGCAGT | proteinase | |
| | GAACAGAATTTGGCTACTCCAGGAGACCTGAATTA | | |
| | CCCGTCGTTCTCTGTTGTTTTTACCGGTGATAGTAA | | |
| | CGGTGTGGTTA | | |
| C326a | GATCCGGGAA TATCGTCTAG AAGAACTCCT | anionic peroxidase | SEQ ID N° 139 |
| | CCATCGCAAC CATCAACTCA GCCAGAGGTT | | |
| | TTGAAGTCAT AGAACAAGCT AAACAAAGAG | | |
| | TAAAAGATAC TTGTCCCAAC ACGCCTGTAT | | |
| | CTTGCGCAGA CATCTTAGCT ATTGCTGCTC | | |
| | GTGATTCTGT TGTTTA | | |
| C326b | TAACAGAAGAAGAAGAGATGCCGGCCCTAGGTTGT | arginine | SEQ ID N° 140 |
| | TGCGTAGACGCTACTGTTTCCCCTCCTCTCGGCTAT | decarboxylase | |
| | GCCTTCTCTCGGGATAGCTCTCTTCCCGCGCCGGAG | | |
| | TTCTTTTACCTCCGGCGTACCTCCTACAAACTCCGCC | | |
| | GCCGGTTCCCATTGGTCTCCGGATC | | |
| C326c | GATCCGGGCCGGTTCGGGTTTCGTCAACTTTACTTGA | putative protein | SEQ ID N° 141 |
| | ATCCGGAAATGTGCTTCCCATTACTCAGGACTCATC | At5g66860 [A. | |
| | GTTAAACTAAGAAGTAAGATGACTGTACTAGCACT | thaliana] | |
| | CCTATAACTAAAAAGTAACTAGACTCATTCATCAA | | |
| | TATCACTCGCTCTCTCTCTGGTTA | | |
| C327a | GATCCGGGTTGTATTAGATATGGTTTATTACGTTA | cytochrome b | SEQ ID N° 142 |
| | TTTTGTACTTTATTTTGAACTTCATTTCTGTTTGATT | | |
| | GGTTCTACTAATTTGAATTGGTTACTCAGGACTCAT | | |
| | CAGTCCAGTGGTTCAGTGCCTAGTTTTCAAATTGAA | | |
| | GGTCGGGTGTTA | | |
| C327b | GATCCGGCAT GTCTGCTCGA CAAATGGGGA | 60S ribosomal | SEQ ID N° 143 |
| | GGGAGCTGCT ATTAGTATAC TCAGGACTCA | protein L21 | |
| | TCACGAAAAG GCAACCCCTA GGACCCAAAC | | |
| | CAGGTTTCAT GGTTGAAGGC GCTACATTGG | | |
| | AGACTGTTAC CCCCATACCA TATGATGTGG TTA | | |
| C328a | GATCCGTCGG TCAGAGTGGG AGGGGCCCGC | putative protein | SEQ ID N° 144 |
| | AAGCACATGT CGAAAATCAG GATTGATGTC | At4g24290 [A. | |
| | AATGCTGATC AGCACCCCTT TCAGTACGAA | thaliana] | |
| | ACTAAATCAA CCACAGAAGC CAGCTAAGGT | | |
| | GGACCTGAAC TCCGCAGTTT ATCCTGGCGG | | |
| | TCCACCTTCA CCGGCAAGGG CGCCAAAGAT | | |
| | GTCGCACTTT GTCGATACAA CAGAAATGGT | | |
| | AAGAGGACCT GAGGAGTCAC CTGGCTACTG | | |
| | GGTGGTAACT GGTGCAAAGC TATGTGTAGA | | |
| | AGATAGTAGG ATAAGAATGA AAGTGAAGTA | | |
| | CTCGCTCTTA | | |
| C328b | GATCCATGCT TGGTGGTATT GGTTCTACCA | putative protein | SEQ ID N° 145 |
| | TAGCTCAAGG GATGGCCTTT GGTACTGGAA | AC087851 [Oryza | |
| | GTGCTGTGGC ACACAGGGCT GTAGATGCGG | sativa] | |
| | TCATGGGTCC ACGCACCATT CAACACGAAA | | |
| | CTGTTGCTTC CGAGGTACCT GCTGCAGCAG | | |
| | CAGCTCCTAC AACCATCGGT GCTGGGTCTG | | |
| | ATGCTTGCAG TATGCACTCT AAAGCGTTCC | | |
| | AAGACTGCAT CAATAGCTCT GGAAGCGACA | | |
| | TTGGCAAGTT TCAATTCTAC ATGGATATGT | | |
| | TGCCCGAGTG CAGGAGGAAC TCAATGCTGA | | |
| | ATGCTTA | | |
| C329a | GATCCGGCTA TGTTGCTGAT CAATCTGGTT | putative protein | SEQ ID N° 146 |
| | ATGGCATGGT TGATCCTTCT CAGCATTATT | At3g63460 [A. | |
| | ATCCGGAGCA ACCATCCAAG CCGCAGCCAA | thaliana] | |
| | GCATTTCGAA CAGTCCCTAT GCCGAGAA | | |
| C329b | ATGGTTACTGGTTTCTATAGCCAAAAGCAAAGAGG | ambiguous hit | SEQ ID N° 147 |
| | CTTTGGTGAGAAAGATGAAGCTTTTTGGAGGGTAT | | |
| | TGCTGCGTTTTTTTTGTTTGGCTTCTCTCCGGATC | | |
| C330 | ACGGGGGGGG GGGGGGGGGG GGACTTGAAG | ethylene-responsive | SEQ ID N° 148 |
| | ACTGGGAAGC TCCATTAACG AGCTCCGACA | element binding | |
| | ACTCAACAGC CTCTGATTTA AGCCGAAGCA | factor | |
| | ATAGCATTGA GTCCAACATG TTTCCTAATT | | |
| | GCTTGCCCAA TGAATATAAT TATACAGCTG | | |
| | ATATGTTTTT TAACGATATC TTTAATGAAG | | |
| | GCATTGTTGG CTATGGATTT GAGCCAGCTT | | |
| | CTGAATTTAC ACTCCCCAGT ATCAAATTGG | | |
| | AGCCAGAAAT GACTGTACAA TCACCTGCAA | | |
| | TATGGAATTT ACCGGAGTTT GTGGCGCCGC | | |
| | CGGAGACGGC GGCGGAGGTG AAACTGGAAC | | |
| | CACCGGCGCC GCAAAAGGCA AAGCATTATA | | |
| | GGGGAGTGAG AGTGAGGCCG TGGGGGAAGT | | |
| | TTGCAGCGGA AATTAGGGAT CCGGCAAAGA | | |
| | ATGGGGCAAG GGTGTGGCTG GGTACGTATG | | |
| | AGACGGCAGA GGACGCAGCG TTTGCTTATG | | |
| | ACAAGGCGGC GTTTCGCATG CGGGGGTCAC | | |
| | GTGCATTGCT TAATTTCCCG TTAAGGATTA | | |
| | ATTCTGGTGA GCCTGATCCC ATTAGAGTTG | | |
| | GTTCTAAAAG GTCATCAATG TCGCCGGAGT | | |
| | ATTCTTCTTC TTCATCGTCG TCGGCGTCGT | | |
| | CGCCGAAGAG GAGGAAGAAG GTATCTCAAG | | |
| | GGACGGAGCT AACGGTGTTA TAGGTCCCAA | | |
| | CTGGGTTCTG TGTAGTGATT AAGAAAAATA | | |
| | GAATTAGTCG AGGGAATTTG TTTTTTACTT | | |
| | GGCTGAAGTA ATGAATTTGT TATTTATTTA | | |
| | TTTTTTGACT GTGGTTGAAA TTGAATCAAA | | |
| | AAAAAAAAAA AAAAAGTACT AGTCGACGCG | | |
| | TGGCCTAGTA GTAGTAGA | | |
| C331 | GGGTGACACT ATAGAATACT CAAGCTATGC | putative protein | SEQ ID N° 149 |
| | ATCCAACGCG TTGGGAGCTC TCCCATATGG | At3g62270 [A. | |
| | TCGACCTGCA GGCGGCCGCG AATTCACTAG | thaliana] | |
| | TGATTAGCGG ATAACAATTT CACACAGGAA | | |
| | ACAGCTATGA CCATTAGGCC TATTTAGGTG | | |
| | ACACTATAGA ACAAGTTTGT ACAAAAAAGC | | |
| | AGGCTGGTAC CGGTCCGGAA TTCCCGGGAT | | |
| | GTGTCCTTTT CCCAATGTTG ATCATGCTGC | | |
| | TTGTCCCAGT GCGCCAGTAT TTGCTTCCCA | | |
| | AGTTTTTCAA AGGAGGACAT TTGCAAGATT | | |
| | TAGACGCTGC AGAATACGAA GAAGCTCCTG | | |
| | CAATAGCTTA CAATATGTCC TATGGAGATC | | |
| | AAGATCCTCA GGCAAGACCT GCCTGCATTG | | |
| | ATAGTAGTGA AATTCTTGAT GAGATAATCA | | |
| | CAAGAAGCCG TGGGGAGATC CGGCATCCAT | | |
| | GCAGCCCAAG AGTGACTAGT TCCACTCCTA | | |
| | CCAAACTTGA GGAAATCAAG TCTATGCACA | | |
| | GCCCACAGTT AGCACAAAGG GCTTACAGTC | | |
| | CAAGAGTCAA TGTACTAAGA GGAGAAAGGA | | |
| | GCCCCAGATT GACGGGCAAG GGACTTGGAA | | |
| | TAAAGCAAAC TCCTAGCCCC CAGCCATCTA | | |
| | ATCTGGGTCA AAATGGTCGT GGTCCGTCTT | | |
| | CTACCTAG | | |
| C332 | GAGATGTCGTTTCTTGGAATTCCGATGGGACGGCG | putative heat shock | SEQ ID N° 150 |
| | TTTGTTGTGTGGCAGCCGGCGGAATTTGCTAGAGA | transcription factor | |
| | TTACTTCCAACTCTCTTCAAACATAGCAACTTCTCC | | |
| | AGCTTTGTCCGGCAGCTCAATACCTATGTATGTTAT | | |
| | CCTTCTATTTACTGTCTAAAAAAATTTATTCTTATTC | | |
| | CGTGTTTGCATTA | | |
| C333 | GATCCGATGA AAACGATGTC GTTGTAATCG | ferric | SEQ ID N° 151 |
| | GCGGTGGTCC CGGCGGCTAT GTGGCGGCGA | leghemoglobin | |
| | TCAAGGCCGC TCAGCTCGGG CTGAAAACTA | reductase | |
| | CTTGTATTGA GAAACGTGGT ACCCTCGGTG | | |
| | GTACTTGCCT TA | | |
| C334 | GGGGCAAGGGAGTGGCTGGGTACGTATGAGACGG | ethylene responsive | SEQ ID N° 152 |
| | CGGAGGACGCAGCGTTGGCATACGACAAGGCGGC | element binding | |
| | GTTTCGCATGCGGGGGTCACGTGCATGGATTA | factor | |
| C335c | GATCCGTCAA AACCCTCGGC AACTTTGTCA | 40S ribosomal | SEQ ID N° 153 |
| | AGGCAACCTT TGATTGTTTA | protein S2 | |
| C336a | GATCCGTTCG TGTATCCTGT GTTTCAAGCT | cytochrome P450 | SEQ ID N° 154 |
| | GGACCTAGGG TTTGTTTAGG GAAGGAAATG | | |
| | GCATTCTTGC AGATGAAGAA GGTGGTTGCC | | |
| | GGAGTTCTAC GGCGGTTTAG GGTGGTTCCG | | |
| | GTGGTGGAAA AAGGTGAAGA GGAGCCAGTG | | |
| | TTGATAGCTT ACCTTACTAC TAGGATGAAG | | |
| | GGTGGTTTCT TGGTGAGGAT TGAGCAAAGG | | |
| | ACAAATTGAT AGGACCCACA CTCCCTTCCC | | |
| | TTACAATAAT AAAATCTCCG TTA | | |
| C336b | GATCCGTACT GTACTTTTGA GCATTCAAGC | ubiquitin | SEQ ID N° 155 |
| | ACTTTTGAGT GCTCCAAACC CGGATGATCC | conjugating enzyme | |
| | ACTCTCTGAA AACATTGCAA AGCACTGGAA | | |
| | GTCAAATGAG GCTGAAGCTG TTGAAACGGC | | |
| | CAAGGAGTGG ACACGCCTAT ATGCTAGTGG | | |
| | TGCATGAAGA CATAGCAACG AGATATTCAA | | |
| | AAATAACAAA AATTATGGAA TGTATTCTAT | | |
| | TGACTTGCTT ATCAATATGA CACTTCGGAC | | |
| | GGCTGTTA | | |
| C338 | GGGAGGGGCCCGCAAGCACATGTCGAAAATCAGG | putative protein | SEQ ID N° 156 |
| | ATTGATGTCAATGCTGATCAGCACCCCTTTCAGTAC | At4g24290 [A. | |
| | GAAACTAAATCAACCACAGAAGCCAGCTAAGGTGG | thaliana] | |
| | ACCTGAACTCCGCAGTTTATCCTGGCGGTCCACCTT | | |
| | CACCGGCAAGGGCGCCAAAGATGTCGCACTTTGTC | | |
| | GATACAACAGAAATGGTAAGAGGACCTGAGGAGT | | |
| | CACCTGGCTACTGGGTGGTAACTGGTGCAAAGCTA | | |
| | TGTGTAGAAGATAGTAGGATAAGAATGAAAGTGAA | | |
| | GTACTCGCTCTTA | | |
| C339 | TAAGCAGCTC AATTCCGATC TTCACTGGTC | putative serine-rich | SEQ ID N° 157 |
| | TGAGACGGCC CTCTGTTCAA GTACCCCTTC | protein | |
| | TTCTACTCGA GCCTCGGCAG AGCCTTTTTG | | |
| | ATCTCATTCG TATTCTAAGG AATTCTAAAG | | |
| | GACTCTTTCA TATTGCACCG GAGCTGGAAA | | |
| | AGATTGGACT ATTCCCTAGC GAGACAACA | | |
| C34 | AACATTCGCATTAGCAACAAAACATTCCTACACAT | ambiguous hit | SEQ ID N° 158 |
| | CGTAACAGAATCAAGCATTCATAATATTGTAATAG | | |
| | AACCAAAACAAAATGAAAGAAGTAATTCACCACCA | | |
| | AAAATGGAAACCTCGAACCAGACCAGAAAACCTG | | |
| | CCAGAACCGCAACAAAACTCCACAACGGGCCTCAT | | |
| | CGGCACCTCAGATTTGCTCGATTTCTTTTGGAGATG | | |
| | CGACTGCGTG | | |
| C341a | GATCCGTGGC TCTAAGGCTC GGCTCAACTT | putative ethylene | SEQ ID N° 159 |
| | GCCTCACTTA | response element | |
| | | binding protein | |
| C341b | GATCCGTGAT GGACTTCTTC AGGCTTCTGT | hypersensitive- | SEQ ID N° 160 |
| | TTAGCTTA | induced response | |
| | | protein | |
| C347a | GATCCGCAAG GGACCTGCAC CATATAATCT | porin | SEQ ID N° 161 |
| | GGAGGTGCCT ACTTATAGTT TCCTGGAAGA | | |
| | GAACAAGTTA CTTATTGGTT ACTCAGGACT | | |
| | CATCGTAGAC TGCGTAGTGA TCTTCTGTAC | | |
| | AGGGACTATG TCAGTGACCA TAAGTTCACC | | |
| | GTCACTACCT ATAGCTCAAC CGGAGTGGCT | | |
| | ATTACCTCAT CTGGTCTGAA GAAAGGTGAA | | |
| | TTATTCTTAG CCGACGTTA | | |
| C347b | GATCCGCCCAGGTCAAGATGTTACTGTACGAACAG | cytoplasmic | SEQ ID N° 162 |
| | AAACTGGAAAATCCTTCACTTGCACAGTGCGGTTC | aconitate hydratase | |
| | GACACCGAGGTGGAGTTGGCTTATTTCAACCATGG | | |
| | AGGTATTCTGCCATATGTCATTCGTCAGTTGACTAA | | |
| | GCAATAAGGGACCGTTTTGATAATTTGGCCACCTTC | | |
| | ACGAGCTGCTGGTGCTTA | | |
| C348 | TAACCCCAAA AAGACGAATA TTGTGGTGTT | putative ribosomal | SEQ ID N° 163 |
| | CTAACAGCGG CAGATCAAAG AAGAACTTGA | protein | |
| | TGAGCGAAAT CCGCTGACAA AAAAAAGAGA | | |
| | ACTTTTTGAA TTCCGATGCC TAGCGTCCCC | | |
| | TGATAACCTA GGATTAGTGG TGATAGGGCT | | |
| | GATGTGGTAT CTCGGAAACT GGGATTTGAT | | |
| | GGTATCTGTA GAGCGGATC | | |
| C349a | GATCCGCATG ACCTTTGTGA GCAACACCCT | arogenate | SEQ ID N° 164 |
| | GATGTTATTC TCCTTTGTAC TTCAATTATA | dehydrogenase | |
| | TCTACTGAAC CTGTCCTTAG ATCACTCCCT | | |
| | ATTCAAAGGC TAAAAAGAAA CACATTGTTT | | |
| | GTTGATGTTT TGTCTGTTA | | |
| C349b | TAACATTCCC AGCAATCGAT CACAACTACA | putative membrane | SEQ ID N° 165 |
| | AGAAGAGCAA AATAACTATG AGAAGATGTT | protein | |
| | ATCTTCAGCA AATTCAGTCA GACCCATTCT | [Saccharomyces | |
| | TATTACTCCA TTATGTGCCG CTTGCGCAAG | cerevisiae] | |
| | CCCACAGGCA GTGGCGGATC | | |
| C349c | GATCCGCAAA AATCAGAACC TGGAACAATC | nucleoside | SEQ ID N° 166 |
| | AGAGGTGAGT TAGCTGTTGT AGTCGGAAGG | diphosphate kinase | |
| | AACATCATCC ATGGAAGCGA TGGACCCGAG | | |
| | ACTGCCAAGG ATGAGATCAA ACTATGGTTC | | |
| | AAACCAGAAG AGTTGGTTA | | |
| C350 | TTCTCAGCCAGCCGTGGAACTACAAAGGCCACTCC | putative protein | SEQ ID N° 167 |
| | ATCTAAGGCAAAGTATAGACCTCTGGAGACAAGGG | At3g52110 [A. | |
| | GTATCCTTCAAGAACTGGAACAGAGCAGCAATGAA | thaliana] | |
| | GAGAAGAGAAAGGAAGATCAAGGGAAGATGATGA | | |
| | GTAATAATCAACAAGGACAGAGAGGTGGTGCTATT | | |
| | GTTGCTGAAAAAGAAGCTGCTGCTAGAGCTTTGGA | | |
| | TGTCTTCTGGTTCTTGAAACCTTGCACTCTTTCCAG | | |
| | CTGAAATGGTCAAAGCCCACTGCTGCAGAACATTT | | |
| | CATGAAGTGATTCTTTCATACTTA | | |
| C351a | TGACTGCGTAATGATCCGCTATLTTCCACACAGAGG | stromalin 3 | SEQ ID N° 168 |
| | ACACCTATTGGACAATCTTCCACCCTTCCATTTCTG | | |
| | CCGACAGTGTTGAGCTCAAAGAACGGCAAAGGAA | | |
| | AAATGACCCCACTCAATTCCAAACTTCAGTTCGTCA | | |
| | CTTTTCCTCTAAGCAACCCAATTAGCTTA | | |
| C351b | GATCCGCCAA AAATACAATA ATTATGAAGG | ambiguous hit | SEQ ID N° 169 |
| | ATGCGACACG CACACCGAGA CATTTTCGGA | | |
| | GAGTGCGAGC AACATAGGTT GGAATATTTA | | |
| | CAGCCTTAGG AGGCTTCAGG AATAATGTAT | | |
| | AACAACGTTT TCTTTATTGC TTTATTTTCA | | |
| | CTTCTCTTA | | |
| C352b | TAAGGGTTCA ACCTTTAGTT CTTACGATTG | muconate | SEQ ID N° 170 |
| | CGTACCCATT GCATTGGAAT TATACGTAGG | cycloisomerase | |
| | TGGAAACCTT GGATTCCCAG CATAGGCGGA TC | | |
| C352c | TGACTGCGTA GTGATCCACC AAAACCCTTG | 40S ribosomal | SEQ ID N° 171 |
| | GCAACTTCGT TA | protein S2 | |
| C353a | ATGAATCCAG AATACGACTA TCTTTTCAAG | GTP-binding | SEQ ID N° 172 |
| | CTTTTGCTTA TTGGAGATTC TGGTGTTGGC | protein | |
| | AAATCATGTC TCCTCTTGAG ATTTGCTGAT | | |
| | GATTCATATC TTGAGAGTTA CATTAGTACC | | |
| | ATTGGTGTTG ACTTTAAAAT CCGCACAGTT | | |
| | GAGCAGGATG GGAAAACCAT TAAACTTCAA | | |
| | ATTTGGGATA CTGCTGGTCA AGAACGTTTT | | |
| | AGGACAATTA CCAGCAGCTA CTATCGCGGT | | |
| | GCTCACGGCA TAATTGTTGT CTATGATGTA | | |
| | ACCGATCAAG AGAGTTTCAA TAATGTCAAG | | |
| | CAATGGTTGA GTGAAATTGA TCGATATGCA | | |
| | AGTGATAATG TGAACAAACT TCTTGTCGGA | | |
| | AATAAGTGCG ATCTCACAGC GCAGAAGGTA | | |
| | GTTTCCACAG AGATAGCTCA GGCTTTTGCT | | |
| | GATGAGATCG GCATTTCCTT CATGGAAACT | | |
| | AGTGCGAAAA ATGCCACCAA TGTGGAACAG | | |
| | GCTTTCATGG CTATGGCTGC TTCAATCAAG | | |
| | AACAGAATGG CAAGCCAACC AGCATCAAGC | | |
| | AATGCACGGC CTCCAACTGT GCAGATCCGC | | |
| | GGACAACCTG TCAACCAGAA GAGCGGTTGC | | |
| | TGCTCATCTT AA | | |
| C353b | GATCCACCAAAACCCTTGGCAACTTTCGTTTA | 40S ribosomal | SEQ ID N° 173 |
| | | protein S2 | |
| C354 | AATACGATCCCACTATACATATCGATATACATAG | putative | SEQ ID N° 174 |
| | AGATTCACCGACTACATTTCAGCCATCCAGCGATC | oxidoreductase | |
| | CTGATCTATTTGAAAATTGTTAGAATTGATATATCC | | |
| | ATATATCATATTTCTGCGGGCATAAGAGTTTTTTCC | | |
| | TTTATGTTCGGTGGAAATCACATGTTATACTATATT | | |
| | CCAATAAATAGATATCTGTGTTATGATACAAGTCC | | |
| | ACGTTTTCAAAAAAAAATGGATGAGATTGGGTCCC | | |
| | AGCGGATC | | |
| C355a | GATCCGCCGC TAACACCTAA AACACCCCCC | protein kinase | SEQ ID N° 175 |
| | TCCCTTGAAG CTTCTTCTTC TTCGAACCCA | | |
| | CCCACCTCGG CCGTTACCCC TCCTATTA | | |
| C356a | GATCCGCAAC TAATGCTCTT ATCGGTGCAG | glutamate/aspartate- | SEQ ID N° 176 |
| | TCAGTGCTAT AATTTTCTGT GGATACATTG | binding peptide | |
| | TATATGACAC AGACAACCTG ATTA | | |
| C356b | GATCCGCCGC TAACACCTAA AACACCCCCC | extensin | SEQ ID N° 177 |
| | TCCCTTGAAG CTTCTTCTTC GAACCCACCC | | |
| | GCCTCGGCCG TAACCCCTCC TATTA | | |
| C358 | GATCCTAGTT TGGAATATGA GCTCTCTGCT | putative potassium | SEQ ID N° 178 |
| | CTTCGAGAAG CCACAGAATC TGGATTTACA | transporter | |
| | TATTTGCTTG GACATGGGGA CGTGAGGGCG | | |
| | AAGAAAAACT CTTGGTTCAT CAAGAAACTG | | |
| | TCAATAAATT ACTTCTATGC ATTCATGAGG | | |
| | AAGAACTGTA GAGGAGGCGC TGCAACAATG | | |
| | CGTGTTCCTC ACATGAATAT TATCCAGGTG | | |
| | GGAATGACAT ACATGGTTTG ATCTTGGTAC | | |
| | CATTTAGCTT CTTGCTGGCC TTGTAAGTGC | | |
| | TGCATTA | | |
| C359 | CTGTACAAGTGATGAAGTGCCCTTCACGGTTTCCTC | AtSIK-like protein | SEQ ID N° 179 |
| | TGCAAGAACCAGTGGCAGTTGGTGGTAAACATATG | kinase | |
| | TCAAAGTCTCCAAGTATGACTGGAATCATCACCCCT | | |
| | GCGCCAAGGTTGAGTTTCTCCCCTTCCTTACCTATC | | |
| | ACCCGAGGATCGGCTTCTCCCTCAAAGTCTTCTACG | | |
| | CAGCCCTCGTCTCGTCCTTCATTA | | |
| C360 | CCACGCGTCC GCCGAAATTC TGAAGCAATA | putative protein | SEQ ID N° 180 |
| | ACAAAGAATG GGTTGCATCG AAAAGGATCC | At4g14710 [A. | |
| | AGGAGAGGAC GTCGTACAGG CATGGTACAT | thaliana] | |
| | GGATGACAGC GATGAGGACC AGAGGCTTCC | | |
| | CCATCACCGT GAGCCAAAGG AATTTGTGTC | | |
| | TCTTGACAAA CTTGCTGAGC TTGGAGTGCT | | |
| | CAGCTGGAGA CTTGATGCTG ACAATTATGA | | |
| | GACAGAGGAG GAGTTGAAGA AAATTCGGGA | | |
| | AGCTCGTGGC TATTCTTACA TGGATTTCTG | | |
| | TGAGGTTTGC CCTGAGAAAC TACCGAATTA | | |
| | TGAGGAGAAA ATCAAGAACT TTTTTGAAGA | | |
| | ACACCTGCAC ACCGACGAAG AGATCCGTTA | | |
| | CTGTGTTGCA GGAAGTGGTT ATTTTGATCT | | |
| | CCGGGATCGG AATGATGCTT GGATTCGTGT | | |
| | CTGGGTAAAG AAAGGTGGAA TGATTGTTCT | | |
| | GCCTGCTGGA ATTTATCACC GCTTCACACT | | |
| | TGATTCAGAC AATTACATTA AGGCAATGCG | | |
| | ACTCTTTGTT GGTGACCCAA TTTGGACTCC | | |
| | ATACAATCGC CCACATGACC ATCTCCCTGC | | |
| | AAGGAAAGAA TATATTGAAT CGTTTATCCA | | |
| | AGCAGAAGGC GCTGGCCGTG CAGTTAATGC | | |
| | TGCTGCTTAA ATTTACTAGA GGCGAAGAAG | | |
| | TTGAAATCCT TATAGGCTGT AATAAATGTT | | |
| | ACCATATGAT GGTTGTGTGG TTCCTGAAGT | | |
| | GTGCGCCTGG CTCAGCTTGT TGAATGTTGT | | |
| | AATTCGAGCA CTAAATAAAT CTCCTATGGG | | |
| | GATATTGAAC TTAATAGTTA TATACACCTG | | |
| | GAGTCTATGT TGTGAATTTA AACATTTGTG | | |
| | CATGTCGAGT GGTACAATAT TTCCTGTTTC | | |
| | GGGGCGTAAT TAGCTCTGCC ATTTTTGTTG | | |
| | TTGGATTGCA ATGACCTTGA ACTTCTTGAA | | |
| | CTTAAAAAAA AAAAAAAAA | | |
| C364a | GATCCGGGTC ACTTCCCTAC ATTGGGTGGC | probable | SEQ ID N° 181 |
| | AAGTGATGCT TTATTAGTGC TTTTCTCCCA | transcription factor | |
| | CGTCCAAGAG GCAAATTGAC TGAAAAATAA | | |
| C364b | GATCCTCAAG CATTTATTCG CCACTTTTAC | heme oxygenase | SEQ ID N° 182 |
| | AACACATACT TTGCGGATTC AGATGGAGGT | | |
| | CGCATGATAG GGAGAAAGGT GGCTGAAAAG | | |
| | ATACTCTGAC TGCGTAGTGA TCCGGCTATG | | |
| | TTGCTGATCA ATCTAGTTAT GGCATGGTTG | | |
| | ATCCTTCTCA GCATTATTAT CCGGAGCAAC | | |
| | CATCCAAGCC GCAGCCAAGC ATTTCGAACA | | |
| | GTCCTTATGC TGAGAATTAT CAACAACCAT | | |
| C364c | GATCCTCAAG CATTAATTTG CCACTTTTAC | heme oxygenase | SEQ ID N° 183 |
| | AACACATACT TTGCGCATTC AGCTGGAGGT | | |
| | CGCATGATAG GAAGAAAGGT GGCTGAAAAA | | |
| | ATACTCAACA AGAAAGAGCT GGAATTCTGA | | |
| | CTGCGTAGTG ATCTTGGAGT GAATATGGAC | | |
| | GAGGACTACT TACTGCGAAA TGCTAGTAGT | | |
| | CGGTAATTCT TCTTCCTCTG TTGATGCTGT | | |
| | GGAGAGAGCT AGAGCGTGGG G | | |
| C365 | TTGACAGGATCGATCATGCCAAATTCTTCATCATCT | putative protein | SEQ ID N° 184 |
| | TCTTCGCTAATTCCAAACGAGTCCACGCTGATGGA | At1g26190 [A. | |
| | AGAGCTATCTAATGTTGCACCTGGACAACGTCAAA | thaliana] | |
| | TTATACATCAGTTGGACAATCTTAGCAATCTTCTTC | | |
| | GCGACAGGCTAGGAGAACAATCTCGGCAATCAAGA | | |
| | AAAAGCAAGAGAAGAGATATTACCGATATTTGATTC | | |
| | GATCAGAGTGCCTCTCATTGTAACCTTAGCAGTTGG | | |
| | TGGATTGGGATTATTTTTGTTTA | | |
| C366a | GATCCGGGAA GTTTGGTCCG ATAATATTGA | CCR4-associated | SEQ ID N° 185 |
| | TTCTGAATTT GAGCTTATAC GAACAGCTAT | factor | |
| | TGATCAGTAC CCTTACATCT CAATGGATAC | | |
| | TGAATTCCCG GGCGTTATTT TCAAGCCGGA | | |
| | GGTTTGGTCT TTCCAGCAAA ATCGCCGGCG | | |
| | ACATGGACAA CATTATAAGT TGTTACTCAG | | |
| | GACTCATCAA CTAATGAGGA AACCGCGAAA | | |
| | TCTGTATACT TTCTAAAACC CCAAAAGGTT | | |
| | TGCTCTTTCA GTTTTA | | |
| C366b | TAAAGCTAGC GGGGTTAGTG ATATCCTTGT | 6-phosphogluconate | SEQ ID N° 186 |
| | TGACCAGTCC GTGGATAAGA ATCAGTTGAT | dehydrogenase | |
| | TGACGATGTG AGAAAGGCAC TTTATGCATC | | |
| | CAAAATATGT AGCTATGCTC AGGGCATGAA | | |
| | TTTGATAAGG GCAAAGAGCG TTGAAAAAGG | | |
| | ATGGGATTTG AAACTAGGGG TGCTTGCTAG | | |
| | GATTTGGAAG GGTGGTTGTA TTATCCGTGC | | |
| | TATATTTTTG GATCGCATCA AGGGGGCTTA | | |
| | TGACAGAAAC CCGGATC | | |
| C367 | GATCCGGCAT GTTTTTTTAC TCAGGACTCA | ambiguous hit | SEQ ID N° 187 |
| | TCGTTAAAGA ATCAAAGGTT CAAGTGAAAT | | |
| | CATGCCCCGT GCTCCTAAAG TACGCTTTCA | | |
| | TATTTGGGAA CACTTTGAGG TGAAAGAAGA | | |
| | TAACGGAGAA GTTCGCAAAG TAAAGTGCAA | | |
| | GCAATGTGGT CCAGTCTATA ATTTCATCCA | | |
| | AAGAGGGATG GCACATATTG TTTA | | |
| C368b | GATCCCGAGC AGGAGAGCGA TAACATTGTT | ankyrin like protein | SEQ ID N° 188 |
| | TTAGTCGTGC AAAAGAAGTT GTGGCTCACA | | |
| | AGTGGAAGCA TCAGAGATAC AGAATAGACA | | |
| | GTAGAGTTTG AACACTTCTT CCTGACTCTG | | |
| | CCTTTAGGGA | | |
| C369 | GATGAAGAAGCTGCAATTGCTTATGATAAAGCGGC | ethylene-responsive | SEQ ID N° 189 |
| | TTATCCAATGCGCGGTCCAAAGGCTCATTTA | transcription factor | |
| C4 | GTTTGACAAT GCCTACTTCA AAAATTTACA | peroxidase | SEQ ID N° 190 |
| | GCAAGGTATG GGACTATTCA CATCATGATC | | |
| | AAGTGCTTTA CACGGACGGG CGGTCCAAGG | | |
| | GAACTGTCGA CATTTGGGCT AGTAACTCAA | | |
| | AAGCATTCCA AAACGCATTC GTCACTGCAA | | |
| | TGACAAAGCT GGGCCGTGTT GGTGTGAAAA | | |
| | CTGGGAGGAA TGGAAATA | | |
| C401 | GATCCTATAG CCAACCTAAC AATTTACCCC | putative protein | SEQ ID N° 191 |
| | TCTTCGGATC GGTTCTTGTT GGAAAAGATT | At2g44230 [A. | |
| | CAAAAGGAGA CGCGCTAAAG ATCCCAATTG | thaliana] | |
| | ACTATACACT TGTATGGAGT AGTGAGAACT | | |
| | TGAATATCAA GCAGGATAGT GTTGGCTATA | | |
| | TTTGGATGCC AATTCCTCTT GAAGGCTATA | | |
| | AAGCCGTAGG CCACGTTGTA ACAACGTCGC | | |
| | CTCAAAAGCC TTCTCTTGTC ATAATTCGTT | | |
| | GAGTTCGTTA TATTTTA | | |
| C402 | GGTGCTTATATTGTTAGACAGGAGGCAAAGAGTGG | S-adenosyl-L- | SEQ ID N° 192 |
| | GGGCGCCTCAGGACTTGCTCGCCGTTGTCCTGTGCA | methionine | |
| | GGTTCCTTATGCTATCGGTGTGGCTGAACCACTTTC | synthetase | |
| | CGTGTTTGTTGACACTTACAAGACTGGAACAATTCC | | |
| | AGACAAGGATATTTTGGCTCTGATCAAGGAGAACT | | |
| | TTGACTTCAGGCCTGGAATGATGTCAATCAATCTTG | | |
| | ACTTGTTA | | |
| C408 | ATGCTCTTCTCCTATTCATTTGACTCACAATGTATC | beta-glucan binding | SEQ ID N° 193 |
| | CTCCATAATTTCTAATGGATTCTCGGGTGTAATACG | protein | |
| | AATTGCTCTCTTGGCTAATTCTGATCGCCAATGTGA | | |
| | GAAAATTCTTGATCAGTACAGCTCGGCTTATCCCGT | | |
| | GTCTGGAAGTGCAACTTTGAGGCCTTTTGGTCTTAG | | |
| | TTACAAATGGGATGTGAACGGTAAAGGCAAGTTTGC | | |
| | TTATGCTTGCTCATCCTCTACATCGCCGACTTCTTTC | | |
| | AACAGCAGATTCTTCAGTAACTATTTTGGATGATTT | | |
| | CAAGTATAGGAGCATGGATGGTGAGCTTTGTTGGCG | | |
| | TTGTTGGAAATTCGTGGGAGCTTGAAACGGATTCA | | |
| | ATTCCAATATCATGGCATTCGGTTA | | |
| C409a | GATCCTACTAAGGTGGACATGAGTGGTGCTTATATT | S-adenosyl-L- | SEQ ID N° 194 |
| | GTTCGACAGGCAGCAAAGAGTGTGGTCGCCTCAGG | methionine | |
| | ACTTGCTCGCCGCTGTATTGTGCAGGTTTCTTATGC | synthetase | |
| | TATCGGTGTGGCTGAACCACTTTCCGTGTTTGTTGA | | |
| | CACTTACAAGACTGGAACAATTCCAGACAAGGATA | | |
| | TTTTGGCTCTGATCAAGGAGAACTTTGACTTCAGGC | | |
| | CTGGAATGATGTCAATCAATCTTGACTTGTTA | | |
| C409b | GATCCTCTGA GGCTATTATG CTTGCTGGAT | glutamate | SEQ ID N° 195 |
| | AGCTTTTCAA GAGAAAATGG CAAAATAAAA | decarboxylase | |
| | TGAAAGCCCA AGGCAAGCCC TGTGACAAGC | | |
| | CCAATATTGT CACTGGTGCC AATGTCCAGG | | |
| | TGTGTTGGGA GAAATTTGCA AGGTATTCTG | | |
| | AAGTGGAGCT AAAGGAAGTA AAGTTGAGTG | | |
| | ATGGATACTA TGTGATGGAC CCTGAGAAAG | | |
| | CTGTGGAAAT GGTGGATGAG AACACAATTT | | |
| | GTGTAGCTGC TATGTTGGGT TCCACACTCA | | |
| | ATGAGATAAA TTTGAAGATG TTTA | | |
| C410 | GATCCTCAAG GCCCCAAAAT TTGATATCGG | 40S ribosomal | SEQ ID N° 196 |
| | CAAGCTGATG GAGGTTCATG GTGACTATTC | protein S3a | |
| | AGAAGATGTT GGCGTGAAGT TGGATCGACC | | |
| | AGCTGATGAG ACCGTTGCTG AGGCAGAACC | | |
| | TGAGATTCCT GGAGCTTAGA CTTGTTTGAT | | |
| | TTGGATTCTG TCTGAATATG GTGCTTGTCT | | |
| | TCTAAATTTA TGAATTTGTT TTAGTTGAGG | | |
| | TGTCAAAGGC GCGGCCTAAC AAAATATTGG | | |
| | ATATCTTTCT TTGGTTACGT TTGATGTTA | | |
| C414c | TAAGCATACA TAGAAGTTAC ACTGCTTTCA | DNA polymerase ? | SEQ ID N° 197 |
| | TCTCACTCGT TGTAGTGCAG ATCATACACT | | |
| | GGCTATCTTT AGCACCTAGA GAATGAAGCA | | |
| | TCATCTGATG CCTTTACTGA ATTTGCTTTT | | |
| | CAAAACTTCC TGTAATTGCT AGGATC | | |
| C417a | TAAGCACCGTTTAGGAGATTTATTCTACCGTTTGGT | vacuolar H+- | SEQ ID N° 198 |
| | GTCCCAAAAGTTCGAGGATC | ATPase | |
| C418 | CCTTGGTGGAGCTTGCGGTTACGATAACCCTTATG | expansin | SEQ ID N° 199 |
| | ACGCCGGATTTGGAGTAAACACAGCGGCATTGAGT | | |
| | AGCGCACTGTTCAGAAATGGAGAAGCTTGTGGAGC | | |
| | TTGCTACACAGTAAGATGCAACCGCAAACTCGATC | | |
| | GTAAGTGGTGCCTCCCACATGGGGCCGTCACTGTG | | |
| | ACGGCCACCAATTTTTGCCCTCCGAACAACCACGG | | |
| | AGGGTGGTGTGATGCACCACGACAACACTTTGACA | | |
| | TGTCCATGCCCGCTTTCCTTCGCATTGCTCGACAAG | | |
| | GCAATGAAGGCATTGTTCCTATTCTCTACAAAAGG | | |
| | GTGTCATGTAGGAGAAGAGGAGGAGTACGTTTCAC | | |
| | ATTA | | |
| C419 | GGATATGAGCTCTCTGCTCTTCGAGAAGCCACAGA | putative potassium | SEQ ID N° 200 |
| | ATCTGGATTTACATATTTGCTTGGACATGGGGACGT | transporter | |
| | GAGGGCGAAGAAAAACTCTTGGTTCATCAAGAAAC | | |
| | TGTCAATAAATTACTTCTATGCATTCATGAGGAAGA | | |
| | ACTGTAGAGGAGGCGCTGCAACAATGCGTGTTCCT | | |
| | CACATGAATATTATCCAGGTGGGAATGACATACAT | | |
| | GGTTTGATCTTGCTGCCATTTAGCTTCTTGCTGGCC | | |
| | TTGTATGTGCTGCATTA | | |
| C420 | CAAGTGGACAGAAGTGGTGCTTATGTTTGTGAGACA | S-adenosyl-L- | SEQ ID N° 201 |
| | GGCAGCAAAGAGTGTGGTTGCTGCAGGACTTGCTC | methionine | |
| | GCCGCTGTATTGTCCAGGTTTCTTATGCAATTGGTG | synthetase | |
| | TGGCAGAACCACTCTCCGTGTTTGTTGACACTTACA | | |
| | AAACCGGAACCATTTCCAGACAAGGATATTCTGGCT | | |
| | CTGATCAAGGAGAACTTTGACTTCAGGCCTGGAAT | | |
| | GATGGCAATTA | | |
| C421 | CCAATCCGATATAGCCGATGGCTTCCATGAATAT | acyl-CoA oxidase | SEQ ID N° 202 |
| | ATTAGGCCACTACTCAAGCAGCAACTGCATACTGC | | |
| | TCGACTGTGAAGGAGAGTTGCATATATTTATAGC | | |
| | TGTTGTATTGTGCTGTGCCAATAAACTAAAATTGA | | |
| | AATATCATCTTTCTTTTGGATGATGGCCTCCTTTAT | | |
| | GACTTACATAGCGGTGATTA | | |
| C422 | GACAAAACACTTGGATCCTGACAATTATCTGCTGA | putative annexin | SEQ ID N° 203 |
| | TACCCAGCACTAGGAATGTTCATCAGCTTAGAGCA | | |
| | ACTTTTGAGTGCTATAAGCAAAATTACGGATTCTCC | | |
| | ATCGACCAGGACATTA | | |
| C423a | ACTAGTGATTGACTGCGTAGTGATCCTGCTGGTCCG | spermidine synthase | SEQ ID N° 204 |
| | GCTCAAGAGCTTGTGGAAAAACCATTCTTTGCAAC | | |
| | GATAGCAAGGGCATTA | | |
| C423b | ACTAGTGATTGACTGCGTAGTGATCCTAAGAAAAT | putative protein | SEQ ID N° 205 |
| | TGCCCGTGTGATGGACCGACGACTTGAAGGTGAAT | kinase | |
| | ACCCGATTA | | |
| C425 | GGTGCTATTACAATTTTGGACACATCAAGTGATCCA | vacuolar H(+)- | SEQ ID N° 206 |
| | AGGACACTTGCTGTTGCTTGCTATGATCTATCACAG | ATPase subunit-like | |
| | TTCATTCAGTGCCATTCTGCTGGGCGAATCATAGTG | protein | |
| | AATGACCTCAAAGCTAAGGAGCGCGTAATGAAACT | | |
| | GTTGAACCACGAGAATGCAGAGGTCACAAAAAATG | | |
| | CCTTACTCTGTATCCAAAGGCTTTTCCTAGGTGCCA | | |
| | AGTATGCTAGCTTTTTGCAGGTTTA | | |
| C426a | GATCCTCAAG GCCCCTAAGT TTGATATTGG | 40S ribosomal | SEQ ID N° 207 |
| | CAAGCTGATG GAGGTTCATG GTGATTATTC | protein S3a | |
| | AGAAGATGTT GGTGTGAAGT TGGATCGGCC | | |
| | AGTTGATGAG ACAGTGGCAG AGGCAGAACC | | |
| | CGAGGTTCCT GGAGCGTAGA CTCGTTTCGT | | |
| | GCTTCCGAAA TATGTGTTCG AATATGGTGA | | |
| | TAGTCTTTAG AGCCTCACAT TGTTTA | | |
| C426b | GATCCCACCAGATCAGCAGAGGCTCATATTTGCTG | ubiquitin | SEQ ID N° 208 |
| | GTAAGCAGCTGGAGGATGGGCGCACCCTTGCAGAT | | |
| | TACAATATCCAAAAGGAATCCACACTCCACCTTGT | | |
| | GCTTCGCCTTCGTGGTGGTGACTATTGAGGATTGAA | | |
| | GTGCTGCTGCTGGGGTTTTACATAAGATGCCTGCTT | | |
| | CTTTGTTCTAATGGTTCTGTTGTTA | | |
| C428a | GATCCTGATG TTACTGCCCG CCCTAAAGCT | putative protein | SEQ ID N° 209 |
| | CTTGAGTGCA ATCTCATCTT TA | At1g27760 [A. | |
| | | thaliana] | |
| C428b | GATCCTCCAA GGAGATAGCT TTGGCATCTC | putative protein | SEQ ID N° 210 |
| | ATTTTCTTGG AATTTTGGCT TTA | At3g09350 [A. | |
| | | thaliana] | |
| C429 | GATCCTGCTGGTTGGCTAGAATGGGATGGTAATTTT | putative | SEQ ID N° 211 |
| | GCTTTA | pectinesterase | |
| C430 | GCTCATTACAATTTTGGACACATCAAGTGATCCAA | vacuolar H(+)- | SEQ ID N° 212 |
| | GGACACTTGCTGTTGCTTGCTATGATCTATCGCAGT | ATPase subunit-like | |
| | TCATTCAATGCCATTCTGCTGGGCGAATTATAGTGA | protein | |
| | ATGACCTCAAAGCTAAGGAGCGCGTAATGAAACTG | | |
| | TTGAACCACGAAAATGCAGAGGTCACGAAAAATGC | | |
| | CTTACTCTGTATCCAAAGGCTTTTCCTAGGTGCAAA | | |
| | GTATGCTAGCTTTTTGCAGGTTTAGTTCTCATCGAA | | |
| | GGGTTTGATTGTTCAGACGATGAAAACTAGACATA | | |
| | TCTTGTTATTTCATTGAAACAAAAGGAGTTTGATCG | | |
| | TGTTCGTGTTA | | |
| C431a | GATCCTGCAC GTCTGCCTGC TTTTCATTGT | monodehydro | SEQ ID N° 213 |
| | TGTGTCGGTA CGAATGAGGA AAGGTTGACC | ascorbate | |
| | CCGAAGTGGT ACAAGGAACA TGGCATTGAA | reductase | |
| | TTGGTCCTTG GAACTCGTGT AAAATCAGCT | | |
| | GACGTGAGAC GGAAGACACT GTTGACTGCA | | |
| | ACTGGTGAGA CCATAACCTA CAAGATTCTC | | |
| | ATAGTGGCAA CTGGTGCTCG GGCTTTGAAG | | |
| | CTTGAAGAGT TTGGAGTGAG TGGATCAGAT | | |
| | GCTGATGGTG TATGTTATTT ACGAGATTTG | | |
| | GCTGATGCAA ACAGGCTGGT TA | | |
| C431b | GATCCTCTGAGGCTATTATGCTTGCTGGATTAGCTT | glutamate | SEQ ID N° 214 |
| | TCACGAGAAAATGGCAAAATAAAATGAAAGCCCA | decarboxylase | |
| | AGGTAAGCCCTGTGACAAGCCCAATATTGTCACTG | | |
| | GTGCCAATGTCCAGGTGTGTTGGGAGAAATTTGCA | | |
| | AGGTATTTTGAAGTGGAGCTAAAGGAAGTAAAGTT | | |
| | GAGTGATGGATACTATGTGATGGACCCTGAGAAAG | | |
| | CTGTGGAAATGGTGGATGAGAACACAATTTGTGTA | | |
| | GCTGCTATCTTTGGGTT | | |
| C432 | AAACCGGTGCGATTTGAAAATACTGCTGGCGATCT | isoflavone synthase- | SEQ ID N° 215 |
| | TACAGGAAAATCACTATCAGGTCATTCCTTTCGGTT | like protein | |
| | CAGCAACAAGAATGTGTCCAGGGAATGTCGATGGG | | |
| | TTGAGTTA | | |
| C433b | GATCCTGCTGTAATGGGAATTGGCCCAGCCGTTGC | 3-ketoacyl-CoA | SEQ ID N° 216 |
| | GATACCAGCTGCTGTTA | thiolase | |
| C434a | TAAGCAGCGATGACCTCTTTGAAAGTGGAAGCTCA | putative protein | SEQ ID N° 217 |
| | AGTGATGATGCTGATGACGAGTTGACTGATAAAAG | AT5g43720 [A. | |
| | TGCAAGAGAACAAGCTTCTAGTACATCAGTGAAAG | thaliana] | |
| | CAGCTTTCTAGCATGTCCAGCGATGAAAAAAATCAG | | |
| | AGGCAAATATCCGCCCGTGCTCTAATGCCACCACC | | |
| | TCGTCCTTCGAGCAAGTCATTTAGTCATTCAGTAAA | | |
| | TAAAAAATCACGGTTTGGAGGATC | | |
| C435b | GATCCTCAAAATGGACTGTCAAGGAAGTTGCTGAA | mutator transposase | SEQ ID N° 218 |
| | TGTGTTACTCAGGACTCATCAAGCGGGGAAATAAA | | |
| | AAAGAAGCAAAACAGATGCTCCATATGCAAAACG | | |
| | ACTAGCCACAAAAGAACTACTTGCAAGAAGAGAAC | | |
| | TGAAGGAACAAGCAACTCCATTGTGGCTTA | | |
| C436a | TAAGGCATCA TATATACATC ATCTCGATGC | porin | SEQ ID N° 219 |
| | ATTGAAGAGG AGTGCTGCTG TGGGTGTAAT | | |
| | CACTAGAAGG TTCTCTTCAA ATGAGCACAC | | |
| | ATTTACAGTT GGAGGATCC | | |
| C436b | TAAGCATGGAAACCGCCTTTGTCCTATCTGCAGATG | retroelement pol | SEQ ID N° 220 |
| | CAAATGGAAGGAAATCCCTCTCCAATTTCCCACCTT | polyprotein | |
| | CAGTACTGATGTAAACGGTATCAATAATCCCGCG | | |
| C438 | GTTTAAGACATTTGATCTTAGCTACTTCAAGCTTTT | peroxidase | SEQ ID N° 221 |
| | GCTCAAGAGGAGAGGTCTGTTCCAATCTGATGCAG | | |
| | CCTTA | | |
| C439a | GATCCTGAGA AAGCTGTAGA AATGGTGGAT | glutamate | SEQ ID N° 222 |
| | GAGAACACTA TTTGTGTAGC TGCTATCTTG | decarboxylase | |
| | GGTTCCACCC TTACTCAGGG GTCATCAATC | | |
| | ACTAGT | | |
| C439b | GATCCTCCAA ACCTGAAGAC CAATGCAGTC | putative protein | SEQ ID N° 223 |
| | GAACAACCAG AATGCAAGGG AGAGAAGGTT | At4g09150 [A. | |
| | GATCTGTTCT TA | thaliana] | |
| C441a | GATCCTCAGCAATTCTAATGGTTCACAAGGCCAGA | Na+/H+ antiporter | SEQ ID N° 224 |
| | AAGAACGGGCTTCCCTTTTGGAATAAAGGACAAGT | | |
| | AGGGGAATCGAACCAAGTCATTGTAGCATTTGAGA | | |
| | CATTCGGACAACTCAGTAAGGTGTCAATTCGACCA | | |
| | CAACTGCAATCTCCGCTATGACAAGTATGCACGA | | |
| | GGACATAATTGCTAGCGCGGAGAGAAAAAGAGTTT | | |
| | CAATGATAATTTTACCGTTCCATAAACATCAGAGA | | |
| | ATTGGCGGACAATTTGAAACGACACGAGCTGATCT | | |
| | TAGACTTGTCAATCGAAGAGTTCTACAACACGCAC | | |
| | CATGTTCTGTTAGCATATTA | | |
| C441b | TGATGTTGAT ATCGCGACTC ATATACATGT | putative protein | SEQ ID N° 225 |
| | CAAGGATGAT GGACCTAAAA GGAGTATACT | At5g04740 [A. | |
| | GCATGTTGAA ACTGCTGATC GATCTGGTTT | thaliana] | |
| | GCTGGTGGAA GTCGTCAAAA TAATGGCTGA | | |
| | CATTAGCATT GATGCTGAAT CAGGAGAGAT | | |
| | TGATACAGAA GGTCTAGTTG CGAAGGGCAA | | |
| | GTTCTATGTC AGTTACAGAG GGGCAGCATT | | |
| | ACTCAGGACT CATCGATGAG TCCTGAGTAA | | |
| | CCACAAATGC CAAACCAAAA GAGCCAATAA | | |
| | ATTATACCTT ACATTGAACT GCCATTCTCA | | |
| | AAAAATGGCA CTANGAACTA ATACACACTG | | |
| | TTCGTTGATG GGGTAAAGCA AAAAAATAGG | | |
| | CAAATACTAG GGGAACCATA CAACATCAGC | | |
| | CTAGATACTA TGCAGTTAGT CAGGTTCCTC | | |
| | CATCCTTGTA CCCCCAGCAT CAGCTTCAGG ATC | | |
| C442 | ATGTTGGACAACCTTTAGCTCAGTTACTTTATCACT | cytochrome P450 | SEQ ID N° 226 |
| | TCGATTGGAAACTCCCTAATGGACAAACTCACCAA | | |
| | AATTTCGACATGACTGAGTCACCTGGAATTTCTGTT | | |
| | ACATGAAAGGCTGATCTTATTATGATTGCCACTCCT | | |
| | GCTCATTCTTGATTA | | |
| C443a | GATCCTAGTTTGGAATATGAGCTCTCTGCTCTTCGA | potassium | SEQ ID N° 227 |
| | GAACCCACAGAATCTGGATTTACATATTTGCTTGG | transporter | |
| | CATGGGGACGTGAGGGCGAAGAAAAACTCTTGGTT | | |
| | CATCAAGAAACTGTCAATAAATTACTTCTATGCATT | | |
| | CATGAGGAAGAACTGTAGAGGAGGCGCTGCAACA | | |
| | ATGCGTGTTCCTCACATGAATATTATCCAGGTGGG | | |
| | ATGACATACATGGTTTGATCTTGGTACCATTTAGCT | | |
| | TCTTGCTGGCCTTGTAAGTGCTGCATTA | | |
| C443b | GATCCATGCA GATATTCCAT GGGGCGATTT | glyceraldehyde-3- | SEQ ID N° 228 |
| | AGGTGCAGAT TATGTTGTTG AATCTTCTGG | phosphate | |
| | TGTTTTCACA ACCGTTGAGA AGGCTTCAGC | dehydrogenase | |
| | ACATAAGAAG GGTGGTGCAA AAAAGGTCGT | | |
| | AATCTCAGCT CCATCAGCTG ATGCACCTAT | | |
| | GTTTGTGGTA GGAGTGAATG AGAGAACTTT | | |
| | CAAAACCACC ATGGATGTTG TTTATAATGC | | |
| | TAGCTGTAGT ACCAATTGCC TTGCTCCCCT | | |
| | TGCCAAGGTG GTTCATGAGG AGTTTGGCAT | | |
| | TGTTGAAGGA TTA | | |
| C444 | GATCCTCAAG CATTTATTTG CCACTTTTAC | heme oxygenase 1 | SEQ ID N° 229 |
| | AACACATACT TTGCGCATTC AGCTGGAGGT | | |
| | CGCATGATAG GGAGAAAGGT GGCTGAAAAG | | |
| | ATACTCAATA AGAAAGAGCT GGAATTCTAC | | |
| | AAATGGGACG GTGACCTTTC TCAGCTGCTG | | |
| | CAGAATGTTA GAGAGAAGCT GAATAAAGTT | | |
| | GCAGAAAACT GGACTAGAGA GGAGAAGAAT | | |
| | CATTGTTTGG AAGAGACGGG GAAGTCATTTC | | |
| | AAGTTCTCAG GGGAAATCCT CCGATTA | | |
| C445 | GATCCTCTCA TCATTGTCCA GGAGGTCTGT | putative inorganic | SEQ ID N° 230 |
| | TGCTGCTCAC CCTTGGCACG ATCTTGAGAT | pyrophosphatase | |
| | TGGACCTGAA GCTCCAAAGG TTTTCAATGT | | |
| | TGTCATTGAG ATTACAAAAG GTAGTAAAGT | | |
| | CAAATACGAG CTTGACAAGA AAACTGGTCT | | |
| | CATTA | | |
| C446a | TAATGGAAGA TGCACCACTG GAATGAGCAA | cytochrome c | SEQ ID N° 231 |
| | AGAAAAGTTA GGTCATTTTA TGACTTGCTG | oxidase subunit 5c | |
| | GAGAAAGGTG AAATAAGTTT AGTCGCAGAA | | |
| | GAATAATTTT TCGAGGATC | | |
| C446b | TAATGGATGATACTGCTGAGGCAAAAGCTTGTCAA | putative protein | SEQ ID N° 232 |
| | GACGAAGTGAATGCTATTCTGGGAGAGAAGCTATC | At5g09260 [A. | |
| | TGCTGATTATGAAGAGGAAGTTTTAGCACAATTTG | thaliana] | |
| | AGGATC | | |
| C447 | GATCCTCATGACATATGTGAACAACATCCTGACAT | arogenate | SEQ ID N° 233 |
| | CGTCGTACTCTGCACTTTCCATTA | dehydrogenase | |
| C448 | GATCCTGGTC GCCTGACAGG CAAGAGAGAT | catalase 3 | SEQ ID N° 234 |
| | TTATCTGCAG ATGGATTA | | |
| C449a | GATCCTGCTG TTTTTACTGG GGATACATTG | glyoxalase II | SEQ ID N° 235 |
| | TTTATTGCTG GTTGTGGTAA GTTTTTTGAA | | |
| | GGCAGTGCAG AACAAATGTA TCAGTCACTG | | |
| | TGTGTGACAC TAGGTTTCTT GTCAAAGCCA | | |
| | ACTCGGGTGT ATTGTGGCCA TGAGTACACA | | |
| | GTAAAAAATT TGCAGTTTGC TTTA | | |
| C449b | GATCCTGAGG GTGCTCATTA CAATTTTGGA | vacuolar H(+)- | SEQ ID N° 236 |
| | CACATCAAGT GATCCAAGGA CACTTGCTGT | ATPase | |
| | GCTTTGCTAT GATCTATCGC AGTTCATTCA | | |
| | ATGCCATTCT GCTGGGCGAA TTATAGTGAA | | |
| | TGACCTCAAA GCTAAGGAGC GCGTAATGAA | | |
| | ACTGTTGAAC CACGAAAATG CAGAGGTCAC | | |
| | GAAAAATGCC TTACTCTGTA TCCAAAGGCT | | |
| | TTTCCTAGGT GCAAAGTATG CTAGCTLTTT | | |
| | GCAGGTTTAG TTCTCATCGA AGGGTTTGAT | | |
| | TGTTCAGACG ATGAAAACTG GACATATCTT | | |
| | GTTATTTCAT TGAAACAAAA GGAGTTTGAT | | |
| | CGTGTTCGTG TTA | | |
| C449c | TGACTGCGTAGTGCTCCTGACGGTTATTGGATCGAG | glyoxalase I | SEQ ID N° 237 |
| | ATTTTTGGCACTAAACCTATCAAAGAAGTTGCTGAT | | |
| | GCTGCTTCTTGATTCAGGGGCTCTTCGAGTGTCTAT | | |
| | CACGAGTGTTGATCAACTCAGCTATCTGTTGAAGA | | |
| | GAGAGTTTCTCGTAAACAGCGTTTTCTTTCCAGGTTA | | |
| C450 | GATCCTGGTG TTAGCAACAA TGAAGATGAG | putative protein 66b | SEQ ID N° 238 |
| | GATGTTGAGG ATATCAATGT TGCAGAGGAC | [Daucus carota] | |
| | GATATGATGG ATGATGTGCT TGACGTGGAT | | |
| | GATAATAACC AGAGGAGTGA TGAAATTGTA | | |
| | AAAGTTGAAG CCGGTAATGG TAGTACACAG | | |
| | ATTGATCAGC AGAAGATATG CATCTCTTAT | | |
| | CTCTATTAAA GGTTTAGTTT GTGTTTA | | |
| C451a | GATCCTGCTG TAATGCCAAT TGGCCCAGCC | 3-ketoacyl-CoA | SEQ ID N° 239 |
| | GTTGCGATAC CAGCTGGTGT TA | thiolase | |
| C452a | GATCCTGATAGAACTGAATCCGAGGATTCTGATGA | putative SR protein | SEQ ID N° 240 |
| | TTCAATATAGCCGAGGACATTTTTCAGCAGACAAT | | |
| | GATTAGTTAGCTACAAAAGCTGTTTTTGGCAAGTG | | |
| | GTTACCAAGTCTCCGCCATTGATATAGTTACTTCAT | | |
| | GGTTA | | |
| C452b | GATCCTGTTT GTGGAAGTGC CCATTGTGCT | PHZF-like protein | SEQ ID N° 241 |
| | TTGGCTCCTT ATTGGCATAA AAAGCTTGGC | | |
| | AAATGTGACT TTGTTGCTTT AGCGGCCTCA | | |
| | ACTAGAGGTG GCGTTGTGAA CGTGCATCTA | | |
| | GACGAGGAGA ATCAGAGGGT ACTTCTGAGA | | |
| | GGGAAAGCTG TTGTTGTTAT GGAAGGTACT | | |
| | CTTCTAGTTT A | | |
| C452c | GATCCTGAAC TTCCCCCTGA AATGAGAGAA | mitochondrial | SEQ ID N° 242 |
| | GCTCATCGTT ACAAGCTTTC AAAATTGCCA | ribosomal protein | |
| | AGGAACAGTT CTTTTACCCG AATCAGAAAT | S14 | |
| | CGGTGCGTTT TCACTGGTCG GCCACGTGCT | | |
| | GTGTATGAGA AGTTTAGAAT GTCGCGTATT | | |
| | GTGTTCCGTG GTTTGGCTGC TCGCGGTGCT | | |
| | TTGCAAGGTG TTTA | | |
| C453 | TTTCATACCATGGCGATTTGAAAATACATCTGTTTGA | cytochrome P450 | SEQ ID N° 243 |
| | TCTTACGGGAAATCACTATCAGTTCATTCCTTTTGG | | |
| | TTCAGGAAGAAGAATGTGTCCTGGAATGTCGTTTG | | |
| | GTTTA | | |
| C454 | ACAGCTATGA CCATTAAGCC TATTTAGGTG | putative | SEQ ID N° 244 |
| | ACACTATAGA ACAAGTTTGT ACAAAAAAGC | phosphatase 2C | |
| | AGGCTGGTAC CGGTCCGGAA TTCCCGGGAT | | |
| | CTCTCAGTTT TTTTCATCCA TTCCTCTTCA | | |
| | GCCAATCCCA AGAGGGTCAT CATTTGCAGC | | |
| | TTCTACTATT CATTCAGGCC CTATCCCGGC | | |
| | CCGTATTTCT AGTACGTACC CTTGCTCGGG | | |
| | CCCGATCGAG AGGGGATTCA TGTCCGGCCC | | |
| | GATTGAGCGG AGCTTCACCT CGGGCCCGTT | | |
| | GGAGAACCAG TATGATCATA TCCAAAGGTA | | |
| | CAAGCCCAAG TCCAAGAAAT GGGGTTTAAT | | |
| | TAAAAGTTTA AAGAAAGTGT TGTCAAATTC | | |
| | CTTTTTGGGG TTTAATAAAG AAATGAATTT | | |
| | GGTAGAGAAG AATAATAATA ATGAAGTTAA | | |
| | TGTTCAAGGG AGTAATAGTC ATCATAGTAA | | |
| | TGTTGGAAAT AGTTTGAGTA GTCAGAATAG | | |
| | TTTGGTTGAT GATGATGATG AGGGAAATGA | | |
| | CTCATTTAGA GGCCAAAATG TGCAATGGGC | | |
| | TCAAGGTAAA GCAGGGGAAG ACAGAGTACA | | |
| | TGTTGTGATT TCTGAGGAAC ATGGTTGGGT | | |
| | TTTTGTAGGG ATATATGATG GATTTAATGG | | |
| | ACCTGATGCT ACTGATTTTC TGTTAAACAA | | |
| | TCTTTATTCA AATGTCTATA AAGAACTCAA | | |
| | GGGATTGCTA TGGAATGATA AGTTAAAAAC | | |
| | CCCCAAGAAT TCGACGAGCA ACGAGACTGT | | |
| | TCCGTTAAGA AACTCGGGTT TTAAGGTGGA | | |
| | ACATTTTGTT CAAAATCAAG AATTAGATCA | | |
| | GAGGGAGAAA CTTGATGGGG TTGTTGGTGT | | |
| | TGACCATTCT GATGTATTGA AGGCTTTATC | | |
| | TGAAGGGTTG AGGAAAACCG AGGCGTCGTA | | |
| | TTTGGAGATT GCTGATATGA TGGTAAAGGA | | |
| | GAATCCTGAA TTGGCTTTAA TGGGATCTTG | | |
| | TGTTTTAGTA ATGTTGCTTA AAGATCAGGA | | |
| | TGTTTATTTG TTGAATGTTG GAGATAGTAG | | |
| | AGCTGTTTTA GCTCAAAATC CTGAGTCTGA | | |
| | TATTTCTATT AGCAAATTGA AAAGGATAAA | | |
| | TGAGCAGAGT GTAAATAGCA TTGATGCACT | | |
| | CTATCGAGCT GAATCTGATC GCAAACATAA | | |
| | TCTAATTCCT TCTCAACTTA CTATGGATCA | | |
| | TAGCACATCT ATTAAAGAGG AAGTAATTAG | | |
| | GATTAGAAGT GAGCATTTGG ATGATCCTTT | | |
| | CGCGATTAAA AATGATAGAG TGAAAGGTTC | | |
| | CTTGAAAGTT ACTCGAGCTT TCGGGGCAGG | | |
| | ATATCTCAAA CAGCCCAAGT GGAATAATGC | | |
| | ACTTCTAGAG ATGTTCAGAA TTAACTACAT | | |
| | TGGGAATTCG CCTTACATCA ACTGTTTACC | | |
| | ATCGCTTTAC CACCACACTC TTGGTTCGAG | | |
| | AGACAGATTT TTGATCTTAT CATCTGATGG | | |
| | TCTTTACCAA TACTTCACAA ATGAAGAAGC | | |
| | AGTCTCAGAA GTAGAGACCT TTATGTCTAT | | |
| | ATTCCCCGAG GGAGATCCTG CACAACATCT | | |
| | CGTCGAAGAA GTGTTATTCA GAGCTGCTAA | | |
| | GAAAGCTGGA TTGAACTTCC ATGAGTTGCT | | |
| | CGATATACCT CAAGGAGATC GTAGGAAGTA | | |
| | CCATGATGAT GTTTCAATTA TCATTTTGTC | | |
| | CTTCGAAGGA AGGATATGGA AATCATCGTT | | |
| | GTAAATCAGC TAGACACAGG AATTTTTATA | | |
| | TTTTACCCTC AGAAATCAGG AAAAAAAGAA | | |
| | AGTACATAGA AAAAATCGAG CTAATTTTGC | | |
| | TGTTAACCGT TGTTTACCCA ATTTTAGCAG | | |
| | TAGTGTTTAT AGTATACAGT CTAGGCTGCT | | |
| | CGATAAAAGA TAGCGAGGCT GAGGTTTCTT | | |
| | GATCCAGAGA TTGTAAAATT GCCAATAAAC | | |
| | TTATAACAAC CCCTGCCTCT TCTACATTCA | | |
| | AATGTTATTA GGACATGGTA AGTTTTGTAA | | |
| | CAGATGGTGC TCCTTGTATA CATTCTGGAG | | |
| | TTCCATTTCA CAAAAAAAAA AAAAAAAAAA | | |
| | AAAAAAAAAA AAAA | | |
| C456 | AAACCGGTTGCGATTGGAAAATACTTCTGGTGCTCT | isoflavone synthase- | SEQ ID N° 245 |
| | TACAGGAAAATCACTATCAGGTCATTTCCTTTCGGTT | like protein | |
| | CAGGAAGAAGAATGTGTCCAGGGAATGTCGTTGGG | | |
| | TTTAGTTA | | |
| C457 | TCGGGTATTG AAGCACAAGA ATGGGAAGTT | acetyl Co-A | SEQ ID N° 246 |
| | GGGTGTTGCA GGAATCTGCA ATGGGGGAGG | acetyltransferase | |
| | AGGCGCATCT GCTCTTGTTG TAGAGCTCAT | | |
| | GCCTATAAGG ATGGTGGCAC GTTCATCGCT | | |
| | TTGAAACTGG AATAGTTTGT ACTATATTTA | | |
| | CGTCTAGCTG CTGCACAGTT GCATGCCTGC | | |
| | TGAGTTCTGC CACATTGCGT CAAAAGTAGT | | |
| | GAGGTATCTG AATGCTTGTA TCCATTATGT | | |
| | AAAACCATAT AAGCAATAAC CTAATAATAC | | |
| | CATGAAAATC GAGCAAACAC TTGTTTCCCT TA | | |
| C458a | GATCCTGGAG AATACTGGAG AGCTGTGATG | specific tissue | SEQ ID N° 247 |
| | AACGATGAGC CAATGCCTGA AGCAATCAAA | protein | |
| | CATCTTATGC CTCAGCATTC TGTTCCTCTC | | |
| | TCCATAGAGA AAACTGATTG TTACACATTA | | |
| | CCTTCTACTG GAGGTGAAGC CTTTGAACCA | | |
| | AGGCCTAATC TATCTGTCTA CCACGATGAC | | |
| | GCCAAGCTGA AAGAAGCTGA GAAATTATTA | | |
| | TTTATGAAAG ATTTTGAGCC AAGGCCTACT | | |
| | ATAACTGGTT ATCATAATAA TGATGCTGGT CTTA | | |
| C458b | GATCCTGTAA TGAAGGAAGA AATTGACAGG | SKP1-like protein ? | SEQ ID N° 248 |
| | GAGGTTGAGG ATTTTGCTAG GAGACTGAAC | | |
| | TCTGTTTGGC CAGAAAGAAT GCAGGAGATT | | |
| | TTGTCTTTGG GTCAAGAGAG GAGGCCTGTA | | |
| | CCACTATCTG TGAATGGGAA TGGTTCCCTA | | |
| | AAGAGATATA CGGGTTTGGA TGGGAGATAA | | |
| | TGGTTCAAAT GGTGGATGAT GAATCTTTTG | | |
| | GCTTCAGTCG AGCTTACTCA GGACTCATCA | | |
| | TCACTGGTTT TGTTATTACA TAGTGTGTTT GCTTA | | |
| C461 | GATCCTGATCCTAGACATTATTTACCTCTTTACCTT | gene feebly protein | SEQ ID N° 249 |
| | AGACCAGCAACCTGATATGTTTTATAGGATGTGCA | | |
| | CTTTGTAACCTTTGTATGAGATGAATATGTAACATG | | |
| | GTGTACGTAAAGTTTGAAAGTATAATATGTAAGAT | | |
| | CACGTAAATCTATAGGTAAGGCTTA | | |
| C462 | GATCCTGGTAGTTTCAAGACATTTGATCTTAGCTAC | putative peroxidase | SEQ ID N° 250 |
| | TTCACAGCTTTTGCTCAAGAGGAGAGGTCTGTTCCA | | |
| | ATCTGATGCAGCCTTA | | |
| C463a | GATCCTGAGA AAGCTGTAGA AATGGTGGAT | glutamate | SEQ ID N° 251 |
| | GAGAACACTA TTTGTGTAGC TGCTATCTTG | decarboxylase | |
| | GGTTCCACCC TTA | | |
| C463c | GATCCTGGAT GCAGGCGGGT TTTTATCTAG | ADP-ribosylation | SEQ ID N° 252 |
| | TTATTTTTTT CTTCTCAAGT CAGTGTGGTT | factor | |
| | ATGAACATCT CCTTTA | | |
| C464 | GATCCTGATAAACCAACATTATCGTAGAGAATGTTT | histamine-releasing | SEQ ID N° 253 |
| | TCTCTGTTTCTCCCTCTGAAGAACTTGCTTA | factor homolog | |
| C465 | TAATCCAAAGTAGCAGATAATATCATAAATGCGCG | putative protein | SEQ ID N° 254 |
| | GAAGAACAACCCAACACAGCTCGATACCAGGGTGT | kinase | |
| | CACTAGTCAAGAGCATCTATAAAACATAATACAAG | | |
| | TCTGAAGAGTCTATAACTATTACAAATGTCTGATAC | | |
| | AAGATAGAAATGATAAAGAGGGAGAAACACATGA | | |
| | CTACGGACATCAAACAACTACCTCGTGGTCTCTAA | | |
| | ATGTGCTAGGAGCTCTCAACTTACACTTGCAGGATC | | |
| C466 | GAAGCTGGGCACAATGAGCCTAGCTTGGTAGCAAG | putative | SEQ ID N° 255 |
| | ACTTGTGAACTTACTCAGATACTATGCTGCTGGGCT | transcription factor | |
| | CGATTCTATTGGTTTCAGCCTTCCACCATACAGCCC | SCARECROW | |
| | TTGCAGGATTA | | |
| C467 | ATCCTGTAG AGAAGGGATA TGTGGGTCCT | succinate | SEQ ID N° 256 |
| | GTGCTATGAA TATTGATGGT TGCAATGGAC | dehydrogenase iron- | |
| | TTGCTTGTTT GACTAAGATC GATTCGGGTG | protein subunit | |
| | CTGAATCGAC GACTACGCCG TTGCCACATA | | |
| | TGTTTGTGAT TA | | |
| C469a | GATCCTCTAC ATGAAAATGC AAATTTCATG | putative protein | SEQ ID N° 257 |
| | AATGTGAAAT GGTATACTTT GCTTCGTAAG | AT5g08550 [A. | |
| | TATGGACTCT CTACAGATGA AAATCCAAAT | thaliana] | |
| | AGCTTTGATG ACGCTGATGC CAATCCTGTT | | |
| | CAATTGGTGG TGAAACTTGC AATGGCCATT | | |
| | CTACATAACC GGTTAGCTCA GTGCTGGGAT | | |
| | GTGTTTAGCA CCCGTGAGAC ACAGTGTGCT | | |
| | GTATCTGCCA TAAATCTGTT GTTA | | |
| C469b | GATCCCAAGA GACTGGTTGA ATACTACAAA | putative glutathione | SEQ ID N° 258 |
| | AACCGTTTTA TGGCCTAGAA TTTCAAAACG | S-transferase | |
| | GTTTGTCAAC CATTGGTGAA ACTGCGAATG | | |
| | AAGCACGCGC TGTATAAGTA TGTCATGGAG | | |
| | TTCTACAGAA TTGTTGATTA GTAATAGATA | | |
| | AATAAATTGG TCATGTCCTT TTTTTTATCT | | |
| | GTAGAATTGT GAATTATTTT TGGGGTTTGG | | |
| | TGTTTATGCT AGGGACTTGG ATTA | | |
| C471a | GATCCTTTTCTGAAAAAATTCTTTTTCCAACGGTTTAC | hexose transporter | SEQ ID N° 259 |
| | AAGAGAACAAAGGATCAAGGATTGAACAGTAATT | | |
| | ACTGCAAGTATGATAATCAAGGGCTGCAGCTATT | | |
| | ACTTTCATCTTTATATCTGGCCGGTTTA | | |
| C471b | GATCCTTACAGGTGGTTCAGTCATAGAATCTGAGG | aldehyde | SEQ ID N° 260 |
| | GTAACTTTGTGCATCCAACAATTGTTGAAATATCTT | dehydrogenase | |
| | CAAAAGCTGAAGTTTGTGAAGGAAGAATTGTTTGCT | | |
| | CCAGTTCTTTATGTAATGAAGTTTA | | |
| C472 | GATCCTTCAC TGTGTAATCA AACAAAAAGA | quinolinate | SEQ ID N° 261 |
| | TGTAAATTGC TGGAATATCT CAGATGGCTC | phosphoribosyltrans | |
| | TTTTCCAACC TTATTGCTTG AGTTGGTAAT | ferase | |
| | TTCATTATAG CTTTGTTTTC ATGTTTA | | |
| C474 | TGCGTAATCAAACAAAAAGATGTAAATTGCTGGAA | quinolinate | SEQ ID N° 262 |
| | TATCTCAGATGGCTCTTTTCCAACCTTATTGCTTGA | phosphoribosyltrans | |
| | GTTGGTAATTTCATTATAGCTTTGTTTTCATGTTTCA | ferase | |
| | TGGAATTTGTTACAATGAAAATACTTGATTTATAAG | | |
| | TTTGGTGTATGTAAAATTCTGTGTTA | | |
| C475 | TAACGTTGGTTCTCCAAGGGGAATTTCAGGCGAGC | putative lipid | SEQ ID N° 263 |
| | GAGGCAGTGACATGCAGTGCCTCGCAGCTAAGTGA | transfer protein | |
| | GTGTGTGGGGGCGGTGACGTCGTCACAGGCACCAT | | |
| | CTTCGGCATGTTGCAGCAAAATGAGGGACCAACAG | | |
| | CCTTGTCTGTGTGGGTACATGAAGGATC | | |
| C476a | TGTCTGGATC AAACCTTGCT GCCCCATATC | MAP kinase | SEQ ID N° 264 |
| | CTCTCTCCTT CCTAACATGG TGGGGTGGCT | | |
| | ATGTCTGTCC CCACTATTCC CACGTGCTTT | | |
| | CTCCTCCCCA CTTATATAAA CACAAATTTC | | |
| | ACTGAAGAGG AGAAGAATCC ATTTCCATTC | | |
| | CAACAAATCC AAACGGACCC GACCCGATTC | | |
| | ACCCCACCAC ATGGCCTTAG TCCGAGAACG | | |
| | TCGACAGCTC AATCTCAGAC TTCCCTTGCC | | |
| | GGAACCCTCC GAACGCCGCC CTCGTTTCCC | | |
| | CTTACCCCTC CCTCCTTCCA TCTCCACCAC | | |
| | CACAACTGCT CCTACCACTA CTATCTCCAT | | |
| | CTCGGAACTC GAAAAGCTTA AGGTTCTCGG | | |
| | TCACGGAAAC GGCGGAACTG TGTACAAAGT | | |
| | CCGCCACAAA CGCACATCCG CAATCTACGC | | |
| | TCTCAAAGTC GTTCACGGCG ATAGCGACCC | | |
| | CGAGATTCGC CGTCAAATCC TCCGTGAAAT | | |
| | CTCCATCCTT CGCCGGACGG ATTCTCCTTA | | |
| | CGTCATCAAG TGCCACGGTG TCATCGACAT | | |
| | GCCCGGCGGC GACATCGGTA TCCTTATGGA | | |
| | GTACATGAAC GTCGGCACAC TAGAAAGTCT | | |
| | TTTAAAATCA CAAGCAACTT TCTCCGAACT | | |
| | TAGCTTAGCA AAAATCGCTA AGCAAGTACT | | |
| | TAGCGGACTC GACTACTTAC ACAATCACAA | | |
| | AATCATTCAC AGAGATTTAA AACCTTCGAA | | |
| | CCTTCTAGTA AATCGCGAGA TGGAAGTAAA | | |
| | AATCGCCGAT TTCGGAGTGA GTAAAATCAT | | |
| | GTGCAGGACT TTAGATCCTT GCAATTCATA | | |
| | CGTTGGAACT TGTGCTTATA TGAGCCCAGC | | |
| | AAGGTTTGAT CCAGACACTT ATGGAGTTAA | | |
| | CTACAACGGT TACGCAGCTG ATATTTGGAG | | |
| | TTTGGGCTTG ACTTTAATGG AACTATATAT | | |
| | GGGCCACTTT CCGTTCTTGC CACCTGGACA | | |
| | GAGACCGGAC TGGGCTACGC TAATGTGCGC | | |
| | CATATGCTTC GGTGAGCCGC CCAGTTTGCC | | |
| | TGAAGGGACG TCGGGAAATT TCAGAGATTT | | |
| | TATCGAGTGT TGTTTACAGA AAGAGTCCAG | | |
| | TAAAAGGTGG AGCGCTCAGC AACTTTTGCA | | |
| | ACATCCGTTT ATACTGAGCA TCGATTTGAA | | |
| | GTCCACGTAA AAAGGGACAG AGCAAAGCTG | | |
| | AAGACTGGGA AATTGAATAG TTCCGAGTTG | | |
| | TTTGTAAATA GAGAACGGGA CCTTCTTTTT | | |
| | TTTTTTGAAC TTTTTGGGTT AACTTTTTTG | | |
| | TATATTCTTC AACTATGAAT CTGTGAAATC | | |
| | AGAATCATTC TCTGTATCTG GAAAAAGTGC | | |
| | CCATTTTCCA TAGCAAAAAA AATCATCTGT | | |
| | GGAATTTTGA GACTTAATGA ATTCAATCTT | | |
| | TTTCCAACAA AAAAAAAAAA | | |
| C476b | GATCCTCGTG AGGTTGCTGC TGCTAAAGCA | succinyl-CoA ligase | SEQ ID N° 265 |
| | GATTTGAATT ATATTGGCTT GGATGGAGAA | | |
| | ATTGGTTGCA TGGTTA | | |
| C477 | CCAGCTATGA CCATTAGTGC CTATTTAGGT | putative zinc | SEQ ID N° 266 |
| | GACACTATAG AACAAGTTTG TACAAAAAAG | transporter | |
| | CAGGCTGGTA CCGGTCCGGA ATTCCCGGGA | | |
| | TTTTTTCTAT TCCGTGATCC CCTTTATCTC | | |
| | TTCCCCTTTT TCTCCTTTTT CTTCTTCGTT | | |
| | TAGGTATATA CCCCATATAT ATAGCCTATA | | |
| | AACCATATAG CTATATAAAA CTCTACATCT | | |
| | ATTTTGAGAA TTTGATGATT TGGGTCGGCT | | |
| | AAAAATACAA TCTTTTTAAT ACTCTTTTGA | | |
| | AATCTTGGCA CAAATTTGTG AGATGGAGAC | | |
| | GCAGAACCTG GAACGTGGAC ATGTAATTGA | | |
| | GGTACGTTGT GACATGGCAG CTCAAGAAAA | | |
| | GGGGACTAAA ATCTGTGGTT CAGCACCGTG | | |
| | TGGATTCTCA GATGTTAACA CCATGTCTAA | | |
| | GGATGCACAG GAGAGATCAG CATCCATGAG | | |
| | GAAACTTTGC ATCGCGGTTG TCCTCTGCAT | | |
| | CATATTTATG GCTGTTGAGG TTGTTGGTGG | | |
| | TATTAAAGCC AACAGTCTGG CAATATTGAC | | |
| | CGACGCTGCT CATCTACTAT CAGATGTTGC | | |
| | AGCTTTTGCA ATATCCTTGT TTTCACTCTG | | |
| | GGCAGCAGGA TGGGAAGATA ATCCACGCCA | | |
| | GTCCTATGGG TTTTTCAGAA TCGAGATACT | | |
| | CGGGGCATTA GTTTCTATCC AAATGATATG | | |
| | GATTCTAGCT GGGATCCTTG TTTATGAAGC | | |
| | CATTGCTCGA CTTATTCATG ATACAGGTGA | | |
| | AGTTCAAGGC TTCCTCATGT TTGTGGTGTC | | |
| | TGCATTTGGA TTAGTAGTGA ACCTCATCAT | | |
| | GGCACTCTTG TTAGGTCATG ATCATGGCCA | | |
| | CGGCCACGGC CATGGCCACA GCCACGGTCA | | |
| | TGACCATGAA CACGGCCATA ATCATGGCGA | | |
| | GCATGCTCAT AGCAATACTG ATCATGAGCA | | |
| | CGGCCATGGT GAGCATACGC ATATACATGG | | |
| | AATTAGCGTT AGCCGACACC ATCACCATAA | | |
| | TGAGGGACCT TCGAGCCGAG ATCAACACTC | | |
| | GCACGCACAT GATGGAGATC ACACCGTGCC | | |
| | TCTACTTAAG AATTCATGTG AGGGTGAAAG | | |
| | TGTATCAGAA GGTGAAAAGA AAAAGAAGCC | | |
| | CCAGAACATA AATGTTCAGG GAGCTTATCT | | |
| | TCATGTAATC GGAGATTCTA TTCACAGCAT | | |
| | AGGGGTGATG ATTGGGGGAG CTATTATATG | | |
| | GTATAAACCA GAGTGGAAAA TCATCGATCT | | |
| | AATTTGCACT CTCATTTTCT CTGTAATTGT | | |
| | GCTCGGGACA ACCATTAGGA TGCTTCGGAG | | |
| | TATTCTTGAA GTATTAATGG AGAGTACGCC | | |
| | CAGAGAAATT GATGCAACAA GGCTCCAGAA | | |
| | GGGGCTCTGT GAGATGGAGG ACGTTGTCCC | | |
| | AATCCATGAA TTGCACATAT GGGCAATTAC | | |
| | AGTCGGCAAA GTGCTCCTGG CTTGCCATGT | | |
| | CAAGATTAAG TCCGACGCTG ATGCTGACAC | | |
| | GGTGCTGGAT AAGGTGAT | | |
| C478 | ATATGTTACAGGGTCCATGCAGAGCGCTATTTGGCT | sucrose transport | SEQ ID N° 267 |
| | GATCTGTCCGGCGGAAAAGCCGGGAGGATGAGAA | protein | |
| | CATCAAAGGCCTTCTTCTCCTTCTTCATGGCCGTCG | | |
| | GAAACGTCCTCGGTTACGCCGCCGGTTCCTACTCCC | | |
| | GCCTCTACAAAATCTTCCCCTTCTCTAAAACCCCAG | | |
| | CCTGTGACATCTACTGCGCCAACCTCAAATCATGTT | | |
| | TCTTCATCGCCGTCTTCCTTCTACTCAGCTTA | | |
| C479 | TGTGTAATCAAACAAAAAGATGTAAATTGCTGGAA | putative protein | SEQ ID N° 268 |
| | TATCTAGATGGCTCTTTTCCAACCTTATTGCTTGAG | AAK58573 | |
| | TTGGTAATTTCATTATAGCTTTGTTTTCATGTTTCAT | [Acidianus sp.] | |
| | GGAATTTGTTACAATGAAAATACTTGATTTATAAGT | | |
| | TTGGTGTATGTAAAATTCTGTGTTACTTCAAATATT | | |
| | TTGAGATGTTGAATATCATGTTCTTA | | |
| C480 | TCCAAGAGTCTACCACGAGCTAATTCCGAATGTAG | gamma- | SEQ ID N° 269 |
| | TTCTGTACGAGAACTGGACGTGCATCGATGGCGAT | glutamyltransferase- | |
| | CATATTGAACTCTCGGACGAGAAAAAGGCATTTCT | like protein | |
| | TGGAAGAGAGGGGTCATCAACTCGAGGCACATAAC | | |
| | GGAGGAGCCATCTGTCAGCTAATTGTTCAAAACCT | | |
| | TCCAAATTCTCCCTTA | | |
| C481 | GATCCTTCAC TGTGTAATCA AACAAAAAGA | quinolinate | SEQ ID N° 270 |
| | TGTAAATTGC TGGAATATCT CAGATGGCTC | phosphoribosyltrans | |
| | TTTTCCAACC TTA | ferase | |
| C482 | GATCCTTGGC AGACAAACAG GGTCGAAAGC | putative protein | SEQ ID N° 271 |
| | GGGCTTGTGT CACGTACTGC ATCACTTACA | AT4g27720 [A. | |
| | TTTTGAGCTG TATGACCAAA CATTCTCCTC | thaliana] | |
| | AGTACAAAAT TTTGATGTTG GGCCGTATAT | | |
| | TAGGAGGAAT TGCCACCTCT CTCCTATTCT | | |
| | CAGCCTTTGA ATCTTGGCTT GTTGCAGAGC | | |
| | ATAATAAGAG GGGTTTTGAT CAACAATGGC | | |
| | TATCATTA | | |
| C483a | GATCCTTTGG GCAAAGGTCG AGATGGAACT | receptor-like protein | SEQ ID N° 272 |
| | GCTTTCTCTC AGGAAGTATT TGAGAGCTTT | kinase | |
| | ATGTTCAATT TGGATGAAGT TGAGTCTGCT | | |
| | ACACAGTATT TTTCAGAGGC AAATTTGTTA | | |
| | GGGAAGAGTA ATTTCACAGC CGTTTATAAA | | |
| | GGGACACTGA GGGATGGGTC TTCTGTTGCT ATTA | | |
| C483b | GATCCTTTAC AAACAGAGTA GAAAGATGCA | mutator-like | SEQ ID N° 273 |
| | GTGAGACATG AATTACATTG ATTTTGGTTT | transposase | |
| | TGGCATTCTT TTCTCGCAAG ATATGTTGTA | | |
| | AGCATAGTAT CAGTAGGTCA TTATTCCGAT | | |
| | TTTCCCCTCA ATTGGGGAAA GGGAGGAGGT | | |
| | GTGTGACCTT GGTCACGGTT GTACCATTA | | |
| C483c | GATCCTTGGGCCCGATGTCCATGAGGTGGATTACG | delta-1-pyrroline-5- | SEQ ID N° 274 |
| | TTGCATGGGTTTGTGATCAAGATGCATATGCATGTA | carboxylate | |
| | GTGGTCAGAAGTGTTCAGCTCAATCAATATTGTTCA | dehydrogenase | |
| | TGCATGAGAATTGGGGTAGAAGCTCTCTCTTAGAC | | |
| | AAAATGACCGAGCTTGCTGCAAGAAGAAAGTTGGA | | |
| | TGATTA | | |
| C484a | AAAACATCAT GAATAACACC ACCTTTTCCG | C3HC4-type RING | SEQ ID N° 275 |
| | TCCAAATTTC CGACACCGGA GGTTTCCTCG | zinc finger protein | |
| | GATCGGGAAA AATCGGAGGA TTCGGCTACG | | |
| | GAATTGGTGT TTCAGTAGGT ATTCTTATTT | | |
| | TAATTACAAC AATAACCCTC ACTTCCTATT | | |
| | TTTGTACTCG AAATCAAACA TCAGAGTTAC | | |
| | CAACAAGAAG ACAAAGAACA ATTAATCGAA | | |
| | ACGAGCTTTC TGGACATTGT GTGGTTGATA | | |
| | TTGGGCTCGA TGAAAAAACC CTTTTGAGTT | | |
| | ATCCCAAGTT GTTGTACTCT GAAGCTAAGG | | |
| | TCAATCATAA GGACTCAACA GCTAGTTGTT | | |
| | GTTCCATATG TTTAGGAGAT TACAAGAAAA | | |
| | AAGACATGCT TCGATTGTTG CCAGATTGTG | | |
| | GACATTTGTT TGACTTGAAA TGTGTGGATG | | |
| | CTTGGCTCAT GTTGAATCCA AGTTGTCCAG | | |
| | TTTGTAGAAC ATCTCCATTG CCAACACCAC | | |
| | AATCTACTCC TTTGGCTGAG GTTGTTCCTT | | |
| | TGGCAACTAG ACCTTTGGGA TGA | | |
| C484b | GATCCTTGTG CCCCTTCCGG AGCCAGAAGC | katanin | SEQ ID N° 276 |
| | AAGGTGCGCC ATGTTTGAAG AATTACTACC | | |
| | ATCACTGCCT GAAGAGGAGT CACTTCCATA | | |
| | TGATTTATTG GTAGAAAAGA CAGAAGGTTT | | |
| | TTCCGGTTCT GATATTCGGT TGTTGTGCAA | | |
| | GGAGGCTGCC ATGCAACCAT TA | | |
| C485 | CTTGGTAGTGCGCTTGGGCTGTTCGGTGTTATTGTG | putative vacuolar | SEQ ID N° 277 |
| | GGAATTATTATGTCAGCTCAAGCATCTTGGCCATCC | ATP synthase | |
| | AAGGGTGCGTAAGGCTTCATATTATGTGCTTGCTAT | proteolipid subunit | |
| | TGCTCCGGACTCATCA | | |
| C5 | GATCCCAAAA ATAAGTACCA ACTTCTTTGC | ambiguous hit | SEQ ID N° 278 |
| | TATGGTTTTT TGTGGAGAAC ATTTCACATC | | |
| | TTTTTCCCTG GGGATATATA CTGTCCTGTC | | |
| | ATTGAATCTA ACAATGTCTT CTTCAACTTT | | |
| | CTTGGCCGCT CACTCCCCTC TGCTCAGCCT | | |
| | CCCCCACAAC CTTCTAAGAA AACAAACAAA | | |
| | ACACAAAATA CTCAATCAGC AGGTGGTTTA | | |
| C6 | GATCCCAAAG AAAGAATGCC AATTTCGGAT | transposase-like | SEQ ID N° 279 |
| | TACGGTCCTA ATATTCGAGA CGAAGTAAGG | protein | |
| | AGATATTATA TAAACAAAGG GCCTTGTCAA | | |
| | CCGATTGGTC ATGCGTTTCC TAAAACTAAG | | |
| | ATTGGGAGTA AAATGCGTCC ATTTAGTCCC | | |
| | ACTTGGTTTA | | |
| C7 | GATCCCATCG ATTATTTGGT TTTCCGGTGA | putative protein | SEQ ID N° 280 |
| | GGATTCAATC CATCGAGGTT CCATCGTGGT | AT5g44010 [A. | |
| | CTCCGGCTTA CGGTCTATTT GTGTTCAACT | thaliana] | |
| | ATAGTGTCGC ATTTTTCTTG TAAACTAGTT | | |
| | GGAATATCTT TA | | |
| C8a | GATCCCAATT TTTCAGAATT GCTACTCTCA | phosphate/phospho | SEQ ID N° 281 |
| | GTATTGTCTT TTGTGGGTCT GTTGTGGGTG | enolpyruvate | |
| | GCAATATTTC TTTA | translocator-like | |
| | | protein | |
| C8c | GATCCCATTA TATCCTACCG CAATTTTTCA | putative protein | SEQ ID N° 282 |
| | GGGTGAAATT GATGGTGAAG GGATGAGTTT | At1g10410 [A. | |
| | TGTCTTGTAC TTTA | thaliana] | |
| C9 | GTGCTGTTCC AAGTAATGCC TCTGACAATG | pyrophosphate- | SEQ ID N° 283 |
| | TATATTGCAC GCTTCTTGCT CAAAGTTGTG | dependent | |
| | TTCATGGAGC AATGGCAGGG TCCACAGGTT | phosphofructo-1- | |
| | ACACCTCGGG GCTTGTCAAC GGTCGCCAGA | kinase-like protein | |
| | CTTATATTCC ATTCAATCGT ATAACCGAGA | | |
| | AGCAAAATAT GGTGGTTATA ACTGACAGGA | | |
| | TGTGGGCACG TCTTCTTTCG TCAACCAATC | | |
| | AGCCAAGCTT CTTGTGCCCG AAAGATGCTT | | |
| | GAAGAGGTTA | | |
| MAP2 | ACAGCTATGA CCATTAGGAC CTATTTAGGT | putative protein [A. | SEQ ID N° 284 |
| | GACACTATAG AACAAGTTTG TACAAAAAAG | thaliana] | |
| | CAGGCTGGTA CCGGTCCGGA ATTCCCGGGA | | |
| | TGTTACTTGA CGTGTTTTCT TTTCTTTTAC | | |
| | TCTCCGCCAA TTCAAGACTT CTCAAAGTAC | | |
| | TTTCTCATCT AAAGCAAAAT GTCCGACGGA | | |
| | GGATTAACGG TTTTGGACGG ATCACAGCTG | | |
| | AGAGCCGTCA GCCTATCGTT ACCGTCATCG | | |
| | GACGGCAGCT CAGTCACCGG AGCTCAGCTT | | |
| | CTCGATTTCG CTGAATCCAA AGTCTCAGAG | | |
| | TCGCTCTTCG GCTTCTCATT GCCGGATACT | | |
| | CTCAAGTCCG CCGCTCTCAA ACGCCTCAGC | | |
| | GTCGCCGATG ACCTTAATTT CCGCCGTGAA | | |
| | CAGCTCGATC GTGAAAATGC CTCGATCATT | | |
| | CTCCGAAATT ACGTCGCTGC CATTGCAGAC | | |
| | GAACTCCAAG ATGATCCTAT AGTCATTGCA | | |
| | ATTTTGGATG GGAAAACTCT TTGTATGTTT | | |
| | TTGGAAGATG AAGACGACTT TGCCATGTTG | | |
| | GCTGAGAATC TTTTCACTGA TTTAGACACA | | |
| | GAAGATAGAG GAAAGATCAG AAGAAATCAA | | |
| | ATACGGGATG CTCTCATTCA TATGGGTGTT | | |
| | GAAATGGGAA TTCCTCCTCT TTCAGAGTTT | | |
| | CCTATACTAA GTGACATTTT AAAGAGGCAT | | |
| | GGAGCTGAAG GAGAGGACGA ACTGGGGCAA | | |
| | GCCCAATTTG CACATTTACT TCAGCCTGTG | | |
| | CTTCAGGAGC TGGCAGATGC TCTTGCTAAG | | |
| | AACCCTGTGG TTGTAGTGCA GAAAATCAAG | | |
| | ATCAATAATG GTTCCAAATT AAGAAAGGTT | | |
| | TTGGCTGATG AAAAGCAACT AAGTGAGACA | | |
| | GTAGAGAAGA TAATGCAGGA AAAGCAGGAT | | |
| | GAGAAGGATA GTCTAAGTAA CAAAGATGCC | | |
| | ATTCGGTGTT ATCTCGAGAA AAATGGAGCA | | |
| | TCATTGGGCT TGCCACCTCT GAAGAATGAT | | |
| | GAAGTGGTGA TTCTTCTATA CGACATTGTA | | |
| | TTAGGTGATA TAGAAAATGG AAAGACCGAT | | |
| | GCAGCATCAG ATAAGGATGA AATCTTGGTT | | |
| | TTCCTGAAGG ATATCCTTGA GAAATTTGCA | | |
| | GCTCAACTTG AAGTTAACCC AACTTTCCAT | | |
| | GATTTTGACA ATTGAAGTTA TATACACCCT | | |
| | CTCAAGATAA GTTATACCAG AAAGATCATA | | |
| | TATATGTATT TTAGCCTTTG CTTTTGGTGC | | |
| | CAAGGCAACT TATAGTGTTT AATTTTTATA | | |
| | TTGTAGAATA ACAAGTATTC ATGAGACAGA | | |
| | TAAATCAAAC CCATTTCATT TGCATTTCAA | | |
| | AAAAAAAAAA GGGCGGCCGC TCTAGAGTAT | | |
| | CCCTCGGGGG GCCCAAGCTT ACGCGTACCC | | |
| | AGCTTTCTTG TACAAAGTGG TCCCTATAGT | | |
| | GAGTCGTATT ATAAGCTAGA CACA | | |
| MAP3a | ATCCAGAATT AATAAACCCT AGTAAGTGAA | ethylene-responsive | SEQ ID N° 285 |
| | AGTGAAAGAA ACTACTCATC CAAATATCTA | transcription factor | |
| | TAGAAAAGTA AATGAATCCC GCTAATGCAA | | |
| | CCTTCTCTTT CTCTGAGCTT GATTTCCTTC | | |
| | AATCAATAGA AAACCATCTT CTGAATTATG | | |
| | ATTCCGATTT TTCTGAAATT TTTTCGCCGA | | |
| | TGAGTTCAAG TAACGCATTG CCTAATAGTC | | |
| | CTAGCTCAAG TTTTGGCAGC TTCCCTTCAG | | |
| | CAGAAAATAG CTTGGATACC TCTCTTTGGG | | |
| | ATGAAAACTT TGAGGAAACA ATACAAAATC | | |
| | TCGAAGAAAA GTCCGAGTCC GAGGAGGAAA | | |
| | CAAAGGGGCA TGTCGTGGCG CGTGAGAAAA | | |
| | ACGCGACACA AGATTGGAGA CGGTACATAG | | |
| | GAGTTAAACG GCGGCCGTGG GGGACGTTTT | | |
| | CGGCGGAGAT AAGGGACCCG GAGAGAAGAG | | |
| | GCGCGAGATT ATGGCTAGGA ACTTACGAGA | | |
| | CCCCAGAGGA CGCAGCATTG GCTTACGATC | | |
| | AAGCCGCTTT CAAAATCCGC GGCTCGAGAG | | |
| | CTCGGCTCAA TTTTCCTCAC TTAATTGGAT | | |
| | CAAACATTCC TAAGCCGGCT AGAGTTACAG | | |
| | CGAGACGTAG GCGTACGCGC TCACCCCAGC | | |
| | CATCGTCTTC TTCATGTACC TCATCATCAG | | |
| | AAAATGGGAC AAGAAAAAGG AAAATAGATT | | |
| | TGATAAATTC CATAGCCAAA GCAAAATTTA | | |
| | TTCGTCATAG CTGGAACCTA CAAATGTTGC | | |
| | TATAACTGTA TTTAATTTGG AAGGAATTAA | | |
| | TTAAGGTTAT TCTATGTCTT TGTATTAGAA | | |
| | TTTAGAATAA TTCCCTAAAG CTCCTGAAGA | | |
| | ACGAAACTTG TAAACATCTC TCTGTCTCCG | | |
| | TATCATGTTC TAATTTAACA TGAAATTACA | | |
| | TGAGCGCAAA AAAAAAAAAA AAAA | | |
| MAP3b | TTGGGGGAGG TTCGCGGCGA AGATAAGGGA | AP2-domain DNA- | SEQ ID N° 286 |
| | CCCGGAGAGA AGAGGCGCGA GATTATGGCT | binding protein | |
| | AGGAACTTAC GAGACCCCAG AGGACGCAGC | | |
| | ATTGGCTTAC GATCAAGCCG CTTTCAAAAT | | |
| | CCGCGGCTCG AGAGCTCGGC TCAATTTTCC | | |
| | TCACTTA | | |
| MAP3c | TTGGGGGAGG TTCGCGGCGG AGATGGAAGC | putative protein | SEQ ID N° 287 |
| | ACTTATGGAG GCCAAAGGGG TGAGCAAGTA | At5g28830 [A. | |
| | TATCGAAGTG CCAGGTGCTC TCCTTCCCCA | thaliana] | |
| | GGAAGAGTAT CCTGAAATAG TTGCAGAACA | | |
| | GCTTTACAGG TTTCTGCAAG AGAAGTTTGA | | |
| | GCTTCAGGCT TA | | |
| MAP4b | TTGGGGGAGG TTCGCGGCGG AGATGCACTC | calmodulin-related | SEQ ID N° 288 |
| | CGTTATGAAG GGCATTGGAG AGAAGTGTTC | protein | |
| | GCTTA | | |
| MAP5 | GGCCGTGGGGGAGGTTTGCGGCTGAAATAAGGGAC | AP2-domain DNA- | SEQ ID N° 289 |
| | CCGGAGAGAAGAGGCGCGAGATTATGGCTAGGAA | binding protein | |
| | CTTACGAGACCCCAGAGGACGCAGCATTTGGCTTAC | | |
| | GATCAAGCCGCTTTCAAAAGCCGCGGCTCGAGAGC | | |
| | TCGGCTCAATTTTCCTCAC | | |
| MC101 | TAAAGGCGCC GACTATGCTG CATCATTCTG | putative protein | SEQ ID N° 290 |
| | GGCTGAGGTA TTTGATGGGG TGAGGCAGAG | At3g06150 [A. | |
| | AGGGTTGACA CCACCAGAAG TAATATATAG | thaliana] | |
| | GACCACAGTCACCACAGGCG GATACGCTAG | | |
| | AAGATTGGCA TTCAATCCAA ATAAAATGGA | | |
| | GGCCTTCAAT GGGGTAGTCT TGGATAAGTT | | |
| | GAGGGCATAT GGTTTAGTTG ATCGCGTCAT | | |
| | TGATGATTTC GACATGACTT ATCCTTGGCA | | |
| | CTATGATAACCGATGCAATG ACGGGGTGCA | | |
| | TTATGGCCGT GCTCCTGCCA AG | | |
| MC102 | TAAAGGTGGA GAATATTTTG GTGATGGGAC | carbonic anhydrase | SEQ ID N° 291 |
| | ACAGCTGCTG TGGAGGTATA AAAGGACTCA | | |
| | TGTCTATCCC TGATGATGGC TCCATAGACA | | |
| | GTCATTTCAT CGAAGAATGG GTCAAAATCT | | |
| | GTTTGATATC AAAGGCAAAG GTAAAGAGAG | | |
| | AACATGGCGA CAAGGATTTC ACTGAACAAT | | |
| | GTACAATATT GGAGAAGGAGGCAGTAAATG | | |
| | AATCACTAGC CAACTTACTG ACATATCCAT | | |
| | TTGTGAGGGA AGCTGTG | | |
| MC104 | TAACCTTGGA AAGACATGGG AGAAGCTGCA | P40-like 40S | SEQ ID N° 292 |
| | AATGGCTGCG AGGGTTATTG TTGCTATTGA | ribosomal protein | |
| | GAATCCAAAG GACATAATTG TGCAATCAGC | | |
| | CAGGCCCTAT GGCCAGAGAG CTGTCTTGAA | | |
| | GTTTGCTCAA TACACTGGCG CAAGTGCCAT | | |
| | TGCTGGCCGT CACACTCCCG GTACTTTTAC | | |
| | CAACCAGCTT CAGACTTCAT ACAGTGAGCC | | |
| | CCGACTCCTC ATTCTCACTG ACCCAAGAAC | | |
| | TGATCACCAG CCTATCAAGG AAGCTGCACT | | |
| | TGGGAACATC CCTACTATGG CTTTCTGTGA | | |
| | CACTGATTCA CCGATGCGCT ATGTTGACAT | | |
| | TGGTATCCCT GCCAATAACA AAGGGAAGCA | | |
| | CAGTATCGGT GTTCTTTTCT GGCTCTTAGG | | |
| | AAGGATGGTA CTGCAGATGC GCGGTAGCAT | | |
| | TCCTCAGGGA CACAA | | |
| MC105 | TAACAGACGT TGATGATATG ATGTTATGGG | alanine acetyl | SEQ ID N° 293 |
| | CAGGCGACGA TCGAGTAACT AGGACCATCC | transferase-like | |
| | GATGGAAAAC TTTGACCTCG AAAGAAGAGG | protein | |
| | CATTGGCCTT CATCAAGGAA GTGTGTATAC | | |
| | CTCACCCCTG GCGTCGATCA ATATGCATCG | | |
| | ATGACCGATC GATCGGGTTT GTATCAGTAT | | |
| | TTCCTGGATC AGGTTATGAT AGAAGCCAAG | | |
| | GTGTCATAGG ATATGATATT GCAGTTGAAT | | |
| | ATTGGGGGCA GGGGATTGCT ACAAATGCTA | | |
| | TCAAAATGAC AATCCCTCAA GTGTACAATA | | |
| | ACTTTCGTGA AATAGTAAGG CTTCAGGCAT | | |
| | TAGCTAATGT TAAGAATAAG GCATCCCAAA | | |
| | GGGTGTT | | |
| MC106 | AATTCCCCCATGTGCATGCCTGAGTGCACAAACAG | putative late | SEQ ID N° 294 |
| | GAAGGCGAATTGCAATCACCCCGGAGCAGCATGCT | embryogenesis | |
| | TGGATC | protein | |
| MC107a | TAACCCAATTTTGTTGCCAAAGAAAACTGGAGGTG | histone H2A-like | SEQ ID N° 295 |
| | AAAAGGCTGGCAAAGAACATAAATCTCCTTCCAAA | protein | |
| | GCAACCAAATCTCCTAAGAAGGCTTAGATTTAGTG | | |
| | GCTGTTATAAGCCTCTTGCTTTTCTATCTTTATTTGG | | |
| | ATC | | |
| MC107b | TAACACGGGAATGATACCAGAGATACAGGCTACAG | proline transport | SEQ ID N° 296 |
| | TCAGACCACCTGTAATTGAGAACATGTTGAAAGCT | protein | |
| | CTGTTCTTTCAGTTCACAGTGGGAGTTGTGCCCTTG | | |
| | CATGCTGTTACTTATATAGGTTATTGGGCTTATGGA | | |
| | TC | | |
| MC108 | TAACAACCCC ATTTGGAATA GCACTTGGAA | putative metal | SEQ ID N° 297 |
| | TTGGTTTATC AAAAGTGTAT AGTGAAAATA | transport protein | |
| | GTCCAACAGC ACTA | | |
| MC109 | CGTTCGTGGGACCTACAAGGGGCGCGAGGGCAAAG | putative 60S | SEQ ID N° 298 |
| | TCGTTCAAGTGTACCGTCTGAAATGGGTAATTCACA | ribosomal protein | |
| | TTGAACGCAGTAACACGTGAGAAGGTTACTC | | |
| MC113 | AGTAAAGGTG CAGAATATTT TGGTGATGGG | putative carbonic | SEQ ID N° 299 |
| | ACACAGCTGC TGTGGAGGTA TAAAA | anhydrase | |
| MC114c | GATCCAGCAG AGTCGGAGGT TGCCGGATTT | putative beta- | SEQ ID N° 300 |
| | CCTTCAGAGT GTAAACTTGA AGTACGTTA | ketoacyl-CoA | |
| | | synthase | |
| MC115 | TAAGCACCCT AGTATTTCTG CATACATGGG | putative | SEQ ID N° 301 |
| | ATCAAGACTC GCTGGGAAAG TTTTGGCAAC | Dihydroorotase | |
| | CTTTGTGCGC GGAAATCTTG TATACAAGGA | | |
| | GGGAAATCAT GCTTCTCTTG CATGTGCTCT | | |
| | CCCAATTCTG CATAGATAGT TAGTGCATGA | | |
| | GCCTATCAGT AACTCCACCA ACTTACCATA | | |
| | TATCATCCAA ATTATTTCTT CTGTGCAATC | | |
| | TTCATGTTCT TTGTTGTGTC CCTTTGACAT | | |
| | TCTTGGAGAT GACCATATGG CATGATATAC | | |
| | AGATGGAATT GGTGACTTCC ATCATTT | | |
| MC116 | TAAGCAACCC GAAACCCGAT CCGAACCATT | putative protein | SEQ ID N° 302 |
| | CAACTCGGAC TAAGTCGGTT CGGACCGAGG | At1g71780 [A. | |
| | TTCCGGAGGT CAAGGTCCAC CTGTATCGGC | thaliana] | |
| | AAGGCAAGGG TCCTATCGAC GAATTCACGA | | |
| | TGCCCTTAGG TGGATGGGAC CAGGATCAGC | | |
| | TGGAGGTTCG TGAAATTCTC GACAAATACG | | |
| | GGTTCAAATC GGTCTATGCA TTCAAACCGG | | |
| | ATACGGGTCG GGGCGTTCCC ATCAGATTCA | | |
| | ACCCCCGTAA CGGCCGATCT A | | |
| MC118 | TAAGGTATTT GTGAAGTCTT ACTATTTTCC | N-acetyl-gamma- | SEQ ID N° 303 |
| | ACAAGGAGAG ACTGCTTCAA GATTTTTTGT | glutamyl-phosphate | |
| | GGAAGAGTTT TGTTTGCTGA GTTTGTAATT | reductase like | |
| | TCTGTAGAAG TATTCCCGTG TATCCTGGCG | protein | |
| | TAGTTTTCAG ACGTACCCTA TATTTGATTG | | |
| | CTAATTTTAT GCCTCAGAAG GAGATTATGT | | |
| | GCCATAGATA AAGTTGAACA GGGGGGTGGA TC | | |
| MC121a | AGTCCTATGTGATTGCAAGAGACCGATTTCTTGTTC | putative arginine | SEQ ID N° 304 |
| | AAAATGGAAAAATGTTTCCTGGTGGCGGAAGAATA | methyltransferase | |
| | CACATGGCACCATTTAGTGACGAATATTTGTATATG | | |
| | GAAATAGCAACTAAGGCGACCTTTTGGCAGCAACA | | |
| | AAACTACTTTGGGGTTGACTTGACACCCTTGCACGG | | |
| | ATC | | |
| MC121b | GCGACTTCCGCTTTCGGTACAGTGCAATCTTCTACC | 6,7-dimethyl-8- | SEQ ID N° 305 |
| | TCGTGCAACAACTGTAAATCCCACACAACTGCACT | ribityllumazine | |
| | CTCCTCTTTACTCTTTGTCTCTGCCTTTCCACAGACA | synthase | |
| | AAGCATAACCTCTTCACCTGCACTATCATTCACCCA | | |
| | ATCTCAAGGTTTAGGGTCTGCAATTGAGAGACATT | | |
| | GCGACCGGTCGGATC | | |
| MC123 | TAAGCAAAGA GAGGCAGCTT GGTTTGCTGG | putative protein | SEQ ID N° 306 |
| | TTCTGTGAGA TCAAGACTAC AGTATTTGGG | At2g46580 [A. | |
| | GCCCACTCCA GGACTTCCTT CTCTAGATGA | thaliana] | |
| | GCAACCATTG CACGACTCGT TGGATC | | |
| MC124 | CGGGCCCAATTTGCCCTATAGTGAGTCGTATTAAA | putative protein | SEQ ID N° 307 |
| | AGCAGGCAAGCCTGTTGGTGGGTTCAAGATAGGTA | At1g50570 [A. | |
| | GACAATCTGGGGAATGGACGGGTTAAAATTTTCAT | thaliana] | |
| | CCGTACTTCCATCAGAGAGTTATCTTACATGCAGG | | |
| | TTTTTCTCTGCAAGGAAACATGGGTTGGTGGATGCT | | |
| | GTTGTGAGATGTAAAAGCTCCGAGCGGACAGCTGT | | |
| | TGTCGCCCTTCCTGGTGGAATTGGTACCCTTGACGA | | |
| | GATTTTTGAGATTATGGCTTTGAT | | |
| MC125a | TAATCTCAAT GCATCTTTGT TTGTTTGAAT | acyl carrier protein | SEQ ID N° 308 |
| | TTGTTCATCA AAATCAAAGG TACACTTGCT | | |
| | CCTTGTCATT TGACTAGTTC AAGGTTGTAG | | |
| | AATTTTGATC CTCTTGAGAG AGGCAATAAT | | |
| | CAGACTCTTT GGAAGACCAG TTGCTCAGGC | | |
| | TTTGCCATTG AGGATTATAT CATCCTTTTG | | |
| | TTGCTTTTCT GGAAGACATG ACTCAGTATT | | |
| | TATTCTGTTG CCGTCYLTCC TCTTATAATA | | |
| | TTCGAATGCC ACAAATTCAA GCTTGGTTTG | | |
| | ATTGTTGCAC TGATTTGAAA AATCTGTCTA | | |
| | GTCTGGCTCA TGAACTTGTG AAGCTGATGC | | |
| | TGGATC | | |
| MC125b | TAATACAGAA GCCTTACTCT ATTGTGTACT | putative protein | SEQ ID N° 309 |
| | TCCATTCTGC TGCAACCTTA CAGATTCAAC | At1g69340 [A. | |
| | CAGATCTAGG ATTGATGAAG AGAATACAAC | thaliana] | |
| | AAATACTCGG TCGCAAGCAC CAGCGCAACC | | |
| | TTCATGCGAT ATATGTTCTT CACCCTACTT | | |
| | TTGGACTGAA GAGTGCAATA GTTGCACTAC | | |
| | AGCTCTTTGT GGATTATGTG GTATGGAAAA | | |
| | AAGTAGTGTA TGTAGATCGT CTTCTGCAAC | | |
| | TATTCCGCTA TGTTCCTCGT GAACAGCTAA | | |
| | CCATCCCAGA TTTTGTATTC CAGCATGATT | | |
| | TGGAAGTAAA TGGAGGGAAG GGCCTAATTG | | |
| | TGGATC | | |
| MC126 | TAATGGATGC TGCAACGCAA GGTGCCCTAC | putative protein | SEQ ID N° 310 |
| | AAGCAGGGAA GCCTGTTGGT GGGTTCAAGA | At1g50570 [A. | |
| | TAGGTAGAGA AGCTGGGGAA TGGACGGCTT | thaliana] | |
| | CAAATTTTCA TCCGTACTTG CCATCAGAGA | | |
| | GTTATCTTAC ATGCAGGTTT TTCTCTGCAA | | |
| | GGAAACATGG GTTGGTGGAT GCTGTTGTGA | | |
| | GATGTAAAAG CTCCGAGCGG ACAGCTGTTG | | |
| | TCGCCCTTCC TGGTGGAATT GGTACCCTTG | | |
| | ACGAGATTTT TGAGATTATG GCTTTGATTC | | |
| | AACTCGAACG AATTGGATC | | |
| MC129 | TAAGCAACCC GAAACCCGAT CCGAACCATT | putative protein | SEQ ID N° 311 |
| | CAACTCGGAC TAAGTCGGTT CGGACCGAGG | At1g71780 [A. | |
| | TTCCGGAGGT CAAGGTGATG AGTCCTGAGT | thaliana] | |
| | AATGACAACA ATATAGCATC ATTGGTAGG | | |
| MC130a | GATCCAAGAAGCTCTTTTGCCTAGCCTTATGAGTAA | G protein beta | SEQ ID N° 312 |
| | TTTTATGTTTCCTTCTGTGTTTTTCTTACAGATCTTT | subunit-like protein | |
| | TCCGCAGTAGAAGTTTTGTTTGGATTA | | |
| MC130b | TGAGTATGTG GTGTGTTTGT CCAAAAGGTA | putative protein | SEQ ID N° 313 |
| | GATTTATTGA AAAGTATCAA GCAGCTCAAG | AT3g45540 [A. | |
| | TGTAGATGTG GTCATCTAAC AAATGGTGGA TC | thaliana] | |
| MC203 | TAAAGGTGCA GAATATTTTG GTGATGGAAC | carbonic anhydrase | SEQ ID N° 314 |
| | ACAGGTGCTG TGCAGGTATA AAAGGACTCA | | |
| | TGTCTATCCC TGATGATGGC TCCATAGACA | | |
| | GTCATTTCAT CGAAGAATGG GTCAAAATCT | | |
| | GTTTGATATC AAAGGCAAAG GTAAAGAGAG | | |
| | AACATGGCGA CAAGGATTTC GG | | |
| MC204 | ATGTATGGTA GATCAGGGCT TGATCGATTT | putative protein | SEQ ID N° 315 |
| | AAGAAAGCTC AGTCATTGGA GCCATTTCAG | AT5g47790 [A. | |
| | GTGTCTGCGA ATTCAGCTGC TAAACCAGCA | thaliana] | |
| | TTGCAGCCTA CTACAAAGGC GGTTACACAT | | |
| | CCTTTTCCAG CATATGCACA ATCCACAACA | | |
| | TCTCATCAAC AAACTCAATA CGTAAATCCA | | |
| | CAACCTGCTT TGCAGAAATC CGTGGCGGCA | | |
| | GATGCAACCG CTTCTACAGT GCCAACTCAT | | |
| | CATGTCACTC ATGGAGGGGG ACAATCAACT | | |
| | TGGCAGCCTC CTGATTGGGC TATTGAGCCA | | |
| | CGTCCAGGAG TTTATTATCT TGAGGTGATC | | |
| | AAGGATGGTG AGGTACTCGA TCGAATTAAT | | |
| | TTGGATAAGC GAAGGCATAT CTTTGGACGG | | |
| | CAGTTTCATA CTTGTGATTT TGTCCTTGAT | | |
| | CATCAGTCAG TCTCACGCCA GCATGCTGCT | | |
| | GTGATTCCTC ACAAAAATGG AAGCATTTAT | | |
| | GTGATTGATT TAGGATCTGC ACATGGAACA | | |
| | TTTGTAGCAA ATGAGAGGCT AACAAAGGAT | | |
| | TCCCCTGTCG AACTTGAGCC CGGACAATCT | | |
| | TTGAAGTTGG CTGTATCAAC AAGGCCTTAC | | |
| | ATCTTGAGAA GGAACAATGA TGCTCTCTTC | | |
| | CCTCCTCCAC GGCAACTGGC AGAAATAGAT | | |
| | TTCCCGCCAC CTCCAGATCC TTCAGATGAG | | |
| | GAAGCTGTTT TGGCTTATAA CACCTTTTTA | | |
| | AACCGCTATG GGCTTATAAG GCCTGATTCA | | |
| | TTGTCAAAAT CAACAGTATC AACTAGTGGG | | |
| | GAGGATGTCA ACTATTCATC TGACAGGCGC | | |
| | GCGAAAAGAA TTAGGAGAAC AAGTGTGTCA | | |
| | TTTAAAGATC AGGTTGGAGG AGAGCTAGTT | | |
| | GAAGTTGTTG GTATTTCGGA TGGAGCAGAT | | |
| | GTGGAGACAG AACCTGGTCC ATTGGGTGTG | | |
| | AAAGAAGGAA GTCTTGTCGG AAAATATGAG | | |
| | TCCCTAATAG AACCTACAGT GATACCGAAA | | |
| | GGGAAAGAAC AGTCCTCTGT AAAGGATGCC | | |
| | ACCGTTACCC GAACAGGTGT ATCGGACATA | | |
| | CTTCAACAGG TATTGTCCAA GGTGAAAAAT | | |
| | CCGCCGAAGG GTGGAATTTA CGACGATCTT | | |
| | TATGGAGAAT CAGCTCCTGC TAAAGGGGGA | | |
| | TTTTGGGCAT ATTCTGATTC CAGTCAAACA | | |
| | GCTTCTACTA ACGACGCTAA AGGAGACTCC | | |
| | CCTTGTTCTT TACGCAGAAT CTTTGGACAT | | |
| | ATCTCAAACA ATGTAGACGA CGATACCGAT | | |
| | GATTTGTTTG GATAG | | |
| MC205 | TAAAGCAGAT TTGCTCAACA TTACTCAACT | putative protein | SEQ ID N° 316 |
| | TTCTGAGTAT AGAAAAGAAG CA | At3g11030 [A. | |
| | | thaliana] | |
| MC207a | GAGTCCTATGTGATTGCAAGAGACCGATTTCTTGTT | putative arginine | SEQ ID N° 317 |
| | CAAAATGGAAAAATGTTTCCTGGTGTCGGAAGAAT | methyltransferase | |
| | ACACATGGCACCATTTAGTGACGAATATTTGTATAT | | |
| | GGAAATAGCAAATAAGGCGACCTTTTGGCAGCAAC | | |
| | AAAACTACTTTGGGGTTGACTTGACACCTTTGCACG | | |
| | GATG | | |
| MC207b | ACTCTCTCTTCCACTGCTCAGACAACAATCGAAATT | heat shock protein | SEQ ID N° 318 |
| | GATTCTCTGTATGAGGGGGTTGACTTTTATCCTACC | 70 | |
| | ATTACTCGTGCTAGATTCGAGGAGTTGAACATGGA | | |
| | TC | | |
| MC209 | TAACAAAACAAGCAGTGGCAAGGAGTTCCCAGTGA | EEF53 | SEQ ID N° 319 |
| | CAGCTTTTGTATTCGCAAGTCCTAAAGTTGGGGATC | | |
| MC210b | TAACGAAGAAAACAACAACAACAATAACAACAAC | putative protein | SEQ ID N° 320 |
| | AACAACAAGCCCAGTGTAATCCCACACGTAGGGAT | AT3g24200 [A. | |
| | C | thaliana] | |
| MC212 | TAAGGAGGCT GTAGAATTGA TCAATGGGAG | quinolinate | SEQ ID N° 321 |
| | GTTTGATACG GAGGCTTCAG GAAATGTTAC | phosphoribosyl | |
| | CCTTGAAACA GTACACAAGA TTGGACAAAC | transferase | |
| | TGGTGTTACC TACATTTCTA GTGGTGCCCT | | |
| | GACGCATTCC GTGAAAGCAC TTGACATTTC | | |
| | CCTGAAGATC GATACGGAGC TCGCCCTTGA | | |
| | AGATGGAAGG CGTACAAAAC GAGCATGAGC | | |
| | GCCATTACTT CTGCTATAGG GTTGGAGTAA | | |
| | AAGCAGCTGA ATAGCTGAAG GGTGCAAATA | | |
| | AGAATCATTT TACTAGTTGT CAAACAAAAG | | |
| | ATCTTGGGAC GGTGAGCTCC GTTTGTGGGA TC | | |
| MC214 | TAAGGTAAGG CACAATAATG TCGTTCCTAT | putative pyruvate | SEQ ID N° 322 |
| | GATGGCTTTG GGAGTCCAAC AACTCAAGAA | dehydrogenase | |
| | AGATTGGCCT AAAGTTGATT ATGAGGATTT | kinase | |
| | GAGAGAAATA CACCAAT | | |
| MC215 | TAAGCCCGAG AGGTTTCTTG GCTCGAAAAT | cytochrome P450 | SEQ ID N° 323 |
| | AGATGTGAAA GGGCAGCATT ATGAGCT | hydroxylase | |
| MC216 | TAACGACTGC AGAATCATCT ATATACGAAG | putative protein | SEQ ID N° 324 |
| | TGCTTGAATC CCATGGATTG CCAATGGGTT | At3g07460 [A. | |
| | TACTTCCAAA AGGTGTGAAG AATTTCACAT | thaliana] | |
| | TAGACAATTC GGGGAAATTT GTAGTCCATT | | |
| | TGGATCAAGC TTGCAATGCT AAATTCGAGA | | |
| | ATGAGTTTCA CTATGATAGG AATGTATCGG | | |
| | GTACAATAAG TTACGGACAG ATCCATGCAC TTT | | |
| MC219 | GGAATCGAACTAATCGCATCGGAAAACTTCACATC | glycine | SEQ ID N° 325 |
| | ATTCGCCGTAATTGAAGCTCTCGGCAGTGCCTTA | hydroxymethyltrans | |
| | | ferase | |
| MC220 | GATCCCTATTTTACAAGAGTGCATTGATGCCATCAC | putative protein | SEQ ID N° 326 |
| | TGAACACCAAAGGCTTCTGTCCTTA | At1g07970 [A. | |
| | | thaliana] | |
| MC222 | TAATAGGTAT AGCATGCCAC AAATCTGGAG | ambiguous hit | SEQ ID N° 327 |
| | TTGAGGTGGT TATTCTTATA CCCCCAAATG | | |
| | CCCCCAGCAT AGCAGCTTAT GGTTCCATTG | | |
| | TTGTTGT | | |
| MC223 | TAATGAGACAATGAGATTATACCCTCCGATACCAC | cytochrome P450 | SEQ ID N° 328 |
| | TTTTATTGCCTCATTATTCAACTAAAGATTGTATT G | | |
| MC225 | TATTGGTACGTCGTAAAATGTGACCGGAAAACCAA | polygalacturonase | SEQ ID N° 329 |
| | CCGGATTA | inhibitor | |
| MC302 | CCCCTATATT TTTCCCCTAT ATCTTTTTCT CCTCCC | poly(A)-binding | SEQ ID N° 330 |
| | | protein | |
| MC304 | TAACGACTGC AGAATCATCT ATATACGAAG | putative protein | SEQ ID N° 331 |
| | TGCTTGAATC CCATGGATTG CCAATGGGTT | AT3g07470 [A. | |
| | TACTTCCAAA AGGTGTGAAG AATTTCACAT | thaliana] | |
| | TAGACAATTC GGGGAAATTT GTAGTCCATT | | |
| | TGGATCAAGC TTGCAATGCT AAATTCGAGA | | |
| | ATGAGTTTCA CTATGATAGG AATGTATCGG | | |
| | GTACAATAAG TTACGGACAG ATCCATGCAC | | |
| | TTTCAGGAAT TGAGGCTCAA GATTTGTTTC | | |
| | TATGGTTTCC AGTGAAGGAT ATTCGGGTTG | | |
| | ATATACCCAG TTCTGGTTTG ATTTACTTCA | | |
| | ACGTTGGCGT TGTATCTAAG CAATTCTCTT | | |
| | TGTCTTCATT TGAGACTCCT AGGGATTGTA CTG | | |
| MC305b | TAACATTGTT TACAGAAGAA AAGCAGGGGG | Plastid-specific 30S | SEQ ID N° 332 |
| | TTATGGACTT ATTATTCCCA AGGAAGATGG | ribosomal like | |
| | TAAGACAAAG TTAGAGCCTG TGGAGGTTGA | protein | |
| | ACTAGAGAAA GAAACGTCGA TGGCAGAATA | | |
| | GAAGGAATTG ATGAAAAGTG ATTAGTTAGT | | |
| | GACCGAGTAC ATTTACTTTG CGTTACGATC | | |
| | ACTTTTGTAG AGAAGGTTTT CTGCTTGAGG | | |
| | ATGTTTTTGC ACCCATCATC TGCGACAGAC | | |
| | TGACGGAGCA CTACGCA | | |
| MC306b | TAACCATGCTCTTACAGGATTCTTTTGAGGATGACA | kinesin like protein | SEQ ID N° 333 |
| | AGGCCAAAATTCTCATGATACTGTGTGCGAGCCCG | | |
| | GATC | | |
| MC307 | TAAGGCTGCT GGTGAAAGAA GTGGCGGATC | putative protein | SEQ ID N° 334 |
| | TCTCGATGGT GTAGCATTTC TCCTAAGTTC | At2g44090 [A. | |
| | AGATTTCCTT GGTGATCCAG CTGCAACTTA | thaliana] | |
| | TGCGGTCGCC GACAGCATCG CTAAGTCGGA | | |
| | TGACGAGGCT GTCGCTCCTG AGCTCAGGTC | | |
| | TTTCCTTCGG GAGCATTGGT CGGAAGCTGC | | |
| | TTTCTCAGAC GGGCTTAGGC AAGGACAAGA | | |
| | ACACTACTTG AATATCGTGC GTATTTTGAA | | |
| | ATGGGGGGAA | | |
| MC308 | TTGGCAGTGAGATTTTTGCGAATGATTGAGGCTGCT | putative Pto kinase | SEQ ID N° 335 |
| | GTCATCTTGTGTGCGCCACTCATGCTTCAAAGAGAC | interactor | |
| | CAGCAATGGGACAGGTAACACTTGTTCCATTTTATT | | |
| | GAATGAAAACCTATGCCAGAAACGCCCTTA | | |
| MC309a | TAATGGTCTA GCATCGGAGG ATGCTCTGGG | polyprotein | SEQ ID N° 336 |
| | ATTTCTTGAG GAGTGTTACT GCATTCTCCG | | |
| | TACTATGGGT ATCTCAGGAT CGAGCGGGTT | | |
| | TTCTTTCACT ACTTTCCAAC TTCGAGGAGT | | |
| | CGCGTATGAT TAGTGGCACA CCTATGAGTT | | |
| | AGACAGTCCA GATGAGGCTG CTTCACTAAC | | |
| | TTGGGCTCAG TTTTCGGAGC ACTAC | | |
| MC309b | GATCCGAGCA TTGTGGAGGC ACTATTTCCA | ADP-ribosylation | SEQ ID N° 337 |
| | GAACACTCAG GGTCTCATTT TTGTGGTTGA | factor-like protein | |
| | TAGCAATGAC AGAGACCGTG TCGTGGAGGC | | |
| | AAGAGATGAA TTGCACAGGA TGTTGAACGA | | |
| | GGATGAGCTT CGGCATGCTG TGCTGCTTGT | | |
| | TTTTGCTAAC AAACAAGATC TTTTCCGCAG | | |
| | TAGAAGTTTT GTTTGGATTA | | |
| MC310a | GATCCGCCGCACAGACCAAAACACCGCCCAGCGTA | zinc finger like | SEQ ID N° 338 |
| | GGCTTTTCATCTTCGTCAATATTAGCAAATTAGAAC | protein | |
| | CCCCACCCATTCTCTTCTTTTTCAACAACAGCCAAC | | |
| | CCTCAGCTGCCGACACACACGCACAGTCGCCGATG | | |
| | GACAGAGAATCAGCGAATGCCATAGCCATTTGCTGC | | |
| | CTCTGCTTCTTCCCATTA | | |
| MC310b | AATGAAAGAATGTTGGAGTCCTATGTGATTGCAA | putative arginine | SEQ ID N° 339 |
| | GACACCGATTTCTTGTTCAAAATGGAAAAATGTTTC | methyltransferase | |
| | CTGGTGTCGGAAGAATACACATGGCACCATTTAGT | | |
| | GACGAATACTTGTATATGGAAATAGCAAATAAGGC | | |
| | GACCTTTTGGCAGCAACAAAACTACTTTGGGGTTG | | |
| | ACTTGACACCTTTGCACGGATC | | |
| MC311a | GATCCGACCA AGGCGTCTTA GCATTGAAGG | eukaryotic initiation | SEQ ID N° 340 |
| | CCTTGAAGCT TTCCGATTCT TTCATGGAAC | factor 3H1 like | |
| | TCTACAAGAG TAACAACTTT ACTGGAGAGA | protein | |
| | AGTTGAGGGA AAAGACTCTT TCATGGGTCG | | |
| | ACATCTTTGA AGAGATACCG ATTA | | |
| MC401 | CAGAATCATC TATATACGAA GTGCTTGAAT | putative protein | SEQ ID N° 341 |
| | CCCATGGATT GCCAATGGGT TTACTTCCAA | At5g19850 [A. | |
| | AAGGTGTGAA GAATTTCACA TTAGACAATT | thaliana] | |
| | CGGGGAAATT TGTAGTCCAT TTGGATCAAG | | |
| | CTTGCAATGG TAAATTGGAG AATGAGTTTC | | |
| | ACTATGATAG G | | |
| MC402 | GATCCTACAATCAACCTGAGAACATGCATAATTTA | putative beta-1,3- | SEQ ID N° 342 |
| | TGTTTTCTTGTAGTGTTTTTCTGATCTGATGAAGGTTT | glucanase | |
| | AGCTACACACCAAGTTTTCTTTTCATTTGCTAACAC | | |
| | CAATGTTCCCACTGAAATGTGGGACAAAAGTAGGA | | |
| | AGCAAAGGGTGAGAGCTGCTTTA | | |
| MC404 | TAACTTCAAT GCGACCAGTG GTGCTCGGAT | nucellin-like | SEQ ID N° 343 |
| | AATACCTCGT TTGGCTCTAG GGTGTGGATA | protein | |
| | TGATCAGTTA CCTGGTCAAT CTCATCATCC | | |
| | TTTAGATGGA GTGCTTGGCC TTGGGAAAGG | | |
| | AAAAGCCAGC ATTGTGTCTC AGCTTCACAG | | |
| | CAAGGGTTTG GTGCGGAATG TGGTAGGCCA | | |
| | TTGCTTGAGT GGCACAGAAG TAGGTTTTCT | | |
| | CTTCTTT | | |
| MC405 | TAACGAGTAT GGCGAAGCCT ATGAATCCCA | NADH | SEQ ID N° 344 |
| | TGCTGAGTTT CGTTAGTTCA AGGCCAGGAT | dehydrogenase | |
| | GGGTCATGCT CTCAAGTTAC TCGTGTATGA | subunit 1-like | |
| | TTTTTTTTAG TCTTGGCAAA TTTTTATGCG | protein | |
| | AGTCTCACCA AAAGATGCAT GTGTGTGTA | | |
| MC406a | GATCCTAGCATTTGAGAAGTTCCTTGAAGAAAACC | trehalose-6- | SEQ ID N° 345 |
| | CATACTGGCGTGATAAAGTGGTTTTGCTGCAAATTG | phosphate synthase | |
| | CTGTGCCAACAAGAACAGATGTTCCTGAATACCAA | | |
| | AAACTTACTAGTCAGGTTCATGAGATTGTTGGACG | | |
| | CATCAATGGCCGGTTGGAACTTTGACTGCAGTGCC | | |
| | TATTCATCATCTGGATCGATCTCTTGACTTTCATGC | | |
| | ATTATGTGCACTATATGCTGTAACTGATGTAGCGCT | | |
| | GGTTTACCTCCTTA | | |
| MC406b | TAAGGGGTTTTGAGTTTTGTTTACTACTACCACTGC | Nicotiana tabacum | SEQ ID N° 346 |
| | TCTCAGAAAAAATGGATTTGATAGTCTAGTTTTTTA | RENT3 repetitive | |
| | CACAAACTCTTTTCAAACTATGTCAAGCACTCTCAC | sequence | |
| | ATATACTCTTTAGAATACTAGGTTCTGCCCCTCTTGT | | |
| | GTGAGCTTTGCCTTGGGACCCTTGAGCTCTCTCTGA | | |
| | ACTTGGACACATAAGAGCTGGTCCTTCCATACTAC | | |
| | ACTTACTCTTGGTTATGCAATCTGGGTGTGAGCACT | | |
| | ACCTAGGATC | | |
| MC406c | TAAGGGAGCT GTTCCAGTTC CAGAGTCAGT | 60S ribosomal | SEQ ID N° 347 |
| | GCTGAAGAAG CAAAAGAGGA GTGAGGAATG | protein L7 | |
| | GGCCCTTGCA AAGAAACAAG AGCTTGAAGG | | |
| | TGCAAAGAAG AAGAGTTCCG AGAACCGGAA | | |
| | ATTGATCTAC AACAGAGCTA AGCAGTATGC | | |
| | TAAGGAATAT GAGCAGCAGG ATAAGGAGTT | | |
| | GATTTGCTTG AAGCGCGAGG GTAGATTGAA | | |
| | GGGTGGTTTC TATGTTGACC CTGAGGCAAA | | |
| | GTTGTTGTTC ATCATTAGGA TC | | |
| MC407 | TAAGGCAGAG ATGTTCTTTG ATAGAGGAGA | putative | SEQ ID N° 348 |
| | ATTGCTTGGA GGCCTTGTGA AAGGAGAAAG | pathogenesis related | |
| | CAATGGTGAA TTGGCATTGG CTGCTTCAAA | protein | |
| | ATGTCCTTTC ATGAAATAAG AGCAAAACCA | | |
| | GCAACTGCTG CTTATTTTCA AGACAAGATC | | |
| | TCAAGAAAG | | |
| MC408 | TAAGCAGGGG AGGAAGTACT GCAAAATTGG | cytosolic pyruvate | SEQ ID N° 349 |
| | TGGCCAAGTA CAGACCTGGA ATGCCTATAT | kinase | |
| | TGTCGGTGGT TGTCCCCGAG ATCAAAACTG | | |
| | ATTCTTTTGA TTGGACTTGC AGTGACGAGT | | |
| | CTCCAGCAAG GCATAGCCTT ATATTCAGGG GAT | | |
| MC409 | TGTATAACCTTTTTGATGTCTCAATTCTTATGCTCTT | putative protein | SEQ ID N° 350 |
| | ATGAATAATACATAACAATTGCCACGAAATTTTCT | At1g80220 [A. | |
| | GAAAGAATAGGTGGCTTA | thaliana] | |
| MC410 | TAATGTTTGG CTACTCTTCT GTACAGCTTC | putative protein | SEQ ID N° 351 |
| | CAACATTGGA CAAGGATAAC CTCCGCGGTG | At4g28910 [A. | |
| | TGGCtTCTCA TCTTCAACAG CTTCACCCTT | thaliana] | |
| | CCCATGGAAG AGGTCCTCTG GGTTCAGATA | | |
| | TGCAGAAAGA TGGACCAAAT ATTTCTCAAG | | |
| | CTACTACGTC ATCTATTCCG CACAAGTCAT | | |
| | CTGATTCTGT ACAATATGAT GGGAGGGCAA | | |
| | TGGAGCATGT GAAAGGCAAT GGGAGACAGC | | |
| | ATAAGGCAGA AGAAACTTCC AATTCTCGAG | | |
| | GGGAGGAAAA TGTGAAAGGA AGCAACATAA | | |
| | GCTTCAGGGC AAAAGACCCT CCTGACCAGC | | |
| | CCAGAGCAGA AGCAGTTCCT TCTAATTTTC | | |
| | AACTATTAGG CCAGGTCTTG CTGCAGAT | | |
| MC412 | TAATCGCATT GAAGCACGGA GTGAGCAGTT | RNA polymerase I, | SEQ ID N° 352 |
| | TGACATGTAC ATGCTGTTGG ATGTGAACAC | II and III 16.5 kDa | |
| | TGAGATATAT CCTATGCGCG TCAAAGAGAA | subunit | |
| | ATTTATGATG GTTTTAGCAT CTACTTTGAA | | |
| | CTTGGATGGG ACACCAGATA CTGGTTATTT | | |
| | CATTCAGGGT AACAAGAAAT CACTTGCTGA | | |
| | CAAGTTCGAA TATGTC | | |
| MC413 | TACCTGTGGTTGGATCGGTATAGTCGCCACGGTCAC | putative esterase | SEQ ID N° 353 |
| | TCGCTTGACCTACTGTCACTGGGCTACCTAAAGTCA | | |
| | ACACCACGTTATTACCCACTACCGGAACACCGGTT | | |
| | ACAGTCACCAATTGACCACCAGCAGTCACTGTAAA | | |
| | GCTACCTGTTGTTGGCAAGTGCAGTGGATTA | | |
| MC414 | TAACGAGTAT GGCGAAGACT ATGAAGCCCA | putative calcium | SEQ ID N° 354 |
| | TGATGAGTTT CGTTAGTTCA AGGCTAGGAT | binding protein | |
| | GGGTCATGCT ATCAAG | | |
| MT101 | ATGAGAGTTC GAATCCACCA AACAATGGCG | GTP-binding-like | SEQ ID N° 355 |
| | ACCGTTATGC AGAAAATCAA AGATATCGAA | protein | |
| | GATGAGATGG CTAAGACCCA AAAGAACAAA | | |
| | GCTACTGCTC ATCATCTCGG TTTGTTAAAG | | |
| | GCAAAACTGG CAAAACTTCG AAGGGAGCTT | | |
| | CTTACACCTA CATCAAAAGG TGGTGGTGGA | | |
| | GCTGGAGAAG GTTTTGATGT TACAAAAAGC | | |
| | GGTGATGCAA GAGTGGGTTT AGTGGGCTTT | | |
| | CCTTCAGTTG GAAAGTCGAC ACTCTTGAAC | | |
| | AAATTGACTG GAACTTTTTC TGAGGTTGCT | | |
| | TCATATGAAT TTACCACCTT AACGTGCATT | | |
| | CCTGGTGTCA TCATGTATCG AGGAGCTAAA | | |
| | ATCCAGTTGT TGGATCTCCC AGGAATTATT | | |
| | GAGGGTGCCA AGGATGGAAA AGGTAGAGGA | | |
| | AGGCAGGTTA TCAGTACTGC AAGGACTTGC | | |
| | AATTGTATAC TTATTGTTCT TGATGCAATA | | |
| | AAACCAATTA CTCACAAACG TCCCATCGAG | | |
| | AAAGAGCTTG AGGGATTTGG CATCAGGTTG | | |
| | AACAAGGAAC CACCTAATCT GACATTCAGG | | |
| | AGGAAAGAGA AGGGTGGGAT CAATTTAACA | | |
| | TCAACAGTGA CCAATACTCA TTTAGACCTC | | |
| | GACACCGTAA AGGCCATATG CAGCGAATAC | | |
| | AGAATACATA ATGCTGATGT TCATCTTAGG | | |
| | TATGATGCAA CTGCTGATGA CCTTATTGAT | | |
| | GTCATTGAAG GCAGTAGAGT ATACACACCT | | |
| | TGCATCTATG TTGTGAACAA AATTGATCAA | | |
| | ATCCCAATGG AAGAGCTGGA GATTCTGGAT | | |
| | AAACTTCCCC ATTATTGTCC GATCAGTGCT | | |
| | CATTTGGAAT GGAATCTTGA TGGCTTGCTG | | |
| | GAGAAGATTT GGGAATATCT CAGTCTAACC | | |
| | CGTATATACA CTAAGCCGAA GGGAATGAAT | | |
| | CCAGACTATG AGGATCCAGT AATTCTATCA | | |
| | TCAAAGAGGA GGACAGTGGA GGACTTCTGC | | |
| | GACAGAATCC ACAAGGATAT GGTTAAACAA | | |
| | TTCAAATATG CGCTGGTTTG GGGTTCAAGT | | |
| | GCAAAACACA AACCTCAGAG GGTGGGCAGG | | |
| | GAACATGAAC TAGAAGATGA AGACGTCGTC | | |
| | CAAATCATCA AGAAGGTGTG A | | |
| MT102 | TAAAAGGGAG AGAGCAGAAC GTGAGGCTTT | ubiquinol-- | SEQ ID N° 356 |
| | GGGAGCTTTG CCTCTCTATC AGCGGACAAT | cytochrome-c | |
| | TCCATGAAGA AATCAAATCT CCCTTGAAGC | reductase-like | |
| | TTTTTCGATT GAGAATAATT ACTGTGTTGC | protein | |
| | TTGTAGATGA GCTTTGCCTC TGTATCAGTC | | |
| | GTACAATTCC ATGAAGAAAT CGAATCTCCC | | |
| | TATAAGTTTT TC | | |
| MT103 | TAGCAACTTTGACAGGTGTCAATGTCGGTGACAAT | pathogenesis related | SEQ ID N° 357 |
| | GCAACAGCACAACGAGGTGATTATGCCTTCAGTTT | like-protein | |
| | CACAGTAAATTGATCGATATTGGGCTATCGATCAA | | |
| | TATGCCTTCAGTTTCAGAGTAAATTGATCGATATTG | | |
| | GGCTATCTTTGTTTCTGAAGCTGCATTGTTGAATCT | | |
| | TTTCATCGGATATCCTTCTTGTTGTTCATTCTGTAGC | | |
| | CTAGCTAATTGTGGACTTTCTATTATCGTGTCTTTTT | | |
| | CGTAATATTGCAAGATC | | |
| MT104a | ACAACAATCGAAATTGATTCTCTGTATGAGGGCCTT | heat shock protein | SEQ ID N° 358 |
| | GACTTTTATCCTACCATTACTCGTGCTAGATTCGAG | 70 | |
| | GAGTTGAACATGGATC | | |
| MT106a | GATCTAGTGG CCGGTGAATC ACTGATCAAA | ribosomal protein | SEQ ID N° 359 |
| | GAGCAGATTT TAGAGAGATT CTTCATCGAT | | |
| | CTAGTGGCCG GTGAATCACT GATCAAAGAG | | |
| | CGAGCAGCCG GCAGGTTTAG CCAGAACTCG | | |
| | TCGATCAC | | |
| MT106b | GATGAGTCCTGACTAAACTAATCGATTTGGGTGGC | putative ubiquinone | SEQ ID N° 360 |
| | AATGATAGAAGGTAGTCGTCTTCGGTTGAAAGGGT | biosynthesis protein | |
| | GGCAGCAGGCTGCTGTTGCAGTTGGTTCTGCATTTG | | |
| | GGGCGTTGCTAGATCA | | |
| MT108 | TAACCACAGA TTTCTCAAGC TGAATCATCA | water channel | SEQ ID N° 361 |
| | TGTAGCAAAG ATCAAAA | protein | |
| MT109 | TAACGTGCTC GGAGAACCTG T | ATP synthase beta | SEQ ID N° 362 |
| | | subunit | |
| MT110 | TAAGGAGTTG TCACTGGAGC AGGAATCGTT | putative protein | SEQ ID N° 363 |
| | CATCGTAAAG AGTGACCCCA AAAGCTCAGG | At1g79140 [A. | |
| | TACCAAGAGA AAAAAAGGGA GTGCCTCATT | thaliana] | |
| | AGAGCATATT AGTACGGGGT CTGACCTTGA | | |
| | TTTCACTGCT CAAATTGATG AAAATGATGT | | |
| | TAGAAAGAAA CTCTCTGAGC ATTACTTGCT | | |
| | GCTTCATGAC ATAGCTGAAA ATGAAAGAGT | | |
| | AAGAGGGGAA TTGGCTCGGA CAACATTGTC | | |
| | TCTGAAGCTG CACGAACAAT ATAAAAAGCA | | |
| | GAAGAAAAGA AGAACATAGT AGGCATCTG | | |
| MT111 | TAAGAGCTGT GGAAAAGGTC TGTTGGAATC | putative annexin | SEQ ID N° 364 |
| | TATTCTGAAG GTGGTTATCT GGTGCATTGA | | |
| | TTCACCAGAG AAACATTTTG CTGAGGTTGT | | |
| | CAGAGCCTCG ATTGTCGGGA TAGGAACTGA | | |
| | TGAGGATTCT CTAACAAGAG CCATTGTAGC | | |
| | TCGAGCTGAA GTTGATATGA TGAAAGTAAG | | |
| | GGGAGAGTAT TTCATCGCGA ACAAGACCAG | | |
| | TCTTGATAAT GCAGTTATTG GTGATACATC | | |
| | AGGTGATTAC AGGAAGTTCC TGATGACACT | | |
| MT112 | TAAGGGCTTC ACAAATGTGA ATCTCAAAAC | glucose-1- | SEQ ID N° 365 |
| | TACGTGTATC CTGGCATTGC AGAAAAAAGC | phosphate | |
| | AGCTATGCTA GCAGGTTTTT TAGCGCCTCA | cytidylyltransferase | |
| | AGCATGAGCA ATATGAATTG TTCCAGTTCT | like protein | |
| | ATGGCATGTC ATGTTATTAT ATCTTCACGC | | |
| | CGATGACAAA ATAATTGAAT GCAGGAAGAA | | |
| | GCTCCTGGTG CTGCCAGAGT ACAAGTTTAC | | |
| | GACTATTTCA A | | |
| MT113a | GATCTACAGTGTTTTTCAGGCTTCAAATTCATCAAC | putative DNA | SEQ ID N° 366 |
| | ATCTCACAAAGGAGCTGTTGCTGTTAGGCAGCCTT | replication licensing | |
| | ATATTAGAGTTGTTGGAATGGAAGAAACGAATGAG | factor | |
| | GCCAATTCTCGAGGGTCAGCCAACTTCACAGTAGA | | |
| | TGAGAAAGAAGAATTTCAGAAATTTGCATCCGATA | | |
| | AGGATGCTTATGAAAAGATATGCTCAAAGATTGCT | | |
| | CCCTCAATATTTGGGCATGTTGATGTAAAGAAAGC | | |
| | TGTAGCATGCCTTTTATTTGGAGGGTCAAGGAAGTT | | |
| | CTTGCCCGATGGTGTAAGATTA | | |
| MT113b | GATCTACCAA CCTGAAAATC TGACGCATCC | putative protein | SEQ ID N° 367 |
| | CCCCATGCTG CCCATTCAGG CATAGGCCTG | At1g07990 [A. | |
| | TCTCCAAAGG GGTCATCATT GTCTGACGTC | thaliana] | |
| | TCAAAGCGGG AGAAACCCAT GTCATCTGAC | | |
| | ACACCTGCTT TCTCGCTTTG TTGACTGAAG | | |
| | TTACCACCAT TGACAGAATC CAATCCACTA | | |
| | ACAGCATTTG AAGTAGAAGT GGGTGTTGCA | | |
| | TCTGTAGAAT TTTTGCTCTC AGCCAATTCG | | |
| | TCCTCCTCTC CTACTACTAC CTCGTCATCA | | |
| | CTATTGCTAT TCCCATCATT TGATGTTCCA TTA | | |
| MT113c | GATCTATCAGCAAAAGGAAATCTCTTGTGTATGTTT | putative protein | SEQ ID N° 368 |
| | ATACTTATAAGATTCAAGATGCTGATCTATTGCAAG | AT3g10420 [A. | |
| | TTGCAACTGTTATGGGGCTTGACGAAGAAGTTGAA | thaliana] | |
| | GTAACAGATGATATTGGTATTGCGGATGCTATTCTA | | |
| | GCATCTAGTGCTGAAATGAAGCAGAATCCTTGGAT | | |
| | TCGTAGTGTTGCCAAATCTCATCAAGTTTCTGTCTT | | |
| | TGTTGTAAAGTCAAGTACCATGGCCCAAATGGTGA | | |
| | AAGCTATCCGTATGATTCTTGGAATGGATTCCATTC | | |
| | ACTCAAAACAGCCATTA | | |
| MT114 | TAATAACAACGAGAGCAGTCACATCATTCATGTTC | 26S proteasome | SEQ ID N° 369 |
| | CTGCTGGTCCTAATGCTCTCTCTGATGTGCTTATAA | regulatory subunit | |
| | GTACTCCTATTTTCACTGGTGATGGTGAGGGTGGAA | S5A | |
| | GTGGATTTGCAGCAGCAGCTGCAGCGGCTGCCGCT | | |
| | GGTGGAGTGTCTGGGTTTGACTTTGGTGTAGATC | | |
| MT115b | GGGATGGAGAGAAATTTTCTCAAGTATGTGTACTGG | fatty acid | SEQ ID N° 370 |
| | TCAAATGGTAAGGAGACAGATGATCCAACTGCGAA | hydroperoxide lyase | |
| | TGATAAACAGTGTCCTGGTAAAGATC | | |
| MT202 | ACCCCGGCTCGAACAGGAGGAGTACGCCATGCTAA | putative protein | SEQ ID N° 371 |
| | TGTGCCTTGGATGATCCACATATAAAGGTCAGGCG | rps12 [Oenothera | |
| | CCGATGAGCACATTGAACTATCCATGTGGCTGAGA | elata subsp. | |
| | GCCCTCACAGCCCAGGCACAACGACGCAATTATCA | hookeri] | |
| | GGGGCGCGCTCTACCACTGAGCTAATAGCCCGACG | | |
| | TGCGAGCCTCCCACTGGGGGCCCGCTATGCCAAAA | | |
| | GCGAGAGAAACCCCATCCCTCTCTTTCCTTTTTTCG | | |
| | CCCCCATGTCGCCACACGGGGGGAACATGGGGACG | | |
| | TAAAAAAGGGGGGCCTATCAACTTGTTCCGACCTA | | |
| | GGATAATAAGCTCATGAGCTTGGTCTTACTTCACCG | | |
| | GCGAGAAAGGAAAGAAGACTTCCATCTCCAA | | |
| MT203a | GATCTCCATCCAGTAATTGACCTCAAAATGTAAGC | maturase-like | SEQ ID N° 372 |
| | CCAACAAAAAAAAAAAAAAAAACCTTGCCCCTCAT | protein | |
| | TAACCCTCCAAATTGGGGAAATAACGGGGGGCGGG | | |
| | ATTTTCCTCACAGTGGTCACTTGAAAATCCAAAAA | | |
| | ATGGCCGATCGGGTGTACCTAAAAGGGGGATAATG | | |
| | TCGGCCTACCAGGCATGTGTTGGCTAAGTTCCCTTT | | |
| | TCACATGAAATCCCATTCTTCATACCCTTCTTTTGCT | | |
| | TTTCCCACAGTTTCATAATTGGCCTTATAAACATGT | | |
| | TTTTGTTTTTTTTTTGCCCCGGCTTTTTTTTAACTGG | | |
| | CATGGGCTTCCTTTTT | | |
| MT203b | TAAACGAAGA TGAGAAGAAA CTGTAACTTG | putative protein | SEQ ID N° 373 |
| | GAGCTCACGC TTATGCCTCC TTCTTTTTCT | At2g34600 [A. | |
| | TTTTCTCCTA AGAATTGCAC TACCCCTTAC | thaliana] | |
| | TTCTCAACGG ATAGGGAGGA TAAAGAAAGC | | |
| | ACAGAAGAGA AACAACCACA GCAGCTAACA | | |
| | ATATTTTACA ATGGAAAATT TGTGGTTTCT | | |
| | GATGCTACTG AACTTCAGGC TAAAGCAATA | | |
| | ATATATCTGG CAAGTAGAGA AATGGAGGAG | | |
| | AAAACAAAAA TCCGGTCACC AATTTCAGAA | | |
| | TCATCATCAC CAATTTCAGA GCCTTTCATCA | | |
| | CCATTTTTAC AATCTCCAGC TTCTGATCTTT | | |
| | TCTATGAAGA GATC | | |
| MT204 | TAAAGTACTA ATTCCTATTT ACAATGCTCA | protein kinase-like | SEQ ID N° 374 |
| | CTGCAGTATT TCTGAGCAGG CTCTTTTCTA | protein | |
| | ATTTAGTATC AGCTGAGTTT TTGCTTATGT | | |
| | TTACTTTTTA CTCAGGCAAG GTTCTTCTTT | | |
| | CAACAATTGA TATCAGGGGT TAGCTACTGC | | |
| | CATTTC | | |
| MT205 | TAAACTCGGC ACCTCCACCA ACTCCAAGTC | allene oxide cylase | SEQ ID N° 375 |
| | ATTTTACTGC AAGAGCCAGA GCGGCTCAAC | | |
| | TGATTCCTAA ACAACTAAAG TTCAAGAGGT | | |
| | AAGTG | | |
| MT207 | TAACGTGACG GATTCGCAGC TGTACGATCT | poly(A)-binding | SEQ ID N° 376 |
| | GTTCAACCAA GTCGGTCAGG TTGTTTCGGT | protein | |
| | TAGGGTTT | | |
| MT208 | TAAGCACATA ACCTACCTTA TTGAGCAGAA | 60S ribosomal | SEQ ID N° 377 |
| | CAAAGCACAG TTGGTGGTTA TTGCTCATGA | protein L7A | |
| | TGTGGACCCA ATAGAGTTAG TCGTGTGGCT | | |
| | GCCAGCATTG TGCAGAAAGA TGGAAATTCC | | |
| | GTACTGCATC GTGAAGGGAA AAGCACGTTT | | |
| | AGGATCGATC GTGCACAAGA AAACTGCTTC | | |
| | GGCTCTATGC TTGACAACTG TGAAAAATGA | | |
| | AGATAAAATG GAGTTCAGCA GAATTTTGGA | | |
| | GGCAATCAAG GCAAACTTCA ATGACAAGTA | | |
| | TGAGGAAAAC AGAAAGAAAT GGGGCGGTGG | | |
| | TGTCATGGGA TCCAAATCAC AAGCCAGAAC | | |
| | CAAGGCGAAA GAGAGGGTTC TCGCCAAGGA | | |
| | AGGAGGACAG AGAATGAACT AGAGCTTCTA | | |
| | TTTTATGTTG CTGTTTGGGT TAGACCTACA | | |
| | AATTTTGTGT TTTTGATTCG C | | |
| MT209b | TAAGGTTCGA TGACGCTAGG ATTATAAGGA | Tyl-copia-like | SEQ ID N° 378 |
| | AGATTTGTAT GTTATTACCG AATGTTGTTC | retrotransposon | |
| | CGAGTCCCGG ATGAGATC | | |
| MT210a | GATCTCGTCGCCTTCCACGTCTATTCCTTCAGCTGT | putative protein | SEQ ID N° 379 |
| | TTCCTCTTTTCTAGCCTCATTGCTTTGTGCCTTA | AT5g05950 [A. | |
| | | thaliana] | |
| MT211 | TAATCGTGGA ACAGGTCAGA TTATTCCAAC | translation | SEQ ID N° 380 |
| | TGCACGACGT GTAGCCTACT CTTCTTTCCT | Elongation Factor | |
| | TATGGCGACA CCCAGGCTTA TGGAACCTGT | 2-like protein | |
| | GTATTATGTG GAGATCCAAA CACCCATGGA | | |
| | TTGTCTCTCT GCTATATACA CCGTGTTGTC | | |
| | TCGCAGGCGT GGACATGTTA CTGCTGATGT | | |
| | TCCTCAACCT GGGACACCTG CCTACATCGT | | |
| | CAAGGCATTT TTACCTGTGA TCGAGTCCTT | | |
| | TGGTTTCGAA ACCGACTTGA GGTATCACAC | | |
| | CCAAGGGCAG GCGTTTTGTC TTTCAGTGT | | |
| MT212 | TAATCAGACT AGTGTCCGGG ACCAGGTCCT | lipase-like protein | SEQ ID N° 381 |
| | TGAAGAGGTA AAAAGATTGG TTGAGGAATA | | |
| | TAAGAATGAA GAGGTGAGCA TAACAGTAAC | | |
| | CGGCCATAGC CTAGGTGCAT CACTTGCAAC | | |
| | CCTAAATGCA GTTGACATAG CTTTCAATGG | | |
| | AGTCAACAAA ACAAGCGAAG GCAAGGAATT | | |
| | TCAAGTGACA GCTTTTGCAT TCGCAAGT | | |
| MT214a | ACAACTGTGT GGATTGTTTT AGCCCAACCC | putative | SEQ ID N° 382 |
| | TGTTAT | phytosulfokine | |
| | | peptide precursor | |
| MT301b | TAAAGTCCCTGTCAGATATCTGAAGGAAGATAAAC | putative GDP- | SEQ ID N° 383 |
| | CTCACGGGTCTGCTGGTGGCCTTTATTATTTCAGAA | mannose | |
| | ATTTGATCATGGAGGAACTTCCGTCTCACATTTTTC | pyrophosphorylase | |
| | TGCTAAACTGCGACGTGTGCTGCAATTTTCCACTGC | | |
| | CAGAGATGCTTGTTGCCCATAGAAGATATGGTGGA | | |
| | ATGGGTACATTGCTAGTTATCAAGGTTTCGGCTGAA | | |
| | TCAGCCAACCAGTTTGGAGAGTTGGTTGCAGATC | | |
| MT301c | TAAAAACAGG TGCAAGCATC CCATAGTGAT | putative protein | SEQ ID N° 384 |
| | TGTAGTTGAG ATGGACCGCA TATTGCGGCC | At1g19430 [A. | |
| | TGGTGGTTGG GCAATTATAC GTGACAAGGT | thaliana] | |
| | CGAAATACTT GATCCGCTAG AGAGTATACT | | |
| | GAGAAGCTTG CATTGGGAGA TACGAATGAC | | |
| | ATTCGCAAAA GATAAGGAAG GCATCCTTTG | | |
| | TGCACAAAAG ACCATGTGGA GACCTTGATG | | |
| | AATGGAGCAA ATCTTTCGCT TTCCATTTTC | | |
| | CAGATC | | |
| MT302a | GATCTAATAC CAGTATTCAG TTGTGGAAGT | calmodulin-like | SEQ ID N° 385 |
| | AATCTCTTCG AGATTC | protein | |
| MT302b | ATGGTAAGGAGACAGATGATCCAACTGCGAATGAT | divinyl ether | SEQ ID N° 386 |
| | AAACAGTGTCCTGGTAAAGATC | synthase like | |
| | | protein | |
| MT303 | CTTATTATGCTTTTGCTCGTTTA | | SEQ ID N° 387 |
| MT305a | GATCTGGTAA TTTTGGAAGG GATGGGCCGA | putative protein | SEQ ID N° 388 |
| | TCTTTGCATA CCAACTATAA TGCAAAGTTC | C42D8.3 | |
| | AAATGTGATG CTCTAAAGCT TGCTATGGTG | [Caenorhabditis | |
| | AAGAATCAGC GGTTGGCACA AAAGTTGGTT A | elegans] | |
| MT305b | GATCTGTACA TGTCATCGAC ATTACTAAGA | putative protein | SEQ ID N° 389 |
| | GTTGCTGGTG AACACAACTC TGTTGTAGCA | At2g32340 [A. | |
| | GTTGTTGGGA AGGGTCACCT GCGTGGAATC | thaliana] | |
| | AAGAAGAACT GGAAACAACA CATTGAGGTT A | | |
| MT306a | TGCGTCTGGCTATGGAAGTTTTGGACCATCTTCTTG | polygalacturonase | SEQ ID N° 390 |
| | GTCAAACATTTTCGGGTTGTGGCAAACATTCTTCAG | inhibiting like | |
| | TCACCTGAGAAATCTAGTGGTTTTTGAAATGATAGT | protein | |
| | TAACCTTGTAGTTACTCAAGACTCA | | |
| MT307a | TAACTGAACT TGGATTTTCG CAAGACGGTT | hypothetical protein- | SEQ ID N° 391 |
| | ATCAGTTATT TTGTGATAGT CAAAGTGCTA | common tobacco | |
| | TCCACCTTGC GAAGAACGCC TCATTCGATT | retrotransposon | |
| | CCAGATC | Ttol | |
| MT307a | TAACCACACCCCAAATAGACCCGTCATTCTTCAAC | ambiguous hit | SEQ ID N° 392 |
| | CAGCACCCCACCCCCGAGTCATCTCCTTCGTCGAAC | | |
| | CTCCAGCGACCACTCCCTGAGCCAGATC | | |
| MT308a | TAAACAGAAG ATAGCTGATG AAATACTAGC | putative protein | SEQ ID N° 393 |
| | AACTTTGAGA GGTGTCAATG TCGGTGACAA | AT4g09810 [A. | |
| | TGCAACAGCA CAACGAGGTG ATTATGCCTT | thaliana] | |
| | CAGTTTCAGA GTAAATTGAT CGATATTGGG | | |
| | CTATCGATCA ATATGCCTTC AGTTTCAGAG TA | | |
| MT308b | TAACCGGGATGCATTTTGCCACAACAACCTTGATG | putative protein | SEQ ID N° 394 |
| | ACTATTGTTCTTAGGTGGCTCGGATACATCCAAGCT | At4g33380 [A. | |
| | TCTCATTTACCCCTTCCAGATC | thaliana] | |
| MT309 | TAAGGCACCA TCAGTTTTTG ATATCAAGAA | 40S ribosomal | SEQ ID N° 395 |
| | TGTTGGCAAA ACCCTCGTTA CTAGGACTCA | protein S3a | |
| | GGGTACCAAG ATTGCTTCAG AGGGCCTAAA | | |
| | GCATAGAGTA TTTGAAGTGA GTCTGGCTGA | | |
| | TCTTCAAAAG GATGAGGATC AGGCTTTCAG | | |
| | GAAGATCAGG TTGAGAGCTG AGGATGTGCA | | |
| | AGGAAAGAAT GTCCTCACAA ACTTCTGGGG | | |
| | GATGGATTTC ACAACAGACA AGTTGAGGTC | | |
| | ACTGGTTCGC AAATGGCAGA CTTTGATTGA | | |
| | GGCCCATGTA GATGTCAAAA CTACAGACAG | | |
| | CTATACCTTG AGGATGTTCT GCATTGCTTT | | |
| | TACAAAGAAG CGTCCAAACC AGCAGAAGCG | | |
| | TACGTGTTAT GCTCAGAGCA GCCAGATCCG | | |
| | TCAGATC | | |
| MT311a | AGAACCTAACAATCTTTACAACCTTCACTCTTACAA | putative ribosomal | SEQ ID N° 396 |
| | ACACTCTGGGCTAGCAAACAAGAAAACTGCGACTA | protein L28 | |
| | TCCAGGCTGAGGGGAAAGATAACTCTGTGGTGCTT | | |
| | GCCACATCGAAGACCAAGAAGCAAAACAAGCCTTC | | |
| | AACTTTGCTGAACAAATCTGTGATGAAGAACGAAT | | |
| | TCCCCAGAATGACCAAGGGTGTAACCAACCAGGGT | | |
| | GCAGACAACTACTACAGGCCAGATC | | |
| MT311b | TAAGGATAGG ATTGGTTATA GTATGATTAC | cytosolic cysteine | SEQ ID N° 397 |
| | GGATGCTGAG GAGAAAGGCC TGATCAAACC | synthase | |
| | TGGCGAGAGT GTCCTCATTG AACCTACAAG | | |
| | TGGAAACACT GGAGTAGGAT TGGCATTTAT | | |
| | GGCTGCTGCT AAAGGCTACA AACTCATCAT | | |
| | AACGATGCCT TCTTCAATGA GTCTTGAGAG | | |
| | GAGAATTATT CTGCGTGCTC CTGGTGCTGA | | |
| | GTTGGTGCTT ACAGATC | | |
| MT401 | CTGAGTAAAG GGAATCAAAT ATGAAGCAAA | probable glutathione | SEQ ID N° 398 |
| | GGAGGAAAAC TTATCTGATA AAAGCCCTTT | S-transferase PARA | |
| | GCTTCTGGAG ATGAACCCTG TTCACAAAAA | | |
| | GATCCCTATT TTGATTCACA ATAGTAAAGC | | |
| | CATTTGTGAG TCTCTAAACA TTCTTGAGTA | | |
| | CATTGATGAA GTCTGGCATG ACAAATGTCC | | |
| | ATTACTTCCT TCTGATCCTT ACGAAAGGTC | | |
| | ACAAGCCAGA TTCTGGGCCG ACTATATTGA | | |
| | CAAGAAGATA TATAGGACAG GAAGAAGAGT | | |
| | GTGGAGCGGT AAAGGTGAAG ATCAAGAAGA | | |
| | AGCAAAGAAG GAATTCATAG AAATACTCAA | | |
| | GACTTTGGAA GGAGAGCTTG GAAATAAAAC | | |
| | TTACTTTGGT GGTGATAATC TGGGTTTTGT | | |
| | GGATGTGGCT TTGGTTCCCT TTACTAGTTG | | |
| | GTTTTATTCT TATGAGACTT GTGCAAGCTT | | |
| | TAGTATAGAA GCAGAGTGTC CAAAGCTGGT | | |
| | GGTATGGGCA ACAACATGTA GGAGAGCGAG | | |
| | AGTG | | |
| MT402b | CAAAATCCAGCCCCATAACTCCACCACGTATTCGA | myocyte enhancer | SEQ ID N° 399 |
| | GTCTTGCCGACGAGCTTTCCGTTAGTAGATC | factor 2A like | |
| | | protein | |
| MT402c | TGTACACTGGTCAGTTTATTTACTGCGGTAAAAAA | 60S ribosomal | SEQ ID N° 400 |
| | GCTAATCTAATGGTGGGTAATGTGTTGGCACTTAG | protein L2 | |
| | ATC | | |
| MT403a | TAAACAGAAGATAGCTGATGAAATACTAGCAACTT | putative protein | SEQ ID N° 401 |
| | TGAGAGGTGTCAATGTCGGTGACAATGCAACAGCA | AT4g33380 [A. | |
| | CAACGAGGTGATTATGCCTTCAGTTTCAGAGTAAA | thaliana] | |
| | TTGATCGATATTGGGCTATCGATCAATATGCCTTCA | | |
| | GTTTCAGAGTAAATTGATCGATATTGGGCTATCTTT | | |
| | GTTTCTGAAGCTGCATTGTTGAATCTTTTCATCGGA | | |
| | TATCCTTCTTGTTGTTCATTCTGTAGCCTAGCTAATT | | |
| | GTGGACTTTCTATTATCGTGTCTTTTTCGTAATATTG | | |
| | CAAGATC | | |
| MT403b | GATCTTGGAG ATGGCTTCAT GCAGCGAAGA | GPAA1-like | SEQ ID N° 402 |
| | CCGTGTTTAT TGTCCACTTT TGGGGTGCCG | protein | |
| | TTGTAACATT GCTTCCGCAC TTTCTGTCTC | | |
| | TAGTACCAGA TTCCGCACCT CTGACCAACC | | |
| | TCATAACCTG GATCATGCTT TCAGCGTCCA | | |
| | GTCTCTTGAT CTGACAAGTG ATTCTGGGTT | | |
| | CCTCCTTGAG TCTTCCATCC ATGACCCATA | | |
| | CTCGAGGAAT GGAATGGACT CTTTTGAAAT | | |
| | CAGTGACAAT TGCTGGTGCC TGTACTGGAC | | |
| | TTTGCATAAT GTCAGTC | | |
| MT407 | TGAGTCTTGA GTAATGCATA TATATAGCAC | probable glutathione | SEQ ID N° 403 |
| | AGGAAGAAGA GTGTGGAGCG GTAAAGGTGA | S-transferase PARA | |
| | AGATCAAGAA GAAGCAAAGA AGGAATTCAT | | |
| | AGAAATACTC AAGACTTTGG AAGGAGAGCT | | |
| | TGGAAATAAA ACTTACTTTG GTGGTGATAA | | |
| | TCTGGGTTTT GTGGATGTGG CTTTGGTTCC | | |
| | CTTTACTAGT TGGTTTTATT CTTATGAGAC | | |
| | TTGTGCAAAC TTTAGTATAG AAGCAGAGTG | | |
| | TCCAAAGCTG GTGGTATGGG CAAAAACATG | | |
| | TATGGAGAGC GAGAGTGTCT CAAAGTCCCT | | |
| | TCCTCATCCT CACAAGATC | | |
| MT409 | GGATGGAGAGAAATTTCTCAAGTATGTGTACTGGT | allene oxide | SEQ ID N° 404 |
| | CAAATGGTAAGGAGACAGATGATCCAACTGTGAAT | synthase | |
| | GATAAACAGTGTGCTGGTAAAGATC | | |
| MT410a | CTGTGTTTTA TATGTTCTTT GAGCAATATC | putative protein | SEQ ID N° 405 |
| | TGCAGCATAT GGAGGACAGC CCTAATTA | At1g42470 [A. | |
| | | thaliana] | |
| MT410c | TAATCTGGAT GCAATTGAAG CCCTTGCCAC | putative NADH- | SEQ ID N° 406 |
| | GGACAACATT GTGTCAAAAG ATGCTTTGAC | ubiquinone | |
| | TTTTGAAGAT CACTTCGCAG T | oxireductase | |
| T1 | GATCTCAGAAGTTAGGACATACGTTCCTAACGTTGT | lipase-like protein | SEQ ID N° 407 |
| | CGCTGGGATTATGAGAGGCATCAAAGATGTGATTC | | |
| | AGCTCGGAGCCACGCGCTTTTTGGTTCCAGGAATTT | | |
| | ACCCACTCGGGTGCTTGCCGCTTTATCTCACATCAT | | |
| | TTCCTGACAATAATACAGGCGCGTACGATCAAATG | | |
| | GGTTGCTTGAGGAACTACAACGAGTTCGCTTCGTAT | | |
| | CATAATAGATACGTGAGCAGAGCTATCGCGA | | |
| T101 | ATCCAGACAAACGACCTGAAATGGATGAGGTAGTG | kinase like protein | SEQ ID N° 408 |
| | AAATTGGTGGAAGCAATTGACACGAGCAAAGGAG | | |
| | GAGGGATGATACCCGAAGACCAAGCTGGTGGCTGT | | |
| | TTCTGCTTTGCTCCTACCAGGGGTCCATAATCTCTC | | |
| | TTTACTATATTTTTCTTTAGCCCCGTTGGATGGTTACT | | |
| | TAAGACTCAT | | |
| T103 | TAAGGATGTC AAAGGTTGTG ATGATGCTAA | cell division protein | SEQ ID N° 409 |
| | GCAAGAGCTT GAGGAGGTTG TTGAGTACCT | FtsH protease-like | |
| | CAAAAATCCT GCTAAGTTCA CTCGGCTTGG | | |
| | GGGAAAGTTG CCGAAGGGCA TTCTTTTGAC | | |
| | TGGAGCTCCT GGAACAGGAA AAACCCTCCT | | |
| | TGCCAAGGCT ATCGGTGGAG AAGCAGGGGT | | |
| | GCCTTTCTTT TATAAGGCAG GCTC | | |
| T104 | ACTGGGAAAAAACCTGATCTATTGATTCAGCTTCCT | heat shock protein | SEQ ID N° 410 |
| | AATCCACCGAGGAGTCCTGCTGCTCAAGCAGTGAA | 101 | |
| | AAAGATGAGGATTGAAGAAATAGTGGACGATGAT | | |
| | GAAATGGAATACTGCTGAGGCCGTAAAATCACTGG | | |
| | GGTAAAATGAAGAGAAGAATACTTCACTTA | | |
| T106 | TTCGGCGAGA TGTTGATCAA TTTCGTACCG | fructokinase | SEQ ID N° 411 |
| | ACGGTCTCCG GCGTTTCCCT TGCCGAGGCT | | |
| | CCGGGGTTCT TGAAGGCTCC GGGCGGTGCA | | |
| | CCGGCAAACG TCGCCATCGC AGTGACTAGG | | |
| | CTGGGGGGAA AGTCGGCGTT CGTCGGGAAA | | |
| | CTCGGCGACG ATGAGTTCGG CCACATGCTC | | |
| | GCCGGGATAC TCAAACAAAA CGGCGTCCAA | | |
| | GCCGACGGGA TCAGCTTCGA CAAGGGCGCG | | |
| | AGAACGGCGT TGGCGTTCGT GGCTCTACGC | | |
| | GCCGACGGAG AGCGTGAGTT CATGTTCTAC | | |
| | AGGAATCCCA GTGCCGATAT GCTGCTCACT | | |
| | CCCGACGA | | |
| T107 | TAAACCCAGA GACCTACCAA CTTTTTGACG | 5′-adenylylsulfate | SEQ ID N° 412 |
| | CAGTAGAGAA GCACTATGGA ATCCGCATTG | reductase | |
| | AGTACATGTT CCCTGATGCA GTTGAAGTTC | | |
| | AGGCCTTAGT AAGGAACAAG GGCCTCTTCT | | |
| | CTTTCTACGA AGATGGCCAC CAAGAGTGCT | | |
| | GCCGTATAAG GAAAGTTCGA CCTGTTGAGG | | |
| | AGAGCACTCA AAGGCTTTAC GTGCGTGGAT | | |
| | CACAG | | |
| T109 | AGCTCTCTGGGTCCCTACCGACGCTGAGGTGCGAA | small subunit | SEQ ID N° 413 |
| | AGCATGGGGAGCGAACAGGATTAGATACCCTGGTA | ribosomal RNA | |
| | CTCCATGCCGTAAACGATGAGTGTTCGCCCTTGGTC | | |
| | TACGCGGATCAGGGGCCCAGCTAACGCGTGAAACA | | |
| | CTCCGCCTGGGGAGTACGGTCGCAAGACCGAAACT | | |
| | CAAAGGAATTGACGGGGGCCTGCACAAGCGGTGG | | |
| | AGCATGTGGTTTA | | |
| T112 | GATCTTACTGATGATATTGTTTCTGAATATAGGAAC | anionic peroxidase | SEQ ID N° 414 |
| | AGTCCTCGCGCATTTGCCTCTGATTTTGCTGCTGCT | | |
| | ATGATTAGAATGGGAGATATTAGTCCCCTAACTGG | | |
| | TCAAAATGGGATCATAAGAACTGTCTGCGGCTCCC | | |
| | TAAATTGATCATTCAAAAGCTTATTACATGTATTTT | | |
| | GTATTTATTTGATTCTTTA | | |
| T113a | GATCTCATGG CGAAAGCAAG CTATGTGCTT | putative protein | SEQ ID N° 415 |
| | ATAATTGTAT TGGTGTATTC TGACATACCG | At1g57600 [A. | |
| | CGGATTGAAG TTGTTCTAAT TTTATAGGAA | thaliana] | |
| | CTATGATTTG ATTTTAGGCA TTTGTAACTG | | |
| | GAGAAAGATG AATTGTATAA ATAATAACTT | | |
| | CAGCTGGAGC TCGTATCATG TATCATTTA | | |
| T113b | GATCTATGGTTTTGTCTTGGAACTCAAGCACAAGCT | auxin-regulated | SEQ ID N° 416 |
| | TGGTCTTGCTTGAACAAGAAACACTTCTTACCTACT | glutathione-S | |
| | GCAGAAACCAATCATGTCCTTCGTCCCTAGTTGTTC | transferase | |
| | AAGCATCAATTTATCAATATTGTTGCTACTCTGTCT | | |
| | ATAAATTTTATGGTTTGGTGTAATTTAGTCTTTA | | |
| T116a | GATCTACAAGGGATTTTGGTGAGAGTACAAAAGGA | putative protein | SEQ ID N° 417 |
| | GATGCATGCATCTTTTGTGTGGCTATTTCAGCAAGT | At1g07280 [A. | |
| | ATTTTCACATACACCTACTTTA | thaliana] | |
| T116b | GATCTACAGG ATGGTTTTGG CAAATCATGG | putative protein | SEQ ID N° 418 |
| | AACTCAAAGT TATTGTCAAA GATTATCAAG | AT3g58130 | |
| | GAGGAAATTG CCAATTGTGA TATTTGATTG GTTTA | (permease-like) | |
| | | [A. thaliana] | |
| T117a | GATCTACGAA GCCTCTATTG AATGTTATAT | zinc finger-like | SEQ ID N° 419 |
| | GAACTGAAGT ATGATGTTCT TGCTTTA | protein | |
| T119 | ACATTCTGAGAATGTTGAATTGGATAAAGTGAACC | galactinol synthase | SEQ ID N° 420 |
| | TTGTACACTATCGTGCAGCGGGATCAAAGGCATGG | | |
| | AGGTACACAGGGAAAGAAGAAAATATGCAAAGGG | | |
| | AGGACATAAAATTACTGGTGAAGAAGTGGTGGGAC | | |
| | ATTTACAAGGACGAATCATTGGACTACAAGAATGC | | |
| | GGGTGCTGTTA | | |
| T12 | GATCTCATAA GTCGATTGCC AACTTTCAAA | zinc-finger like | SEQ ID N° 421 |
| | TACAGGACCG GATTCTTCTC GAAGAAAAAG | protein | |
| | AAAATGGGAG AGTGTGTTAT ATGTTATGCT | | |
| | GCATACAGAA GCGGAGATAT GTTGACCACT | | |
| | TTACCTTGTG CACACATGTT TCATTCAGAA | | |
| | TGTATAAACC GCTGGCTTA | | |
| T121 | GATCTACATT AGTAACCCTG AGATCACAGT | putative peroxidase | SEQ ID N° 422 |
| | GCCCAAAAAA TAGCAAAATC GATTCAGCTG | | |
| | TCTATTTCTC ACCAGGATAT GGTTCTAACT | | |
| | ACACATTCTC CAATACATTC TATGAAAAAG | | |
| | TTGTTGCTCA CGAATCTGTT CTTAGAGTTG | | |
| | ATCAGCAACT ATCATATGGA GCTGACACAA | | |
| | GTGAACTAGT TA | | |
| T123 | GGCACTCTCACAGAATTCTGCACCTCATCAAGCTGT | Mobl-like protein | SEQ ID N° 423 |
| | CCAACAATGTCTGCAGGGCCAAAGTCCGAGTATCG | | |
| | TTGGCCTGATGGAGTTA | | |
| T124 | TAACAGAAGC GCGACATTTT GGACACAAGA | receptor-like protein | SEQ ID N° 424 |
| | TTTGACGAAC CATATTACTT GGACAGGTTC | | |
| | CATATCCAAA TACAAGTGAA CTTCTCTATA | | |
| | CAGCT | | |
| T126 | TAAGACAAGT CTTAGTGGAT CATGCCCTAT | phenylalanine | SEQ ID N° 425 |
| | CGAATGGCGA CATGGAGAAG AATTGTAGCA | ammonia-lyase | |
| | CTGCAATTTT CCATAAAATC AGTGCAGTTT | | |
| | GAGGAAGAAT TGAAGATTGT TTTGCTTAAG | | |
| | GAAATGGAGA GTGCTAGATG TAAGTTGGAG | | |
| | AACGGCAAGC CCACAATT | | |
| T13 | GACTGCGTAGTGATCTCAGAACCAGTCATTCTGTGT | methionine S- | SEQ ID N° 426 |
| | TGCTTTGCTTGGAGGATTGTATCTGAAGATGCTTAC | methyltransferase | |
| | AGCTGGAATTAGTTTTGGATTTTCTGCCTCTAGACCA | | |
| | TCCTGCTTTA | | |
| T130 | TAAGGTTGAG TGCACAATAC CAAAGGACGA | NADH-glutamate | SEQ ID N° 427 |
| | TGGCTCGTTG GCAACTTTTT TGGATTCAGG | dehydrogenase | |
| T133 | TAATCGGGAA ATAATGGCAG ATGCTGAATA | RNA-binding | SEQ ID N° 428 |
| | CAGGTGCTTC GTCGGTGGGG GAGCATGGGC | protein | |
| | TGGCACCGAC CAAACACTTG GGGATGCTTT | | |
| | TTCTCAGTAC GGTGAAAT | | |
| T139 | CAGTCAGGGGGGCATGGCTACAATGTCCCGGAGAA | putative global | SEQ ID N° 429 |
| | GAGTATGCTTTAGAAGTGAAACTATCAGAGATGCCG | transcription | |
| | GGAAGTTTACCTGTTGCGGCTCAGGCTCCTGTATCTG | regulator | |
| | CCATGGCTTTTCAAAGAACATCATTTGAAACAGCTTA | | |
| | GAGCACAATGTCTTGTGTTTTTGGCTTTTAGGAATG | | |
| | GTTTA | | |
| T14 | GATCTCAATC AGAGAGCAAT GGCACGTTTC | glutathione S- | SEQ ID N° 430 |
| | TGGGCTAACT TTTTGGATGA AAAGTGTTTG | transferase | |
| | CCAAAGATGA AGGAACTTTG TTATGAAAGC | | |
| | AACAATGAAG TAAGGGAGAA AGCCAGGGGA | | |
| | GAACTTCATG AACTCCTTA | | |
| T141a | GATCTAGCAT GTGTCACTTA TTTGTATTTG | GTP-binding | SEQ ID N° 431 |
| | TCTCTAGACC TATGCAATTC AGCAGTTCTC | protein | |
| | CTTTTGGGGA ACAACTCTTC TAAGCGCATA | | |
| | CTATCAGTTG ATTTC | | |
| T142 | CCCATCGTCGAATTGTCCATGCTGCTGATATGACT | L-aspartate oxidase- | SEQ ID N° 432 |
| | GGCAGAGAGATTGAAAGAGCCTTATTAGAGGCAGT | like protein | |
| | GTTTA | | |
| T144 | GACACCATTGCTTTTTACAGAGTGCAGTGTCATCTG | putative | SEQ ID N° 433 |
| | CAAAATATTTCATTCGACACGTTTCAAATCAAAAC | cyclopropane-fatty- | |
| | ACCCTGACTCGAGCTCGTCGGAACATCTCTCGTCAC | acyl-phospholipid | |
| | TATGACCCGAGTAATGAACTCTTCTCGCTATTCCTA | synthase | |
| | GATCAGACAATGACATACTCATGTGCAATTTTCAA | | |
| | GAGTGAAGAGGAAGACTTGAAAGTTGCACAGGAG | | |
| | AGGAAAATTTCTCTTCTCATTGAAAAGGCAAAAGT | | |
| | TAGCAAGGAACACCACATTCTAGAGATAGGATGTG | | |
| | GTTGGGGAAGTTTGGCCGTGGAAGTTGTTA | | |
| T145 | GATCTAGTGT CGTGGTCCCT CGGAATTTCA | putative E2 | SEQ ID N° 434 |
| | GATTACTTGA GGAACTTGAA CGCGGTGAAA | ubiquitin- | |
| | AGGGTATTGG AGATGGGACC GTGAGCTATG | conjugating enzyme | |
| | GGATGGATGA TGGAGATGAT ATTTATATGC | | |
| | GTTCCTGGAC TGGCACCATT ATTGGTCCTC | | |
| | ACAATTCCGT TCATGAAGGT CGCATTTATC | | |
| | AGTTGAAGTT ATTCTGCGAC AAAGATTATC | | |
| | CAGAGAAGCC ACCAAGT | | |
| T146 | AATGGGTGCAAGTGTGGATCAAACTGCACCT | putative type II | SEQ ID N° 435 |
| | | metallothionein | |
| T147a | TAACATAAAACTAAAAACAGATAAGGTTCATATCA | putative protein | SEQ ID N° 436 |
| | CACAAGCAAGAAATCCCAAAAGGAGGGTTCACCTC | OSJNBb0072E24 | |
| | ACAAGTATAACAAACTTGAACATACAATTCCAAAC | [Oryza sativa] | |
| | ACTTGCTTTCTTTCAATCATTCTTGCCTGAAACATTT | | |
| | CCAGGAACATTCAAAACACTAGATC | | |
| T148 | CTTATTATGTGGACAATTCTGAACCACAGTGGACA | putative membrane | SEQ ID N° 437 |
| | CCTTGGTTGGTTCCAATGATTGTGGTTGCCAATGTA | protein | |
| | GCCATGTTTATTGTAATCATGTTCGTTACTCA | | |
| T149a | GATCTAGGTA CATTGAGCTA TTTCCTTCAC | ribonucleoprotein- | SEQ ID N° 438 |
| | AGCCAGATGA AGCTAGACGA GCCGAGTCAA | like protein | |
| | GGTCACGACA GTGATGCTAA TTATTTCTGG | | |
| | CGGAGCATTT TTAGGCATCA TATATTTCGT | | |
| | CCACCTCTTC TCTTGGGGAT ATTGTAGCAG TTGTT | | |
| T150 | GATCTAGGAA GAGAGAGAGA GAGGGAGCTG | serine/threonine | SEQ ID N° 439 |
| | ACCCATAACT CAGGCAGTTG ATCGGAAAAG | protein kinase | |
| | AGATGGGGTG GTCGTTCTCG GGGTTGAATG | | |
| | CTTTATGCGA CGCCGTTA | | |
| T151a | GATCTAGACA GAGAGGGCAG CCAACTTCAA | prohibitin-like | SEQ ID N° 440 |
| | CATTGCTCTA GATGATGTGT CCATAACAAG | protein | |
| | CCTGACTTTT GGAAAGGAAT TTACAGCTGC | | |
| | AATTGAAGCA AAACAAGTGG CTGCTCAAGA | | |
| | AGCTGAAAGA GCAAAGTTTG TTGTGGAAAA | | |
| | AGCTGAGCAA GATAAGCGAA GTGCTGTTAT | | |
| | CAGAGCTCAG GGTGAGGCTA AGAGTGCCCA | | |
| | GCTTATTGGT CAAGCGATTG CCAATAATCC | | |
| | GGCATTTCTC ACACTCAGGA AAATCGAAGC | | |
| | AGCAAGAGAG ATTGCCCAGC CTCTCTCACA | | |
| | TGCAGCAAAC AAGGTGTACT TGAG | | |
| T151b | GATCTAGGAA ACTTTCCCGT CACTTTTTTG | ambiguous hit | SEQ ID N° 441 |
| | CCCAAATTCT TGAAGCTCCA ACCACTACCA | | |
| | CCTCACAATA CTTATATCAA TGGATAGAGC | | |
| | TCCTCAAGAC CTAGCTATTG ATGCCAATTT | | |
| | TACCATGAAA ATCCGGCGAT CAAAATCCGG | | |
| | CCAAATTCCG GCGACCTCCC CGAACACCCT | | |
| | CTTTTGGCAT ACCACCATTT TTTCGGCCAC | | |
| | TTGAATTATA AAATGGTAAT TTTCGGACCA | | |
| | TGTAAACTCA TAAAATCGAG TTGGAATGAA | | |
| | AGATAATGAC GCTGAGAAAT ATTAGTAGCT | | |
| T153 | TAAGCATATA GCTTTTCCTT CTGAGCCAGG | lectin-like protein | SEQ ID N° 442 |
| | ATCACACTTC ACACTAACCG AATCTCGCAT | | |
| | AGAATCCATA AATGAAGAAA GCATCTCAAT | | |
| | TGGAGAAAAG TTTGTTTTCC CGGGGAATTT | | |
| | GCTTGTCAAC GAAATTCCAC TCATAAGTAG | | |
| | GTTCACATCG TGATCTAAGT TCCATTTCCC | | |
| | ATCGAGAGGT GAGTGATACT GGTAGGAGAG | | |
| | TCCTATTCTT CTCCTTGGGT TATGAAAGAA | | |
| | TTCAATAGCT CCGGGCTCCC TCACTGC | | |
| T154 | TAAGGCTGCC TACGAAGCAA TCTCAGATTT | putative reverse | SEQ ID N° 443 |
| | TACATGCAAT AAAAAAGACT ACTCTTGGCT | trancriptase | |
| | CTGGAAAATC AACTCCCTAA ACAAATTGAA | | |
| | ATATCTCCTC TGGACAATCA TTTGGGACAG | | |
| | GTTACCCACA AAGCATATGG GGGCCAAAAG | | |
| | AGGGATTTGC CATGACGACA CTTGTAACAT | | |
| | ATGTAATAGG GAGCCTAAGA ACATAGAACA | | |
| T158 | GGGCAAACGTGCTGGGAATAAATCTGAATGTGCCA | glycine-rich protein | SEQ ID N° 444 |
| | CTCTCTCTTAGCCTTGTTCTCAACAACTGTGGAAGG | | |
| | AATCCTCCTACTGGCTTCACTTGCTAAGCGCAAGTA | | |
| | CCCGATTA | | |
| T160b | GATCTCTTGC CTCGTGCAGA CATGCTTGAT | putative | SEQ ID N° 445 |
| | TCTCGTCCTT TGGCCACTCC TCTTACTAGT | retroelement pol | |
| | GGTACCGAGC TTCCCAATGA CTGCGTAGTG | polyprotein | |
| | ATCTAGGGCG GGTTCTGTTG ATGTGTACAT | | |
| | ATAATAAGAT CACATCTAGA TTATGGATTC | | |
| | TCTTTGAGGA TAAGTTTCAC TTTTTGTTCC | | |
| | TACCTTTTTG TAGTAAATTT | | |
| T164 | AGGCTGGTACCGGTCCGGAATTCCCGGGATATCGT | par peptide | SEQ ID N° 446 |
| | CGACCCACGCGTC | | |
| | CGATATTCTCAAACAAAAAGAATGGAGAGCAACA | | |
| | CGTGGTTCTGCTAGATTTCTGGCCAAGCTCTTTTGG | | |
| | TATGAGGCTAAGAATTGCATTGGCCTTAAAGGGAA | | |
| | TCAAATATGAAGCAAAGGAGGAAAACTTATCTGAT | | |
| | AAAAGCCCTTTGCTTCTGGAGATGAACCCTGTTCAC | | |
| | AAAAAGATCCCTATTTTGATTCACAATAGTAAAGC | | |
| | CATTTGTGAGTCTCTAAACATTCTTGAGTACATTGA | | |
| | TGAAGTCTGGCATGACAAATGTCCATTACTTCCTTC | | |
| | TGATCCTTACGAAAGGTCACAAGCCAGATTCTGGG | | |
| | CCGACTATATTGACAAGAAGATATATAGCACAGGA | | |
| | AGAAGAGTGTGGAGCGGTAAAGGTGAAGATCAAG | | |
| | AAGAAGCAAAGAAGGAATTCATAGAAATACTCAA | | |
| | GACTTTGGAAGGAGAGCTTGGAAATAAAACTTACT | | |
| | TTGGTGGTGATAATCTGGGTTTTGTGGATGTGGCTT | | |
| | TGGTTCCCTTTACTAGTTGGTTTTATTCTTATGAGAC | | |
| | TTGTGCAAACTTTAGTATAGAAGCAGAGTGTCCAA | | |
| | AGCTGGTGGTATGGGCAAAAACATGTATGGAGAGC | | |
| | GAGAGTGTCTCAAAGTCCCTTCCTCATCCTCACAAG | | |
| | ATCTATGGTTTTGTCTTGGAACTCAAGCACAAGCTT | | |
| | GGTCTTGCTTGAACAAGAAACACTTCTTACCTACTG | | |
| | CAGAAACCAATCATGTCCTTCGTCCCTAGTTGTTCA | | |
| | AGCATCAATTTATCAATATTGTTGCTACTCTGTCTA | | |
| | TAAATTTTATGGTTTGGTGTAATTTAGT | | |
| T168 | GATCTATCCA TGGAGTGAAT TTCGCATCAG | putative lipase | SEQ ID N° 447 |
| | GTGGAGCTGG CTGTTTA | | |
| T17 | GATCTCAATG GTGAATTGAC CTTGAAACAA | annexin | SEQ ID N° 448 |
| | GTAGTTCAAT GCCTTTGCTC ACCTCAATCC | | |
| | TACTTCAGCA ACATTTTGAT CGCGTCCTTA | | |
| T171 | ATGGACATTTGTGTACGAGAAGAAACCTGAAGAAA | wound-induced | SEQ ID N° 449 |
| | CCCCAGAGCCTCTCGTTTTGTTGGCTTATGCCCTAC | vacuolar membrane | |
| | ATGTGACCAAAGATGTAGAGAGTCACCTTCTCAAG | protein Sn-1 | |
| | TAATCTAATCTATGCTATTCAATGGTTCATAGCCAT | | |
| | ATATATATGTATGTTA | | |
| T172 | TGGGAGCTGAAAATGGCCTGATTGTTAGCGATAGC | protein phosphatase | SEQ ID N° 450 |
| | ATCATTCAGGGAAATGAAGAAGACGAGATTTTATC | 2C | |
| | TGTTGGAGAGGATCCTTGTGTAATTAATGGGGAGG | | |
| | AGTTGTTGCCACTGGGCGCTAGCTCGGAGTTGAG | | |
| | TGCCAATTGCTGTTGAAATCGAGGGTATTGACAAT | | |
| | GGTCAAATACTTGCCAAAGTCATAAGTTTGGAGGA | | |
| | AAGGAGTTTTGAGAGAAAGATCAGTAATCTGTCCG | | |
| | CCGTTGCTGCTATCCCAGATGATGAAATTACTACTG | | |
| | GCCCTACGCTAAAGGCATCCGTAGTGGCTCTTCCGT | | |
| | TGCCTAGTGAGAATGAACCTGTCAAAGAAAGTGTC | | |
| | AAGAGTGTGTTTGAATTGGAATGCGTGCCACTCTG | | |
| | GGGCTCTGTATCTATCTGTGGAAAGAGACCAGAGA | | |
| | TGGAGGATGCTCTTATGGTTGTTCCTAATTTCATGA | | |
| | AAATACCTATCAAAATGTTTATTGGTGATCGTGTGA | | |
| | TTGACGGACTAAGTCAACGTTTGAGTCACCTGACA | | |
| | TCTCATTTTTATGGTGTATATGATGGTCATGGAGGA | | |
| | TCTCAGGTTGCGGATTATTGCTGCAAACGCATTCAT | | |
| | TTAGCATTAGTTGAGGAGTTAAAACTTTTCAAAGAT | | |
| | GATATGGTGGACGGGAGTGCAAAGGACACACGTCA | | |
| | GGTGCAGTGGGAGAAGGTCTTTACTAGTTGCTTTCT | | |
| | CAAGGTTGACGATGAAGTTGGGGGGAAAGTGAAC | | |
| | AGTGATCCCGGTGAAGACAACATAGATACCACTAG | | |
| | CTGCGCCTCTGAACCTATTGCCCCGGAAACTGTGG | | |
| | GGTCCACTGCGGTTGTAGCGGTGATATGTTCATCTC | | |
| | ATATTGTAGTTTCTAATTGTGGGGATTCAAGAGCAG | | |
| | TCCTTTATCGTGGCAAAGAAGCAATGGCACTGTCA | | |
| | ATTGATCATAAACCAAGCAGAGAAGATGAGTATGC | | |
| | TAGAATTGAAGCATCTGGTGGCAAGGTCATTCAGT | | |
| | GGAATGGACATCGTGTTTTTGGCGTCCTTGCAATGT | | |
| | CAAGATCTATTGGTGACAGATACTTGAAACCATGG | | |
| | ATTTATACCCGAACCAGAAATTATGTTTGTACCACG | | |
| | AGCCAGAGAAGACGAATGCCTAGTTTTAGCTAGTG | | |
| | ACGGGTTGTGGGATGTCATGTCAAATGAGGAAGCT | | |
| | TGTGAAGTAGCTAGACGACGAATTCTGCTATGGCA | | |
| | CAAAAAGAATGGGACTAATCCTCTGCCGGAAAGGG | | |
| | GCCAAGGAGTTGATCCTGCTGCACAAGCAGCAGCA | | |
| | GAGTATCTCTCGACGATGGCTCTTCAAAAAGGTAG | | |
| | CAAAGACAATATATCTGTGATTGTGGTGGACCTTA | | |
| | AAGCTCAAAGGAAGTTCAAGAGCAAATGTTAAGAG | | |
| | ATGACAATGTTCACCCGCACTTTGGTTTTTAGTATA | | |
| | AATCTATATACGGCTATGGGGTATAATCTCATTATT | | |
| | ACATAACTCGGTCCATCCATTTTTTTATGGGCTTAA | | |
| | GGTCTGTGTATGAGAATAGTGTTTAGCATGTATTTA | | |
| | TAGAAAAACAGTTTAACAAATGACGTTTATCCAAA | | |
| | TTTTTGGTGTTGTTATGCCAGCAAGTGGCTATGTAA | | |
| | ATTGAGCATGTTGTAGCAATATCAAAGATGCAAGT | | |
| | TCTTTGTTTAAAAAAAAAAAAAAAAAAA | | |
| T177a | TGACTGCGTAGTGCTCTATATGGCAATAGATTTGAA | leucine-rich repeat | SEQ ID N° 451 |
| | GGCAACATTCCCAAGCCTTTTGCTAAATTGAAGTCT | protein | |
| | CTTAGATTTTTGCGGTTA | | |
| T177c | GATCTATACCAGAAGGAGCTGTTGTATGTAATGTG | 60S ribosomal | SEQ ID N° 452 |
| | GAGCATAAAGTGGGAGATCGTGGTGTTTTTGCTAG | protein L2 | |
| | ATGCTCTGGTGATTATGCCATTGTTATCAGCCACAA | | |
| | CCCTGATAATGGTACCACTAGGGTTA | | |
| T178 | CTGGAATCAATTGCTTCCTCTGCGGTGCGGGCAGC | pyruvate kinase-like | SEQ ID N° 453 |
| | GATTA | protein | |
| T18 | TCAAAAACAA CTTTTATTGT GTTCATGGTT | pathogenesis-related | SEQ ID N° 454 |
| | TTAGCCGTGG CCCATTCTTC ATTAGCCCAA | protein | |
| | AACACTCCCA AAGATATCGT TATTGTCCAC | | |
| | AACAAAGCCC GTGCAGAAGT TGGTGTCCCA | | |
| | CTCCCACCAT TA | | |
| T2 | TGAGTGAGCT TCATTATCTA CAAGCTTCCA | putative cytochrome | SEQ ID N° 455 |
| | TTTATGAAAG TATGAGACTT TACCCTCCTA | P450 | |
| | TCCAATTTGA TTCAAAGTTT TGTTTAGAAG | | |
| | ATGATATTTT ACCTGATGGG ACTTTTGTGA | | |
| | AGAAAGGAAC AAGGGTTACG TATCATCCTT | | |
| | ATGCAATGGG AAGAATGGAA GAATTATGGG | | |
| | GTTGTGATTT | | |
| T20 | GATCTCATTTCGATCCTCACCACCCTCATCTGGCTA | 13-lipoxygenase | SEQ ID N° 456 |
| | GCTTCAGCACAACATGCTTCGCTGAATTTCGGCCAG | | |
| | TACCCATACGGCGGCTACGTCCCCAATCGGCCACC | | |
| | TCTCATGCGTAGATTA | | |
| T201 | GATCTCGCTT CGGGATCATT CCCCAAGAGC | MRP-like ABC | SEQ ID N° 457 |
| | CAGTCCTTTT TGAAGGAACT GTGAGAAGCA | transporter | |
| | ACATTGACCC CATTGGACAA TATTCAGATG | | |
| | ATGAAATTTG GAAGAGCCTC GAACGCTGCC A | | |
| T203 | TCATCGAAATAATGAGTCACCATTGATATCGACAC | chloroplast putative | SEQ ID N° 458 |
| | ATCTCCGATCGCCAAACGCTCGGGAGTTCCTCTAT | protein 1708 | |
| | CAATCCTTTTCCTTCTTCTTGTTGCTGGATATCTCGT | [Nicotiana tabacum] | |
| | TCGTACACATATTGTCTTTGTTTCCCGGGCCTCTAG | | |
| | TGAGTGACAGACAGAGTTCGAAAAGGTCAAATCTT | | |
| | TGATGATTCCATCATCTATGATTGAGTTGCGAAAAC | | |
| | TTCTGGATAGGTATCCTACATCTGAACCGAATTCTT | | |
| | TCTGGTTA | | |
| T204 | GATCTCGAGC TCAGATTACA AAGCAAATCA | putative protein | SEQ ID N° 459 |
| | AGCATTTGTT TGGCAAGGAA CTAGAAATCG | At3g46190 [A. | |
| | GAACCGCGAA AATGACAACC TCTTGAACCG | thaliana] | |
| | AAACCCATTG ATAAAACCTC GACAAACCTC | | |
| | ACCTACCTCA ACTCCCATGC TTTATGGTTG | | |
| | TGTTTTTTGG TAGAAGAAAT GGTGTTTCGG | | |
| | AGCTAAAGTG AGGAGCTGTT TCGAACAAGG | | |
| | CTTCAGCTGC GTTATTGACT GATTTTTTGG | | |
| | TGAGTTTCGG GGTTA | | |
| T205a | AGATGTGACAGCCCGTTAGATTTACGTCATAAGAG | putative apoptosis | SEQ ID N° 460 |
| | GGCTGGCGTCGAGCCGCTTGGATAGATTTGATCGA | inhibitor like | |
| | CCCCAGGTGCATCCTTGGGGAATTCCTGTGTTCGT | protein | |
| | CAAGGTCTAAGCCGATTTATTCCTGGCCGGACGT | | |
| | CGACAGGTTTTGAGGGAAGTGACTGACCCGAGATC | | |
| T205b | GATCTCGAAC TAGCGATCTC AAATTTCACC | aklanonic acid | SEQ ID N° 461 |
| | TCCAGTTCCA CCGAAAATTA CCGTTCTGCT | methyltransferase | |
| | TGTGAAGCTA CTACTAGCAC GATTCCCGAA | like protein | |
| | GAAGTGGAAA CCGGACTTGT TGTCGGTGGG | | |
| | CCCCATGGAC CGCCGCCAGG ACTCGCTGGA | | |
| | AGATTATTAC TCTGCCGTTT TCAA | | |
| T206a | GCTATAAACCAGACACAAATATCTCCATCTGGGAG | non-photosynthetic | SEQ ID N° 462 |
| | GCAGCATACCAATCTGAAGGTGCATTTCTTGACGA | ferredoxin | |
| | CGATCAAATGGAGAAGGGTTATTTGCTGACTTGTA | | |
| | TTTCATACCCGAGCATC | | |
| T207b | TAACGATGTC AAAAAATTTC TGTCGGAGAC | phosphatidylinositol- | SEQ ID N° 463 |
| | AGAATCAGAG ATTATAATCC TCGAGATC | specific | |
| | | phospholipase-like | |
| | | protein | |
| T208 | TGAGTAACGTGAGGGAAACTGCTCTTCCTTCAGTA | putative protein | SEQ ID N° 464 |
| | ATTGCACAATACCCCGAGATC | AT4g02990 [A. | |
| | | thaliana] | |
| T21 | TATCGATTAT TCATACAGTG AGAGCATAGC | cyclophilin | SEQ ID N° 465 |
| | TTAAAAACTC CACAGAAATT TCTAGAAGAG | | |
| | AGTGAGAGAT GGCAAATCCT AAGGTTTTCT | | |
| | TCGACCTTAC CGTCGGCGGT CTACCGACCG | | |
| | GCCGTGTGGT GATGGAGTTG TTCAACGATG | | |
| | TAGTTCCGAA AACAGCGGAT AACTTCCGAG | | |
| | CACTCTGTAC CGGAGAGAAA GGCGTCGGAA | | |
| | AGTCCGGCAA GCCGTTACAC TACAAAGGAT | | |
| | CATCATTTCA CCGTGTGATT CCTGGATTTA | | |
| | TGTGTCAAGG AGGTGATTTC ACTGCTGGAA | | |
| | ACGGTACCGG CGGTGAATCG ATCTACGGCG | | |
| | CCAAATTCGC CGACGAGAAT TTCGTTAAAA | | |
| | AGCATACTGG ACCTGGAATT CTCTCTATGG | | |
| | CCAATGCTGG ACCTGGAACT AACGGATCTC | | |
| | AGTTTTTCAT CTGTACGGCC AAAACCGAGT | | |
| | GGCTTGATGG GAAACACGTG GTGTTTGGTC | | |
| | AAGTTATTGA AGGAATGGAC GTGATTAAGA | | |
| | AAGTGGAAGC CGTTGGATCT AGCTCCGGCA | | |
| | GGTGCTCGAA GCCCGTTGTG ATTGCTGACT | | |
| | GTGGTCAACT CTCTTAGATT ATTAATCGTA | | |
| | TCAATTAATG TTAATGATGA TCTAGTCTAG | | |
| | TTAACTATGT GATCGCAGTG TACTGATTTG | | |
| | CTGGTTTTCG TTTTTTTTTT AGCCTTTTCC | | |
| | TTTTTGAGAT TGTGGGTCGG GTTTCGGGCG | | |
| | TACTGTGTCG GGTCTTTACT GTAATTGGTG | | |
| | GTGTTTACTA CTACCAGTGC ATGTTGGAAT | | |
| | TGGAATAAGA TTAGATTTCT CGGTTTAAAA | | |
| | AAAAAAAAAA AAAAAA | | |
| T210 | ACAGCTATGACCTTAGGCCTATTTAGGTGACACTA | putative protein | SEQ ID N° 466 |
| | TAGAACAAGTTTGTACAAAAAAGCAGGCTGGTACC | P0638D12 [Oryza | |
| | GGTCCGGAATTCCCGGGATCTCAAAAAACACGATC | sativa] | |
| | AATGATCCGTACAACTCTCTCTTATCGAGTCCTCT | | |
| | ATTTCCAATAATCACCAAATTACCCCACAAGTTTT | | |
| | CGATTGGATCAATTTAGTGTTTGATCTTTAGCTGT | | |
| | TCTGATCAGTTTATTAGTGGAAATGAAGATAGTGG | | |
| | ATTTGGATGAGTCGTTAATGGAAAGTGATGGCAAT | | |
| | TGTGTAAATACTGAGAAACGGTTGATTGTTGTTGG | | |
| | TGTTGATGCTAAAAGAGCGTTGGTCGGAGCCGGGG | | |
| | CTCGGATCCTTTTTTACCCGACCCTTTTATACAAT | | |
| | GTTTTCCGCAACAAAATTCAATCGGAGTTCAGATG | | |
| | GTGGGATCAAATTGATCAGTTTCTCCTCCTTGGAG | | |
| | CAGTTCCATTTCCCTCGGATGTCCCTCGGTTGAAG | | |
| | CAGCTTGGCGTTGGTGGTGTAATAACACTGAATGA | | |
| | ACCTTATGAAACTTTGGTACCATCATCATTGTACC | | |
| | ATGCCCATGGGATAGACCATCTCGTTATTCCTACC | | |
| | AGAGATTATCTTTTTGCACCCTCTTTCGTGGATAT | | |
| | AAATCGAGCAGTAGATTTTATTCACAGGAATGCGT | | |
| | CCATTGGCCAGACTACGTATGTACATTGCAAAGCC | | |
| | GGAAGGGGAAGGAGCACAACCGTTGTGCTTTGCTA | | |
| | TTTGGTGGAATATAAGCACATGACTCCTCGTGCTG | | |
| | CCCTTGAATTCGTCCGCTCCAGAAGACCTCGAGTT | | |
| | TTATTGGCTCCTTCTCAATGGAAGGCTGTTCAAGA | | |
| | ATTCAAGCAGCAAAGAGTGGCATCTTATGCGCTCT | | |
| | CTGGTGATGCTGTATTGATCACTAAAGCAGATCTC | | |
| | GAAGGCTATCATAGTTCTTCTGATGATAGTCGCGG | | |
| | TAAGGAACTGGCCATTGTGCCTCGAATAGCAAGAA | | |
| | CACAGCCGATGATAGCTAGATTATCCTGCCTCTTT | | |
| | GCATCCTTGAAAGTATCAGATGGTTGTGGACCTGT | | |
| | TACCAGGCAACTGACCGAGGCACGTGCCTGCTAAT | | |
| | CGCAAACTCATCAGCAGCAGCTACCTTGTACAGAA | | |
| | GACCACTGCTTAAATAAGGTCAGAAAGAGTCTTAT | | |
| | ATCTTTGAATCTGTGCTTCAGAGTGAACATCAAGG | | |
| | GATTATGAATAGAAAAAAACAGCTGAAGAGTACTT | | |
| | CAACATTGTGTAAACATGTTCAGAGTATGACTACT | | |
| | GTGGTCATTAGTAAATATTGCATAATTATACTCTT | | |
| | CCCATAATAAAGGGCGGGTATACAGACTTATTCTG | | |
| | AGAAAAAAAAAAAAAAAAAAA | | |
| T211 | TAAGGCAGAA AATAAACTCC TATTGCTTTG | beta(1,3)-glucanase | SEQ ID N° 467 |
| | ATGTGCATGT TACAGTATAT GTTACAAAAG | regulator | |
| | AAAAACTTTC TGTTTATATA GTAGGAGAGT | | |
| | TTCATCCCTA GTATAAGTCT AAAAAGGTAA AAAT | | |
| T213 | CACTCTCTCTTAGCCTTGGTCTCAACAACTGTGGAA | putative | SEQ ID N° 468 |
| | GGAATGCTCCTACTGGCTTCACTTGCTAAGCGGAA | strictosidine | |
| | GTACCCGATTACTCAGGACTCATCATCTACCAGCG | synthase | |
| | CAGGCAATTTGTTGCTGCGACTGCAAGTGGAGATA | | |
| | AGACAGGCAGGCTGATGAAATATTATAAACCAACA | | |
| | AAAGAAGTAACAGTTGCACTAGGAGGCCTA | | |
| T214 | GATCTCGGATTTCTTATTTCATTGCCCTCTTCCTTTA | putative protein | SEQ ID N° 469 |
| | TTCCTCACTGGCTGTTCGTATTA | P0501G01 [Oryza | |
| | | sativa] | |
| T216 | TAAACAATGT TCAGCCTTTC GTTGCAAGTT | amidophospho | SEQ ID N° 470 |
| | ATAAATTTGG ATCAGTTGGT GTTGCCCACA | ribosyltransferase | |
| | ATGGCAATTT TGTGAATTAC CTAGCTCTTC | | |
| | GTGCTGAACT TGAGGAAGAC GGGGCAATTT | | |
| | TCAAGACTAG TTCTGAGACT GAGGTGGTTC | | |
| | TTCACCTTAT TGCTAGATCA AAGAAGGAGC | | |
| | TTTTTCTTTT GAGGATT | | |
| T217 | GATCTAGTGT CATGGTCCCT CGGAATTTCA | putative E2 | SEQ ID N° 471 |
| | GATTACTTGA GGAACTTGAA CGCGGTGAAA | ubiquitin- | |
| | AGGGTATTGA AAATGGGACC GTGAGGTATG | conjugating enzyme | |
| | GGATGGATAA TGCAAATTAT ATGTATATGC | | |
| | GTCCCTGGAC TGGCCCCACT ATTGGCCCTC | | |
| | AGGATTCCGT TGACTGCGTA GTGATCTGTA | | |
| | ACTGCCGAAG ATATCATCTT GCCGCCTCAT | | |
| | GTAGAAAT | | |
| T22 | GATCTCACTC CAAATCACAA TCTCCGCCGT | putative protein | SEQ ID N° 472 |
| | CTGATCCAAT CATGGTGCAC ATTA | At2g35930 [A. | |
| | | thaliana] | |
| T220 | CATCCATCATTATCTTAGGTACACCCGTCCAGCCAG | glucose 6 | SEQ ID N° 473 |
| | GCAACCCTCTTGGAGCTGCCATTGCAATTCTTGGAA | phosphate/phosphate | |
| | CTGTCTTGCATTCACAGGCAAAACAGTGAAGAGTG | translocator | |
| | GAATTTATATATCGCGCAGGAAAGGTGTCGGAGAG | | |
| | AACCGAGAGGTGTTGAGAAAACGTATCCCATAATC | | |
| | CTGAATCTACCCTTACTTGAGGTGGAACATGAAAC | | |
| | TTATTAGTATGTACATAGCAATAATGGGTTACTCAA | | |
| | GACT | | |
| T221 | ACTTTGGTACTCCACGTTGTGGGACCTACTGGTGGA | putative | SEQ ID N° 474 |
| | TTGGCTACCCCACTTGTCCAAGATTTTGAACGCCAA | strictosidine | |
| | CCTCTTCTCTTTCACAAATGATCTGGACATTGATGAC | synthase | |
| | GACGACGATATTATTTACTTCACGGATACAAGCAC | | |
| | AATCTACCAGCGCAGGCAATTTGTTGCTGCGACTG | | |
| | CAAGTGGAGATAAGACAGGCAGGCTGATGAAATAT | | |
| | AATAAATCAACAAAAGAAGTAACAGTTGCACTAGG | | |
| | AGGCCTAGCTTTTGCAAATGGTGTAGCCTTACTCAG | | |
| | GACTCATCAC | | |
| T222a | GATCTCTCCA ATTTCCTCTT CACTGTCGGT | putative protein | SEQ ID N° 475 |
| | GCCAGAATCC CTGCTCAAGT CTTTGGTTCA | At3g56950 [A. | |
| | ATTACTGGGG TTAGGCTCAT CATTGCAGCA | thaliana] | |
| | TTTCCAAACA TAGGACGAGG ACCTCGTTTG | | |
| | ACCATTGACA TCCACCGAGG TGCACTGATT | | |
| | GAAGGGTGCT TGACATTTGC GATTGTTACC | | |
| | ATTTCACTTG GACTTTCCAG AAGAAGTCGT | | |
| T222b | GATCTCTTGC CTCGTGCAGA CATGCTTGAT | retrotransposon- | SEQ ID N° 476 |
| | TCTCGTCCTT TGGCCACTCC TCTTACTAGT | like protein | |
| | GGTACCGAGC TTCCCAAGTT GGATGTCACT | | |
| | TCCCTCTCTG ATCCCACCTA TTTCATTCTT | | |
| | CTATTGAGTC GGCTAACTGT AACTATAAGC | | |
| | TACACGCCTC GAACTCGTAT AAAGATTCTT | | |
| | CCTCTAGGGC CTCCTTTCAC CTT | | |
| T222c | CGACAGAGAGCAGCCCTGAATCTTTGGCTATGTCA | putative nucleic | SEQ ID N° 477 |
| | ACTCCGTTCCTACACATTTCTCGTCCTCTTTCTCCAC | acid binding protein | |
| | ACGAGTACAACCATAAGCCTTATAAATACTGAAAT | | |
| | CTCATCAATAGCTGTGACTTGTAATTGACTAACTAA | | |
| | GCCCATGGCTTCCAACTCTTCCTCCCATAGCCCTCG | | |
| | CACCGTCGAAGAGATC | | |
| T225 | GTGATCTCTTGCTGTATCAAGAGGTATTGGAGATCA | protein phosphatase | SEQ ID N° 478 |
| | GTGTCTTA | 2C | |
| T227 | TAATCCAAAC AAAACTTCTA CTGCGAAGAA | ribosomal protein | SEQ ID N° 480 |
| | GGTCCGCGGT GTAAAGAAGA CCAAGGCTGG | S19 | |
| | TGATGCTAAG AAGAAATAAG TCTTATGCAA | | |
| | ACAAAAATCT CAATTTGGGA TTCTTTTGGT | | |
| | GGCCTATGTA TTTGTCTTGT GGTACTGTTG | | |
| | ATTTTGACTT TGATTTTGGG GCGATTCAGT | | |
| | TATCTTCCCA TGGGGATATC TCATGGAAGG | | |
| | CTTAGAGTAC TTGAGAGTTC TATTAGTTAG T | | |
| T228 | GGCCTTATTCTTTTGCTTTCAGGATTCATCTTACCAC | putative protein | SEQ ID N° 481 |
| | CTACTGATGGCATCGCGCATCATCGACGATCCCTCT | At5g05740 [A. | |
| | GTGTTCCACGAATCATTTCTAGCTGGCGGTATAGCC | thaliana] | |
| | AAGCTTATTCTAGGAGATGCTCTCAAGGAAGGAAC | | |
| | TCCTATATCAGTAAATCCGCTTGTCATATGGGCCTG | | |
| | GGCTGGACTTCTCATTA | | |
| T229a | GATCTCTAGC ACAAAAACGA CCCCCCCCGT | nucleoporin-like | SEQ ID N° 482 |
| | TAGTCATCTT CTCCAGACAA TCCCTAAGTC | protein | |
| | GACGAGTAGC TGCTGCCTCG TCCTTCACTG | | |
| | AGCATCCAAA GTCCAAACGC CGCTGCTTTC | | |
| | GTCTTCAACC CATCGTCCAA CTTCACGTCG | | |
| T25 | TAAATGGAGCAAGGCTAAGCTTGTCTGGTGATCAC | putative | SEQ ID N° 483 |
| | CAGCTCAGTAATGCTGGCCTTGCTGTATCCCTTTGT | folylpolyglutamate | |
| | AAAAGTTGGCTTAGAAGTACAGGAAACTGGAAAA | synthetase | |
| | GGCTGTTTGAAGATGCATATGAGAAAGATGGTCTA | | |
| | CCAGAGGAATTCCTGAGGGGTCTTTCAGCTGCACG | | |
| | TCTTTTCTGGCAGGGGTCAGATTGTTGTTGACCCTCT | | |
| | GATCAACACATCTGGAGGACATAAAAGGTTGTCAG | | |
| | GAGATC | | |
| T27 | GATCTCCCAA TACTGACCGG GGGATAGGAA | transposase-like | SEQ ID N° 484 |
| | GTCCATTGCG AGAATATAGC CCTAATATAC | protein | |
| | GAGATGAACT TAGAAGACGT TATATTCAAA | | |
| | TGGGACCTTG CCAGCCTACG AGTCATGATT | | |
| | TTCCTAAAAC TAAGTTTGGG AAGACAATGC | | |
| | GTCAGTTTTA TCCTGGTTGG TTTTA | | |
| T28 | GATCTCCTAG GAGTGTTAGT GACAAAGATA | nicotinic | SEQ ID N° 485 |
| | GCCCACGTTC TGTGTTTTTG GATCGCAGTT | acetylcholine | |
| | CATCGTCAAA TTCTAGGCGT AGTTCTAGTG | receptor epsilon | |
| | GTACTAGTTC CGAAGCATCC GTACAGTAGC TTTA | subunit | |
| T3 | TGAACCCTTT TTGATGGACT TAAGGGAATA | putative protein | SEQ ID N° 486 |
| | ATTTGGTGAC CCAATCTTCC TCCTCTTGGA | P0529E05 [Oryza | |
| | CTTCAATTTG GACCACCATA TAATTGTAAA | sativa] | |
| | ATTTGGACAA TTTATTTCCT TTGGTCTTGA | | |
| | GCTCTTCCTC TACAATTGAA AGCTTCTTTA | | |
| | TTTGCCATTG AAGTCTAGCA ACCTTAGTAG GCAA | | |
| T30 | GATCTCCCAG AAGGGGTCCA AAGCATCATT | putative protein | SEQ ID N° 487 |
| | GCAGATTCTA GTGAATGTGT GTCAATGGGG | At2g35930 [A. | |
| | GAGGAACAGA GTGAAAGCGG CGGAGACTGA | thaliana] | |
| | CGCGGTTAGA | | |
| T302a | TAAAGCCACAGACAAGACCAACTACATTGGTGCTA | putative protein | SEQ ID N° 488 |
| | ATGATCTTCAAGCTACCTACTCTCTCTATCCAGGAA | At1g76660 [A. | |
| | GTCCTGCTACTACTCTCAGATCACTACGCAGTCAAT | thaliana] | |
| | CCCGCG | | |
| T302b | GATCTGGATT TACCTCCACC TCCCAGGCCT | splicing factor | SEQ ID N° 489 |
| | GGTTTTCCAT CTGTTAGGCC ACTACCTCCA | | |
| | CCTCCTGGGC TTGCGCTGAA TATTCCTAGG | | |
| | CCTCCTAATA CAGTCCAGTA TTCCACCTCC | | |
| | ACCAGTGCTG GGGTTGCTGC TCCACCTCGA | | |
| | CCTCCTATGG TTACTCAGGG GTCATCAATC | | |
| | ACTAGT | | |
| T302c | ACTAGTGATTGACTGCGTAGTGATCTGACTTGTCCG | pectin | SEQ ID N° 490 |
| | CTTTGTATAGATGTGAC | methylesterase | |
| T303b | CGAGTTGAATGAATCAAAGCAAGACGAAGTCAGCA | cysteine-rich protein | SEQ ID N° 491 |
| | GTCCTCGCTCTTGAATCAGATC | | |
| T305 | GATCTGAGGAGAGTTTGCATTTTGGATTTGCGCACG | arabinogalactan- | SEQ ID N° 493 |
| | AGATGTTTATGATTCTAGGATTTATTTTAGTCATCT | protein | |
| | TACTCGGCTGATGTTTATTCGCTTTTGTGACTTTTAC | | |
| | TCGTGGGCGGTGGTGACCGCGTACATGCTATTTATT | | |
| | TGATTTTTACTATGGTTATTGTTTATTGTTA | | |
| T308a | GATCTGATCC AGCAGTTGTT CTTGCATTTG | putative protein | SEQ ID N° 494 |
| | ATATTCAGTG TAATATTGAA TCATTTTATC | BAC19.2 | |
| | AAGTTATCGT TGCTGTCCCT TTTCTTGGTA | [Lycopersicon | |
| | ATCCAGTTGT CTGCTTTGAG ATTTACTCTT | esculentum] | |
| | CTGAATCAAA ATCTTGGAGT TGCTCTTCTT | | |
| | CAGACTGTAT TGAGTTGGAA AATAGCACAA | | |
| | GTCCTCTAAT CTTTGATA | | |
| T309 | ATGGGAACGGCTTCCTGGTTGCACTTGTTGGTACGG | putative protein | SEQ ID N° 495 |
| | ACTCTCTTATGGAGTTTTGACTGGTACTTCCCTTGTT | 4933419D20 [Mus | |
| | CCCCTGTACTTATTCCTAATACTCCAGGAAATATTG | musculus] | |
| | CGTCACTTGGCTTA | | |
| T311 | ATAAACAGCCTTGGATGATTCTTGCTGCTCATCGTG | putative protein | SEQ ID N° 496 |
| | CCCTTGGTTACTCCGCTAATGATTGGTATGCTAAGG | AJ271664 [Cicer | |
| | AAGGCTCATTTGAAGAGCCCATGGGAAGGGAGCAC | arietinum] | |
| | TTGCACAAACTCTGGCAGAAATATAAGGTTGATAT | | |
| | GGCATTTTATGGGCACGTCCATAACTATGAAAGAG | | |
| | TTTGCCCAATTTACCAGAATCAATGTGTGAACAAG | | |
| | GAGACATCACACTACTCGGGCGTAGTGAAAGGAAC | | |
| | AATTCATGTTGAAGTTGGGGGAGGAGGAACCCTTT | | |
| | TGAATAAATT | | |
| T313a | GATCTGAGACCGGGGTTTATCGAGACTGAGTTTTAT | phospholipase D | SEQ ID N° 497 |
| | ACTTCTCCTCAAGTGTTCCATTA | | |
| T314 | CTAAGGGTGCTGCCAGCTTTACCTCCCAAGTCATCA | elongation factor-1 | SEQ ID N° 498 |
| | TCATGAACCATCCAGGACCGATTGGAAATGGATAT | alpha | |
| | GCTCCAGTGCTTGACTGCCACACCTTCCACATTGCT | | |
| | GTCAAGTTTGCAGAAATTTTGACCAAGATCGACAG | | |
| | GCGTTCTGGTAAGGAGATTGAGAAGGAGCCCAAGT | | |
| | TCTTGAAGAATGGTGATGCTGGTATGGTTA | | |
| T315a | GATCTATGGT TTTGTCTTGG AACTCAAGCA | PROBABLE | SEQ ID N° 499 |
| | CAAGCTTGGT CTTGCTTGAA CAAGAAACAC | GLUTATHIONE S- | |
| | TTCTTACCTA CTGCAGAAAC CAATCATGTC | TRANSFERASE | |
| | CTTCGTCCCT AGTTGTTCAA GCATCAATTT | PARA | |
| | ATCAATATTG TTGCTACTCT GTCTATAAAT TTT | | |
| T315b | GATCTTGATA ACAAACGTAA TACTAACATG | putative protein | SEQ ID N° 500 |
| | AAACAAGCTA ATGGAACACA AAATTTACAG | At2g44270 [A. | |
| | AGCAAACAGT GTGGAAGCTT GGACTTTTGA | thaliana] | |
| | ATCATCATAT AACTGTATAA TCGTTGTATA | | |
| | ATTCTCAGTG GTGATCATTG CGATCT | | |
| T319a | GATCTTGCCATCACAGAAAAGGATCATTCTGGGCG | RNase NGR2 | SEQ ID N° 501 |
| | CATGAGTGGGAAAAACATGGGACATGTGCTTATCC | | |
| | AGTTGTCCATGATGAATATGAGTTCTTTTTGACTAC | | |
| | GCTGAATGTTTACTTCAAGTATAATGTTACAGAAGT | | |
| | TGTGCTTGAAGCTGGATATGTACCATCAGATTCCG | | |
| | TAAGTATCCATTACGAGGCATCATTTCATCAATTGA | | |
| | AAATGCTTTCCATGCAACCCCA | | |
| T319b | GATCTCATCA TGAATGTTGG TACTGGTGGC | 60S acidic | SEQ ID N° 502 |
| | GGTGGTGCTG CAGTTGCTGT TGCTGCTCCC | ribosomal protein | |
| | ACTGGTGGTG CCAGTGCCGG TGCTGCAGCT | P1-like protein | |
| | GCTGCCCCTG CTGCGGAGGA AAAGAAGGAA | | |
| | GAGCCTAAGG AAGAAAGTGA TGACGACATG | | |
| | GGATTCAGTC TGTTTGATTA GGAGCTCCTT | | |
| | TCAGTATGAT ATTTGGTTCT TTTTTAGAGA ATTG | | |
| T32 | TAACACAGAG AAAGTAGAAG AAACTACAAA | SGP1 monomeric | SEQ ID N° 503 |
| | ACAAGGACAA CAACAACATG CCAAGAATGG | G-protein like | |
| | ATCATCATAT GGTCTATTTC CTTCATTATG | protein | |
| | ATGATCCTGA TGACGACCCA TCTTCTTCTT | | |
| | TGACCTTGAG ATATGAACCT TCTTCTAAGT | | |
| | CTTGGGAGAT C | | |
| T320a | GATCTCCCTA CCGGTGGGCT TGCTAACGTC | phosphoglycerate | SEQ ID N° 504 |
| | GCTGCAACCT TTATGAATCT GCATGACTAC | mutase | |
| | GAGACACCAA GCGATTACGA GCCAAGCTTG | | |
| | ATTGAGGTTG TTGACAACTA GATATCTCAG | | |
| | AGAATTTAGG AGGGTTGAAA TTTTGGCGCA | | |
| | AGTTGGAAAG TGATAATGAC TACATTCTAT | | |
| | ACTCTTTCCA GTCTATTTGA ATAAGACATT | | |
| | TTTTTGAGCT TATATTA | | |
| T320b | AGTGATCTCCATCGTGACCTTGGTTTTGATAAGAAA | plexus-like protein | SEQ ID N° 505 |
| | GAAGCAGCTGCTCCCTTCCTTCTCCACTCCCAGCAT | | |
| | CAAGCACATTCCTTAGCACAATCAACCAGTCAACA | | |
| | ACCACCCCAAAACAACCTGCAAAACTCAGCAAAAT | | |
| | TCCACCCAAAAACTCCTAGAAGCGCAGTACTTCAG | | |
| | CTCCAGAAAGTCATGAAAACGCAGTTGTAGCACCG | | |
| | TCCCTTTTAGCACCCTTA | | |
| T320c | TAAGGGTGCTAAAAGGGACGGTGCTACAACTACGT | collagen-like | SEQ ID N° 506 |
| | TTTCATGACTTTTTGGAGCTGAAGTATTTTGCTTCTT | protein | |
| | GGAGTTTTTGGATGGAAATTTTGCTGAGTTTTGGAG | | |
| | GTTGTTTTGGGGTGGTTGTTGGCTGGTTGATTGTGC | | |
| | TAAGGAATGTGCTTGATGCTGGGAGTGGAGAAGGA | | |
| | AGGGAGAAGCTGCTTCTTTCTTATCAAAACCAAGG | | |
| | TCCCGATGGAGATC | | |
| T321 | TAAGGAAAAT AAATGACATG CATTTAGAAC | putative protein | SEQ ID N° 507 |
| | CAATATTCAA GAACAGTGAG TTTATCATCT | At2g11600 [A. | |
| | CTCAAAACAT AAACAAAATG AACTTGGCTT | thaliana] | |
| | CAAATAATCC TTGAACAAAA TAGGGAGATC | | |
| T322a | GATCTCGAGAGAATTTATGGCTTCACTCCAAGAAA | putative protein | SEQ ID N° 508 |
| | CCCTCGTGCTGTAAAGCCACCTGATCATTACATAGA | At5g22210 [A. | |
| | ATACATGCGCTTA | thaliana] | |
| T323 | ACAGCTATGACCATTAGGCCTATTTAGGTGACACT | cellulase | SEQ ID N° 509 |
| | ATAGAACAAGTTTGTACAAAAAAGCAGGCTGGTAC | | |
| | CGGTCCGGAATTCCCGGGATGAACATGAGAGGGAA | | |
| | ACCAAGGCTACTGGTTAATCTCTCAACCATTTGACT | | |
| | TTGATCACCAATTAAGCTCAGATACAATGCACTCA | | |
| | GCAAATCATTGGGGAGGATCATTAGAAATCGCGAA | | |
| | CACCGGCGATTCAACGGCGGAGGAATATGACCGGA | | |
| | GTCGGAATTTGGATTGGGACAGAGCATCAGTAAAT | | |
| | CATCATCAAAAACAACAACAGTATAATAACTACGA | | |
| | TCAATATTCTCATCGGCATAATTTAGATGAAACGC | | |
| | ACAGAGTTGGTTATTAGGTCCGCCGGAGAAGAAGA | | |
| | GAAGAAATACGTCGATTTAGGATGTATTTGTTTGC | | |
| | AGCAGAAAAGCATTCAAATATACTATTTATGGAAT | | |
| | TATTATCGCTTTTCTCGTTATCGCTCTGCCTACGATT | | |
| | ATCGCCAAGTCTTTGCCTAAGCATAAAACTCGGCCT | | |
| | TCTCCTCCTGATAATTACACTATTGCCCTTCACAAG | | |
| | GCTCTCCTCTTCTTCAACGCTCAAAAATCTGGAAAA | | |
| | TTGCCAAAAAACAATGAGATTCCATGGAGAGGAGA | | |
| | CTCAGGTTTACAAGATGGATCAAAACTCACAGACG | | |
| | TTAAAGGAGGGTTGATTGGAGGGTATTATGATGCT | | |
| | GGAGATAACACAAAATTTCACTTTCCAATGTCATTT | | |
| | GCAATGACAATGTTGAGTTGGAGTGTCATTGAATA | | |
| | TGAACACAAGTACAGAGCCATTGATGAGTATGATC | | |
| | ATATCAGAGATCTCATCAAATGGGGCACTGATTAC | | |
| | TTGCTTCGTACTTTCAACTCCACTGCCACTAAAATT | | |
| | GACAAAATTTATAGCCAGGTTGGTGGTTCTCTAAA | | |
| | CAATTCAAGAACACCAGATGATCACTACTGCTGGC | | |
| | AAAGGCCAGAAGACATGAACTATGAACGCCCTGTT | | |
| | CAAACAGCTAATTCGGGGCCTGATCTTGCCGGTGA | | |
| | AATGGCAGCAGCATTGGCTGCAGCCTCCATAGXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXXXXXXXXXXXX | | |
| | XXXXXXXXXXXXXXXXXXXXXXACGTAGGAACTG | | |
| | TGGCCCTCGCTATATCTCCTTGGATATTCTTCGCCG | | |
| | TTTTGCCACTTCCCAGATGAATTATATTTTAGGTGA | | |
| | CAATCCCTTGAAGATGAGCTATGTAGTAGGGTATG | | |
| | GAAACAAATTCCCAAGGCATGTACATCATAGGGGT | | |
| | GCATCAATACCCTCTGGTAAAACAAAGTACTCATG | | |
| | CACTGGAGGTTGGAAATGGAGAGATACCAAAAATC | | |
| | CGAATCCTCACAATATTACAGGAGCTATGGTAGGA | | |
| | GGACCTGATAAGTTTGATAAGTTCAAAGACGCGCG | | |
| | CAAAAATTTCAGCTATACAGAGCCAACACTAGCAG | | |
| | GAAATGCAGGACTAGTTGCTGCACTGGTTTCTTTAA | | |
| | CTAGCAGTGGTGGCTATGGTGTTGACAAAAATGCC | | |
| | ATTTTCTCAGCTGTTCCACCCTTATATCCAATGAGT | | |
| | CCACCCCCACCTCCCCCATGGAAACCATAATGTGC | | |
| | AAATTTTGCCTTGAAAACCTGCAGCAGCTTAAATTT | | |
| | TGCCTATATTTGGCTGGCTATATCCATGTACAAAA | | |
| | TTTCGAGAATAAAGAGTTGTTGTAACTCTGTTTATC | | |
| | TTATGACTCCGCGGCTTAATAAAATTCTTGCATTAA | | |
| | TTTCTTTTTAAAAAAAAAAAAAAAAAA | | |
| T324a | GATCTATCAA GTTTGCATGG TGGGTGCCCT | putative prolyl 4- | SEQ ID N° 510 |
| | GTGATTA | hydroxylase alpha | |
| | | subunit | |
| T327 | CTACCGAAGGGTACCTTGCAGAAGAAGGGGAGGA | expansin | SEQ ID N° 511 |
| | ATAGATTTACAATCAATGGGCACTCTTACTCCAAC | | |
| | TTGGTTCCCGTGACCAATGTTGGAGGTGCAGGAGA | | |
| | TGTAAGATCATTGTACATCAAGGGTTCAAGAACTC | | |
| | AGTGGCAACCAATGTCAAGAAATTGGGGCCAAAAT | | |
| | TGGCAGAATAACGCTTACCTCAATGGCCAAAGCTT | | |
| | ATCTTTCAAAGTCACCACAAGTGATGGTCGCACTG | | |
| | TGTTTCTTATAATGCAGCTCCTCATTCCTGGTCCTTT | | |
| | GGCCAGACTTTTACTGGAGGACAGTTCCGTTA | | |
| T328 | CCCTTATTGAGCAAAATCTCGAAGCTTGGGGGTAA | eukaryotic | SEQ ID N° 512 |
| | GGTATCTTCAGCCTCTTCGGTTCCGGAAGTGCCACT | translation initiation | |
| | GTGCCAGCCTGTTCCAGCCTTGGAAAAGCTTGCAA | factor 3 | |
| | CTCTGAGGTTGCTCCAGCAGGTATCTCAGGTGTACC | | |
| | AGACAATCCAGATTGGTAACCTGTCTAAGATGATC | | |
| | CCATTCATTGACTTTGCTGCTATTGAGAAGATCGCT | | |
| | GTTGATGCTGTTAGACATAATTTTGTTGCCGTTA | | |
| T330 | TAACCCCAAA GTTCAAGCAT CTATTGCTGC | BTF3b-like | SEQ ID N° 513 |
| | AAACACATGG GTCGTTAGTG GTTCCCCCCA | transcription factor | |
| | GACAAAGAAA TTGCAGGATA TCCTTCCTCA | | |
| | AATTATTCAC CAATTGGGTC CTGATAATTT | | |
| | GGAGAATTTG AAGAAGTTGG CTGAGCAGGT | | |
| | CCAGAAGCAG GGTGCTGCTG CAGGTACAGG | | |
| | TGAGGCTGCA GGTGCGGCCG CAGCACAGGA AG | | |
| T331 | TTCCAAAAGTACAACAGGTGTTAGAAGTGCGTTCG | RNA polymerase | SEQ ID N° 514 |
| | GTTGATTCAATATCCATGAATCTAGAAAACAGGAT | beta″ | |
| | TGAGGGCTGGAACAAATGTATAACAAGAATTCTTG | | |
| | GAATTCCTTGGGGATTCTTGATTGGTGCTGAGCTAA | | |
| | CTATAGCGCAAAGCCGAATCTCTTTGGTTACTCAGG | | |
| | ACTCATCAAGAGACCCCCCGGGGAATCCCGAGAAT | | |
| | TCTTGTTATACATTTGTTCCAACCCTCAATCCTCTTT | | |
| | TT | | |
| T332b | GATCTGGGTG AGGCAAAGAA AATTCTTGGC | light harvesting | SEQ ID N° 515 |
| | ATGGAGATAA TTAGAGATAG ACATTCAAAG | chlorophyll a/b- | |
| | AAACTCTGTT TATCTCAGAA AGAATATTTG | binding | |
| | AAGCGAGTAC TACAACGTTT TGGCATAGAT | | |
| | GACAAGACTA AGCCAGTTAG TACTCCACTT | | |
| | GCTCCCCATG TTA | | |
| T333b | CCAGCCGCAC CCTCACCACC AAAACTCCAT | putative outer- | SEQ ID N° 516 |
| | CGTCGGACCT CCCTTCACTA CGCAATAGCC | membrane protein | |
| | ATAAATGAAA CTTCACCTCA CACATGCCCT | | |
| | AAGCTCTTCT TCTTCATTGA CAGACCCAGA TC | | |
| T335b | GATCTGGAAC ATGACACAGC TGAGGCGTCT | disease resistance | SEQ ID N° 517 |
| | GCGTCTACTG AGTAGAAACT ATTTGTGTAA | like protein | |
| | GCCTAAA | | |
| T336a | GATCTGGAAA CCCCAAAAGT ATAGAAGCAA | ambiguous hit | SEQ ID N° 518 |
| | TTCTTGAGGT TGAGGATATC ATATAAACTA | | |
| | CTGTACCATT GGATTTCTTT CCATAATTCT | | |
| | TGAGGTTGAA TATCTCAGGC AATCTTTGAT | | |
| | TCATATGGGA A | | |
| T336c | GATCTGGGGA ATACTGACTT AGTGACTTAC | putative protein | SEQ ID N° 519 |
| | AATGTTATGA TGAACCTATA TGCTAAAATG | At5g09450 [A. | |
| | GGAGACCTTG AGAAACTACA GTCGTTAGTG | thaliana] | |
| | CAAGAGATGG AAGATAAGGG AATTGC | | |
| T337 | TAAGGAGAAA CAGAGAAGGA AACTACTGAG | putative protein | SEQ ID N° 520 |
| | AAATGATAAT GCAGAAAACA CACCAATACT | At3g52110 [A. | |
| | | thaliana] | |
| T339 | TGGTGTCGGAAGAATACACATGGCACCATTTAGTG | putative arginine | SEQ ID N° 521 |
| | ACGAATATTTGTATATGGAAATAGGAAATACGGCG | methyltransferase | |
| | ACCTTTTGGCAGCAACAAAACTACTTTGGGGTTGA | | |
| | CTTGACACCTTTGCACAGATC | | |
| T340 | GGTTTCATCACTGGTTTTGACTTTGGAGCTTGATTT | proline | SEQ ID N° 522 |
| | AGTGGAGTTTTCATGCATAGAAATTTCTGAATTTCT | dehydrogenase like | |
| | TCTATTGGAAGCTTGAAGAATAGGAGAAGAGGCGT | protein | |
| | TCCTTTTCCTTGCCTATGTTTTCTCCTCAATCTCCTC | | |
| | CCCTTTTCATTCTCTGTTTTTCCGTCTTTCCCCAGAT | | |
| | C | | |
| T341 | TAATGGAGGGCAAGCTGAGGAGTGGAACTACTCTC | thymidylate kinase | SEQ ID N° 523 |
| | ATTGTTGATCGCTATTCTTATTCTGGGGTGGCATTT | | |
| | TCATCTGCCAAGGGACTTGATATTGAATGGTGTAA | | |
| | GGCCCCAGAAATAGGATTGTTAGCTCCAGATC | | |
| T349 | TGCCAACAGTTCTATGCACATTGGAGATGTCACAA | putative protein | SEQ ID N° 524 |
| | TCCCATATCAAATTGCACAAACAGGGCTCTGGGAT | AT4g24350 [A. | |
| | TGGCTGAAACCAAATGCAACTCTGGAACCAAATGA | thaliana] | |
| | TTTTGCTCAATTTGATTTCAAGAATTATAATGTGCC | | |
| | AAAAGGAGGGGATAACAAGTTGGGGCGTGTTGGTT | | |
| | ATAGCACGGAGCAGTTTTACTCAACTTCAGGGGAG | | |
| | GTCAATGTACCTCAGAGACCAGTTTGGTTTA | | |
| T35 | GATCTCCGTC CGAGTGAATA ATGCATTTCT | putative protein | SEQ ID N° 525 |
| | TTTGGCAGGC AATGAAGAGA ATCGGGTGGA | At1g70660 [A. | |
| | TCAAAAAGGT TTGGTTCTGA AATGTTGTAT | thaliana] | |
| | TTTGTAACTG GAGATTGGAG AAAGAACATT | | |
| | GTAGATGAAA ATGTATATAG CCTTATTGCT | | |
| | CAGATAGTAG CAACTGTTGT CTTA | | |
| T351a | TTTTCGACAAGCTTGATGAAGATGGTGATGGATTA | putative protein | SEQ ID N° 526 |
| | GTAAGTTTAGGTGAACTCAAAGGCCTTCTTGATAA | CG14861 | |
| | GATTGGAGCTTGTACAGATCACTACGCAGTCATAA | [Drosophila | |
| | GATC | melanogaster] | |
| T352 | GATCTGTTGA TGCAGATATG TGGCATGGGA | serine/threonine | SEQ ID N° 527 |
| | ATCAGGATTT GCTATCCTCA AACAATGTCA | kinase-like protein | |
| | CAATCAGTGT ACTAATA | | |
| T353b | GATCTGTCAT TGATGTTCAT TACTACAATC | glucan 1,3-beta- | SEQ ID N° 528 |
| | TTTTCTCTGG CATGTTTA | glucosidase | |
| T354 | TGTCACAATTCCATCTCAAGTCGCTCCAACTGGGCT | putative protein | SEQ ID N° 529 |
| | ATGGGATTGGCTGAAACCAAATGCATCTCTGGAAC | AT4g24350 [A. | |
| | CAAATGATTTTGCTTAATTTGATTCCAAGAATTATA | thaliana] | |
| | GTGCACCAAAAGGAGGGGATAACAAGTTGGGGCG | | |
| | TGTTGGGTATAGCACTGAACAGTTTTATTCAACTT | | |
| T356 | TAACTGAGGC ACAAATGATT GACCACATGT | glycine | SEQ ID N° 530 |
| | CAAAATTAGC TTCAATGAAT AAGGTTTTCA | decarboxylase | |
| | AGTCATATAT TGGGATGGGA TATTATAACA | multi-enzyme | |
| | CTTTTGTACC ACCTGTTATT TTGAGGAATA | complex P subunit | |
| | TTATGGAGAA TCCTGCTTGG TATACTCAGT | | |
| | ATACTCCTTA TCAGGCTGAG ATTTCGCAGG | | |
| | GACGTCTCGA GTCCCTGCTA A | | |
| T357 | CCATTCTTCTCATTTCTGATGTATTTGGATATGAAG | endo-1,3-1,4-beta- | SEQ ID N° 531 |
| | CTCCACTTTTGAGGAAGATAGCAGATAAAGCCGCA | D-glucanase | |
| | GCTGCAGGGTACTTGGTGGTTGTTCCTGATTTCTTC | | |
| | TATGGTGAACCTCTTGATCGCGAGAAACATAACGT | | |
| | ACAGACATGGTTA | | |
| T36 | TGACGTGCGT AGAGATCTCC GAGATTATCT | putative protein | SEQ ID N° 532 |
| | AGATAGTTTC CATGGGCTGG GACTTTTCCT | AT5g13800 [A. | |
| | CTTCCCACCA CTATCAGAAA GCTCACAGAA | thaliana] | |
| | CTTGTATGGC AGAAAATTAG TGCTCCCGAG | | |
| | AGCATTGCGG AGGTGCTTA | | |
| T361 | TAAGCACCAC AATTTGCAGC TGTTACCAGT | photosystem II D2 | SEQ ID N° 533 |
| | CGATCGCGAT CGCGCCTACA TGCGCAGACT | protein | |
| | TCCACATCTG TACCATTGTA CCATAGTAAC | | |
| | CTTGTTCTGT CTCTTTGTTC ACTTAGAAAT | | |
| | GCTATAAATA CTGCATACAG ATGACTATAC | | |
| | ACATTAGCTG ACGCTTGATC ATTCATTGAG | | |
| | GAACCTTGTG GTTTCCACAA TTTTTCACTA | | |
| | AGCAGTCGGC ACATGATGTG TTAGTCAATC | | |
| | CCATATGGCA CTCAAATACT GTGTGCCGTA | | |
| | CATATGGAAT AGGGAACTAA GAGAGTTACA | | |
| | TACGGGAGAT CAATAAGGGC TCAGCAACAG | | |
| | GAGTGTCTTC A | | |
| T362 | ACGATGTGCT CCCGGTCCCG AGTGTCTCGC | 14-3-3 like protein | SEQ ID N° 534 |
| | GCAGTGTGTC ATCCTCAAAA CCAGCCTTGG | | |
| | GTAAAAATGA CAGGTAGGAT GACAATGTTA | | |
| | TGTTATTGTT GGACTTGTGG GAAGTAGTTT | | |
| | GGTCCTTTGA ACTTTGTTGC CGGAAAAGCT | | |
| | ATCTAAAGCA CTTTCTGATT TGGGCTTTCA | | |
| | GGACTTCAGG TCATTTATTC CGCCTTA | | |
| T364a | GATCTGTGGA AAAGGAAAGC TGGAGAAACT | NADH | SEQ ID N° 535 |
| | TGCTGTGCTG TAATTTATGT ACAGTGCTAT | dehydrogenase-like | |
| | TTGGCTGCTC AACTAAGATT GTTTTGATTC | protein | |
| | TCTCTTAGTC TTATGTTATC TTTTTTCTTG | | |
| | AAAATCCTTG CTTTTTCTTT CTTCTCTTGG | | |
| | AGTTGGGGGT CAATATCCTT TGTTTGTGGT G | | |
| T364c | GATCTGTGGA ATGCAATTGG TTCGTAATAT | B12D-like protein | SEQ ID N° 536 |
| | CTGCGGCAAC CCTGAAGTCA GGGTGACCAA | | |
| | GGAAAACAGG GCAGCAGGGG TACTGGACAA | | |
| | TTTTTCAGAA GGGGAGAAAT ATGCTGAGCA | | |
| | TGCTCTTAGG AAGTTTGTCC CCTTCTGTAA | | |
| | AGTTAGCATT TTCTTCTGCT TCCCCGTTTT | | |
| T365a | GATCTGTCGA ACCAGAGTTG GAAATGGAGG | putative protein | SEQ ID N° 537 |
| | AAGAGGATGA TCCTTATCCT CCATCCACTG | At4g11570 [A. | |
| | TGGCCGTTGA TGATGGTTTC TGGTAACATC | thaliana] | |
| | TCTGCAATGT ACAGTAGTTG TGCTTACTCA | | |
| | GGACTGATCG TCTAAGGACT TTTATGAGAC | | |
| | ATTCTCGTGT GTTACAATAC AAATATGACA | | |
| | TCTTTGCCTT A | | |
| T366c | GATCTGTACA AGCAAGACTG GATTGGGAGA | phospholipase D | SEQ ID N° 538 |
| | GGAGGACTAT GCGAATGATG TACACTGACA | | |
| | TAATTCAAGC TCTAAAAGTA AAGGGAATTG T | | |
| T367 | GATCTGTTCT TCAATATAAC AGAACGTCTT | putative protein | SEQ ID N° 539 |
| | TTTTCCTTA | ORF 1901 | |
| | | [Nicotiana tabacum | |
| | | plastid] | |
| T368 | TAATGCTCTC TCTGCACATA CTGGTACATA | putative glyoxalase | SEQ ID N° 540 |
| | AATAATAATA TTACAAAAAA GGATTTTTAC | | |
| | GGTATGTTTG GGTTGTTGGA AAAGGGGTCT | | |
| | AAATTTATGA GGGGTAAAAT CACTCTTTTT | | |
| | GCCGACAATA TCACTCAAAA ACAAATATCT | | |
| | ATCATGTCCA AAGCTAAATT TTCCATCATC | | |
| | AGAGATTCCA CTTCTCGTGA GCAGTTCATA | | |
| | TTTGCACCTC TGCTTCCATT TTCGTGAATG | | |
| | AAATTAGGCA TTGT | | |
| T369 | GTAATATCTGCGGCAACCCTGAAGTCAGGGTGACC | B12D protein | SEQ ID N° 541 |
| | AAGGAAAACAGGGCAGCAGGGGTACTGGACAATT | | |
| | TTTCAGAACGGGAGAAATATGCTGAGCATGCTCTT | | |
| | AGGAAGTTTGTCCGAAACAAGTCTCCGGAGATTAT | | |
| | GCCATCTATCAACGGCTTCTTTAGCGATCCAAAGTG | | |
| | AAGTTTGACATGGATTA | | |
| T37 | GATCTCCAAG CCTAGCTCCA GCACCAGCAC | fasciclin-like | SEQ ID N° 542 |
| | CAGGTCCCGA ATACACAAAC CTAACCGACT | arabinogalactan- | |
| | TACTCTCCGT TGCTGGCCCT TTCCACACAT | protein 7 | |
| | TCCTTA | | |
| T370a | GAAAAAGGGA GAAAAAGACT ACACTTAGGA | putative ankyrin | SEQ ID N° 543 |
| | GCACGTTATT CGCCTATTTG AAGCTAAAAA | protein | |
| | CCTACCCCCA CATCTGAAAA GATCGGGAAT | | |
| | CGAGGATATA TACAGATC | | |
| T370b | GATCTGTCAA AGGCCAAGTA TTTCACAGAT | putative acetone- | SEQ ID N° 544 |
| | GAAGGGTTTG GATCAGTGAA GAGAGTTTAC | cyanohydrin lyase | |
| | ATTGTGTGCA CAGAGGATAA ATGGATACCA | | |
| | GAAGAATTCC AACGATGGCA AATTGACAAC A | | |
| T372b | TAATGCACCA CTAAACAAGC ATGATAGGAG | putative 12- | SEQ ID N° 545 |
| | TACTTTCTAT ATGACAGATC | oxophytodienoate | |
| | | reductase 2 | |
| T372c | GATCTGGAAA GGTGGGTGTA TTATCAGGGC | 6-phosphogluconate | SEQ ID N° 546 |
| | AGTGTTCTTG GATCGGATTA | dehydrogenase | |
| T39G | GATCTCCAAC TGAAATGAAA TGAAGAGGAA | maturase | SEQ ID N° 547 |
| | GACGATGAGT CCTGAGTAAT GTCAGGGGAG | | |
| | GAGGACTTGG GATCGCGTAA AACACAGACA | | |
| | TCGCCATTGC AGACGAATTC GCCAGAGTCT | | |
| | GAGGACTCAG GTGAGAAGCA GCTACAGAAG | | |
| | TTGAACAAAG CCATAGTAGG AATTGAACCT | | |
| | AAGTAAATTA TATATCCCGA TCAAAGAGCT | | |
| | GACGAAAGGA ATGAGCAGAA CGTGGAGTGT | | |
| | AGTGGATATT ATTCGACTAA CGAAGACTCT | | |
| | TGGAATAGTT AGAGTAAAAA GTTCCCAAGA | | |
| | GAGCGTCTTT ATGGCGCGCG TCAATCACAT | | |
| | ACAACAAGGA TCAAGGGAGA TCACTACGCA | | |
| | GTCAA | | |
| T401 | TAACACATAC ACACGCATAA CTCACGAAGT | iron(III) ABC | SEQ ID N° 548 |
| | GGCACGTGTA AAAAAGAATT CCATCGAAGT | transporter-like | |
| | GTTCGAAATT CAAAGGACAC AAAAATCTCT | protein | |
| | CTCTAAAAAT TCTTGAAAGA GCTGGTGGAT | | |
| | GAAACAGATT CTCTTACAAA CACTTTCAAT | | |
| | TCAGACGTAC GATAATTAGC GTGAAGACTT | | |
| | GAAAAGTAGC CACTGCAAAG GAAATGATCC | | |
| | CATTACTGTT AACAAAGGCA TATTC | | |
| T402 | AGAAAAAGTCCGATCACCGGGCGAGGAGTCCGAC | phenylalanine | SEQ ID N° 549 |
| | AAAGAGTCCACACGCAATGTGCAATGGACAAATCA | ammonia lyase | |
| | TTGATCCAATGTTGGAGAGTCTCAAGAGCTGGAAT | | |
| | GGTGCTCCTCTTCCTATCTGTTAGTTGTTTTGCTTGAT | | |
| | TTCGCGCGGCGGGAACTTTTGTTA | | |
| T404 | GATATTCTTGGTGGAGTTTTAGCTGCGTTATGATAC | fatty acid 9- | SEQ ID N° 550 |
| | TTTTGAAATTGAATTTGGAAAGCTCCTGCTTGGTTC | hydroperoxide lyase | |
| | TAAGGTGACTTTCAAGTCAGTAACCAAGGCAACGT | | |
| | CTTA | | |
| T405a | GATCTTAGGG CAGGGCATGA ACAAAGTCTA | lipoic acid synthase- | SEQ ID N° 551 |
| | TCTGTGCTTA | like protein | |
| T405b | GATCTTAGAG TGTCTAGGGT TGGGCCAGGA | putative protein | SEQ ID N° 552 |
| | GGGTCTCTTA | tRNA-Ile [Spinacia | |
| | | oleracea] | |
| T406 | GGACCTGATACGGATACGACAGCCTTTTGGGAGTC | putative protein | SEQ ID N° 553 |
| | GGCGCAACATAGGCCCTTTGTTCTCCAAAACTATAC | At2g36290 [A. | |
| | TCTGGGGCTTGTTTAGTATTGGATTCAATGACTCTT | thaliana] | |
| | TGTTATTGTACAAATTTGAATATTTGTCAATATTAT | | |
| | CAAATGATTGTTTAGTTGCTTTATTCAAGTAATGAA | | |
| | TGGTTATGTGTTA | | |
| T407 | GTTTGAAGATGAAACGTTTGATTTGGAATTTTCTCC | putative protein | SEQ ID N° 554 |
| | TGTTTTTGACCCCGCGCTTTATCCGGAGAAATATGT | At1g24480 [A. | |
| | GTCGGAGATCGAACGGACGTTGAAGGCCGGAGGG | thaliana] | |
| | TTTGTGTTTTGCACGTGGCGTTATCTAGACGGGCTG | | |
| | ATAAGTATTCGGCGAACGATTTGTACAGTGTTGAG | | |
| | CCGTTGAAGAAACTGTTTA | | |
| T408 | GATCTTGAGT TCAATTCCAA AGCCATTTAC | uracil transporter- | SEQ ID N° 555 |
| | CATTATTTAC AACAATGCTT GGTTCTTTAG | like protein | |
| | CTTGTTTTTA GCAGGGGGAC TTTATTGTAT | | |
| | TCTTTCATAT TTGAAGGGGA AAAAGAAAAA | | |
| | TCAAAAGCAC GTAAATCCTT TGCTGCCTAA | | |
| | TGCATCTTAG TGATGTCTCT | | |
| T409a | GCGCGAAACGCGCTATCTGTCGGGGTTCCCCCGAC | hemolysin | SEQ ID N° 556 |
| | CCTTAGGATCGACTAACCCATGTGCAAGTGCCGTTC | | |
| | ACATGGAACCTTTCCCCTCTTCGGCCTTCAAGGTTC | | |
| | TCATTTGAATATTTGCTACTACCACCTAGATC | | |
| T409b | GATCTTGGCC TGTTGACAGA TTTAGCCGTT | putative protein | SEQ ID N° 557 |
| | TTTCATATAA ACTCCAATAG ATTTTCAGGC | At1g49490 [A. | |
| | ACTATCCCAA AATCCTTTTC TAAGCTCCAA | thaliana] | |
| | CTTCTCTATG AACTTGACGT GAGTAACAAT | | |
| | CTTTTGTGTG GTGAATTTCC TTCGG | | |
| T409c | GATCTTGGAC CCAGAAATAT GCCATGGGAT | ubiquitin | SEQ ID N° 558 |
| | GAAAACATTT GGCTTTACTC CCATGAACAT | conjugating enzyme | |
| | CGGGCCTTTA TGCTATAGTA GTAAATAAAA | | |
| | ATAGGCGCGG AGCACAATTT TCTGATATTG | | |
| | GTGTCTTTTG TTATCTGACG TTGTGTC | | |
| T410 | GAGAGAGCTAGAGCGTGGCGTGAAATGTATTTCTT | berberine bridge | SEQ ID N° 559 |
| | GCATAACTATGATAGGTTGGTTC | enzyme | |
| T411c | TCTTGACCAA GATTGACAGG CGT | elongation factor-1 | SEQ ID N° 560 |
| | | alpha | |
| T414 | GATCTTGAAGACTTCTGTGCTTTCCTTTAGTGGCTT | hexameric | SEQ ID N° 561 |
| | TTGTTGTGCTCTGTGTTTA | polyubiquitin | |
| T418 | TCTTCCTCTGTTGATGCTGTGGAGAGAGCTAGAGCG | berberine bridge | SEQ ID N° 562 |
| | TGGGGTGAAAAGTATTTCTTGCATAACTATGATAG | enzyme | |
| | G | | |
| T419 | TGCAGCGATTGCTGGGTTTGAGGTAACTGTCTTGG | collagen-like | SEQ ID N° 563 |
| | CTTAGTAATGCAATTAGTAGTGTCAGACCCTTGTAC | protein | |
| | TAGCTCCGGAACATGAATCTTATATGTATTTATTCA | | |
| | AAGAACATTGCGACAAATCTTTGTTATGAATTGTCT | | |
| | TTCTGTGCGTTGTATGTTTCCTTTGGGTGTATTTCGT | | |
| | ACGAAGGAAATATTTTCCACGAAAAATATTTCCTA | | |
| | GAAAATAAATGGTTTGCTTA | | |
| T420a | GATCTTGCAC TGTAAACACA GTACTTTGGA | putative protein | SEQ ID N° 564 |
| | ATACAATTCA ACTTCTGTTT CCTAAAGAAA | At3g27330 [A. | |
| | TAGAAGCAAG AAAAGCAGCT GGAGCTTTGA | thaliana] | |
| | ATAGTAGAGA AGCTCGACGC AAAAGTCCAG | | |
| | TAAGAGCTGC TACAGCTCAT TCTAACATCT | | |
| | CTAGCAGCAG AAT | | |
| T420b | GATCTTGGCT GCAAGTGGGT CATTCTTGGT | putative | SEQ ID N° 565 |
| | CATTCGGAGA GGAGACATGT AATTGGAGAA | triosephosphate | |
| | AATGATGAAT TTATCGGCAA GAGGGCTGGG | isomerase | |
| | TATGCTTTGA GGCAAGGTGT TGGTGTTATA | | |
| | GCCTGTATTG GAGAGC | | |
| T421 | TGTGTTAGGCTTGGCAAAGCCGAAACCCTTCCCAC | high-affinity nitrate | SEQ ID N° 566 |
| | AGCCATTGTGGCCATCCTCTTGTTCTCCCTTGGAGC | transporter | |
| | TCAAGCTGCATGTGGCGCTACCTATGGTGTCATCCC | | |
| | TTTCGTGTCGCGAAGATGACTAGGCTTA | | |
| T422c | GATCTTGCCA TGGACGTAAT TATCAACAGC | wound-induced | SEQ ID N° 567 |
| | AGCCATATTG GGTCCTG | WRKY-type | |
| | | transcription factor | |
| T423 | TGACTGCGTAGTGATCTTGATGGTGAATTGACCTTG | annexin | SEQ ID N° 568 |
| | AAACAAGTTGTTCAATGCCTTTGTTCACCTCAAGCC | | |
| | TACTTCAGCAACATATTGATCGCGTCCTTA | | |
| T424a | GATCTTGAAT ACTATTCGAA ATTCAGAAGA | H+-transporting | SEQ ID N° 569 |
| | ACTGCGTGGA GGGGCTATTG AACAACTCGA | ATP synthase I | |
| | AAAAGCTCGT TCTCGCTTAC GGAAAGTAGA | | |
| | AAGCGAAGCC GAGCAGTTTC GAGTGAATGG | | |
| | ATACTCTGAA ATAGAACGAG AAAAATTGAA | | |
| | TTTGATTA | | |
| T424b | ACAGCTATGA CCATTAGGCC TATTTAGGTG | auxin-induced | SEQ ID N° 570 |
| | ACACTATAGA ACAAGTTTGT ACAAAAAAGC | protein | |
| | AGGCTGGTAC CGGTCCGGAA TTCCCGGGAT | | |
| | GAAATCACAA CAATGGCCAA AGAGGGAACA | | |
| | AAAGTGCCAA GAATCAAATT GGGTTCACAG | | |
| | GGGCTAGAAG TGTCAGCTCA AGGACTTGGT | | |
| | TGTATGGGTA TGTCCGCTTT TTATGGGCCG | | |
| | CCCAAACCCG AGCCCGATAT GATCCAACTC | | |
| | ATTCACCATT CCATCAACTC TGGTGTCACC | | |
| | TTTCTTGATA CATCAGATGT GTATGGGCCC | | |
| | CACACCAATG AAATCCTACT TGGCAAGGCG | | |
| | TTGAAGGGAG GGGTGAGAGA ACGAGTTGAG | | |
| | TTAGCAACAA AATTTGGAGC TATTTTTGCA | | |
| | GATGGAAAGA TAAAAGTGTG TGGAGAGCCA | | |
| | GCCTATGTAA GGGCAGCATG CGAGGCTAGC | | |
| | TTAAAGCGAC TTGATGTTGA CTGCATTGAC | | |
| | TTGTACTACC AGCACCGAAT TGATACACGC | | |
| | GTGCCTATTG AAGTCACGGT TGGAGAACTT | | |
| | AAGAAGCTGG TTGAAGAGGG TAAAATAAAA | | |
| | TATATAGGTC TATCCGAGGC ATCAGCATCG | | |
| | ACGATTAGAA GAGCACATGC AGTTCATCCA | | |
| | ATAACAACAG TACAATTAGA ATGGTCTCTA | | |
| | TGGTCTAGAG ATGTAGAGGA AGAAATAATC | | |
| | CCTACTTGCA GAGAACTCGG TATTGGGATT | | |
| | GTGGCATACA GTCCACTAGG ACGGGGATTT | | |
| | TTGTCATCCG GTCCAGAGCT GCTTGAAGAT | | |
| | TTGTCAAGTG AAGATTTCCC AAAGCATCTC | | |
| | CCAAGGTTCC AGGCTGATAA TCTTGAGCAT | | |
| | AACAAAATAT TATATGAAAG AATTTGTCAA | | |
| | ATGGCGGCAA AGAAGGGATG TACGCCATCT | | |
| | CAACTAGCCT TGGCTTGGGT ACATCACCAA | | |
| | GGAAATGATG TGTGCCCCAT CCCAGGTACC | | |
| | ACAAAGATCG AAAACCTCAA CCAAAACATT | | |
| | GGAGCTTTGT CAATTAAGTT AACAACAGAA | | |
| | GACATGGTGG AACTTGAATA CATTGCTTCA | | |
| | GCTGATGCAG TCAAAGGTGA AAGAGATGCT | | |
| | TCTGGTGCAA ATCACAAAAA CTCTGATACT | | |
| | CCACCATTGT CAACTTGGAA GGCTACGAGA | | |
| | TAAGATTTTC GCGCACTTTC CACGTTACAA | | |
| | TGTATCTGAA ACATGTTCTT GTTGGAAATA | | |
| | GTAAATATTA TAAAAGTTTA AACAAGTGTC | | |
| | TAGGCTCATT TGTACTGTCG AGTCATCCCA | | |
| | GAATATTCAC TAATCATTGT TCATATAACT TG | | |
| T426b | AGTGATCCTC AAGCATTAAT TTGCCACTTT | heme oxygenase | SEQ ID N° 571 |
| | TACAACACAT ACTTTGCGCA TTCAGCTGGA | | |
| | GGTCGCATGA TAGGAAGAAA GGTGGCTGAA | | |
| | AAAATACTCA ACAAGAAAGA GCTGGAATTC | | |
| | TGACTGCGTA GTGATCTTGG AGTGAATATG | | |
| | GACGACGACT ACTTACTGCG AAATGCTAGT | | |
| | AGTCGGTAAT TCTTCTTCCT CTGTTGATGC | | |
| | TGTGGAGAGA GCTAGAGCGT GGGG | | |
| T426c | GATCCGGGTC ACTTCCCTAC ATTGGGTGGC | probable | SEQ ID N° 572 |
| | AAGTGATGCT TTATTAGTGC TTTTCTCCCA | transcription factor | |
| | CGTCCAAGAG GCAAATTGAC TGAAAAATAA | | |
| T429c | GATCTTCTAACAGTAAATGAAATATGTTGCGACAC | helicase-like protein | SEQ ID N° 573 |
| | ATTTAGAGAATCTGCAGAAAAAAGAGGGTTGTTAC | | |
| | ATTGTGATAACAACTTTGATTGAATGTATGTTAGAG | | |
| | GCTGCATGTTATCAAATGCCTTATAGTTTA | | |
| T430 | CCTACATTGGTCCTCGCCATAACGTATTGGATGACA | putative ABC | SEQ ID N° 574 |
| | GGGCTAAAGCCCCAACTCTAGCCATTCCTTTTGACA | transporter | |
| | CTGCCCGGCCTGCTGAGCTATGTGATTGTTTCACAA | | |
| | GGCCTCCGGTTAGCCCTTGGCGCCTTGATCATGGAT | | |
| | GCTAAACAAGCTTCAACTGTGGTCACTGTCACCAT | | |
| | GCTAGCATTCGTTCTAACAGGAAGGTTCTACGTGC | | |
| | ATAAAGTGCCAGCTTGTGTAGCTTGGATTA | | |
| T431 | GACTGGAATGGCTGATCGTAAGATCGCAATGCCAG | beta-glucosidase | SEQ ID N° 575 |
| | ATGCCATCCCGGATCGTCAGAGAGTGAACTTTTATC | like protein | |
| | GTGGGCACCTTTCGGCAGTTCAAGAAGCCATAGAG | | |
| | CTCGGTGTGAAGATTA | | |
| T432a | GATCTGCAAA CAATGACTGG AAATCTCTTA | phospholipase D- | SEQ ID N° 576 |
| | CTCAGGTAAA GGAGGTAGGA ATATATCTCG | like protein | |
| | CTGGTTGCTC AGATATAGCA AAAAAGGTTG | | |
| | AAATCTACTA TGACAACCTT TGGAAACTTG | | |
| | CCCACCTTGA TGTTCCAGCT TACACAAGAT | | |
| | CAGTTTGGGA TTCACAGTGG CAGATTA | | |
| T434 | AATACGACTC ACTATAGGGC GAATTGGGCC | putative SGP1 | SEQ ID N° 577 |
| | CGACGTCGCA TGCTCCCGGC CGCCATGACG | monomeric G- | |
| | GCCGCGGGAA TTCGATTCTG ATCTCGGCGG | protein | |
| | CGAATTTGCC CCAACTGCAG CAGCAGCTGC | | |
| | TATCTCTTCC TCTATCTTGT GTTTGTGTGC | | |
| | ATGCTGTGGA TCAGTACCCC GTCTACGCAA | | |
| | CTGCAGCAGC AGCTGCTATC TCGTCCTNTT | | |
| | GCTGACTGCG TAGTGATCTT CAAGTTCATT | | |
| | ACAGCAAAGC TCTTCAATTT GCCATGGACA | | |
| | TTGGAGCGTA ACCTTACCAT TGGAGAACCA | | |
| | ATTATTATTT TTAGGTTT | | |
| T436a | GATCTTCACA GTAGCATCAG GTCATACTGA | subtilisin-like | SEQ ID N° 578 |
| | CAGGTGGTTT TCCGGGACTC TGACACTGGG | proteinase | |
| | AAGTGGTCTA AAGATTA | | |
| T438c | GATCTTCAAA TTTCTTTGAT TCTAAAGTAA | N- | SEQ ID N° 579 |
| | TGAAAGAAGC ATTA | hydroxycinnamoyl/ | |
| | | benzoyltransferase | |
| T439 | GATCTTTACG GGCCCTATTT ATTCTTCAAA | acyl CoA reductase | SEQ ID N° 580 |
| | GGAATATTTG ATGACATGAA CACAGAAAAA | | |
| | TTACGTAGAG CAGCGAAGGA GGCTGGTATT | | |
| | GAAATAGACG TGTTCAATTT TGATCCCAAG | | |
| | AGCATCAACT GGGAGGATTA TTTTATGGAC | | |
| | ACTCACGTAC CTGGCGTTGT AAAATATGTA TTTA | | |
| T440 | GATCTTGGAG TGAATATGGA CGACGACTAC | berberine bridge | SEQ ID N° 581 |
| | TTACTGCGAA ATGCTAGTAG TCGTAATTCT | enzyme | |
| | TCTTCCTCTG TTGATGCTGT GGAGAGAGCT | | |
| | AGAGCGTGGG GTGAAATGTA TTTCCTGCAT | | |
| | AACTATGATA GGTTGGTTA | | |
| T441a | GATCTATACC AGAAGGAGCT GTTGTATGTA | 60S ribosomal | SEQ ID N° 582 |
| | ATGTGGAGCA TAAAGTGGGA GATCGTGGTG | protein L2 | |
| | TTTTTGGTAG ATGCTCTGGT GATTATGCCA | | |
| | TTGTGATCAG CCACAACCCT GATAATGGTA | | |
| | CCACTAGGGT TA | | |
| T442 | AAAACACCAATTGTCTGTAAACCTTCAGAAATCGC | ripening-related | SEQ ID N° 583 |
| | CATTGAACGCGCTTTA | hydrolase-like | |
| | | protein | |
| T443 | CCTAAATCTATCAATATGGATGAAAGTTTGGGGGT | cytochrome P450 | SEQ ID N° 584 |
| | TACAGCGAGAAAACGCCACTCTTTGAAAGTAATAC | hydroxylase | |
| | CAAAAAAGGCTTGAGAACTTACGTATTTGAGTTTTC | | |
| | ATAGTTATGTTTTGTGCATATTTTCTTACTTATATTT | | |
| | GGAGTAAACCAGTATTCCTGTTGTGTTATGAACAA | | |
| | GTTGTAGTGCTGCCTACTGGAGTTTGTGTTA | | |
| T446a | GATCTTTACA AGGCAGCCGG GGGATTCAAG | receptor like protein | SEQ ID N° 585 |
| | GTCAGTGAAC TAATTGGAGT TGGAGGCTTT | kinase | |
| | GGTGCTGTTT ATAAGGGTAT TTTGCCTACT | | |
| | AATGGAGCTG AGGTTGCGGT GAAGAGGATA | | |
| | GCAAGCAATT CTCTTCAAGG AATGAGAGAA | | |
| | TTTGCAGCGG AGATTGAAAG CTTAGGCAGG TTA | | |
| T446b | CGACTGGGTAGGGATCTTTGAAGCCGCTAGCAATC | lipoxygenase A | SEQ ID N° 586 |
| | GAACTAAGTTTGCCACATCCAGATGGTGACCAATT | | |
| | TGGTGGCATTAGGAAAGTGTATACCCCAGCTGATC | | |
| | AAGGTGCCGAGGGCTCCATCTGGGAACTGGCTAAA | | |
| | GCTTATGTTGCAGGGAATGACTCAGGTGTTCATCA | | |
| | ACTAATTAGTCATTGGTTA | | |
| T447 | GATCTTTGCAAGGATTTCTGCAAAAGAGAAAGAAT | putative protein | SEQ ID N° 587 |
| | AGAATTCAAGCAACTTCCCCATATCATCACTAGCT | At2g34600 [A. | |
| | CTAACAATTATATTACTAATAATATGTGATGATCTT | thaliana] | |
| | CTATTTCTTTTTACTTTCATTATTTTACTTCTCCTAG | | |
| | TGTGGCTA | | |
| T448 | GATCTTGGAGTGAATATGGACGACGACTACTTACT | berberine bridge | SEQ ID N° 588 |
| | GCGAACTGCTAGTAGTCGTAATTCTTCTTCCTCTGT | enzyme | |
| | TGATGCTGTGGAGAGAGCTAGAGCGTGGGGTGAAA | | |
| | TGTATTTCTTGCATAACTATGATAGGTTGGTTA | | |
| T449 | GATCTTTTCT GGCCAACTCG GGAACCTACA | putative integral | SEQ ID N° 589 |
| | GCTTGCAGCA GCCTCTCTTG GCAATCAAGG | membrane protein | |
| | CATCCAATTA TTTGCTTATG GCCTTATGCT | | |
| | AGGAATGGGC AGTGCAGTGG AAACGCTTTG | | |
| | TGGCCAAGCA TATGGAGCTC ACAGATATGA | | |
| | AATGCTAGGA GTCTACCTGC AAAGAGCAAC | | |
| | AGTAGTACTT TCCTTA | | |
| T454 | GATCTGTGGA ATGCAATTGG TTCGTAATAT | B12D protein | SEQ ID N° 590 |
| | CTGCGGCAAC CCTGAAGTCA GGGTGACCAA | | |
| | GGAAAACAGG GCAGCAGGGG TACTGGACAA | | |
| | TTTTTCAGAA GGGGAGAAAT ATGCTGAGCA | | |
| | TGCTCTTAGG AAGTTTGTCC GAAACAAGTC | | |
| | TCCGGAGATT ATGCCATCTA TCAACGGGTT | | |
| | CTTTAGCGAT CCAAAGTGAA GTTTGACATG | | |
| | GATTA | | |
| T455 | AGTAATCCCA AAGTTTATCA ATCTAGCCAT | putative dTDP-D- | SEQ ID N° 591 |
| | GAGGGGGAAG CCTCTTCCTA TTCACGGAGA | glucose 4,6- | |
| | TGGTTCAAAT GTTAGAAGTT ATTTGTACTG | dehydratase | |
| | TGAGGATGTT GATGCGGCTT TCGAGGTTGT | | |
| | TCTTCACCGA GGAGAGGTTG GTCATGTTTA | | |
| | TAACATTGGA ACTAAGAAAG AGAGCAGGGT | | |
| | GATTGATGTT GCCAAAGAGC ACTACGCAGT CG | | |
| T461 | AAGATTGCGA GAAGTCAAAG AACTGAGGTC | putative 6- | SEQ ID N° 592 |
| | TTTTGATGTT TTCTTTTTAT TTGACCTAAT | phosphogluconate | |
| | TGCCTAAGGT TCTTCCCGTC ATTGAATCTG | dehydrogenase | |
| | GGAGGCTAGA TTCTGTAGTA TCTGTCATGT | | |
| | GGTCGCTCAA ATGTTGGAAC TTTACCTATA | | |
| | TTGTTGTGAA GCCTATTTGT ATCTTTA | | |
| T463a | GATCTTAAGT TATAAGTACG TTTCTTTTAT | chaperone GrpE | SEQ ID N° 593 |
| | TATTTTCTAT AT | type 2 | |
| T463b | GATCTCACCG GGAAAGTGCA CCAGCTGCCA | putative protein | SEQ ID N° 594 |
| | TGCTGTATCA AGTTCA | At2g39440 [A. | |
| | | thaliana] | |
| T464 | TAGCGGATAA CAATTTCACA CAGGAAACAG | epimerase/dehy | SEQ ID N° 595 |
| | CTATGACCAT TAGGCCTATT TAGGTGACAC | dratase-like | |
| | TATAGAACAA GTTTGTACAA AAAAGCAGGC | protein | |
| | TGGTACCGGT CCGGAATTCC CGGGATCTCT | | |
| | TTCTAATCTC TCCGCTGCCT CACTTTTCTC | | |
| | CTCCAAATTT TTAGAGAATG GGAAGCTCAG | | |
| | GTGGCATGGA CTATGGTGCT TACACCTATG | | |
| | AGAATCTTGA GAGGGAACCT TACTGGCCAA | | |
| | CCGAGAAGCT CCGTATTTCC ATTACTGGGG | | |
| | CCGGAGGATT TATTGCTTCC CACATTGCTC | | |
| | GTCGTTTGAA GAGCGAGGGC CACTACATAA | | |
| | TTGCCTCCGA TTGGAAGAAG AATGAGCACA | | |
| | TGACAGAAGA TATGTTCTGT CATGAGTTTC | | |
| | ATCTTGTGGA TCTTAGGGTT ATGGATAATT | | |
| | GCTTGAAGGT TACAAAAGAT GTTGATCATG | | |
| | TCTTCAACCT TGCTGCTGAT ATGGGTGGCA | | |
| | TGGGCTTCAT TCAGTCTAAC CATTCTGTTA | | |
| | TTTTCTATAA CAACACTATG ATCAGTTTCA | | |
| | ACATGATGGA AGCTGCTCGG ATTAATGGTG | | |
| | TCAAAAGGTT CTTCTATGCA TCTAGCGCTT | | |
| | GCATTTACCC CGAGTTCAAA CAACTTGAAA | | |
| | CAAATGTCAG TTTGAAAGAA TCTGATGCAT | | |
| | GGCCAGCAGA GCCTCAAGAT GCTTACGGCT | | |
| | TGGAGAAGCT TGCGACCGAA GAGTTGTGCA | | |
| | AGCATTACAA CAAAGATTTT GGAATTGAAT | | |
| | GTTGTATTGG AAGGTTCCAT AACATCTATG | | |
| | GTCCATTTGG AACTTGGAAA GGTGGAAGGG | | |
| | AAAAAGCTCC TGCCGCGTTT TGTAGAAAAG | | |
| | CCCAAACTGC AGTAGATAAG TTTGAAATGT | | |
| | GGGGAGATGG ACTTCAACCA CGTTCATTCA | | |
| | CCTTCATTGA TGAGTGTGTT GAAGGGGTTC | | |
| | TCAGATTGAC AGAGTCTGAC TTCCGGGAGC | | |
| | CAGTGAATAT TGGAAGTGAT GAGATGGTGA | | |
| | GCATGAATGA CATGGCTGAG ATGGTTATTA | | |
| | GCTTTGAGGA CAAGAAGCTT CCTGTCCACC | | |
| | ACATTCCTGG CCCAGAAGGT GTTAGTGGTC | | |
| | GCAACTCAGA CAACACCCTT ATAAAAGAGA | | |
| | AGCTTGGTTG GGCTCCGACA ATGAGATTGA | | |
| | AGGATGGTTT GAGAATTACA TACTTCTGGA | | |
| | TCAAGGAGCA GATCGAGAAA GAGAGATCTC | | |
| | AAGGAGTTAA TATTGCAAAT TATGGATCGT | | |
| | ATAAGGTGGT GGGCACTCAA GCTCCAGTTG | | |
| | AACTCGGTTC CCTTCGTGCT GCTGATGGCA | | |
| | AGGAATAAGT TCATCCCTTC TATTAATTGG | | |
| | AAGCCAAATC ACTGCTATGA CATTGCTGCT | | |
| | TTATTAATAT GGTTGTCGTA GGTGAATGTG | | |
| | TTAAATTTTC AGTAATTGTT GGCTTTTCTT | | |
| | GGTTTTGAAT CTTGTAATTT AAGCCCCTTG | | |
| | GCTTGTGGGG GGGATGGTTG GATGCTTCAG | | |
| | CTGTATTTAT CAGTTGTTTG AGAAGATCTA | | |
| | TATATGATAA TCCAATAATT GGCAAAC | | |
| T465 | TAATGGCAAA GGGATACAAC CAAAAGAAGG | putative | SEQ ID N° 596 |
| | GTATTGATTA TCAAGGAACA TTCTCATCCG | retroelement pol | |
| | TGGTGAAGAT GGTTACTGTA AGAGGACTAC | polyprotein | |
| | GCAGTCA | | |
| T8 | TAAACGTGGT GGATGTTTCT TATGGAGGAG | putative polypeptide | SEQ ID N° 597 |
| | AGAATGGTTT CAATCAAGCA ATTGAGTTGT | chain release factor | |
| | CTGCTGAGAT C | | |
| T9 | GCATCAGGAA CACACAAGAG AATACTGTAT | putative adenosine | SEQ ID N° 598 |
| | TACACAGGGT GCTGATCCAG TTGTTGTTGA | kinase | |
| | TGAGGATGGG AAGGTGAAAT TGTTCCCAGT | | |
| | TATTCCTTTG CCAAAAGAGA AACTTGTTGA | | |
| | CACCAACGGT GCTGGTGATG CATTTGTGGG | | |
| | AGGATTCCTT GCACAGTTAG TCCAAGGAAA | | |
| | ACCTATTGCA GATTGTGTCA AAGCAGGGTG | | |
| | TTATGCATCG AATGTCATCA TCCAAAGGTC | | |
| | TGGTTGAACA TACCTTAAGA AGCCCGATTT | | |
| | TGAATCACGG ATATTTCCAT | | |
| C168 | TGCAAAATGT TTGCACCTGA AAGAACACAT | putative protein | SEQ ID N° 599 |
| | TGTCCTTGAT GGATC | At3g52140 [A. | |
| | | thaliana] | |
| C187b | TAATCACAAA GGGTTGCTCA TCATAACTAA | putative protein | SEQ ID N° 600 |
| | TAGCTATGCA GTGATTGAGA CAAAGAATGA | P0469E09 [Oryza | |
| | TGGATC | sativa] | |
| C20 | TAATCCAAGT CCCTAGCATA AACACCAAAC | putative glutathione | SEQ ID N° 601 |
| | CCCAAAAATA ATTCACAATT CTACAGATAA | S-transferase | |
| | AAAAAAGGAC ATGACCAATT TATTTATCTA | | |
| | TTACTAATCA ACAATTCTGT AGAACTCCAT | | |
| | GACATACTTA TACAGCGCGT GCTTCATTCG | | |
| | CAGTTTCACC AATGGTTGAC AAACCGTTTT | | |
| | GAAATTCTAG GCCATAAAAC GGTTTTTGTA | | |
| | GTATTCAACC AGTCTCTTGG GATC | | |
| C307 | TAAAGCAAAG ACGTGGTTAC TACAATCTAC | metapyrocatechase- | SEQ ID N° 602 |
| | TTATGCATCA TAGAACTAAT GCATTCTCAA | like protein | |
| | AAGTGTATGG GGTCCTCGGA TC | | |
| C427b | TAAACAATAT TTGTAACATA AAAGTTTCAT | putative protein | SEQ ID N° 603 |
| | CTGCTAAAAT TGTGTGGAAG TGAGTACAGT | At5g12080 [A. | |
| | TTCTATTTGG AGGATCAC | thaliana] | |
| T108 | TAAACAGTGA TGATGATGAT GTGGGCATCT | putative protein | SEQ ID N° 604 |
| | CTGATGAAGA TGAAGAATAT TTCAGAAAGC | P0698A04 [Oryza | |
| | CTCAGGGCAA GCAAAAGAAT AGGGGTGGGC | sativa] | |
| | ATAGTGTAAA ATCTACCAGA GAAATTAGGT | | |
| | TTCTTGCTAC ATGTGCTCGA CGAAAAAGGG | | |
| | GTAGAACATC ATATGAAGAG GAAGAATCAT | | |
| | CAGAACATGA TTTCTGAAAA | | |
| T114a | TAAAGTTAGA TGGAAACGAA CCTTTGCTTG | guanine nucleotide- | SEQ ID N° 605 |
| | TAGCATTATG CATGGTAATA TTATATTGTT | exchange-like | |
| | CACTACACGT TCCTGATGTA GACCCACCTT | protein | |
| | CAAAAACGGG AGAGCGATCA TGTAGATCAA | | |
| | TACGCAGTC | | |
| T147b | TAACAGCATG TTCATTTTCA ATAACTCCTG | putative Athila | SEQ ID N° 606 |
| | TAATGCCTAT TCAACAAATG AAGTTTGAGC | retroelement protein | |
| | ATCAGTTGTT TCAGTGGATG CAGATGCATC | | |
| | TTTAGCTTCC GATGTGCCAG TTGATGATTT | | |
| | TCCTGCACCA CCCGTAATAA ATTTGGTAAT | | |
| | CAAATCTTCT AGATC | | |
| T42 | TAATCATAAA GTTTTGAGGA AGCACCTCAA | putative glucan 1,3- | SEQ ID N° 607 |
| | AAGATCAACT TGTAACAGCA TTGTGGAGAT C | beta-glucosidase | |
| T207a | TAACGATGTC AAAAAATTTC TGTCGGAGAC | phosphatidylinositol- | SEQ ID N° 608 |
| | AGAATCAGAG ATCATAATCC TCGAGATC | specific | |
| | | phospholipase-like | |
| | | protein | |
| T325b | TAAACGAGCA AAAGAATAAT AAGGGACTTA | urdine | SEQ ID N° 609 |
| | GCATACTGGT AGCAAGAACC CCAGATC | phosphorylase like | |
| | | protein | |
| T365b | TAAGCCGTAC ATCAAATTGG TATATATTGG | transcription factor | SEQ ID N° 610 |
| | TCACTCACAA AAGACTTTCT GTACCCTAAC | rush 1 alfa like | |
| | CTTGCCAATG GAAGTGGGCA ATGGTAAATT | protein | |
| | CGTGCGAGAA TCAAATTTCG ACAGATC | | |
| MC311b | GATCCGGAAC GAAGGCGATG AACCTGACTC | putative bZIP | SEQ ID N° 611 |
| | GATCGGAATA TAATATCACC GCAAATGACC | transcriptional | |
| | TCGACTCTCA AATGGCGACC TTGACCGCGA | activator | |
| | AACTACAATG ATTCAAACTC GAAAAATGCT | | |
| | CAATGATGTT CAACCTGCTT TATTA | | |

TABLE 2

Sequences with no homology

| | |
|---|---|
| Seq | Anno- |

| | | | |
|---|---|---|---|
| code | SEQUENCE | tation | SEQ ID N° |

| | | | |
|---|---|---|---|
| C103 | GATCCAAGATAGCCCTATAGGCGTCCGCATTCCCTGGCATCTC | No hit | SEQ ID N° 612 |
| | CCTCCATCCTTTCATCCTGTTTCATTTATGTAATTCAGAAACAG | | |
| | GGTTGTATTTATTTTTGGACCTTGTTTGTAGTATTCCTAGACCG | | |
| | TTTGTGAAGTTGTGACACCAGTTTTGGGTAGTATTTGTTTA | | |
| C110a | GATCCAAGTTGTAAACATTGTGGAAATGGAACATGTAATATAT | No hit | SEQ ID N° 613 |
| | ATAATGCTTA | | |
| C115 | GATCCAAAGGTACGGCTTAGCAAAATTACAGACATGATCTCGC | No hit | SEQ ID N° 614 |
| | TTCACACATTTCCAGAGGCAACAGTAGAAGAAAGATACCATAT | | |
| | TGGAGAGCTAAAGGTTTTATAAAAAGTTGAAGAAGGTTTATAT | | |
| | TAGCCTCATCTACAATCCTGTTGCAGAGATCAACTAAGTGACT | | |
| | TGAAATGCTTTTGTAGACCATTA | | |
| C117b | GATCCAACACGCAACTGTGAGTATTTTTGAAGAGCTCGAACAA | No hit | SEQ ID N° 615 |
| | TATAGAAATTAGAAGTT CACTTTATAT TTGATTA | | |
| C118a | GATCCAAGGAGGGTGGTGTAGCGCCTTCACGTCAAAAGACTA | No hit | SEQ ID N° 616 |
| | ATGAAGTTGTCATTA | | |
| C118b | GATCCAACAGCTCAACAATGAAAGAAACAAAGCAAAAGATAA | No hit | SEQ ID N° 617 |
| | TCTTTTTTTCATTA | | |
| C154 | GATCCACGGCTATAGGTGATGACGATGGCATTGAGATACCTTC | No hit | SEQ ID N° 618 |
| | AGGTTTATTCGAAGGTAGAATCAGTCAAACGCA | | |
| C124a | GATCCACCACAACCCACATTTGATTTGATAGCTCAATCTAAAT | No hit | SEQ ID N° 619 |
| | TGGAAGCAATAGAGGTAATATTTAGGGAGCACCAGTTA | | |
| C155 | GATCCACCAAAACCCTTGGCAACTTCGTTACTCAGGACTCATC | No hit | SEQ ID N° 620 |
| | ACACCAATCCATCCCGAACTTGGTGGTTA | | |
| C129a | GATCCACCATTTGGGAATTTGCTGCAATGTAGGGAAAAGAAAC | No hit | SEQ ID N° 621 |
| | AAAAATTGAAATGTCACACACTGACTGAGGTAATTACAAAATT | | |
| | ATCATTGATCTTTACATTCAAAGTGGCTTA | | |
| C156a | CGACTGCGTAGTGCTCCACTTACCATAGTTTGAGCACGATAGA | No hit | SEQ ID N° 622 |
| | CATTCCGGGATCATCTAGTAAGGATCGCTCATTCAGGAGTTGC | | |
| | TTA | | |
| C156b | GATCCACAAGACATGTTCACCACCAACCGGGTACATGTACCAC | No hit | SEQ ID N° 623 |
| | GATGTTTTGACAAAATGTTGTGATTTTTTTGCTTA | | |
| C156c | TTGGGCAGTAATACGCTAATCAGAGATTAAGCAGCATAAACAT | No hit | SEQ ID N° 624 |
| | ATGAGGCTGATATAGTTATTGTCGCCCACTTAGGGGAAGTTTA | | |
| C158 | GATCCACAAA ATCAAACGGA CTATAACAAA TCCAAAACCC | No hit | SEQ ID N° 625 |
| | TAAGTTTTGAATCTGAAATT CGGGTATAAA AACCCTAGGG | | |
| | ATAGCAAGAA ACGGGGA | | |
| C138a | GATCCAGACAAAACACCTTTGTTATGCTCAGGGTTGAGTAGTT | No hit | SEQ ID N° 626 |
| | TA | | |
| C138b | GATCCAGGTACCTCAGAGCGAGCTGGGCATTAGGTGACTGTTT | No hit | SEQ ID N° 627 |
| | A | | |
| C146 | TGACTGCGTAGTGCTCCAGGGTACAGACGTACAGTCCTTATTC | No hit | SEQ ID N° 628 |
| | ATTCTTCACTTA | | |
| C147a | GATCCAGCACATGCAGAACAACTCATCCCATTA | No hit | SEQ ID N° 629 |
| C171 | GATCCATCCA AATGAGTCGG TGTTAGGAGA ATAGCTGATA | No hit | SEQ ID N° 630 |
| | TACTAACTGCCTTGAACTTTG CCTTCAGCTT GCAGCTCCTC | | |
| | TGCATGTAGT GAGGAAGCTA ATGCAGCTCC ATTTCCATGA | | |
| | ACCATAACAT TGTCACTTCG TGGGATGATATGTGCTTTGA | | |
| | CCATGGTAGC ATGAGGGACA AAATTCTTCA TTGCGTTA | | |
| C179 | GATCCCATTGTTGTCATAAGCGAGACAGAAGAAAAATATCAGT | No hit | SEQ ID N° 631 |
| | CTTTTGAGGATTGTCCTGGTTTATCT | | |
| C180a | GATCCATTACAACAGATAAATTGCAGTGTTCTGTTGGCTTA | No hit | SEQ ID N° 632 |
| C181b | GATCCATATTCATGTATACAATACACTCATCTGGCCTTA | No hit | SEQ ID N° 633 |
| C181c | GATCCATAGGAGGGAAAGTCTGATGCCAGCGCCGCCTTA | No hit | SEQ ID N° 634 |
| C183b | GATCCATGAA AGCTAGGTTG AAGATTTGTA TCAAAAAGGG | No hit | SEQ ID N° 635 |
| | GGCATGATGAATTGAGCATA AAGTTTGCTG CTTCTTGCTG | | |
| | ATGATAGGGG GGAGTGAGCTTTGGCTTGCG TTATTTGTCC | | |
| | TAACTAGCCA ATGGTCTTCT GGTGGCTTCTGGTGATTGGC | | |
| | TAAGTCAAAG CCATGGTAGA TTATTTGTTG | | |
| | CTGGATTGTGCTAAGTGTGC AGTTGGAACA TGTACTGGAG | | |
| | AAAAAACTTT GAGGTGTTGATTA | | |
| C184 | GATCCATTGA ATTTCCAGAA GTGCCCTTAC AACAGCAACA | No hit | SEQ ID N° 636 |
| | GCAACTGCCCCTGTTGCATA AAGAACAACG GCAGCCATCT | | |
| | GAGTCTTTGA GAGTAACAATTGAGGAAAAT GCTCCTATTA | | |
| | TAGAAGAGGG CCCTGCATC | | |
| C185a | GATCCATTCAATTTGTGGAAGCTGTGGTATATTGGACGTTTATG | No hit | SEQ ID N° 637 |
| | AATGGTACGTTCCTTAGTTCTGCCTTA | | |
| C185b | GATCCATGGTTTTGTACTTCGTATGATTTTGAATTACATCTGCT | No hit | SEQ ID N° 638 |
| | GATTA | | |
| C187a | GATCCATAAAGTTACTTGATATGCCATCCTGTCCAGCTATAGA | No hit | SEQ ID N° 639 |
| | GGAGTATCAAATTGAAGCATTA | | |
| C187c | GATCCATGGC CATTATTTTC GCTGTATTAC ACATCCATCA | No hit | SEQ ID N° 640 |
| | ATAAAGGTCCGATTTCTCCG TATTA | | |
| C13 | CACAAACAAA TAAAGCTATT GTCATTCATT ACTCGAAAAA | No hit | SEQ ID N° 641 |
| | GAAAGTACAA CATATCAAAG AGCGATGACA CAAATTATCA | | |
| | GTGATCTCCT ACTGATTCAC AAACCAACTT GTGTTA | | |
| C14a | GATCCCAAAG TAAACAAGCT AGCCACAAAA AGTGCAATTC | No hit | SEQ ID N° 642 |
| | TTGATGTATA GCAGAAAACC CCTTGTTA | | |
| C14b | GATCCCACTGGAAGAAGCTGAGTTACTCAGGACTCATCAGGA | No hit | SEQ ID N° 643 |
| | GGTGTGGCTGTGTTA | | |
| C15a | GATCCCATGA ATTGTGCTGT GACTCAGGAC TCATCATCAT | No hit | SEQ ID N° 644 |
| | TGACAGCTGC TGTTA | | |
| C23a | GATCCCAATT GTAAGTTCAT GTAAATGTAC ATCATCGTTA | No hit | SEQ ID N° 645 |
| | TTTTTTTGCA GGTGCCAAAT TTTCACATAC AGCACCTTGC | | |
| | CTCGTATCTT TTGTCTGATC TTATATTA | | |
| C29b | GATCCCCAACCGCCATGTTGACTTGAATCAAACAAAAAAAAAT | No hit | SEQ ID N° 646 |
| | TGAACAGTTACTAAGTACTTTATAGAGGGCGTTA | | |
| C32 | TGACTGCGTA GTGATCCCCC ATTATGACCA AGTTTGGCAT | No hit | SEQ ID N° 647 |
| | ACATTGTAAC TGAGATATCA TACACTCACA TATTGAAGAG | | |
| | TTATCCTTTT TTAGCTTCAT AAATTGATTC ATTTTGCTTA | | |
| | CTCAGGACTC ATCGTCA | | |
| C33 | GATCGACTGCGTAGTGATCCCCTCCTGCTGATGAAGTGACCGA | No hit | SEQ ID N° 648 |
| | AAATTGCTTAGTGGCATAGCGAAAAAGGCAAGGCGCTTA | | |
| C35 | GATCCCCCAA AAATATACTA TTTTGATGGA TTCGTCACAT | No hit | SEQ ID N° 649 |
| | ACTAGTAATA TTTTTGAAGA ATTCGGGCAA CCTAGAGTAC | | |
| | GAGTGTATTT GTCCATTA | | |
| C36 | GATCCCCAAG TATACTCATG TATACGTGGA CGTCAAGTAA | No hit | SEQ ID N° 650 |
| | TAAAGTGACT CGAAAGTCAA ATGTCGAACC CACAGATACT | | |
| | TACATTA | | |
| C237a | GATCCCGAAC ATTCGATTGG TGAGTTTATG CAGCAGATGT | No hit | SEQ ID N° 651 |
| | GTACAGTGTA CTTTGTTTA | | |
| C204a | GATCCCGGCCACTTTTTAGCTTA | No hit | SEQ ID N° 652 |
| C204b | GATCCCGACCAAACTTATACTTATGAATTAGTCCCTTA | No hit | SEQ ID N° 653 |
| C205a | GATCCCTAAC CTTGTATTAT GCGGCTGTGA CCCGGTTGAT | No hit | SEQ ID N° 654 |
| | ATTTATGACC ATTTCTAGTG TGATTCCGTG TTA | | |
| C205b | GATCCCTGAC CACCGAAAAC CAGCTCCCAT TCACCTCCGA | No hit | SEQ ID N° 655 |
| | TCTCACACGA AAACAGACCC CTTA | | |
| C205c | GATCCCTGGAGCTGCGAACACGCCTTATGCGTTCGGTCTATTC | No hit | SEQ ID N° 656 |
| | TCAGTCCTCCTTGTCGTCCTAGGCATCGTGCTCATTGCTGTTGG | | |
| | CTTGCTATACCTCGGGTTA | | |
| C206 | GATCCCTAGT AGGAATGCTT GTTTGCATCA CGTGCATTTG | No hit | SEQ ID N° 657 |
| | ACTTTGGGGA CTCAACACAG GGGTTGGGTT CGTCTAGGAC | | |
| | AGGTGCACCC AAAATAACAG CTCCATCTTG A | | |
| C207a | GATCCCTAGT AGGAGCGCTT GTTTGTATCA CGTGCTTTTG | No hit | SEQ ID N° 658 |
| | ACTTAGGGAA CTCAACACAT GGGTTGGGTC CGTCTAGGAC | | |
| | ATGTTTACCC GAAACAAAAG ACCATCCTGA TGCATCTTAC | | |
| | CTGCTACGTG TGCATTTATT TGTTTCGGCT TGTTTGTTGA | | |
| | CCGGTTA | | |
| C209 | GATCCCTAGT AGGAACGTTT GCTTGCATCA TGTGCATTTG | No hit | SEQ ID N° 659 |
| | ACTTAGGGGA CTCAACACAG GGGTTGGGTC CGTCTAGGAC | | |
| | AGGTGTACCC GAAATAAAGG CCATCTTCAT ACATCTTACC | | |
| | TACTATGTGT GCATTTATTT CCGGC | | |
| C213a | GATCCCTTTC TCTCAGCTTT CTCCCCCCAA GTCTTGAAAT | No hit | SEQ ID N° 660 |
| | GGTTA | | |
| C216a | GATCCCTAGTAGGAACGTTTGTTGTATCACGTGCATTTGACTTA | No hit | SEQ ID N° 661 |
| | GGGGGCTCAACACAGGGGTTGAGTCCGTCTAGGACAAGTGTA | | |
| | CCCAAAAATAAAAGACCATCCTGAGGTATCCTATGTGCTACAT | | |
| | GCTGCAATCTTCAAGGGTGAAAAGGATCATTGGCGGATCAAT | | |
| | GATGGTTA | | |
| C222 | GATCCCTTTT GTAACGACCC ATCACGTGGT CGCCCCCTCA | No hit | SEQ ID N° 662 |
| | GGATAATGTC TATGCTTTCA AATGCTCTCT TTACTACTCC | | |
| | GCCTTACTCA GGAC | | |
| C227b | GATCCCTAAG CTTTTCACTC ACGTTAGTGA TAGGTGTTTA | No hit | SEQ ID N° 663 |
| | GATAGAGTGA TTTGTGGTAG TTGAAGTTTG AGTTGAGGTT | | |
| | ATTTGAGCAA TGACTCATGT GTGTTTCTCC TTTGTAAGTA | | |
| | ATCTGCCTTG TTTGCTGCAG TTACATAGAA CTCACATTA | | |
| C229 | GATCCCTTAC AAATGACCAG CTGGTTTCAG ATTACTCAGG | No hit | SEQ ID N° 664 |
| | ACTCATCATC ATTA | | |
| C231a | GATCCCTTAC AAATGACCAG CTGGTTTCAG ATTA | No hit | SEQ ID N° 665 |
| C231b | GATCCCTAAT TATTGATGTT TTTTGTTGAT TA | No hit | SEQ ID N° 666 |
| C231c | GATCCCTGGT CTGGGATTCT AGAAGTGCAT TA | No hit | SEQ ID N° 667 |
| C302 | ATAATAGCTGAACAAAGTGATAAAAATCTATGTATCATAAGCG | No hit | SEQ ID N° 668 |
| | GGGACTGCTCCTTTCAACTGGAGCTTTCACACCGCTGTATCTTC | | |
| | TTCAACATGTTCTATTCCCCTATTGGTTATTATAGTCCTGTGAG | | |
| | AAGCATTTTCCAGGAAATAGATCATGTTTTGCTTTA | | |
| C313b | GATCCGAGGG TAGTTTTTCG GTGTTTAGAT ACTCTATATA | No hit | SEQ ID N° 669 |
| | CTTGTTTCTC CAATCCCAAG AGAAGATCGT TCGAGTTCAA | | |
| | CAGTCAGGCG TCCACCTGCA GAATGCGAGT CAACAGTCCA | | |
| | AGGTTATCAA CAGAAGTTAG TCACAATAAA GAAAAAGAGA | | |
| | GACAGGCAAG AAGTAAATCC AAATGCAGAA GTTGATGAAA | | |
| | GATGTGAACT GCTTA | | |
| C314a | GATCCTGAAACTGGATATCGACTGATAAATTATCATCAACGTT | No hit | SEQ ID N° 670 |
| | TTTGCTTGTGTACCATTTCTTTTCCGTAAAAGACATACTGCTTA | | |
| | GTTTTTATGGTCCTACATTCACTGGGGCATAGCGGCAGACTCC | | |
| | CTTA | | |
| C315 | ACGGGGTAGCCTGATAGAGAAGGGACCGCTCTTAGAGGGATG | No hit | SEQ ID N° 671 |
| | ACCAGGGAAGCTTATGCCCTTA | | |
| C317 | ACACATGCTCAAAGGAAAGGCGCGACCCCAGCGAATACCGAT | No hit | SEQ ID N° 672 |
| | GGAGTTTCTGCGCTCCAATGCACTCTAAGGACGTGGAAACTCC | | |
| | ATGCTCGGGTATGGGCGAGTCTTGCATTACTCACAGACTCATC | | |
| | GGCACCATTA | | |
| C318 | GAGAGATTGGAGGTCAACTTCGTCAGATAATCACGAGGAAAG | No hit | SEQ ID N° 673 |
| | ACCAGCAACTACAAGAGACACAAATAGGTCATCAAGACGCAT | | |
| | GCCTAGCTCCTTCTTGTTCAGGCATTA | | |
| C321a | CGACTGCGTAGTGCTCCGAATTGGAGTATTTTTTTGCTAAGTTT | No hit | SEQ ID N° 674 |
| | TTCTTTGGGTCAGAGCTTGTTGTCGCATTA | | |
| C321b | GATCCGATAG TAAAACCAAA TTACTCAGGA CTCATCGTCA | No hit | SEQ ID N° 675 |
| | TTA | | |
| C321c | GATCCGAATTGAAGTATTTTTTTGCTAAGTTTTTCTTTGGGTCA | No hit | SEQ ID N° 676 |
| | GTGCTTGTTGTCGCATTA | | |
| C342 | TTGAATACAAAATCAAGTAGCCGAAGGCTTTAATTGTGAGCCG | No hit | SEQ ID N° 677 |
| | GTCAAGTTCAGCAATACTCAGCTGCGCAAAGCCGTAGAGTGG | | |
| | ATCAAAATGCAACAATTTCCAGTACTACAGAACAATTATTTCC | | |
| | TGTACCTTCATTTA | | |
| C343 | GATCCGCCTC TGGATCTAAG TGGATATGTA CCACTCCCTT | No hit | SEQ ID N° 678 |
| | TACTAGGCAG AACCAAATTC TTCGCTAGCT GATAACTGGT | | |
| | CTCATTGTAT TTCCTCTTTA A | | |
| C344 | TGTCGTTCCCCTTCATGTGGTTTCTGGGAGCCTATCTTGATCTT | No hit | SEQ ID N° 679 |
| | TA | | |
| C347c | GATCCGCCCTGGCCTGTAAGACTGAAACTACTTTTTGACCTAC | No hit | SEQ ID N° 680 |
| | CGAGTAGAAGTCAAGTATCTAACGTACTAAACCCTCTTGTCAG | | |
| | TTTTTTCCTCGTTATTGATTCTCTTGTATGAACAGGACACTATA | | |
| | GACGCCAGTCCCAGTGATTTGATTTTCGACGCAAATCCAGCTC | | |
| | CACATATTGATCAAAATGGCATGGAGCTTCAAGAACTGAACAC | | |
| | CAGGCCTCATCTTGTTA | | |
| C352a | GATCCGCGAG AATGCTGCTG CTTGTTAGTG TCTGTTTGTG | No hit | SEQ ID N° 681 |
| | ATTTGCATAG CTTTTGATAT CTTATCTTAT TGGTACCTGA | | |
| | CCATTAGTCT TA | | |
| C355b | GATCCGCAAG TATACTCATG TATACGTGGA CGTCAAGTAA | No hit | SEQ ID N° 682 |
| | TAAAGTGACT CGAAAGTCCA ATGTCGAACC CACAGATACT | | |
| | TACATTA | | |
| C356c | GATCCCAAGAGTAGCTGCCTTTTAGACGGTGTGATCTAATCGT | No hit | SEQ ID N° 683 |
| | GTGTTTGACTCTATTATGATACCTTCATCTGCTGCATTA | | |
| C357a | GATCCGCCTG GCTCCAAAGC AGAATTTTTG TTGAATCGGT | No hit | SEQ ID N° 684 |
| | TGTATGCTGT TGTCCGCATT A | | |
| C357b | GATCCGCCCCTGCATTCGTGTCAAGTTTCTAAAGCGAGTTTTCA | No hit | SEQ ID N° 685 |
| | AATAATTGCTCTGGTATTA | | |
| C335a | GATCCGTCCCTATCCCTGCCTAGTCTATTTCTTTCCTGGATACT | No hit | SEQ ID N° 686 |
| | GCATTTA | | |
| C335b | GATCCGTGGT TATGCCTCCA CACCTTCTGA AGTAAAAGGT | No hit | SEQ ID N° 687 |
| | CCCTGTTTTA | | |
| C337 | GAAAGGATCACGGATTGGAGCTGTGTCTATCTTGTTATAAGGA | No hit | SEQ ID N° 688 |
| | TTGTGTTGTAATAAATAAGTTCACATGGTTA | | |
| C340 | GGCCCTTCTTCTTGCTATTTTATTGTTAGCTGATATTGCTGCTTT | No hit | SEQ ID N° 689 |
| | GATTGGCTTTCTAAAAATTGTAAAATGCATATTCACGCTCGAA | | |
| | TTTTCAGAGATGTATTTTGGGTGATTGCTTTGTTTATTTTGAGA | | |
| | AGTAGAGATATTGAATTCCACCTTA | | |
| C368a | AACCGGAGATGAATCAACGACGAACTTTGATTGTCCACAAATT | No hit | SEQ ID N° 690 |
| | TGTCCGAGAACGAATCTCTCACCAAGATAACTTGACGTCGAAA | | |
| | ACGACTACGAACGGACGACCAAAGAAGGTGGTCGTTTGGCAT | | |
| | CGTTTA | | |
| C405 | ATCCTTTCCTTTTTGTTCGCGTCATGTTTCAACCGAGCCTAATA | No hit | SEQ ID N° 691 |
| | GTTCTAGGATTCGGTTCTTCTTTCATTAGTTCCCCAAAAATCTG | | |
| | AATTTTACTACTAAGAACTTCATACGAGTTGGTTTA | | |
| C406 | GATCCTATTC GTACGTTTTT TTGAAGCCAT AGTACCAGAA | No hit | SEQ ID N° 692 |
| | TCTATTGTCA TAGGTTTTTT GAGTTTGTTT TTCTTTTATT | | |
| | GCTGTTAGAA TCATATGTTC GGGTGTGACT AAGATAACTG | | |
| | CTTAGTGTCT TTTA | | |
| C411a | GATCCTAGAG AGAGAAAGAG AAAGAGATAG CAGTTGAGTA | No hit | SEQ ID N° 693 |
| | AAGGAGAGAG TCCTGTTTGT TGAAGCTGTA ATGTAAAACG | | |
| | CGTTCTCCCC CTTCCCGCTC TGCTGGTTA | | |
| C411b | CGCGTTGGGAGCTCTCCCTATGGTCGACCTGCAGGCGGCCGCG | No hit | SEQ ID N° 694 |
| | AATTCACTAGTGATATCGAATTCCCGCCGCCGCCATGGCGGCC | | |
| | GGGAGCATGCGACGTCGGGCCCCATTCGCCCTATAGTGAGTCG | | |
| | TATTAA | | |
| C414a | GATCCTGGTGTATACGCTTCACCTCGTCCAAGATACTACTGATT | No hit | SEQ ID N° 695 |
| | GTGGAAAGTGCATGAAAGTCAAAAACACTACTATTTGATACTC | | |
| | ACTTGTATTGTTTTACTATAGAATCAAATGGTGTTAGTATGAAG | | |
| | TGAGGGGCTGCTTA | | |
| C414b | GATCCTAACA CAAAGATTTC GTGATGGTTT TGACCTATGC | No hit | SEQ ID N° 696 |
| | TCGCAACCTT AGACCTCAAC CTCATTGACT CTTATCATCA | | |
| | GTGTATTGTG TTGTACAAGT ATGTGATTCT ATTATCACAA | | |
| | ATGTGTTTCA GTTTCTCCTT TTGCTTA | | |
| C415 | TATATTGGGC ATTGGGTCGC ATGTTGCAGG CTGCCATGCC | No hit | SEQ ID N° 697 |
| | CCATGGCTTC GGTGTGTAGT GATCAGAATT CATATTAGGT | | |
| | CTCAACAATG TGCAGCCTGC TATGTAGCCA CAAATGACTT | | |
| | ATAGCCGCCT TA | | |
| C416 | AAGCTCGGTGTGAGAGCATACACTGGTGCTCATTACTATGTAC | No hit | SEQ ID N° 698 |
| | TCTGGCTTA | | |
| C417b | ACTAGTGATTGATGACCCCTGAGTAAGGCGCTTTCAGTGAGAT | No hit | SEQ ID N° 699 |
| | TCAACAATTAGGACTAAGCGTTACACTCTAGGATCACTACGCA | | |
| | GTCAATCCCGCG | | |
| C427a | GATCCTCAAG CGAATGGGGT CTTCTTGTTG TTTACAAGAG | No hit | SEQ ID N° 700 |
| | TAAGGGCCCA GAACTTTTTA GCCACCATAG TTGTTTA | | |
| C428c | GATCCTCCAAGCAAAATAATTGAAAAGGAGGTGGTAGCTGGT | No hit | SEQ ID N° 701 |
| | CCATCCTTTA | | |
| C433a | GATCCTCAAAGTTTATGTGTTGTTTATTTATATCATTTTTTCTCG | No hit | SEQ ID N° 702 |
| | ATAGTTA | | |
| C434b | GATCCTCATTCATGGAATGGCTTGTTTCTGAGCAATTTGTTGCT | No hit | SEQ ID N° 703 |
| | GTACCCACTTCACCGCTTGCAAAAGACATGAGCCTGTTGGAAA | | |
| | AAATTTACGATTCTATCCTTGTGATGGTGAAAGTATTCATTTAT | | |
| | GATAAATCTACCACTTTTGATTGAATTTCACGATCCAAAATAA | | |
| | AGGATGGTGTTGCATACTATAAGATTTTAGTTTGGAGATCGGT | | |
| | TTCCCTATTGATCTTA | | |
| C435a | GATCCTACAT GAACGTGAAA TGCATTGTAC GTAAGGCTGC | No hit | SEQ ID N° 704 |
| | CATTTTTTTT TACTTTCTTG TGAACCTACT AGGAAGTTGG | | |
| | TTGTGGACTT TATAATATGA TTCCAAGAAG ATAATACTGT | | |
| | TGAAAGCAGC GGGGGAAGAT CTACCAAGCA ATGCATAACA | | |
| | AGAAAAGGTG CCTTA | | |
| C437 | GATCCTGTCCTGATGAAAAGTCATTGGGAATAGTTCCATGTAC | No hit | SEQ ID N° 705 |
| | AATTGGCAATTTGGAGCACAATGAACTGGATTCATGTACTAGT | | |
| | TCTGTTTCGGCCTTA | | |
| C440 | GATCCTGATA AACCAACATT ATCGTAGAGA ATTTTTCTCT | No hit | SEQ ID N° 706 |
| | GTTTCTCCCT CTGAAGAACT TGCTTA | | |
| C451b | GATCCTGGTG TATACGCTCC ACCTCGTCCA AGATACTACT | No hit | SEQ ID N° 707 |
| | GATTGTGGAA AGTGCATGAA AGTCAAAAAC ACTACTATTT | | |
| | GATACTCACT TGTATTGTTT TACTATAGAA TCAAATGGTG | | |
| | TTAGTATGAA GTGAGGGGCT GCTTTA | | |
| C463b | GATCCTGCTTTCCACTAAAAGCTTGTGAACTTTTGGCCTAAACT | No hit | SEQ ID N° 708 |
| | CTTTGTTGCTCAATGATATCATCTGCTTA | | |
| C468 | CGACTGCGTAGTGATCCTGCAGTTGATCCTATTGCTTATACAA | No hit | SEQ ID N° 709 |
| | GCCTTGTTTTTACTGTCACTTTCTTTGCGGGTACATTCCAAGCT | | |
| | GCATTTGGCCTATTA | | |
| C470a | GATCCTTGCATGTTAGTTTACAATATTCTCAAATTACTCAGATG | No hit | SEQ ID N° 710 |
| | TAGTTTACTTTTTCTGTTTCTTTTTCCTCTAGTAAGTATATAAGT | | |
| | TATTTGTTGGAATAAACTCTAGAATGCTTGCTTCTTTATGGCAT | | |
| | ATATTAGCACCTACTTTA | | |
| C470b | TAAACCCAAA ATTGAAAACC AGCTGACACT ACTCGAGTTT | No hit | SEQ ID N° 711 |
| | TTTGTTTTTT TGTTTTCTAG TTTTGAATAT CCTATCAGTA | | |
| | TGTGTATTTT CAGTATTTTT GATGCAGAGA AAATGAGTTT | | |
| | TCAAAATCTG GTTTTCTAGT GAAGGAAGGA TC | | |
| C473 | GATACAACGTGATATATTGACAGAATTGTGTTTCGGTTATCAT | No hit | SEQ ID N° 712 |
| | ATAAACATTATATAGGTTCTGCTTA | | |
| T114b | GATCTACCG TGTGTGCTTC TTAGCCTATT GAAAATCGGA | No hit | SEQ ID N° 713 |
| | TTGCATTTTG CTCTAGGCTT ATGATCTTGT TTTAGCTTGC | | |
| | TCCTATTGGT GTTTATTTTT TACTATGTTT TATGTATTA | | |
| T117b | GATCAACCAT GTGTGATTCT CAGTAAATCC GATTGCATAA | No hit | SEQ ID N° 714 |
| | TATATTTTGG ATAGTTTA | | |
| T120 | AGTTTGCTTTACGAGATTTCCTAGTTATTATCCTTTGAGTCTGT | No hit | SEQ ID N° 715 |
| | TGTTCTTTTTTATATCGACTTTTACCTTCTAGTTTTGCACAACAA | | |
| | TGTCTAGCTTTTTTTGTTATTGCCCTTTCTATTTTGTATTTGAAA | | |
| | AGGTGTGTTA | | |
| T125a | GATCTACCAACTCGGGGGTTTATTTACTGTCATTCGTTACTCAT | No hit | SEQ ID N° 716 |
| | GACTCATCA | | |
| T125b | GATCTACCAACTCGGAGGTTTATTTACTGTCATTCGTTACTCAG | No hit | SEQ ID N° 717 |
| | GACTCATCA | | |
| T131 | GATCTACCGTGTGTGCTTCTTAGCCTATTAAAAATCGGATTGCA | No hit | SEQ ID N° 718 |
| | TTTTGCTCTAGGCTTA | | |
| T136 | ACTAGTGATTGACTGCGTAGTGATCTACGTTGCGTTTGGTTGG | No hit | SEQ ID N° 719 |
| | ATGAAAATAGTTGTGGCATACACTTTCTTTTCATGATTTTGGAT | | |
| | TA | | |
| T138a | GATCTACAAA CTTGCAGAGG TGAGAGCAAC ATGGATTTAT | No hit | SEQ ID N° 720 |
| | CCTTTTCCTT GGATTATTTA | | |
| T138b | GATCTACAAA CTTGCAGAGG TGAGAGCACC ATGGATTTAT | No hit | SEQ ID N° 721 |
| | CCTTTTCCTT GGATTATTA | | |
| T141b | GATCTAGTAT GTAATTTCTC TAGTACCATA TTTGCGATTT | No hit | SEQ ID N° 722 |
| | TCCCATTATC TTTGTTTGTA GTCTGTATAT TATAGTAAGA | | |
| | AATTGAATAA CAAAAGACAT AGAAA | | |
| T149b | GATCTAGAAATATATACCTTGGAGTTTCAGAGCTAACACACGC | No hit | SEQ ID N° 723 |
| | AGAATTGGGGTTGTAAATAGTGCAAGTAGCAAATCTGTAATAA | | |
| | TTGTTTAGTGTACTCATCACCCTTCTGCTAGTTCAAAGTGGCTC | | |
| | AGTTCAATACAAATTCAAAACTTTTGTTA | | |
| T160a | GATCTGATAT TGCAGGTTTA GCCAAATCAT GGTCTCTCTT | No hit | SEQ ID N° 724 |
| | GGGCTGGCTG GAGTCCTCCG ACCTAGATNA AGTCCCTGAC | | |
| | TGCGTAGTGA TCTAGGGCGG GTTCTGTTGA TGTGTACATA | | |
| | TAATAAGATC ACGTCTAGAT TATGGATTCT CTTTGAGGAT | | |
| | AAGTTTTACT TTTTGTTCCT ACCTTTTTGT AGTAA | | |
| T169 | AATTGGTGGACAGTATTATAGGCTCAAATATAGGCGAATGCCT | No hit | SEQ ID N° 725 |
| | TCGAGCCCCCAACTGCACTGAAAGTCAGAACAATGACTTCAAA | | |
| | GGCACCCCTTGGAACTATATACAACATGTGCAATGCAAACTTG | | |
| | TGTTTGAGTGTGAAATACCATGGATGCAAGTTATCTTTTGAGCT | | |
| | TACTCTTCTATTTCATTCATTTCTGTAATGTCCTGAATACAATCT | | |
| | TATATTCTGCCTAGTAGAGAAGCCCTTCCTCCCCTCTCTTATGT | | |
| | TGTTA | | |
| T173 | CGATACTCCAGCAAAGAAGAGAAAAAGCCAGTTTTGGCATCA | No hit | SEQ ID N° 726 |
| | AGGGTTCAAATCGAAGTTCCAAGAGTAGTATTTTTCCTCAGAT | | |
| | AACTACTGATAGTGATCTTTGGGTGGAGGCTCATATTTAGAGG | | |
| | GATATCTTTATCTAGCACAACTGGATGTCACACTGATAGTGAT | | |
| | CTTTCTTGGGTTGTTCTTGTGGAGGAAATTCACCTTGCGATTC | | |
| | CTTA | | |
| T174 | GGTTCATACAGTCCAAGACTTATGTGATCTAAATCCAGAATCG | No hit | SEQ ID N° 727 |
| | TAGTAGCTGATTCAAAGTCGCGTGAACAACTTCTTCCATGCTC | | |
| | CCAGACTGTACAGAAACTGTTGCAGACCTTCACCTTA | | |
| T177b | GTGCTCTATCCCCACAAAATTCCATTTTTCTTCACCTTAGCTTC | No hit | SEQ ID N° 728 |
| | TTTATTTTGGCCGTAGAAACCAGTAGCTCATAGCTATGTGAAC | | |
| | CCTCTTCCCTTACCACCTTA | | |
| T7 | GATCTCACCC GGTGCTGCTC CAAGGCAACT CAATAATCAA | No hit | SEQ ID N° 729 |
| | AGAAGAAAAT GAAGTGGTCC TCTTGCTGGA AATACAATTA | | |
| | CTGTCGTTTC GATTTA | | |
| T10 | GATCTCATCT CAACAGCGGA CATGAACAAG CACATACTTT | No hit | SEQ ID N° 730 |
| | GCCCTAATAT TGAAGTGGAC AGTTGGTTA | | |
| T26 | GATCTCCATC GATCGAGTCA GAAAGATCAT TGTACATGTG | No hit | SEQ ID N° 731 |
| | CCAATTAGTA ACCAGTGTTT AGATCAACTA TGGTGTTATT | | |
| | TTTGGGTCTT ATGTTGAATA ATTATTTGAA GCTTTAGTAC | | |
| | ATTTGATGTT GTAATTGTGG AGTACTTGTA TTTTTTATAC | | |
| | AATATCTTTT ATGTTTA | | |
| T31 | GATCTCCTACAGTCCTTGCACGTTTATCTTTTTGTTTCTTCTTTT | No hit | SEQ ID N° 732 |
| | TGGGATTTA | | |
| T34 | GATCTCCTCCAAAATCCTTGTAAGAAATAATGCTACAAGCTTA | No hit | SEQ ID N° 733 |
| | TGAATCCATTTTCTGGTTA | | |
| T40 | GATATTAAAATGAGGAGATTTACCACTCTCTTGACTATGTATA | No hit | SEQ ID N° 734 |
| | CTAATGAAATTATCTCCATATTGAATGGGGATGTAATACCTTT | | |
| | GTCTCTTGATTACTCAAGACTTAT | | |
| T202 | GATCTCGGCA TGTATCAAGT CAAGACCGGT TGATTAGCCA | No hit | SEQ ID N° 735 |
| | ATCAGGAGAT TTCCTTCTGT ATTTA | | |
| T206b | GATCTCGGAG TGAATATGGA CGACGACTAC TTACTGCGAA | No hit | SEQ ID N° 736 |
| | ATGCTAGTAG TCGTAATTCT TCTTCCTCTG TTGATGCTGT | | |
| | GGAGAGAGCT AGAGCGTGGG GTGAAATGTA TTTC | | |
| T209 | GCTTGAAGACTAACTTGGAAACCATGCTTTCGCCCTCTAACCC | No hit | SEQ ID N° 737 |
| | AGCTTTTAACCAAAACTGTCTGGAACAGCTGCTTTCAAGCATC | | |
| | AAGAATTGATGATGCCCCCCTTAGGACAGCAACTGGGCCCCCG | | |
| | GTAAATGGAACGGGCTGGAAAGAAACAACAATAGCAACTCCT | | |
| | TCTAAAACCCCAGGGAAAGGGGAGATAGAAAGACTATTCACT | | |
| | ACAGCGGAGAGAGTCTATTTGATGGTAAGAGCTATAGGAGCC | | |
| | CTACTTA | | |
| T218a | GATCTCTGGT TCAAACTAGA TTCTGGTTCA ATTTTGGTTC | No hit | SEQ ID N° 738 |
| | GCTTTA | | |
| T218b | GATCTCTTGAGAGAGAAGTCGTATGGTCAGTGATTTCCAGTTA | No hit | SEQ ID N° 739 |
| | GTTTA | | |
| T219 | GATCTCTAGT AGGAACGTTT GCTTGCATCA TGTGCATTTA | No hit | SEQ ID N° 740 |
| T223 | TTCTCCTTATCATCACTAGTTTAGTTCCATTTGTACATACCTTTT | No hit | SEQ ID N° 741 |
| | GTAATTCGCGGGGAGAAAATTGGATAGGTGGATTACTAAGCAT | | |
| | AACACTACTGTATCACTTA | | |
| T224 | ACACATTGTTCGGAGCCCAATTGCTGTGAGATTCCTCTTTTTCT | No hit | SEQ ID N° 742 |
| | AGAAAAAGGAAATGATGGCGCTAATCTCAGCGACATGCTGAT | | |
| | TTTTCATTTGTAATAAAACATTTTCACATCATTTTTGCTTA | | |
| T229b | AGTTGGACGATGGGTTGAAGACGAAAGCAGCGGCGTGTGGAC | No hit | SEQ ID N° 743 |
| | TTTGGATGTTCACTGAATGACGAGGCAGCAGCTGCTCGTCGAC | | |
| | TTAGGGATTGTCTGGAGAAGATGACTAACGGGGGGGGTCGTTT | | |
| | TTGTGCTAGAGATCACTACGCAGTCAGGAAGTGACTGACCCC | | |
| T230 | GTGATCTCTCTTGAAATCTATAATGAGACGTTGACAGAAATAA | No hit | SEQ ID N° 744 |
| | GCAATAGATGTGTTATGAGATGTGTTTTCCGCTCTCATTA | | |
| T231 | GATCTCTATT GAATATGGAA TTGAAGATAT GATTTGTTCT | No hit | SEQ ID N° 745 |
| | TGTTGTATTT ATGTCCAGAT TTCGTGTATT A | | |
| T303a | ACTAGTGATTGACTGCGTAGTGATCTGATTCAAGAGCGAGGAC | No hit | SEQ ID N° 746 |
| | TGCTGACTTCGTCTTGCTTTTGGTCCATTCAACTCGTTTA | | |
| T304b | GATCTGAGGTTGCTGATTTAGATTATGATGACTTTGAGGCTGA | No hit | SEQ ID N° 747 |
| | CTTTCAGGTCTTTA | | |
| T306 | GATCTGATAACAGGGTTGGGCTAAAAGAAGCCAAACAGTTGTT | No hit | SEQ ID N° 748 |
| | GATGGCTTAGCTAGAATATAGGTTATTGAAAGTTT | | |
| T307a | GATCTAGGTG CTTCTGGATA ATCTACACGA ACTTCTGGTT A | No hit | SEQ ID N° 749 |
| T307b | TGACTGCGTATTGATCTGAGCAAGCAGTACAGTACAATGATTG | No hit | SEQ ID N° 750 |
| | GAATATTGTTA | | |
| T307c | GATCTGATGC TGAGAAATGA GACGGGGTCG TTTGGTAGTT A | No hit | SEQ ID N° 751 |
| T308a | GATCTGATAT TGCAGGTTTA GCCAAATCAT GGTCTCTCTT | No hit | SEQ ID N° 752 |
| | GGGCTGGCTG GAGTCCTCCG ACCTAGATGA AGTCCCTTCT | | |
| | GATAACGGAG TTGGTGTTCC TGCTTGTGCC ACGGAGCAAG | | |
| | GACTAGACTG ATCAATTGCA TTAGTGGATT CAAAAATTTC | | |
| | GATCTCACTG ACTGAATCAA GAAGCATGTC AAGCT | | |
| T310a | GATCTGACTA CTCTGGATTC ATTACTAGTA ATATGATTT | No hit | SEQ ID N° 753 |
| T310b | GATCTGACTA CTCTGGATTC ATTACTAGTA ATTGATTTT | No hit | SEQ ID N° 754 |
| | GTATTGAAAC CAGAGAGA | | |
| T313b | GATCTGATTCATGTTCCCACATGATACGTAGGCAACCCACATC | No hit | SEQ ID N° 755 |
| | AGGTTCGATCACCATTA | | |
| T315c | GATCTGGCCG GCTAAACTAA TATTGGCGCC ACCACTTCCT | No hit | SEQ ID N° 756 |
| | CAAACTTTAT CTTCTCTGTT ATTGGCCAGA AGAGAGAGAC | | |
| | CTGGCTGGAT TTTTACTCTT TCTGGCAGTC TTTGTGATTT | | |
| | TCTCTCTATG ATTCGCTGGA GAATATATTT TTCCAGTGAC | | |
| | CTTTTGTGCA TGATATTTA | | |
| T316a | GATCTAGCAT CCAATGGACC AAGTTCTCTG GTGACAGCCA | No hit | SEQ ID N° 757 |
| | ATTATTGTGA ACGCTTCTTC TGGTTGAGTT GCTGGTTATA | | |
| | CTAGGTAGTT CTCTGTATAC CAGCTCCTTG TTA | | |
| T316b | GATCTATGTG GCTATGATTT TTGCTTAGTA GTTGAATTGT | No hit | SEQ ID N° 758 |
| | ATTATTTTCA TTCTGCAATC AAGTACTGCT ATCTTTTATT | | |
| | TCTCGGTTTT CATCAAATGT TTGCTCTTCC TTA | | |
| T317a | GATCTCATGA ATGCATAGTA GAATACTGTT GTCTTTGCTT | No hit | SEQ ID N° 759 |
| | ATGTTA | | |
| T317b | GATCTGGGGT TCGCAGCAAC TTTTGAAGAA GAAGAAACTT | No hit | SEQ ID N° 760 |
| | AGGTTTA | | |
| T322b | GATCTAGGAGTTTGTCATCTAAGTAAATCAGTTTTGTATCCTTG | No hit | SEQ ID N° 761 |
| | ATTTTTGCCATACAGAGAGACCAGGGTAAAATGCGCTTA | | |
| T322c | GATCTGTGTGCCTAATGATTTTTGCCTAGTAGATGGATTATATT | No hit | SEQ ID N° 762 |
| | ATTTTCATTCTGCAATCAAGTAATACCTATCTTTTATTA | | |
| T324b | GATCTGGTGG CTATTCTTCC TCAAGGTCCT CGATTA | No hit | SEQ ID N° 763 |
| T324c | GATCTACGAA ACGGTGTGTT GTATTCTTGT TCATTA | No hit | SEQ ID N° 764 |
| T325a | GATCTGGTAA CGGATTTGGC TCCGTTGGAG TCGGACAAAA | No hit | SEQ ID N° 765 |
| | ACACTCTCAG CTTTA | | |
| T326a | GATCTGGGCA TGAGGTTCGC AAGCATGTCA TGTTATTGAA | No hit | SEQ ID N° 766 |
| | ATCTGCATTT A | | |
| T326b | GATCTGGGCA TGAAGTCTTC AAGCATGTCA TGTTATTGAA | No hit | SEQ ID N° 767 |
| | ATCTGCATTT A | | |
| T332a | GATCTGGGTC TGTCAATGAA GAAGAAGAGC TTAGGGCATG | No hit | SEQ ID N° 768 |
| | TGTGAGGTGA AGTTTCATTT ATGGCTATTG CGTAGTGAAG | | |
| | GGAGGTCCGA CGATGGAGTT TTGGTGGTGA GGGTGCGGCT | | |
| | GGTGTGAAGA TGGAGCTAGG TTCTAGGTTT TTTTTGGTGT TA | | |
| T333a | GATCTGGGTC TGTCAATGAA GAAGAAGAGC TTAGGGCATG | No hit | SEQ ID N° 769 |
| | TGTGAGGTGA AGTTTCATTT ATGGCTATTG CGTAGTGAAG | | |
| | GGAGGTCCGA CGATGGAGTT TTGGTGGTGA GGGTGCGGCT | | |
| | GGTATGGAGA TGGAGCTAGG T | | |
| T333c | GATCTGATCC AGCAGTTGAT CTAGCATTTC ATATTCAGTG | No hit | SEQ ID N° 770 |
| | TAATGACTGC GTAGTGATCT GGGTCTGTCA ATGAAGAAGA | | |
| | AGAGCTTAGG GCATGTGTGA GGTGAAGTTT CATTTATGGC | | |
| | TATTGCGTAG TGAAGGGAGG TCCGACTATG CAGTTTTGGT | | |
| | GGTGAGGG | | |
| T335a | GATCTGGTGC GTAGTAACCT GTGCTTTGTT CGAATTCGAG | No hit | SEQ ID N° 771 |
| | GTGCAATCAC ATTCAAGGAA AAATAATATA ATACAAACGA | | |
| | CTTTTTCTTT TTCTACCTTG CTTCAATTTT TACTTCGTAT | | |
| | ATCATAAATT AGTGGTTTAT TTGTTATGTT TCATCACGTT | | |
| | TTGATAATTT TATTGATTA | | |
| T336b | GAATCCACCTACCTAATAGCAAGAACAATTGAATTTGACCGAA | No hit | SEQ ID N° 772 |
| | CAGAGTTCTGAAATTGAGGGGAAGCCCCAACACCGTCTCCCTC | | |
| | CCCGCTAATTCCATTTCTCTAAATTACA | | |
| T338a | GATCTGGGGAAAAAGGGAAACAAAAAAAAAAGCAGAGAAGG | No hit | SEQ ID N° 773 |
| | AAATCTGCCGCCTCCCTAAATGACTAACTCCTCCTTA | | |
| T338b | GATCTGGGTG AAAAGTCAGA AAGCAGCAAA GCAATGTTCT | No hit | SEQ ID N° 774 |
| | TTTCTTCCCA AGACGCAACA ACTCAATAGC CATGAAAACG | | |
| | CATTGCTTA | | |
| T338c | GATCTGGGGG AAAAGCCAGA GAGCAGCAAA GCAACGTCCC | No hit | SEQ ID N° 775 |
| | TTTCTTCCCA AAATGCAGTA GCTCAATAGC CATGAAAACT | | |
| | CACTCCTTA | | |
| T343 | GATCTGGAGATGCAATTTTTGATAACCAGCAGTTCTATTCAATT | No hit | SEQ ID N° 776 |
| | TTGTGCAGTCCTTGCTGGTTGTTTCTTTCTCCATTTTTTTTTGTT | | |
| | CTTGTGAACCATTA | | |
| T345 | ATTGGGCTCCACTGCTATAGGCGCCTGCTGCAGTTTCGGTATC | No hit | SEQ ID N° 777 |
| | AGACAACTTGTCTGATTTTGATGGCATTACTCAG | | |
| T346 | GATCTGGAGA TCAGGAAATG TTCTAAAATC TCCCTCAATT | No hit | SEQ ID N° 778 |
| | ACGCTCTTGG GCTCTTGATT TTAGGTGCTC TTGGTCATCC | | |
| | ATTA | | |
| T350a | GATCTGTTCA AGTTGGCCGA TTAGTCCATC CTTTTTACTG | No hit | SEQ ID N° 779 |
| | AATAACATAC AACTTTGTGC TTCTTTTTAC ATGAATAAAA | | |
| | TACTAGAGAT GTCTTTTCTC AACATTGTT | | |
| T350b | GATCTGTAGA GAAGGCTCGC TCCTAAATTA TATTTCTTTC | No hit | SEQ ID N° 780 |
| | ATTTACCTTT TCTTTCTGCT AATTTCCTTT TCCGTGGTCT | | |
| | CTTTTACTTT TTTCTTGGGA GGGGAAGATG GGAGTGGGGT | | |
| | GTCACCTTCC CTTGTC | | |
| T350c | GATCTGTGAG ACTTAGTAAG AAGCATGGCT GGTTTTTCAT | No hit | SEQ ID N° 781 |
| | ATGTACAGCC CATCTCATTT TAGTGTAGAA TAAGCATGAG | | |
| | GTATGGTTCA TACGCTAATA GCACATTGAA TGGTAAATTT | | |
| | TAGGTTTCC | | |
| T351b | GATCTGTAAA TATGTTACAT ATTAGGAGTA TAATGTTTTC | No hit | SEQ ID N° 782 |
| | ATTACTAAAG CATGTAAATA TGTTGCTCCG GGCTTTGGTC | | |
| | TATTAGTAAG AGCGCAATGC GTA | | |
| T353a | GATCTGTCAT TGATGTTCAT TACTCCAATC TTTTCTCTGA | No hit | SEQ ID N° 783 |
| | CATGTTTA | | |
| T358 | GATCTGTAAA TATGTTACAT ATTAGGAGTA TAATGTTTTC | No hit | SEQ ID N° 784 |
| | ATTACTAAAG CATGTAAATA TGTTGCTCCG GGCTTTGGTC | | |
| | TATTAGTAAG AGCGCAATGC GTA | | |
| T359a | CCATCAGCTAAATTATCTCGAATTTCAATAGTGGTACTCAC | No hit | SEQ ID N° 785 |
| T359b | CATCTGTAAA TATGTGACTG ACTGCGTAGT GATCTGTGGA | No hit | SEQ ID N° 786 |
| | AACTGCTGAT CCGGTAATTC TCAGAGA | | |
| T359c | GATCTGTACT GTACATGTCA AAAAGGGAC | No hit | SEQ ID N° 787 |
| T360a | GATCTGTGAG GGG | No hit | SEQ ID N° 788 |
| T360b | GATCTGTTGA GAAATATGCT AATAA | No hit | SEQ ID N° 789 |
| T360c | GATCTGTCAA AGGCCAA | No hit | SEQ ID N° 790 |
| T364b | GATCTGTGGA AAAGGAAAGC TGGAGAAACT TGCTGTGCTG | No hit | SEQ ID N° 791 |
| | TAATTTATGT ACAGTGCTAT TTGGCTGCTC AACTAAGATT | | |
| | GTTTTGATTC TCTCTTAGTC TGATGTTATC TTTTCTCGTG | | |
| | ACAATCCTTC CTTTTTCTTT CTTCTCTTGG AGTTGGGGGG | | |
| | TCAATATCCT TTGTTTGTGG TG | | |
| T366a | GATCTGTCGA AATTTGATTC TCGCACGAAT TGTACCATTG | No hit | SEQ ID N° 792 |
| | CCCACTTCCA TTGGCAAGGT TAGGGTACAG AAAGTCTTTT | | |
| | GTGAGTGACC AATATATA | | |
| T366b | GATCTGTGAA TATGTTGCTA TTATATTTAC GCACATCTTA | No hit | SEQ ID N° 793 |
| | GATTTCGCTT TTCTTTCTGT TCTGAATCTC T | | |
| T371 | TTTATCCGCACGAGGCTTCTGGAATGTAATGGCAGCTGATACA | No hit | SEQ ID N° 794 |
| | TTGATGTAAATGTAATGCATTGTGCTTCTCAACCAAAGTACAC | | |
| | TTCCGGGGAGGTCATTA | | |
| T372a | TGACTGCGTAGTGCTCTGTGAGAGGCCATTTGGATCATATATG | No hit | SEQ ID N° 795 |
| | TTGCTATCCATTGCATTA | | |
| T462 | GCTAAATGATTTCTAATGGGATGGGCATGCTCCCCACTGCTCT | No hit | SEQ ID N° 796 |
| | ATGATTTATTATAGTCATACTCTGTTTCGTACCTGGCCGCTAGC | | |
| | CTTTCGCTTCCTCCTTGTACTAGATTTTGACCTTGAATTCCCCCT | | |
| | GAAAGCGAGAACGCACTATATGCCTTTA | | |
| T403a | GATCTTATAG CTAGATGTTG GGTTTTGACA ATTGAACTCT | No hit | SEQ ID N° 797 |
| | TATCATTGTA TTTGAGTTTG GACTGTCATG ATGAAACTTG | | |
| | ATGAAAACCT GCTTAGTCGA ATCAGTAGCA AAAT | | |
| T403b | GATCTTGGAG TGAATATGGT CGACGACTAC TTATTGCGAA | No hit | SEQ ID N° 798 |
| | ATGCTAGTAG CAGTAGTCCT TCTTCCTCTG TTGATGCTGT | | |
| | GAAGAGAGCT AGAGCG | | |
| T403c | GATCTTAGAG TGAATATGGA CGACGACTAC TTACTGCGAA | No hit | SEQ ID N° 799 |
| | ATGCTAGATA GTCGTAATTC TTCTTCCTCT | | |
| T466a | GATCTTATAT GAGCTATGTC AATTTTGATC GGCTTCTTCT | No hit | SEQ ID N° 800 |
| | GGATTA | | |
| T466b | GATCTTATAT GAGCTATGTC AATTTTGATC GGCTTCTTCT | No hit | SEQ ID N° 801 |
| | GGATTA | | |
| T429a | GATCTTCCAG GATTATTATT GTCTTCCGCT GCGTGTTACG | No hit | SEQ ID N° 802 |
| | AACACCTATA CGCAATCGTA CATTATGGAC CATAAAACCG | | |
| | ATCCCCCTAA TCTTGAATAA AAAATCCATG CTATTTTTTG | | |
| | TTGTCATTCC ATTTA | | |
| T429b | GATCTGGCTGATAGTGCAAAAGATTCAACTATTATTGACATAT | No hit | SEQ ID N° 803 |
| | GTTGCAACATGTACCATGTGTGGTTTGATCATGGCGCCTAGA | | |
| | TGGAAGTGATGCTATAGTAAATAGACTTCACTTGTTTCATGCT | | |
| | ACTTA | | |
| T432b | GATCTTCAAC TATCTCAACT GCTGTAGTGC AAAAGCTTGA | No hit | SEQ ID N° 804 |
| | AGTTCATGGG ATTGATTTGT TCCAACTTGT TTGTAATGAT | | |
| | AAATATATCA ATGTGATTTC TCCTATATAT GTTTTGAGGG | | |
| | ACTTTTCCAA GAAAAAGGAA AAGTGTGGAT TTTATGATTG | | |
| | TGGTGACTGG TAATTA | | |
| T432c | TGACTGCGTCTTGATCTTCAACTATTTCAACTGCTGTAGTGCAA | No hit | SEQ ID N° 805 |
| | AAGGTGAACTTCATGGGATTGATTTGCTCCAACTCGTTTGTAA | | |
| | TGATAAATATATCAATGTGATTTCTCCTATATATGTTTTGAGGG | | |
| | ACTTTTCCAACAAAAAGGAAAAGCGTGGATTTTATGATTGTGG | | |
| | TGACTGGTAATTA | | |
| T433a | GATCTTCCAG AACAGCCATC CACC | No hit | SEQ ID N° 806 |
| T433b | GATCTTCCAG AACAGCCATC CACCAGTGTA AACAAATACA | No hit | SEQ ID N° 807 |
| | AATCAAGGTC CCAATGATGA ATGTGTTCA | | |
| T436b | GATCTTCCAAAATACAGCTAGGAACTAACCACTCAATAGATCA | No hit | SEQ ID N° 808 |
| | TCTCCAATAAATTTTGCGCCTTCCTTCCTTATTA | | |
| T437 | TAGGGAATAGGAAGATGTACAAGAGGCAATATGGAGCACAAT | No hit | SEQ ID N° 809 |
| | GAACTGGATTCATCTACTATGTTCTTTCGGCCTTA | | |
| T438a | GATCTACATG TCTAATGTAG TTGGGGATTT ACCTTATCCT TA | No hit | SEQ ID N° 810 |
| T438b | GATCTTCTGCAAAGGTAGCAGCTTCCTACTAACCAGATATTA | No hit | SEQ ID N° 811 |
| T411a | TGTATTTCTGCGGCGGGGGGGGGGGGACCTTTGAAAATACCAA | No hit | SEQ ID N° 812 |
| | AAACACCCCTTATTTGCCCATTGATTTTGGTTTTAAAAATCA | | |
| T411b | GATCTTGCAT GAATACGGAA TATATACTTT GTGCACCGAA | No hit | SEQ ID N° 813 |
| | GTGCCCTTCC CTTCTTGGTT GCTA | | |
| T416 | GATCTTTGGGTTCTCATGGATCGTGGGGACTATGAACTTTGAAA | No hit | SEQ ID N° 814 |
| | GGGCTTTA | | |
| T417 | GACTACTTACTGCGAAATGATAGTAGTAGTAATTCTTCTTCCTC | No hit | SEQ ID N° 815 |
| | TGTTGATGCTGCGGAGAGACCTAGAGCGTGCCGTGAAATGTAT | | |
| | TTCTTGCATAACTATGATAGGTTGGTTA | | |
| T422a | CATATGGACCAAACTTGTTCTGAGTTTTTGCTAGATTGAGACTG | No hit | SEQ ID N° 816 |
| | CATGGTCCTCTC | | |
| T422b | GATCTTGCCA TGGACTAATT ATCAACAGCA GCCATATTGG G | No hit | SEQ ID N° 817 |
| T426a | GATCTTGGAG TGAATATGGT CGACGACTAC TTATTGCGAA | No hit | SEQ ID N° 818 |
| | ATGCTAGTAG CAGTAGTCCT TCTTCCTCTG TTGATGCTGT | | |
| | GGAGAGAGCT AGAGCGTGGG GTGAGA | | |
| T428a | GATCTTGGAG GAATAGAAAG AGCGTTGTAT ATTGCTCGCA | No hit | SEQ ID N° 819 |
| | CTTTCTATAG TTTTGATTA | | |
| T428b | GATCTTGGAG TAATACAAAT AACGTTGTAT ATTGCTCGCA | No hit | SEQ ID N° 820 |
| | CTTTCAATAG TTTTGATTA | | |
| T441b | GATCTTTGGACAAAGTTTGGGGAAATGATTGCTCTGCTCTTT | No hit | SEQ ID N° 821 |
| | GTTGTTGGTTA | | |
| T450a | GATCTTTGCATAGTTCGGAAAAATCGAGAATAGACTAAATAAA | No hit | SEQ ID N° 822 |
| | CTAACGTTCTCTTTTTTCTTTCTTTCTCTTTTTTTTTTTACCTTA | | |
| T450b | GATCTTTGCGTAGTTCGAGAAGATCGAGAATAAAGGAAACAA | No hit | SEQ ID N° 823 |
| | ACTAACGTTCTAATTTTCTTTTCTTTTTTTTTCTTTTTTTACCTTA | | |
| T452a | GATCTTTGCGTGTTGCACTACAGATTTTTAGGACCTTCTGACTT | No hit | SEQ ID N° 824 |
| | GTTA | | |
| T452b | GATCTTTCAG TGTTGTACTC TTCCCTGCTT TA | No hit | SEQ ID N° 825 |
| T456 | GATCTAAGTAGAGCAGGGTTCTAGATGCCTAGGATGCTTTCTT | No hit | SEQ ID N° 826 |
| | GGGTGAATCTGCCTTTTCCTCTTGCTGCCTATCTCTGTGGCAGC | | |
| | TCCAGAGAATGGTGATTGTCTGTTGTTTGAAGCTGCATTA | | |
| T459a | GATCTTTGATATTGGTAGCTTGTGAGTTGAAGACTAAGGCTTA | No hit | SEQ ID N° 827 |
| | TTAGTAAAAATAATACATGTATTAGCCTTTGTATTA | | |
| T459b | GATCTTTAGA GAACTATAAG TTTTACTTCT GTTTCTTGAC | No hit | SEQ ID N° 828 |
| | CGTTTTTGAT TTTGTGTTAT TGAGATATAC TTGCAATTAC | | |
| | TCAGGACTCA | | |
| T460a | GATCTTTACTTGATTGCTACTCCTTGTGTCGCGTCTTGATTA | No hit | SEQ ID N° 829 |
| T460b | GATCGTTACT TGATTGCTAC TCCTTGTGTC GCGTCTTGAT TA | No hit | SEQ ID N° 830 |
| MC103 | TAAACATGCG GAAGTCCAAA GATAATACCA CTACCTAGCC | No hit | SEQ ID N° 831 |
| | CACATTGATC CGGTGTCACA AGTCAAGAGC CTCTAATACA | | |
| | AGTCTGAACG ACCTAATACA TAAATAATCT AGGAATGTGG | | |
| | AAAGTAATAA GATATGAAGG AGCAATCCGG GTCTACGGAT | | |
| | TACATGCAGC TACTTCGATA ACTCCGGCAA ATG | | |
| MC114a | TAACGTATCA GCTTTGTTTT TTCCACGGTT CCACCTAAGT | No hit | SEQ ID N° 832 |
| | AGCTATGTTT CTTGGATC | | |
| MC114b | TAACTAAGGGAAAGAAGAGAAGAACAGAAATGACCTATAGCT | No hit | SEQ ID N° 833 |
| | ACATTAGGCATGGATC | | |
| MC119 | TAAGAGGAGT GCAGCTTTTG CTCAAGTTTC AGATTCTCAG | No hit | SEQ ID N° 834 |
| | CCCATAACAC ATCCTGAGAC TCTTGTTTGT GAGAACAAAC | | |
| | AACTTCATTC TGAAGGAGGG GTTCCCAGTA TTACCAAGGA | | |
| | GCAGTTTGAT CAGCCTTTGA CTCTTCTTCA ACAGTCCAAA | | |
| | GTCTCAC | | |
| MC130c | TAATGAGGATGTGGTGGCTCTGTACAAAAGGTAGACTGATTGA | No hit | SEQ ID N° 835 |
| | GAAGTATCAAACAGCTCAAGTGTAGATGTGGTCATCTAACAAA | | |
| | TGGTGGATC | | |
| MC202 | TAAACATACAATGACTGGGCTGTTATAGCAGGGGTTTCGGGAC | No hit | SEQ ID N° 836 |
| | TTCTTTTGTTGGGTTTGTTTTGTTCAAGTTAGTAGTGAAGTTCA | | |
| | GCTCGAGTTCAACTCTTATCTGGACTCTATTGCTTTGGGATC | | |
| MC210a | TAACAATCAG ACTGCATCAA ATTTCTACCT AGGCCTACAA | No hit | SEQ ID N° 837 |
| | TAATTTGAGT GTGGTCATGG GATGGGATC | | |
| MC301 | TAAACGATGC CCAACGACCA CCTTCTTTGG TCGTCCGTTC | No hit | SEQ ID N° 838 |
| | GTAGTCGTTT TCGACGTCAA GTTATCTTGG TGCGAGATTC | | |
| | GTTCTCGGAC AGATTTGTTT ACAATCAAAG TTCGTCGTTG | | |
| | ATTCATCTCC GGTTAGTTGT TTTTGAGTTT TATTTTTGTC CAA | | |
| MC305a | TAACTGAACT TTATATAAAC TGTGCCGACA CCCTTCTCTC | No hit | SEQ ID N° 839 |
| | TTCACCTCCG GGGATGTGCT TACTGGTTGA GACTCCCTAT | | |
| | TCTGTTAGTG TCATACCTTG AAATAAGAAA GAGGCCGGAC | | |
| | AAGTTACGAA GCCAGATGGC CTTTTGGTTC CCGGTAAGTT | | |
| | GCCCCCTCCT CGACTCGAGT TGTCCGCTCG GGTACATAGT | | |
| | CTAAAACACT GACCCAGGTT TTGAACATAG AATAACGTGA | | |
| | CTTCATGCCG GATC | | |
| MC306a | TAACATGTTGGACGCGGATATACCTGTTCCAAATATACCAGAG | No hit | SEQ ID N° 840 |
| | AGACCAATTTCTCTCATTGCGGATC | | |
| MT104b | AAGTCATAAAGAGGACTGAAAATTGCCAGAACCCTGAAGGAG | No hit | SEQ ID N° 841 |
| | CTCCAGGATGACATCTGGCCAGAGCCTACTTGCTGCTGGGGCT | | |
| | GCACAAGCTGGGGGATC | | |
| MT115a | GTAATTGCTCATGTCCTATGCCTTTGGAAAGACATCCAAATGG | No hit | SEQ ID N° 842 |
| | CTATGAGATTATATGCCCTCGTTAGACTTTGCCGGCAGATC | | |
| MT116 | AACATGTACC GGGATTCTCA AAGAAACAAG TCATAGCTAC | No hit | SEQ ID N° 843 |
| | ACCAGATGTT GATCATGTTC TTTTAGGAAT CTCGAAGAGA | | |
| | TTACTTCC | | |
| MT117a | TAATGTTATG ACTTGTGGGA GGGATTGTGT TTACAATGAT | No hit | SEQ ID N° 844 |
| | TGTAAAGATG ATTGTTGGAT TTGCTGTAGA TGTGTTAGAT C | | |
| MT117b | TAATGTAGGT ATTGTGGGGT GGTAGTGGTT GGAGCTTCGA | No hit | SEQ ID N° 845 |
| | GAATTTGGGC AAAAAAGTGA CGGGAAAGTT TTCTAGATC | | |
| MT118 | GAAGAACGGGATTCAAAAGGTAATTTCATTACTCAG | No hit | SEQ ID N° 846 |
| MT209a | TAAGGACGAG GAGGTAAAGG GGATTATTGG GTGTTAGTGT | No hit | SEQ ID N° 847 |
| | GGGGTCAAGG AGACAGGCTA GGGCTTGGAG GGGAGATC | | |
| MT210b | TAAGGGAAAA GATAATTTTA CTCCAGGACC AGAAGAAACT | No hit | SEQ ID N° 848 |
| | CAAAGACTGG TATGGAAAAT TTTGAGATC | | |
| MT213 | GTCCCGAACTGTGCGTCTAGGCGGGTGGGGACACGGGGAGAA | No hit | SEQ ID N° 849 |
| | GGGGCACGATGGTTTTACCCAGGTTGGGGCCCTTTGGAGGGGG | | |
| | GTAAAACCCTCCTCCTGGTTGATTACTCAGGGCTCATT | | |
| MT214b | TTTAACCCAACCCTGTTATCAG | No hit | SEQ ID N° 850 |
| MT301a | TAAACAGCCCGAAAATCACCCAAAGACACTCTCTAAACTATCC | No hit | SEQ ID N° 851 |
| | AAAACATCGGCTTTGAATCACCCCAAAACCACGTTTTACGCAG | | |
| | CTAAAATACAGCACTAAACTCCCCAAAAAAGGGTCGATGTCG | | |
| | CACCATATTTGTCAACAAACAGAGCTTCGCTTCAACTGTATAA | | |
| | GATCACTCACGTTCAGTCGCGTTTTTTTTTTTAGTTGGGTTCAA | | |
| | GGTTTCCGACGTGGGTCTCGGGTCAGTAGTTTGTTTGTACGAA | | |
| | AGTTTTAGCAGATC | | |
| MT303a | AAGTGGCACTTTA | No hit | SEQ ID N° 852 |
| MT303a | CTTATTATGCTTTTGCTCGTTTA | No hit | SEQ ID N° 853 |
| MT304a | TAACGATTAT CCGTTTGGAA ACACTAGCAA AACCTGACGC | No hit | SEQ ID N° 854 |
| | CGGGACTCGC GAAAAATCGG AATAAGCCAA CAGGAATTCG | | |
| | TAGACCAAAA CTCGAACATA CGGGGAACCT CAAATCCTCG | | |
| | AACGCGGACC AGATC | | |
| MT304b | TAACATACAGTACGAATTTTGTTACTTTATTACTTTGACAGCAA | No hit | SEQ ID N° 855 |
| | TGCAAGGGAAAGCAGCCACAAGTTGGTAAGAAATAAAAAAA | | |
| | GGCCAAGAACACTAGTTGATGAGGATGTTGAGGACACCATGG | | |
| | CCAGATC | | |
| MT304c | TAACCAACTGAAACAAAACTGACACTATCATTGCATACAACCT | No hit | SEQ ID N° 856 |
| | ACTGTCTACTATTGTTTTAAGTTTCTCTTCATTTTGTATTTTGAT | | |
| | GTAATTGTATTATGGGACCACGTTTGTCACCGACCCTCTCCAG | | |
| | ATC | | |
| MT305c | TAACAACCAGAGTTCAAACGATAAAAGGGTGTGGTTGATTTAT | No hit | SEQ ID N° 857 |
| | ACCCAACCCACTGAAACTTGAAAAATATACACTACTAGTCAAG | | |
| | CCATCAAGCAATCCAGAAATGCAGAGGAGCCCAGATC | | |
| MT306b | GCGTAGTGCTCTGTCGCTAATGGGGTTTATGCTGCGATTTTTCT | No hit | SEQ ID N° 858 |
| | TTCCTGTCAAAAACTATGTGGACTAGGAGTGGAGTGCGTCCTC | | |
| | TACAACAATATCTGAGTTACATCCGATCGGGTCACTCAAGACT | | |
| | CA | | |
| MT306c | TAACGGAAGGAGAAAGGTGGATATTATTGTGGTGTGGCCTTTT | No hit | SEQ ID N° 859 |
| | GTCTTTGGTTTTCTATTCTATTGAGCCCTAAAAATAGTGATATC | | |
| | TTGGTCTGATGTTCCCTGTTGCAGATC | | |
| MT308c | TAACCTTTTT TAGCAAGTAA TTAGATTACT AATTTCATTT | No hit | SEQ ID N° 860 |
| | TTTAAAAACG GTTACCCGGA GTTTTCTTTT TTTTTTTTTA | | |
| | CACTTGCCAG ATC | | |
| MT312 | TAAGGGTGAG CATGGAACTC GGGTGATATG GTAGCCTGTG | No hit | SEQ ID N° 861 |
| | AA | | |
| MT313 | TAATGCTGAT GA | No hit | SEQ ID N° 862 |
| MT402a | GGTGAATTGGCATTGGCTGCTCCAAAATGTCCTTCCATGAAAT | No hit | SEQ ID N° 863 |
| | AAGAGCAATACCAGCATCTGCTGCTTATTTTCAAGACAAGATC | | |
| MT408 | TAATGTGTAA TCAGTAGCAT CTATGTGCAA CTTTGATGGT | No hit | SEQ ID N° 864 |
| | TTTTGTCACA TCCAAGTAGT GAACACATTC ATCATTGGGA | | |
| | CCCTGATTTG TATTTGTTT | | |
| MT410b | TAATTGTATA TGAGTAACAA AAAAGAGTTA GTTGTATTTT | No hit | SEQ ID N° 865 |
| | ACTTTATCAC CGATTTCCCG AACTACGCAA GATC | | |
| MAP1 | CTGCCACCTCCAATCTCCAGGCATATACAGTCGCCAATTGCTT | No hit | SEQ ID N° 866 |
| | CTCCTCTTCCTATGTGTGCCTCTTCAAGTTCATGTACATGATCC | | |
| | ATCTTCCTCTAATTTCTCTGGGCAAGATAATCATAACTATATCT | | |
| | TCACAACCTGAATCTAACTCCTCTGACCAAGATATACCAAAAC | | |
| | CCATCTCCAATTTTGAACCATATCCATATCC | | |
| MAP4a | TTGGGGGAGG TTTGCGGCGG AGATAAAATG AAAAAAAAGA | No hit | SEQ ID N° 867 |
| | AATGGAAGTG AACTTGAAGT TA | | |
| BMAP2a | TAAGGTTCAAAGCCAGCTATTACAGTTAGTTGTGTGAGCTTAT | No hit | SEQ ID N° 868 |
| | TCGCTCAACCTTAGCGGTAGGACATCTGGTGCGTCGGCTGCAT | | |
| BMAP3 | CTCCGACTCGATGTTGTCCTTGGATTTGGATTCCGGAGAGATC | No hit | SEQ ID N° 869 |
| | AAATGGTACAAACAGCTTGGAGGATATGACATCTGGACCGTCG | | |
| | GCTGCATA | | |
| T304a | GATCTGAGGT TGCTGATTTA GATTATGATG ACTTTGAGGC | No hit | SEQ ID N° 870 |
| | TGACTTTCAG GTCTTTA | | |
| T226 | GATCTCTA GTGTGAGTCA AAAATGATCA TAATATGAGT | No hit | SEQ ID N° 871 |
| | TTTGCCGGAG GCTTGGTTCA ATGAAAAATC GTTGTTTCAG | | |
| | TTGAGGTTCA TTCTTTTTTA CTGTTTCGCT CCTAATAAAT | | |
| | TTTTATTGTC AGTTGTCTTC TGATTTTTGC TGTTTTGTCC | | |
| | TATTCATTGT TGTGTTAGTA TTTTTGTTGA ATGTTGCATT | | |
| | GTTTTCTTTG TTTGAAAATT TCAATACGTT GGCCCTATCC | | |
| | TATTTTTGTA ATTTGTTTGG ATTATAATTG TATTGGTGTA | | |
| | GAAGATAAAA TTGTTCCATT A | | |

TABLE 3

Primers used to amplify the NsPMT2 promoter

| | |
|---|---|
| Primer Code | Sequence |

| | |
|---|---|
| FwP ALGG52 | 5′-AAAAAGCAGGCTCGAGGAGTGGAATACGAACAAA-3′ |
| RvP ALGG53 | 5′-AGAAAGCTGGGTTTTCCAAATTAAACTAAGCAAATTG-3′ |

TABLE 4
Jasmonate induction of the NsPMT2 promoter in
transgenic BY-2 cell line 7, represented as
GUS activity in units/mg protein/minute.

| Time (h) | +DMSO | +MeJA |
|---|---|---|
| 0 | 0.2 ± 0.3 | 0.8 ± 1.0 |
| 4 | 0.2 ± 0.3 | 2.0 ± 0.3 |
| 8 | 0.2 ± 0.3 | 6.4 ± 0.3 |
| 14 | 0.2 ± 0.3 | 29.1 ± 1.9 |
| 24 | 2.9 ± 0.6 | 92.2 ± 6.4 |

TABLE 5
Induction of the NsPMT2 promoter in transgenic BY-2 cell
line 7, double transformed with pK7WGD2-C330, represented
as GUS activity in units/mg protein/minute.

| Line | Time (h) | +DMSO | +MeJA |
|---|---|---|---|
| BY-2 line 7 | 0 | 0.0 ± 0.0 | 0.0 ± 0.0 |
| | 24 | 0.9 ± 0.1 | 399.0 ± 56.4 |
| | 48 | 6.0 ± 0.8 | 663.0 ± 33.6 |
| BY-2 line 7-C330 | 0 | 0.9 ± 0.1 | 1.0 ± 0.1 |
| | 24 | 6.4 ± 0.1 | 276.7 ± 55.9 |
| | 48 | 128.6 ± 0.3 | 347.8 ± 2.0 |

TABLE 6
A: Measurement of nicotine alkaloids in BY-2 reporter cell
line in the presence and absence of synthetic auxins, in
the presence and absence of MeJA. B: Measurement of nicotine
alkaloids in BY-2 reporter cell line supertransformed with
an expression vector comprising C330, in the absence of
2,4D, without and with the elicitor MeJA.

| Reporter cell line (line | | | | |
|---|---|---|---|---|
| 7) + expression vector | | Anatabine | Anabasine | Nicotine |
| comprising the C330 gene | | mg/g DW | Mg/g DW | mg/g DW |
| −2,4D + DMSO | 0 h | 0.036 | ND | 0.010 |
| | 24 h | 0.018 | ND | 0.005 |
| | 48 h | 0.115 | 0.003 | 0.271 |
| −2,4D + MeJA | 0 h | 0.038 | ND | 0.008 |
| | 24 h | 2.065 | 0.099 | 0.271 |
| | 48 h | 3.541 | 0.297 | 0.283 |

TABLE 7

| | | |
|---|---|---|
| Seq code | SEQUENCE | SEQ ID N° |

| | | |
|---|---|---|
| MAP3 | MNPANATESF SELDFLQSIE NHLLNYDSDF SEIFSPMSSS | SEQ ID N° 872 |
| | NALPNSPSSS FGSFPSAENS LDTSLWDENF EETIQNLEEK | |
| | SESEEETKGH VVAREKNATQ DWRRYIGVKR RPWGTFSAEI | |
| | RDPERRGARL WLGTYETPED AALAYDQAAF KIRGSRARLN | |
| | FPHLIGSNIP KPARVTARRS RTRSPQPSSS SCTSSSENGT | |
| | RKRKIDLINS IAKAKFIRHS WNLQMLL | |
| C330 | MFPNCLPNEY NYTADMFFND IFNEGIVGYG FEPASEFTLP | SEQ ID N° 873 |
| | SIKLEPEMTV QSPAIWNLPE FVAPPETAAE VKLEPPAPQK | |
| | AKHYRGVRVR PWGKFAAEIR DPAKNGARVW LGTYETAEDA | |
| | AFAYDKAAFR MRGSRALLNF PLRINSGEPD PIRVGSKRSS | |
| | MSPEYSSSSS SSASSPKRRK KVSQGTELTV L | |
| C484a | MNNTTFSDPN SDTGGFLGSG KIGGFGYGIG VSVGILILIT | SEQ ID N° 874 |
| | TTTLTSYFCT RNQTSELPTR RQRTINRNEL SGHCVVDIGL | |
| | DEKTLLSYPK LLYSEAKVNI KDSTASCCSI CLADYKKKDM | |
| | LRLLPDCGHL FHLKCVDPWL MLNPSCPVCR TSPLPTPQST | |
| | PLAEVVPLAT RPLG | |
| C360 | MGCIEKDPRE DVVQAWYMDD SDEDQRLPHH REPKEFVSLD | SEQ ID N° 875 |
| | KLAELGVLSW RLDADNYETD EELKKIREAR GYSYMDFCEV | |
| | CPEKLPNYEE KIKNFFEEHL HTDEEIRYCV AGSGYFDLRD | |
| | RNDAWIRVWV KKGGMIVLPA GIYHRFTLDS DNYIKAMRLF | |
| | VGDPIWTPYN RPHDHLPARK EYIESFIQAE GAGRAVNAAA | |
| C165 | MFFAHRENTM STLGRLVLIF WLFVVLIINS SYTASLTSIL | SEQ ID N° 876 |
| | TVQQLSSGIQ GIDSLISSSD QIGVQDGSFA YNYLIEELGV | |
| | SESRLRILKT EDEYVSALEK GPHGGGVAGI VDELPYVELF | |
| | LSNNKCIFRT VGQEFIKGGW GFAFQRDSPL AVDLSTAILQ | |
| | RSENGELQRI HDKWLTNNGC SSQNNQADDT QLSLKSFWGL | |
| | FLICAIACVL ALIVFFCRVY CQFRRYHPEP EEPEISEPES | |
| | ARPSRRTLRS VSFKDLIDFV DRRESEIKEI LKRKSSDNKR | |
| | HQTQNSDGQP SSPV | |
| C353a | MNPEYDYLFK LLLIGDSGVG KSCLLLRFAD DSYLESYIST | SEQ ID N° 877 |
| | IGVDFKIRTV EQDGKTIKLQ IWDTAGQERF RTTTSSYYRG | |
| | AHGIIVVYDV TDQESFNNVK QWLSEIDRYA SDNVNKLLVG | |
| | NKCDLTAQKV VSTEIAQAFA DEIGIPFMET SAKNATNVEQ | |
| | AFMAMAASIK NRMASQPASS NARPPTVQIR GQPVNQKSGC | |
| | CSS | |
| MT101 | MRVRIHQTMA TVMQKIKDIE DEMAKTQKNK ATAHHLGLLK | SEQ ID N° 878 |
| | AKLAKLRREL LTPTSKGGGG AGEGFDVTKS GDARVGLVGF | |
| | PSVGKSTLLN KLTGTFSEVA SYEFTTLTCI PGVIMYRGAK | |
| | IQLLDLPGII EGAKDGKGRG RQVISTARTC NCILIVLDAI | |
| | KPITHKRPIE KELEGFGIRL NKEPPNLTFR RKEKGGINLT | |
| | STVTNTHLDL DTVKAICSEY RIHNADVHLR YDATADDLID | |
| | VIEGSRVYTP CIYVVNKIDQ IPMEELEILD KLPHYCPISA | |
| | HLEWNLDGLL EKIWEYLSLT RIYTKPKGMN PDYEDPVILS | |
| | SKRRTVEDFC DRIHKDMVKQ FKYALVWGSS AKHKPQRVGR | |
| | EHELEDEDVV QIIKKV | |
| T21 | MANPKVFFDL TVGGLPTGRV VMELFNDVVP KTADNFRALC | SEQ ID N° 879 |
| | TGEKGVGKSG KPLHYKGSSF HRVIPGFMCQ GGDFTAGNGT | |
| | GGESIYGAKF ADENFVKKHT GPGILSMANA GPGTNGSQFF | |
| | ICTAKTEWLD GKHVVFGQVI EGMDVIKKVE AVGSSSGRCS | |
| | KPVVIADCGQ LS | |
| C476a | MALVRERRQL NLRLPLPEPS ERRPRFPLPL PPSISTTTTA | SEQ ID N° 880 |
| | PTTTISISEL EKLKVLGHGN GGTVYKVRHK RTSAIYALKV | |
| | VHGDSDPEIR RQILREISIL RRTDSPYVIK CHGVIDMPGG | |
| | DIGILMEYMN VGTLESLLKS QATFSELSLA KIAKQVLSGL | |
| | DYLHNHKIIH RDLKPSNLLV NREMEVKIAD FGVSKIMCRT | |
| | LDPCNSYVGT GAYMSPARFD PDTYGVNYNG YAADIWSLGL | |
| | TLMELYMGHF PFLPPGQRPD WATLMCAICF GEPPSLPEGT | |
| | SGNFRDFIEC CLQKESSKRW SAQQLLQHPF ILSIDLKST | |
| MC204 | MYGRSGLDRF KKAQSLEPFQ VSANSAAKPA LQPTTKAVTH | SEQ ID N° 881 |
| | PFPAYAQSTF SHQQTQYVNP QPALQKSVAA DATASTVPTH | |
| | HVTHGGGQST WQPPDWAIEP RPGVYYLEVI KDGEVLDRIN | |
| | LDKRRHIFGR QFHTCDFVLD HQSVSRQHAA VIPHKNGSIY | |
| | VIDLGSAHGT FVANERLTKD SPVELEPGQS LKLAVSTRPY | |
| | ILRRNNDALF PPPRQLAEID FPPPPDPSDE EAVLAYNTFL | |
| | NRYGLIRPDS LSKSTVSTSG EDVNYSSDRR AKRIRRTSVS | |
| | FKDQVGGELV EVVGISDGAD VETEPGPLGV KEGSLVGKYE | |
| | SLIEPTVLPK GKEQSSVKDA TVTRTGVSDI LQQVLSKVKN | |
| | PPKGGIYDDL YGESAPAKGG FWAYSDSSQT ASTNDAKGDS | |
| | PCSLRRIFGH ISNNVDDDTD DLFG | |
| T323 | MHSANHWGGS LEIANTGDST AEEYDRSRNL DWDRASVNHH | SEQ ID N° 882 |
| | QKQQQYNNYD QYSHRHNLDE TQQSWLLGPP EKKKKKYVDL | |
| | GCIVCSRKAF KYTIYGIIIA FLVIALPTII AKSLPKHKTR | |
| | PSPPDNYTIA LHKALLFFNA QKSGKLPKNN EIPWRGDSGL | |
| | QDGSKLTDVK GGLIGGYYDA GDNTKFHFPM SFAMTMLSWS | |
| | VIEYEHKYRA IDEYDHIRDL IKWGTDYLLR TFNSTATK1D | |
| | KLYSQVGGSL NNSRTPDDHY CWQRPEDMNY ERPVQTANSG | |
| | PDLAGEMAAA LAAASIXXXX XXXXXXXXXX XXXXXXXXXX | |
| | XXXXXXXXXX XXXXXXXXXX XXXXXXXXXX XXXXXXXXXX | |
| | XXXXXXXXXX XXXXXXXXXX XXXXXXXXXX XXXXXXXXXX | |
| | XXXXXXXXXX XXXXXXXXXX XXXXXXXXXX XXXXXXXXXX | |
| | XXXXXXXXXX XXXXXXXXXX XXXXXXXXXX XXXXXXXXXX | |
| | XXXXXXXXXX XXXXXXXXXX XXXXXXXXXX XXXXXXXXXX | |
| | XXXXXXXXXX XRRNCGPRYI SLDILRRFAT SQMNYILGDN | |
| | PLKMSYVVGY GNKFPRHVHH RGASIPSGKT KYSCTGGWKW | |
| | RDTKNPNPHN ITGAMVGGPD KFDKFKDARK NFSYTEPTLA | |
| | GNAGLVAALV SLTSSGGYGV DKNAIFSAVP PLYPMSPPPP | |
| | PPWKP | |
| T464 | MGSSGGMDYG AYTYENLERE PYWPTEKLRI SITGAGGFIA | SEQ ID N° 883 |
| | SHIARRLKSE GHYIIASDWK KNEHMTEDMF CHEFHLVDLR | |
| | VMDNCLKVTK DVDHVFNLAA DMGGMGFIQS NHSVIFYNNT | |
| | MISFNMMEAA RINGVKRFFY ASSACLYPEF KQLETNVSLK | |
| | ESDAWPAEPQ DAYGLEKLAT EELCKHYNKD FGIECCIGRF | |
| | HNIYGPFGTW KGGREKAPAA FCRKAQTAVD KFEMWGDGLQ | |
| | PRSFTFIDEC VEGVLRLTES DFREPVNIGS DEMVSMNDMA | |
| | EMVISFEDKK LPVHHIPGPE GVSGRNSDNT LIKEKLGWAP | |
| | TMRLKDGLRI TYFWIKEQIE KERSQGVNIA NYGSYKVVGT | |
| | QAPVELGSLR AADGKE | |
| C127 | MERNVANEAP KATIMAEDYK KDLEFIEEVT SNVDEVQMRV | SEQ ID N° 884 |
| | LAEILSQNAH VEYLQRHNLN GSTDRETFKK VVPVITYEDI | |
| | QPDIKRIAYG DKSPILCSQP ISELLSSSGT SGGESKLIPT | |
| | TEPEIGKRLQ LHKLVMSVLS QVAPDSGKGK GMYFMFISPE | |
| | QKTPGGLIAR FLTTSYYNSP YFNYSRLHNP HCNYTSPTAA | |
| | ILCPDSYQSM YSQMLCGLCQ NNQVLRVGSF FATSFVRAIR | |
| | FLEKHWSLLC NDIRSGTINT QITDPLVREA VMEVLKPDPT | |
| | LADFIEVECT KDSWQGIITR LWRNTKYVDV IVTGSMSQYI | |
| | PILDYYSNNL PLISTLYASS ESHFGINLNP FCKPSDVSYT | |
| | LIPTMCYFEF LPYRGNSGVI DSISMPKSLN EKEQQQLVDL | |
| | ADVKIGQEYE LVVTTYSGLY RYRVGDVLQV AGYKNNAPRF | |
| | NFLCRENYVL SIGADFTNEV ELQNAVKNAV GNLVPFDSQV | |
| | TEYTSYVDIT TLPSHYVIFW ELNANDSTLV PPSVFEDCCL | |
| | TIEESLNYFY REGRASNESI GPLEIRVLEI GTFDKLMDYC | |
| | MSLGASMNQY KTPRCLKYAP LIELLNSRVV SSYFSPMCPK | |
| | WVPGYKKWDG NN | |
| C175 | MERSVANEAP KATIMVEDYK KNIEFIEEVT SNVDEVQMRV | SEQ ID N° 885 |
| | LAEILSQNAH VEYLQRYNLN GRTDRETFKK VVPVITYEDI | |
| | QPDIKRIAYG DKSPILCSQP ISELLSSSGT SGGESKLIPS | |
| | TEAALGRRLQ LLKLLMSVMS QVAPDFGKGK GMYFMFISSE | |
| | QKTPGGLLAR FFTTSFYKSP YINCGYPCRK FTSPTATILC | |
| | QDSYQSMYSQ MLCGLCQNQE VLRVGSLFAT GFIRGIRFLE | |
| | KHWSLLCNDI RNGTINTQIT DPSVREAVME ILKPDPKLAD | |
| | FIEAECSKDS WQGIITRLWP NTKYVDAILT GSMSQYLPIL | |
| | DYYSNSLPLI STLYGSSECH FGINLNPFCK PSEVSYTLIP | |
| | TMCYFEFLPY HGNSGVIDSI SMPKSLNEKE QQQLVDLADV | |
| | EIGQEYELVV TTYSGLYRYR VGDVLRVAGY KNNAPRFNFL | |
| | CRENVILSIG ADFTNEVELQ NAVKNAVGNL MPFDSQVTEY | |
| | TGYVDITTIP SHYVIFWELN ANDSTPVPPS VFEDCCLTIE | |
| | ESLNYFYREG RASNASIGPL EIRVVEIGTF DKLMDYCSSL | |
| | GASMNQYKTP RCVKYAPLIE LLNSRVVSRY FSPMCPKWVP | |
| | GYKKWNNTS | |
| T424b | MAKEGTKVPR IKLGSQGLEV SAQGLGCMGM SAFYGPPKIPE | SEQ ID N° 886 |
| | PDMIQLIHHS INSGVTFLDT SDVYGPHTNE ILLGKALKGG | |
| | VRERVELATK FGAIFADGKI KVCGEPAYVR AACEASLKRL | |
| | DVDCIDLYYQ HRIDTRVPIE VTVGELKKLV EEGKIKYIGL | |
| | SEASASTIRR AHAVHPITTV QLEWSLWSRD VEEEIIPTCR | |
| | ELGIGIVAYS PLGRGFLSSG PELLEDLSSE DFPKHLPRFQ | |
| | ADNLEHNKIL YERICQMAAK KGCTPSQLAL AWVHHQGNDV | |
| | CPIPGITKIE NLNQNIGALS IKLTTEDMVE LEYIASADAV | |
| | KGERDASGAN HKNSDTPPLS TWKATR | |
| T164 | MESNNVVLLD FWPSSFGMRL RIALALKGIK YEAKEENLSD | SEQ ID N° 887 |
| | KSPLLLEMNP VHKKIPILIH NSKAICESLN ILEYIDEVWH | |
| | DKCPLLPSDP YERSQARFWA DYIDKKIYST GRRVWSGKGE | |
| | DQEEAKKEFI EILKTLEGEL GNKTYFGGDN LGFVDVALVP | |
| | FTSWFYSYET CANFSIEAEC PKLVVWAKTC MESESVSKSL | |
| | PHPHKIYGFV LELKHKLGLA | |
| MAP2 | MSDGGLTVLD GSQLRAVSLS LPSSDGSSVT GAQLLDFAES | SEQ ID N° 888 |
| | KVSESLFGFS LPDTLKSAAL KRLSVADDLN FRREQLDREN | |
| | ASIILRNYVA AIADELQDDP IVIAILDGKT LCMFLEDEDD | |
| | FAMLAENLFT DLDTEDRGKI RRNQIRDALI HMGVEMGIPP | |
| | LSEFPILSDI LKRHGAEGED ELGQAQFAHL LQPVLQELAD | |
| | ALAKNPVVVV QKIKINNGSK LRKVLADEKQ LSETVEKIMQ | |
| | EKQDEKDSLS NKDAIRCYLE KNGASLGLPP LKNDEVVILL | |
| | YDIVLGDIEN GKTDAASDKD EILVFLKDIL EKFAAQLEVN | |
| | PTFHDFDN | |
| C1 | MATKVYIVYY SMYGHVEKLA EEIKKGAASV EGVEAKLWQV | SEQ ID N° 889 |
| | PETLSEDVLA KMSAPPKSDV AVITPQELAE ADGIIFGFPT | |
| | RFGMMAAQFK AFLDATGGLW RTQQLAGKPA GIFYSTGSQG | |
| | GGQETTPLTA ITQLVHHGMI FVPIGYTFGA GMFEMEKVKG | |
| | GSPYGAGTFA GDGSRQPSDL ELQQAFHQGK YIAGIAKKLK | |
| | GAA | |
| T210 | MKIVDLDESL MESDGNCVNT EKRLIVVGVD AKRALVGAGA | SEQ ID N° 890 |
| | RILFYPTLLY NVFRNKIQSE FRWWDQIDQF LLLGAVPFPS | |
| | DVPRLKQLGV GGVITLNEPY ETLVPSSLYH AHGIDHLVIP | |
| | TRDYLFAPSF VDINRAVDFI HRNASIGQTT YVHCKAGRGR | |
| | STTVVLCYLV EYKHMTPRAA LEFVRSRRPR VLLAPSQWKA | |
| | VQEFKQQRVA SYALSGDAVL ITKADLEGYH SSSDDSRGKE | |
| | LAIVPRIART QPMIARLSCL FASLKVSDGC GPVTRQLTEA RAC | |
| C112 | MSSASTENRS LWTEIRESIR SILKANCGHF HTLFILFLLP | SEQ ID N° 891 |
| | IFFSLVVYPS FHLALFHPDY DFTQPVQFSH FLSSHFEIIV | |
| | PIVFTLFLVL LFLCAVATTT YSALHVSYGR PINLVSSIKS | |
| | IRNSFFPLLS TFIVSHTIFI SIALVFSLVL VFLVQVLQTL | |
| | GLIELKYDSN HFLFLVIPAL IVLVPVLIWL QVNWSLAYVI | |
| | AVVESKWGFE TLRRSAYLVK GKRSVALSMM LLYGLLMGIM | |
| | VVLGAMYLVI MDAAKGRQWR SSGVILQTAM SSITSYLMMS | |
| | QFLVGNVVLY LRCNDLNGEK LPLEIEHLLL HQSLANDHPP | |
| | PMLSASTKNL SLWTEVVESA MSIFKANSGH FHALSILFLL | |
| | PISFFLVVYP SFHLALFHPN YDFISFAQPH LFLSNFEIIV | |
| | PTSYSLFLVL LFLCAVATTT YSAVHASYSR PINLVLSIKS | |
| | IRKSLFPLLS TLLVSHTIFI SITLVFTLVL TILVQILQPL | |
| | GLIEIKYDSD HFLLLAIPAL VVLVPVLLWL HVNWSLAYVI | |
| | AVIESKWGYE TLRRSSYLVK GQRWVAFGIY LYYGLSMGIM | |
| | MVCGSMFFVI MGVAKGNKWR SLDVIIQTAL VSVMGYLTMN | |
| | QYLVANVVLY MKCKDLSVEK LQSETGGEYV PLPLDEKNQA | |
| | LE | |
| C454 | SQFFSSIPLQ PIPRGSSFAA STIHSGPIPA RISSTYPCSG | SEQ ID N° 892 |
| | PIERGFMSGP IERSFTSGPL ENQYDHIQRY KPKSKKWGLI | |
| | KSLKKVLSNS FLGFNKFMNL VEKNNNNEVN VQGSNSHHSN | |
| | VGNSLSSQNS LVDDDDEGND SFRGQNVQWA QGKAGEDRVH | |
| | VVISEEHGWV FVGIYDGFNG PDATDFLLNN LYSNVYKELK | |
| | GLLWNDKLKT PKNSTSNETV PLRNSGFKVE HFVQNQELDQ | |
| | REKLDGVVGV DHSDVLKALS EGLRKTEASY LEIADMMVKE | |
| | NPELALMGSC VLVMLLKDQD VYLLNVGDSR AVLAQNPESD | |
| | ISISKLKRIN EQSVNSIDAL YRAESDRKHN LIPSQLTMDH | |
| | STSIKEEVIR IRSEHLDDPF AIKNDRVKGS LKVTRAFGAG | |
| | YLKQPKWNNA LLEMFRINYI GNSPYINCLP SLYHHTLGSR | |
| | DRFLILSSDG LYQYFTNEEA VSEVETFMSI FPEGDPAQHL | |
| | VEEVLFRAAK KAGLNFHELL DIPQGDRRKY HDDVSIIILS | |
| | FEGRIWKSSL | |
| T172 | GAENGLIVSD SIIQGNEEDE ILSVGEDPCV INGEELLPLG | SEQ ID N° 893 |
| | ASSELSLPIA VEIEGIDNGQ ILAKVISLEE RSFERKISNL | |
| | SAVAAIPDDE ITTGPTLKAS VVALPLPSEN EPVKESVKSV | |
| | FELECVPLWG SVSICGKRPE MEDALMVVPN FMIUPIKMFI | |
| | GDRVIDGLSQ RLSHLTSHFY GVYDGHGGSQ VADYCCKRIH | |
| | LALVEELKLF KDDMVDGSAK DTRQVQWEKV FTSCFLKVDD | |
| | EVGGKVNSDP GEDNIDTTSC ASEPIAPETV GSTAVVAVIC | |
| | SSHIVVSNCG DSRAVLYRGK EAMALSIDHK PSREDEYARI | |
| | EASGGKVIQW NGHRVFGVLA MSRSIGDRYL KPWIIPEPEI | |
| | MFVPRAREDE CLVLASDGLW DVMSNEEACE VARRRILLWH | |
| | KKNGTNPLPE RGQGVDPAAQ AAAEYLSTMA LQKGSKDNIS | |
| | VIVVDLKAQR KFKSKC | |
| C477 | METQNLERGH VIEVRCDMAA QEKGTKICGS APCGFSDVNT | SEQ ID N° 894 |
| | MSKDAQERSA SMRKLCIAVV LCIIFMAVEV VGGIKANSLA | |
| | ILTDAAHLLS DVAAFAISLF SLWAAGWEDN PRQSYGFFRI | |
| | EILGALVSIQ MIWILAGILV YEAIARLIHD TGEVQGFLMF | |
| | VVSAFGLVVN LIMALLLGHD HGHGHGHGHS HGHDHEHGHN | |
| | HGEHAHSNTD HEHGHGEHTH IHGISVSRHH HHNEGPSSRD | |
| | QHSHAHDGDH TVPLLKNSCE GESVSEGEKK KKPQNINVQG | |
| | AYLHVIGDSI HSIGVMIGGA IIWYKPEWKI IDLICTLLFS | |
| | VIVLGTTIRM LRSILEVLME STPREIDATR LQKGLCEMED | |
| | VVPIHELHIW AITVGKVLLA CHVKIKSDAD ADTVLDKV | |
| C331 | MLIMLLVPVR QYLLPKFFKG AHLQDLDAAE YEEAPAIAYN | SEQ ID N° 895 |
| | MSYGDQDPQA RPACIDSSEI LDEIITRSRG EIRHPCSPRV | |
| | TSSTPTKLEE IKSMHSPQLA QRAYSPRVNV LRGERSPRLT | |
| | GKGLGIKQTP SPQPSNLGQN GRGPSST | |

### REFERENCES

Altschul, S. F. et al. Gapped BLAST and PSI-BLAST: a new generation of protein database search programs. Nucleic Acids Res. 25, 3389-3402 (1997).
Bachem, C. W. B. et al. Visualization of differential gene expression using a novel method of RNA fingerprinting based on AFLP: analysis of gene expression during potato tuber development. Plant J. 9, 745-753 (1996).
Eisen, M. B., Spellman, P. T., Brown, P. O. & Botstein, D. Cluster analysis and display of genome-wide expression patterns. Proc. Natl. Acad. Sci. USA 95, 14863-14868 (1998).
Nagata, T., Nemoto, Y. & Hasezawa, S. Tobacco BY-2 cell line as the “HeLa” cell in the cell biology of higher plants. Int. Rev. Cytol. 132, 1-30 (1992).
Sambrook, J., Fritsch, E. F. & Maniatis, T. Molecular Cloning, A Laboratory Manual, 2nd ed. (Cold Spring Harbor Laboratory Press, Cold Spring Harbor, N.Y.; 1989).
Sharan, M., Taguchi, G., Gonda K., Jouke, T., Shimosaka, M., Hayashida, N. & Okazaki, M. Effects of methyl jasmonate and elicitoron the activation of phenylalanine ammonia-lyase and the accumulation of scopoletin and scopolin in tobacco cell cultures. Plant Science 132, 13-19 (1998).
Smith, N. A., Singh, S. P., Wang, M., Stoutjesdijk, P. A., Green, A. G. & Waterhouse, P. M. Total silencing by intron-spliced hairpin RNAs. Nature 407, 319-320 (2000).
Tavazoie, S., Hughes, J. D., Campbell, M. J., Cho, R. J. & Church, G. M. Systematic determination of genetic network architecture. Nature Genet. 22, 281-285 (1999).
Van der Fits, L., Deakin, E. A., Hoge, J. H. & Memelink J. The ternary transformation system: constitutive virG on a compatible plasmid dramatically increases Agrobacterium-mediated plant transformation. Plant Mol. Biol. 43, 495-502 (2000).
Vos, P. et al. AFLP: a new technique for DNA fingerprinting. Nucleic Acids Res. 23, 4407-4414 (1995).
Creelman, R. A:, Tierney, M. L. & Mullet, J. E. (1992): Jasmonic acid/methyl jasmonate accumulate in wounded soybean hypocotyls and modulate wound gene expression. Proc. Natl. Acad. Sci., 89:4938-4941.
Darvil, A. G. & Albersheim, P. (1984): Phytoalexins and their elicitors—a defence against microbial infections in plants. Annu. Rev. Plant Physiol. 35: 243-275.
Flores, H. E., Protacio, C. M. & Signs, M. W. (1991): Plant nitrogen metabolism. Recent Adv. Phytochem., 23: 329.
Furuya, T., Hisashi, K. & Syono, K. (1971): Regulation of nicotine biosynthesis by auxins in tobacco callus tissues. Phytochemistry, 10: 1529-1532.
Gundlach, H., Mueller, M. J., Kutchan, T. M. & Zenk, M. H. (1992): Jasmonic acid is a signal transducer in elicitor-induced plant cell cultures. Proc. Natl. Acad. Sci., 89: 2389-2393.
Hibi, N., Higashiguchi, S., Hashimoto, T. & Yamada, Y. (1994): Gene expression in tobacco low-nicotine mutants. Plant Cell, 6:723-735.
Imanishi, S., Hashizume, K., Nakakita, M., Kojima, H., Matsubayashi, Y., Hashimoto, T., Sakagami, Y., Yamada, Y. & Nakamura, K. (1998): Differential induction by methyl jasmonate of genes encoding ornithine decarbocsylase and other enzymes involved in nicotine biosynthesis in tobacco cell cultures. Plant Mol. Biol. 38:1101-1111.
Ishikawa, A., Yoshihara, T. & Nakamura, K. (1994): Jasmonate-inducible expression of a potato cathepsin D inhibitor-GUS gene fusion in tobacco cells. Plant Mol. Biol., 26:403-414.
Linsmaier, E. M. & Skoog, F. (1964): Organic growth factor requirements of tobacco tissue cultures. Physiol. Plant., 18: 100-127.
Mandujano-Chavez, A., Schoenbeck, M. A., Ralston, L. F., Lozoya-Gloria, E. & Chappell, J. (2000): Differential induction of sesquiterpene metabolism in tobacco cell suspension cultures by methyl jasmonate and fungal elicitor. Arch. Biochem. Biophys. 381: 285-294.
Nagata, T. & Kumagai, F. (1999): Plant cell biology through the window of the highly synchronized tobacco BY-2 cell line. Methods Cell Sci. 21: 123-127.
Ohta, S., Matsui, O. & Yatazawa, M. (1978): Culture conditions for nicotine production in tobacco tissue culture. Agric. Biol. Chem., 42: 1245-1251.
Reinbothe, S., Mollenhauer, B. & Reinbothe, C. (1994): JIPs and RIPs: The regulation of plant gene expression by jasmonates in response to environmental cues and pathogens. Plant Cell 6: 1197-1209.
Scaramagli, S., Franceschetti, M., Michael, A. J., Torrigiani, P. & Bagni N. (1999): Polyamines and flowering: spermidine biosynthesis in the different whorls of developing flowers of Nicotiana tabacum L., Plant Biosystems, 133: 229-237.
Strunz, G. M. & Findlay, J. A. (1985): Tobacco Alkaloids, Related Compounds, and Other Nicotinic Acid Derivatives. In: A. Brossi (Ed.) The Alkaloids, Chemistry & Pharmacology, vol 26. Academic Press, New York, pp. 121-151.
Suzuki, K., Yun, D. J., Chen, X.-Y., Yamada, Y. & Hashimoto, T. (1999): An Atropa belladonna hyoscyamine 6β-hydrolase gene is differentially expressed in the root pericycle and anthers. Plant Mol. Biol., 40: 141.
Swiatek, A., Lenjou, M., Van Bockstaele, D., Inzé, D. & Van Onckelen, H. (2002): Differential effect of jasmonic acid and abscisid acid on cell cycle progression in tobacco BY-2 cells. Plant Physiol. 128: 201-211.
Verpoorte, R., van der Heijden, R. & Memelink, J. (1998): Plant biotechnology and the production of alkaloids: prospects of metabolic engineering. In: The Alkaloids, Chemistry and Pharmacology, vol 50. G. A. Cordell (Ed.), Academic Press, New York, pp. 453-508.
Verpoorte R. (2000) Secondary metabolism. Metabolic Engineering of Plant Secondary Metabolism. In: Verpoorte R and Alfermann A W, editors. Kluwer Academic Publishers, Dordrech-Boston-London, pp. 1-29.

What is claimed is:

1. An isolated polypeptide that modulates the production of at least one secondary metabolite in an organism or cell derived therefrom wherein said polypeptide is selected from the group consisting of:
(a) a polypeptide encoded by a polynucleotide comprising SEQ ID NO: 1 through 611 or SEQ ID NO: 612 through 871;
(b) a polypeptide comprising a polypeptide sequence having a least 60% identity to at least one of the polypeptides encoded by a polynucleotide sequence having SEQ ID NO: 612 through 871;
(c) a polypeptide comprising a polypeptide sequence having a least 90% identity to at least one of the polypeptides encoded by a polynucleotide sequence of SEQ ID NO: 1 through SEQ ID NO:610, or SEQ ID NO:611;
(d) fragments and variants of the polypeptides according to (a), (b) or (c) that modulate the production of at least one secondary metabolite in an organism or cell derived thereof.

2. The isolated polypeptide of claim 1 wherein said isolated polypeptide is selected from the group consisting of SEQ ID NOs: 872, 873, 874 through 895 and polypeptide sequences having at least 90% identity to SEQ ID NO: 872, 873, 874 through 895.

3. An isolated polynucleotide selected from the group consisting of:
(a) a polynucleotide comprising a polynucleotide sequence having at least one of the sequences SEQ ID NO: 1 through SEQ ID NO: 611 or SEQ ID NO: 612 through 871;
(b) a polynucleotide comprising a polynucleotide sequence having at least 60% identity to at least one of the sequences having SEQ ID NO: 612 through SEQ ID NO: 871;
(c) a polynucleotide comprising a polynucleotide sequence having at least 90% identity to at least one of the sequences having SEQ ID NO: 1 through SEQ ID NO: 611;
(d) fragments and variants of the polynucleotides of the foregoing (a), (b) or (c), modulating the production of at least one secondary metabolite in an organism or cell derived thereof.

4. A recombinant DNA vector comprising at least one of the polynucleotide sequences of claim 3.

5. A transgenic plant or a cell derived therefrom transformed with the recombinant DNA vector of claim 4.

6. A method of identifying genes, the expression of which modulates the production of at least one secondary metabolite in an organism or cells derived from said organism, said method comprising the steps of:
(a) performing a genome wide expression profiling of said organism or cells on different times of growth,
(b) isolating genes whose expression is co-regulated either with said at least one secondary metabolite, or with a gene known to be involved in the biosynthesis of said secondary metabolite,
(c) analysing the effect of over- or under-expression of said isolated genes in said organism or cell on the production of said at least one secondary metabolite, and
(d) identifying genes that can modulate the production of said at least one secondary metabolite.

7. The method according to claim 6, wherein steps (a) to (d) are preceded by a step of inducing the production of said at least one secondary metabolite in said organism or cell.

8. The method according to claim 6 wherein said secondary metabolite is an alkaloid or phenylpropanoid.

9. The method according to claim 7 wherein said secondary metabolite is an alkaloid or phenylpropanoid.

10. A method of modulating a cell, comprising using the polynucleotide of claim 3 to modulate the biosynthesis of secondary metabolites in the cell.

11. A method of modulating the biosynthesis of alkaloids in a cell, said method comprising:
using a polynucleotide comprising SEQ ID NO: 10, 11, 19, 20, 35, 40, 41, 47, 65, 67, 70, 88, 89, 97, 98, 101, 102, 103, 106, 107, 108, 117, 118, 120, 121, 123, 124, 126, 128, 130, 131, 132, 136, 137, 142, 143, 144, 145, 146, 147, 148, 152, 154, 155, 159, 160, 161, 162, 163, 175, 176, 177, 181, 182, 183, 189, 197, 202, 207, 208, 209, 210, 217, 219, 220, 221, 233, 235, 236, 237, 239, 240, 241, 242, 243, 244, 261, 262, 264, 265, 268, 70, 272, 273, 274, 278, 279, 299, 300, 302, 303, 304, 305, 306, 316, 317, 318, 320, 321, 326, 329, 331, 332, 333, 334, 341, 344, 348, 349, 350, 351, 354, 355, 356, 358, 372, 373, 374, 375, 377, 382, 390, 391, 392, 395, 403, 405, 406, 414, 417, 418, 419, 420, 424, 430, 434, 439, 440, 441, 445, 446, 456, 463, 478, 485, 491, 497, 507, 508, 510, 518, 519, 527, 529, 531, 532, 534, 567, 569, 570, 575, 577, 579, 587, 593, 594, 598, 599, 601, 603, 608, 612, 613, 618, 619, 620, 628, 636, 642, 643, 647, 648, 649, 652, 653, 654, 655, 656, 657, 659, 660, 662, 664, 670, 671, 674, 675, 676, 677, 679, 680, 682, 683, 695, 696, 700, 701, 703, 707, 709, 710, 711, 712, 714, 719, 724, 727, 729, 732, 734, 735, 740, 741, 744, 746, 748, 749, 750, 751, 753, 754, 755, 757, 758, 759, 760, 761, 762, 763, 764, 766, 767, 772, 777, 784, 794, 809, 810, 811, 816, 817, 822, 823, 826, 827, 828, 829, 830, 832, 833, 834, 836, 837, 839, 840, 841, 850, 854, 855, 856, 858, 859, 861, 864, 865, 488, 489 and/or 490 or fragments or homologues thereof to modulate the biosynthesis of alkaloids in the cell.

12. A method of modulating the biosynthesis of phenylpropanoids in a cell, said method comprising:
using a polynucleotide comprising SEQ ID NO: 3, 4, 5, 7, 15, 17, 21, 23, 29, 30, 32, 33, 39, 42, 44, 45, 46, 48, 49, 50, 51, 8, 61, 62, 72, 74, 79, 84, 92, 94, 95, 104, 105, 125, 134, 150, 170, 171, 179, 180, 184, 194, 195, 200, 201, 203, 204, 205, 213, 214, 215, 218, 245, 249, 250, 251, 252, 254, 255, 266, 275, 276, 281, 282, 285, 286, 287, 289, 291, 298, 301, 308, 309, 310, 311, 312, 313, 315, 319, 323, 324, 335, 343, 361, 363, 364, 370, 379, 380, 383, 384, 385, 386, 398, 401, 402, 407, 415, 416, 423, 432, 433, 437, 443, 444, 447, 448, 450, 451, 452, 455, 457, 460, 461, 462, 471, 474, 486, 487, 493, 494, 499, 500, 501, 502, 503, 504, 505, 506, 517, 522, 523, 524, 526, 528, 538, 541, 543, 544, 545, 546, 547, 553, 554, 555, 562, 568, 571, 572, 578, 580, 581, 582, 588, 605, 607, 616, 617, 621, 626, 627, 637, 638, 641, 644, 650, 651, 665, 666, 667, 681, 684, 685, 691, 697, 698, 704, 708, 713, 720, 721, 728, 730, 736, 745, 752, 756, 771, 776, 778, 782, 783, 792, 793, 795, 797, 798, 799, 800, 801, 808, 815, 818, 819, 820, 821, 835, 842, 843, 844, 845, 848, 851, 852, 853, 862, 868, 488, 489 and/or 490 or fragments or homologues thereof to modulate the biosynthesis of phenylpropanoids in the cell.
